(19) United States
(12) Patent Application Publication
(10) Pub. No.: US 2024/0383856 A1
(43) Pub. Date: Nov. 21, 2024

(54) INHIBITORS OF TRANSGLUTAMINASES

(71) Applicant: Zedira GmbH, Darmstadt (DE)

(72) Inventors: Ralf Pasternack, Griesheim (DE); Christian Büchold, Karben (DE); Martin Hils, Darmstadt (DE); Martin Stieler, Darmstadt (DE); Uwe Gerlach, Hattersheim (DE)

(21) Appl. No.: 18/572,134

(22) PCT Filed: Jun. 30, 2022
(86) PCT No.: PCT/EP2022/068217

§ 371 Date: Dec. 19, 2023

Related U.S. Application Data
(60) Provisional application No. 63/217,783, filed on Jul. 2, 2021.

(30) Foreign Application Priority Data
Jun. 30, 2021 (EP) 21182956.9
Jul. 1, 2021 (EP) 21183316.5
Dec. 17, 2021 (WO) PCT/EP2021/086674
Jun. 7, 2022 (WO) PCT/EP2022/065435

(51) Int. Cl. C07D 213/76; A61K 31/45; A61K 31/497; C07D 213/74; C07D 401/12; C07D 405/12; C07D 409/12; C07D 417/12

(52) U.S. Cl. CPC C07D 213/76; A61K 31/45; A61K 31/497; C07D 213/74; C07D 401/12; C07D 405/12; C07D 409/12; C07D 417/12

(57) ABSTRACT

The invention relates to the compound of general formula (I) as novel inhibitors of transglutaminases, to methods for producing the inventive compounds, to pharmaceutical compositions containing said inventive compounds and to their use for the prophylaxis and treatment of diseases associated with transglutaminases, in particular transglutaminase 2.

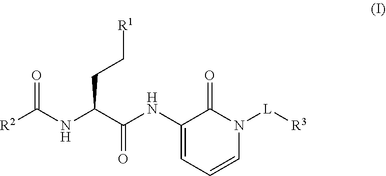

The invention relates to novel inhibitors of transglutaminases, in particular transglutaminase 2, methods for their synthesis and to their use for the prophylaxis and treatment of diseases associated with transglutaminases, in particular transglutaminase 2.

### BACKGROUND OF THE INVENTION

Transglutaminases are part of the class of transferases and according to EC nomenclature they are correctly designated as “protein-glutamine: amine γ-glutamyl transferases” (EC 2.3.2.13). They link the ε-amino group of the amino acid lysine and the γ-glutamyl group of the amino acid glutamine forming an isopeptide bond while ammonia is released. In the absence of suitable amines and/or under certain conditions, deamidation of the glutamine may occur resulting in the corresponding glutamic acid.

Additionally, transglutaminases play an important role in many therapeutic areas such as the cardiovascular diseases (thrombosis and atherosclerosis), autoimmune diseases (celiac disease, Duhring-Brocq-disease, gluten ataxia), neurodegenerative diseases (Alzheimer's disease, Parkinson's disease, Huntington's disease), dermatological diseases (ichthyosis, psoriasis, acne) as well as in wound healing and inflammatory diseases (e.g. tissue fibrosis) (J. M. Wodzinska, Mini-Reviews in medical chemistry, 2005, 5, 279-292).

Celiac disease, a gluten intolerance, however, is one of the most important indications. Celiac disease is characterized by a chronic inflammation of the mucosa of the small intestine. In susceptible patients, the intestinal epithelium is successively destroyed after ingestion of gluten-containing food resulting in reduced absorption of nutrients which again has massive impact on the patients affected and is for example associated with symptoms such as loss of weight, anemia, diarrhea, nausea, vomiting, loss of appetite and fatigue. Due to these findings, there is a large demand for the development of a medicament for the treatment of celiac disease as well as of other diseases associated with tissue transglutaminase (transglutaminase 2, TG2, tTG). The tissue transglutaminase is a central element during pathogenesis. The endogenous enzyme catalyses the deamidation of gluten/gliadin in the small intestinal mucosa and thus triggers the inflammatory response. Therefore inhibitors of tissue transglutaminase are suitable to be used as active agents for medication.

Another very important group of indications for tissue transglutaminase inhibitors are fibrotic disorders. Fibrotic disorders are characterized by the accumulation of cross-linked extracellular matrix proteins. Diabetic nephropathy, cystic fibrosis, idiopathic pulmonary fibrosis, kidney fibrosis as well as liver fibrosis belong to the most important fibrotic disorders to be addressed with the compounds disclosed.

U.S. Pat. No. 9,434,763 B2 discloses pyridinone derivatives having a warhead comprising at least one acceptor-substituted double bond, such as a Michael System, as irreversible transglutaminase inhibitors. Alkylacetamido and arylacetamido pyridinones showed inhibitory activity regarding tissue transglutaminase TG2 in nanomolar range (IC₅₀).

Tse et al. (J. Med. Chem. 2020, 63, 11585-11601) report on replacement of phenyl residues by non-classical bioisosteres, such as cubane and bicyclo[1.1.1]pentane (BCP), in anti-malarial triazolopyrazine compounds in order to alter compound solubility and metabolic stability. The authors further evaluated in vitro antiplasmodial activity of bioisosteric modified triazolopyrazines against the 3D7 strain of P. falciparum. Replacement of phenyl by bioisosteric saturated heterocyclic residues resulted in complete loss of activity. Adamantyl residues as well as other hydrocarbon-caged derivatives led to potency up to 2-9 times lower than the corresponding phenyl triazolopyrazine compounds. In contrast, higher potencies were achieved by replacing phenyl with closo-1,2- and 1,7-carborane isomers. The authors concluded that the effect of non classical bioiostere replacement on biological properties cannot be predicted accurately and that a considerable range of possible bioisosteres has to be tested first in order to identify a suitable replacement leading to the desired properties of a given molecule.

Subbaiah et al. (J. Med. Chem. 2021, 64, 19, 14046-14128) report on bioisosteres of the phenyl ring in lead optimization and drug design. It is noted that bioisosteric phenyl ring replacement with heterocyclic and carbocyclic moieties can lead to enhanced potency, solubility, and metabolic stability while reducing lipophilicity, plasma protein binding, phospholipidosis potential, and inhibition of cytochrome P450 enzymes and the hERG channel. However, this effect depends strongly on the properties of the compound itself and the addressed target.

U.S. Pat. No. 11,072,634 B2 discloses reversible transglutaminase inhibitors comprising an aldehyde, a ketone, an α-ketoaldehyde, an α-ketoketone, an α-ketoacid, an α-ketoester, an α-ketoamide or a halogenmethylketone as warhead. The inhibitors showed inhibitory activity regarding tissue transglutaminase TG2 in nanomolar and micromolar range (IC₅₀).

The objective of the present invention is to provide novel, most probably reversible inhibitors of transglutaminases, in particular transglutaminase 2 and methods for the synthesis of said inhibitors as well as several uses of these inhibitors.

Said objective is solved by the technical teachings of the independent claims. Further advantageous embodiments, aspects and details of the invention are evident from the dependent claims, the description and the examples.

Surprisingly, it has been found that reversible inhibitors having a chemical warhead as disclosed herein inhibit effectively transglutaminases including tissue transglutaminase called transglutaminase 2 or TG2. Herein these terms are used synonymous.

Preferably, such chemical warhead moiety is particularly selected from reversible warheads such as α-ketoamides. The compounds of the present invention act as selective inhibitors of transglutaminase 2.

In order to prove inventiveness of the compounds of the present application, reference compounds were synthesized and tested in comparison to the most similar compounds of the present application. A skilled person might notice compound A8 from our patent U.S. Pat. No. 9,434,763 B2 which we introduce as Ref. 3 to highlight the inventive and preferred features of the claimed compounds. From U.S. Pat. No. 9,434,763 B2, it is clear, that aromatic moieties (C-terminal) restrict the efficacy of those compounds (compare to A1, A8, A37, A44, A47). In sharp contrast, branched alkyl moieties are highly preferred as indicated by more potent compounds (A28, A29, A59, A61, A63, A67, A68, A79).

To illustrate the advantage of branched alkyl moieties over aromatic moieties, we refer to reference compounds Ref. 2 (ZED1227, U.S. Pat. No. 9,434,763 B2) and Ref. 3 (A8, ZED1047). Inhibition data were determined using the classical fluorescent transamidation assay (dansylcadaverine incorporation into methylated casein, DCC-assay) as described [Buchold, C.; Hils, M.; Gerlach, U.; Weber, J.; Pelzer, C.; Heil, A.; Aeschlimann, D.; Pasternack, R. Features of ZED1227: The First-In-Class Tissue Transglutaminase Inhibitor Undergoing Clinical Evaluation for the Treatment of Celiac Disease. Cells 2022, 11, 1667. https://doi.org/10.3390/cells11101667]. Casein is one of the best known high molecular weight (24 kDa) protein substrates for transglutaminases. Please note, the IC₅₀ value of Ref. 3 (A8) published in U.S. Pat. No. 9,434,763 B2 cannot be compared to the present data, relying on a fluorogenic isopeptidase assay. Measured in the DCC-assay, Ref. 2 (IC₅₀=53 nM) is 80-fold more potent compared to Ref. 3 (IC₅₀=4,268 nM).

Accordingly, a person skilled in the art of medicinal chemistry would choose branched alkyl moieties as lead structures, such excluding aromatic moieties, e.g. the phenyl group. It is of common knowledge, that bridged cycloalkyl groups are non-classical bioisosters of the phenyl group. By replacement of the phenyl group in A8 by e.g. an adamantane group, a skilled person would expect similar physico-chemical or biochemical properties excluding to invest efforts. Since aromatic moieties are clearly not preferred, bridged cycloalkyl groups would not be considered improving the compounds.

This is further supported by additional reference compounds. ZED3641 (Ref. 1, as disclosed in U.S. Pat. No. 11,072,634 B2; reversible acting α-ketomethylamide analogue to Ref. 2, ZED1227) is about 10-fold more potent compared to Ref. 6 (compare table 1). Ref. 6 is analogous to compound A8 disclosed in U.S. Pat. No. 9,434,763 B2 with respect to the backbone proving again superiority of branched alkyl moieties compared to aromatic derivatives in combination with reversible acting warheads.

However, surprisingly, replacement of the preferred branched alkyl moieties by bridged cycloalkyl groups further significantly improve the potency of the compounds as shown in table 1. Therefore, we credit bridged cycloalkyl groups as disclosed with an outstanding inventive manner.

In summary, the inventive compounds rated “A” show efficacies of about 100-fold higher compared to Ref. 3 (A8, compare to table1).

Further, compounds with activities rated “B” or “C” are still preferred (lower IC₅₀ values) to Ref. 3 (A8). These compounds can also be considered inventive, since peripheric ligands affect physico-chemical or biochemical properties. Therefore, also less potent compounds might be of high value, depending on the application.

Thus, the present invention relates to compounds of the general formula (I):

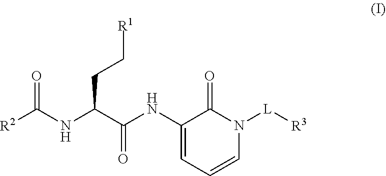

wherein
L represents -L¹- or -L¹-L²-; preferably -L¹-L²-;
L¹ represents —CH₂—, —CH₂CH₂—, —CH₂CH₂CH₂—, —CH₂CO—, or —CH₂CH₂CO—;
L² represents a bond, —NR^{N1}—, —NR^{N1}CH₂—, —NR^{N1}CH₂CH₂—, or —NR^{N1}CH(CH₃)—,
R¹ represents

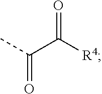

R² represents

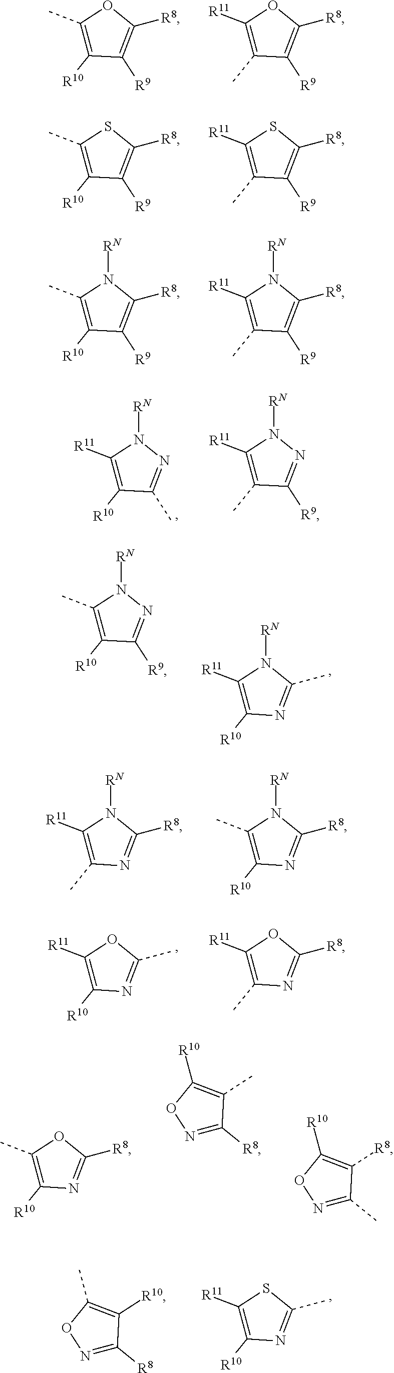

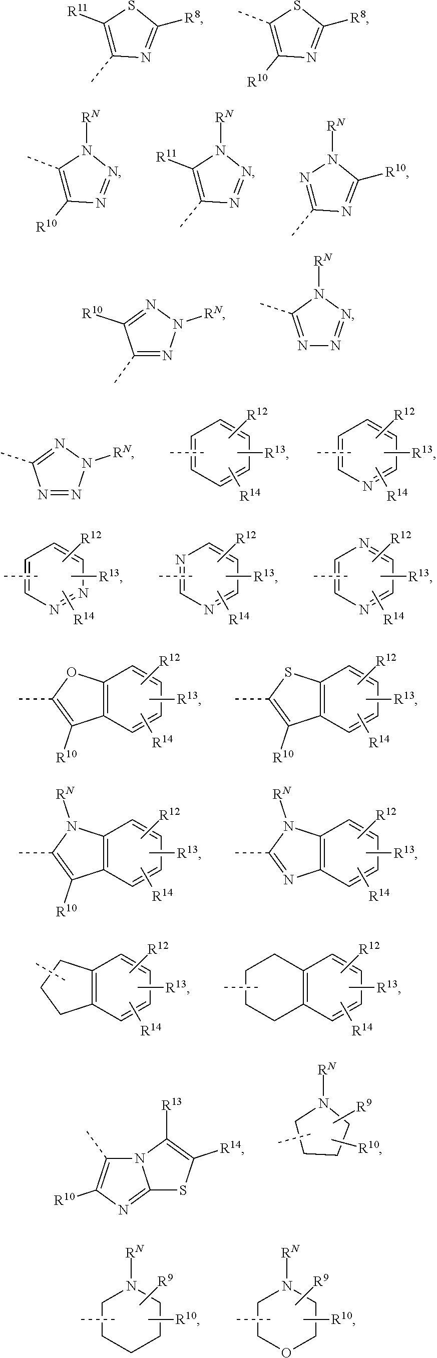

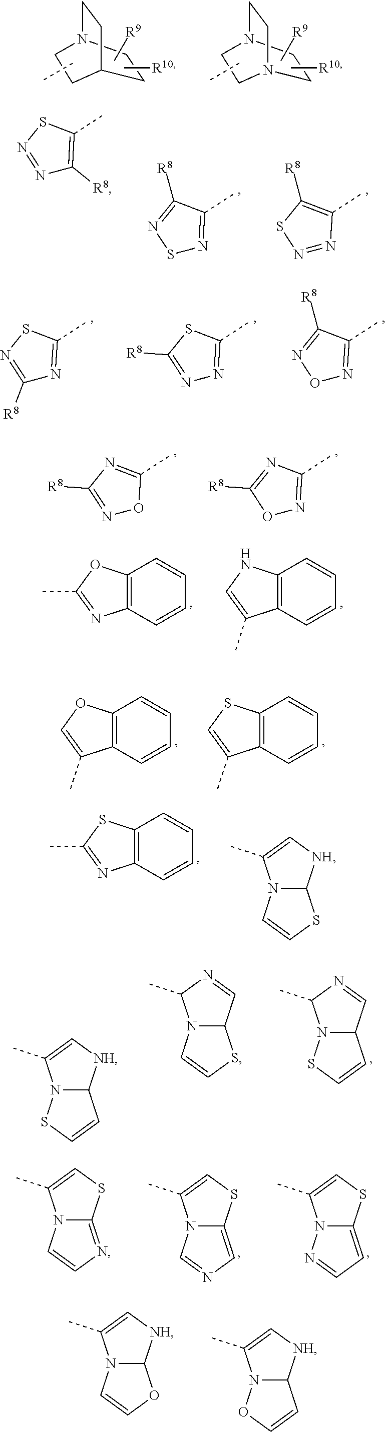

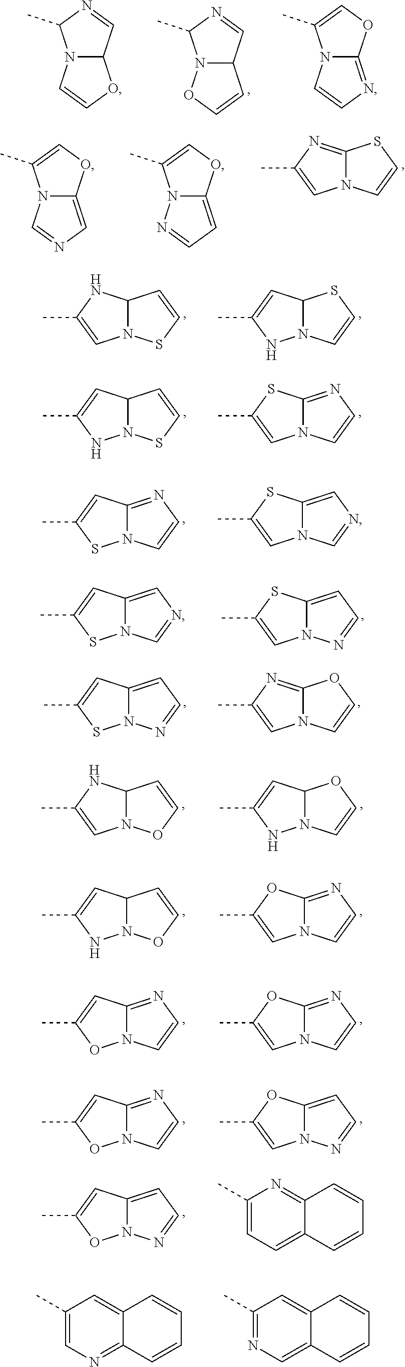

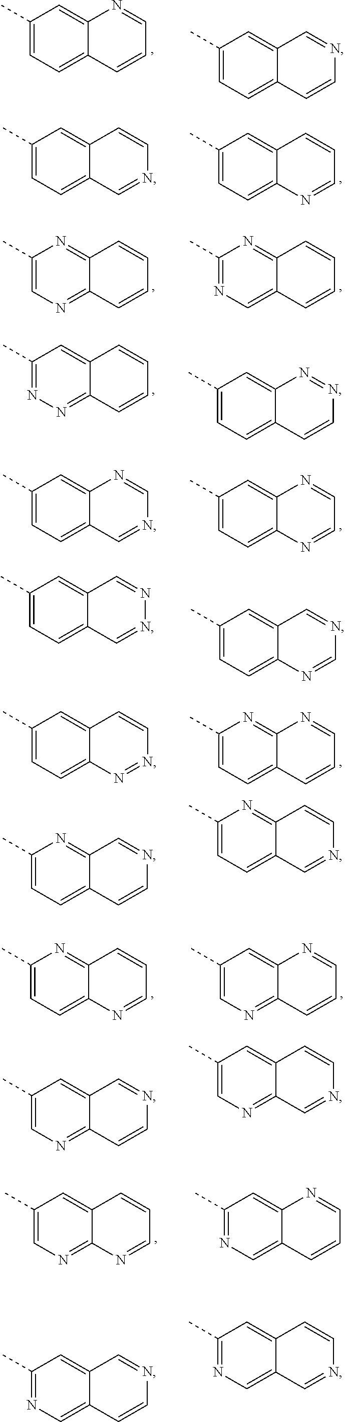

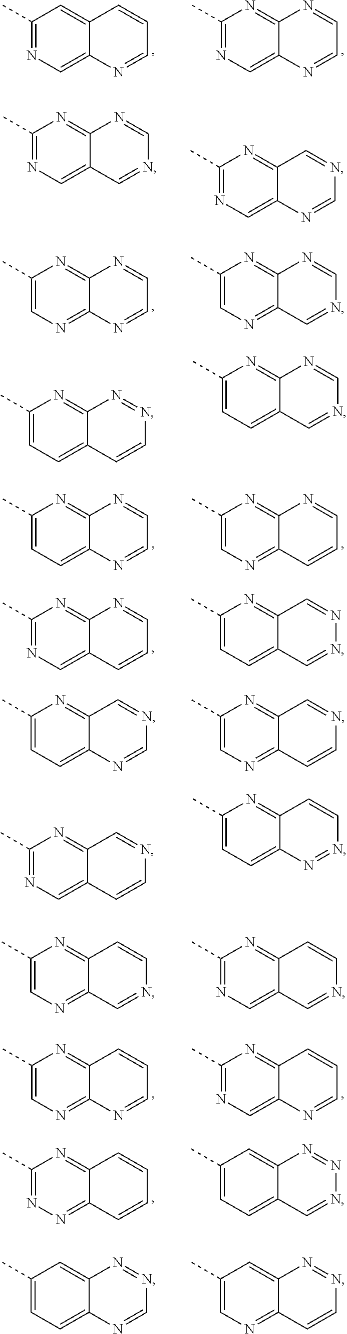

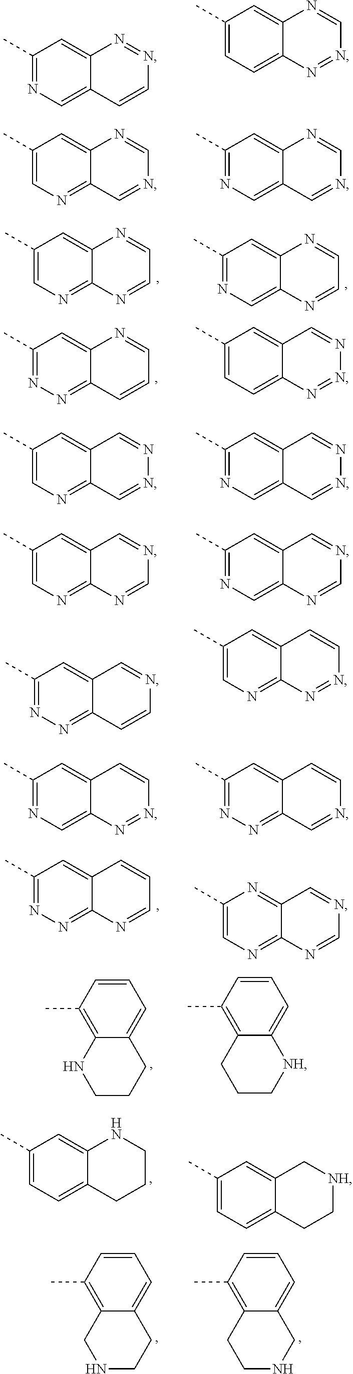

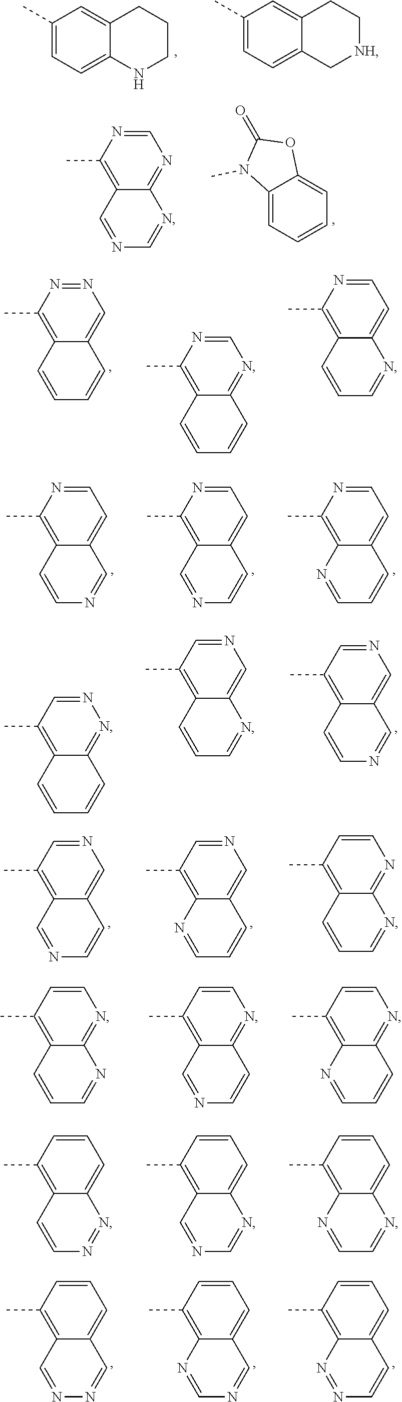

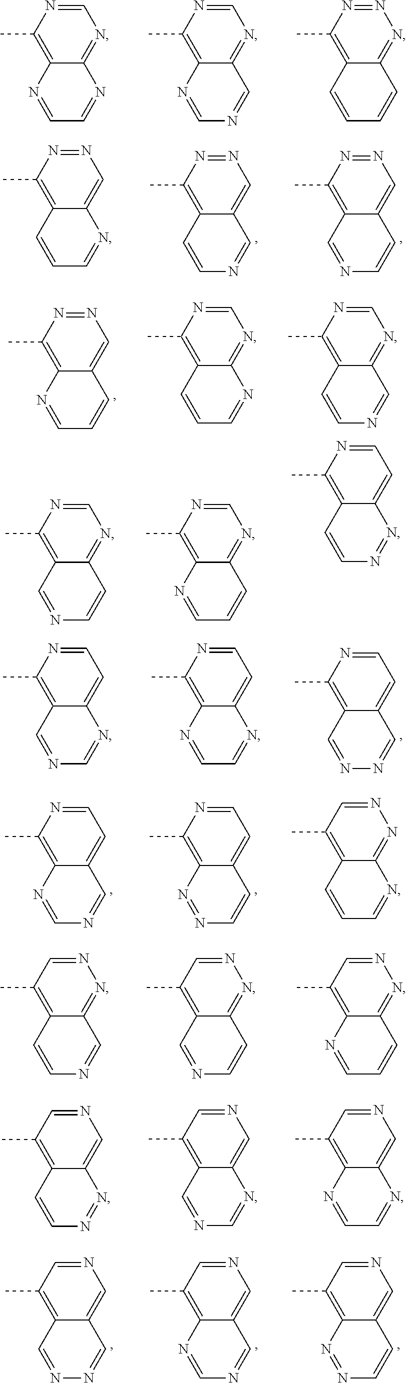

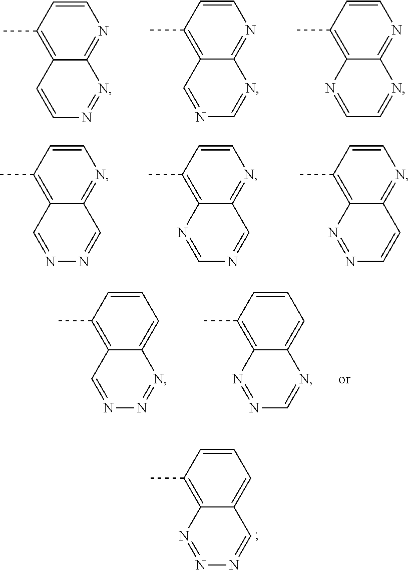

wherein the unsubstituted bicyclic residues can be substituted with 1 to 5 of the substituents R⁹-R¹⁴ and R^{N}; and preferably with 1 to 3 of the substituents R¹¹-R¹³;
R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl and the afore-mentioned residues optionally contain one or more C═C double bond(s) and/or are optionally substituted by one or more of R^{a}, R^{b}, bicyclo[2.1.1]hexyl, bicyclo[2.2.1]heptyl, R^{c}, R^{d}, and R^{e};
R^{a}, R^{b}, R^{c}, R^{d}, and R^{e} represents independently of each other —H, —F, —Cl, —Br, —CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, —CONHCH₃, —CON(CH₃)₂, —CONHC₂H₅, —CH₂CO₂H, —CH₂CO₂CH₃, —CH₂CO₂C₂H₅, —CH₂CONH₂, —CH₂CONHCH₃, —CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, —NHSO₂CH₂CF₃;
R⁴ represents —NR⁶R⁷;
R⁶ and R⁷ represent independently of each other —H, —CH₃, —CH₂CH₂CH₃, —CH(CH₃)₂, —CH₂CH₂CH₂CH₃, —CH₂CH₂CH₂CH₂CH₃, —CH₂CH(CH₃)₂, —C(CH₃)₃, —CH₂CH═CH₂, —CH₂CH═CH(CH₃), —CH₂CH═C(CH₃)₂, —CH₂CH═CHCH₂CH₃, -cyclo-C₃H₅, -cyclo-C₄H₇, -cyclo-C₅H₉, -cyclo-C₆H₁₁, —CH₂-cyclo-C₃H₅, -Ph, —CH₂-Ph, —CH₂-cyclo-C₄H₇, —CH₂-cyclo-C₅H₉, —CH₂-cyclo-C₆H₁₁, —CH₂OCH₃, —CH₂OCH₂CH₃, —CH₂CH₂OCH₃, —CH₂CH₂OCH₂CH₃, —CH₂CH₂NHCH₃, —CH₂CH₂N(CH₃)₂, or —NR⁶R⁷ is —N(C₂H₅)₂,

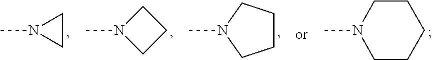

R⁸, R⁹, R¹⁰, R¹¹, R¹², R¹³, and R¹⁴ represent independently of each other —H, —F, —Cl, —Br, —I, —OH, —CN, —NO₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, —CH₂-cyclo-C₃H₅, —CH₂OH, —CH₂F, —CHF₂, —CF₃, —CH₂Cl, —CH₂Br, —CH₂I, —CH₂ CH₂F, —CH₂—CHF₂, —CH₂—CF₃, —CH₂—CH₂Cl, —CH₂—CH₂Br, CH₂—CH₂I, —OCH₃, —OCHF₂, —OCF₃, —OC₂H₅, —OC₃H₇, —OCH(CH₃)₂, —OC(CH₃)₃, —OCH₉, —OCH₂CF₃, —OC₂F₅, —OCH₂OCH₃, —O-cyclo-C₃H₅, —OCH₂-cyclo-C₃H₅, —O—C₂H₄-cyclo-C₃H₅, —CHO, —COCH₃, —COCF₃, —COC₂H₅, —COC₃H₇, —COCH(CH₃)₂, —COC(CH₃)₃, —COOH, —COOCH₃, —COOC₂H₅, —COOC₃H₇, —COOCH(CH₃)₂, —COOC(CH₃)₃, —OOC—CH₃, —OOC—CF₃, —OOC—C₂H₅, —OOC—C₃H₇, —OOC—CH(CH₃)₂, —OOC—C(CH₃)₃, —NH₂, —NHCH₃, —NHC₂H₅, —NHC₃H₇, —NHCH(CH₃)₂, —NHC(CH₃)₃, —N(CH₃)₂, —N(C₂H₅)₂, —N(C₃H₇)₂, —N[CH(CH₃)₂]₂, —N[C(CH₃)₃]₂, —NHCOCH₃, —NHCOCF₃, —NHCOC₂H₅, —NHCOC₃H₇, —NHCOCH(CH₃)₂, —NHCOC(CH₃)₃, —CONH₂, —CONHCH₃, —CONHC₂H₅, —CONHC₃H₇, —CONHCH(CH₃)₂, —CONH-cyclo-C₃H₅, —CONHC(CH₃)₃, —CON(CH₃)₂, —CON (C₂H₅)₂, —CON (C₃H₇)₂, —CON[CH(CH₃)₂]₂, —CON[C(CH₃)₃]₂, —SO₂NH₂, —SO₂NHCH₃, —SO₂NHC₂H₅, —SO₂NHC₃H₇, —SO₂NHCH(CH₃)₂, —SO₂NH-cyclo-C₃H₅, —SO₂NHC(CH₃)₃, —SO₂N(CH₃)₂, —SO₂N(C₂H₅)₂, —SO₂N(C₃H₇)₂, —SO₂N[CH(CH₃)₂]₂, —SO₂N[C(CH₃)₃]₂, —NHSO₂CH₃, —NHSO₂CF₃, —NHSO₂C₂H₅, —NHSO₂C₃H₇, —NHSO₂CH(CH₃)₂, —NHSO₂C(CH₃)₃, —CH═CH₂, —CH₂—CH═CH₂, —C(CH₃)═CH₂, —CH═CH—CH₃, —C≡CH, —C≡C—CH₃, —CH₂—C≡CH, -Ph, —O-Ph, —O—CH₂-Ph,

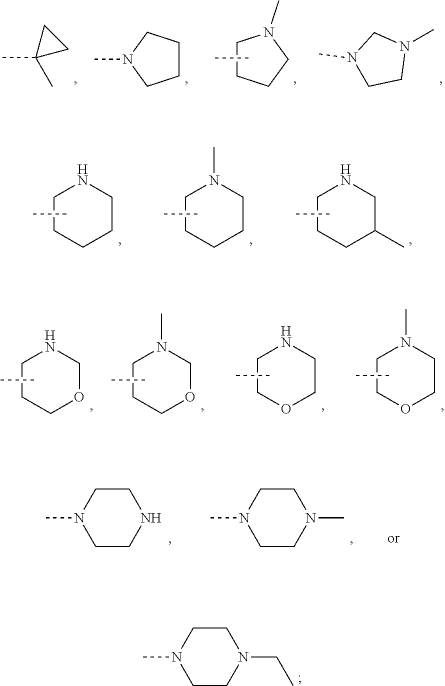

or R⁸ and R⁹ or R⁹ and R¹⁰ can form together one of the following five-membered or six-membered rings:

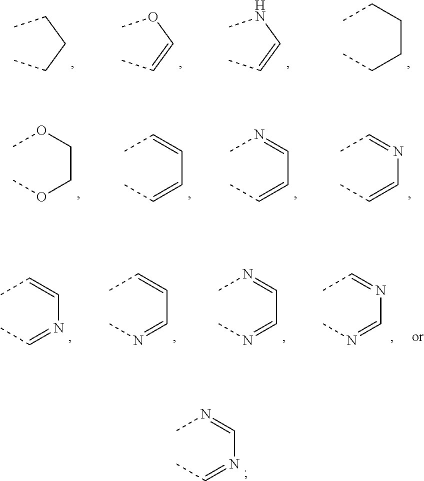

or R¹² and R¹³ or R¹³ and R¹⁴ can form together one of the following five-membered or six-membered rings:

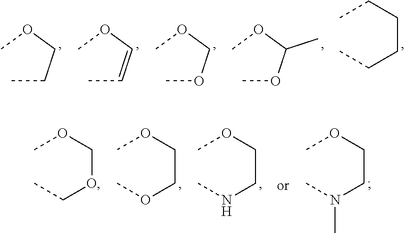

R^{N} represents —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, cyclo-C₃H₅, -cyclo-C₄H₇, -cyclo-C₅H₉, —CH₂-cyclo-C₃H₅, —CH₂-cyclo-C₄H₇, —CH₂-cyclo-C₅H₉, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₂—CH═CH₂, —CH₂—C═CH, —CHO, —COCH₃, —COC₂H₅, —COC₃H₇, —COCH(CH₃)₂, —COC(CH₃)₃, —CO-cyclo-C₃H₅, —CO-cyclo-C₄H₇, —CO-cyclo-C₅H₉, —COOCH₃, —COOC₂H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, —SO₂-cyclo-C₃H₅, or —SO₂C(CH₃)₃;
R^{N1} represent —H, —CH₃, or —CH₂CH₃;
or a diastereomer, an enantiomer, a mixture of diastereomers, a mixture of enantiomers, a racemate, a solvate, a hydrate, or a pharmaceutically acceptable salt thereof.

The inventors have found that the reversible inhibitors of formula (I) disclosed herein having a bridged bicyclic residue R³ exhibit increased potency over the compounds of the prior art. Particularly, it is demonstrated herein, that the inventive compounds have an improved inhibitory activity compared to the known compounds bearing aromatic moieties R³ instead of bridged bicyclic residues. In order to prove inventiveness of the compounds of the present application, known compounds from U.S. Pat. No. 9,434,763 B2 and U.S. Pat. No. 11,072,634 B2 (Reference 1 (E16 from U.S. Pat. No. 11,072,634 B2), Reference 3 (A8 from U.S. Pat. No. 9,434,763 B2), and Reference 6) were synthesized and tested as reference compounds in comparison to the most similar compounds of the present application. To this extent, inhibition data were determined using the classical fluorescent transamidation assay (dansylcadaverine incorporation into methylated casein, DCC-assay) as described in Büchold et al. [Büchold, C.; Hils, M.; Gerlach, U.; Weber, J.; Pelzer, C.; Heil, A.; Aeschlimann, D.; Pasternack, R. Features of ZED1227: The First-In-Class Tissue Transglutaminase Inhibitor Undergoing Clinical Evaluation for the Treatment of Celiac Disease. Cells 2022, 11, 1667. https://doi.org/10.3390/cells11101667]. Casein is one of the best known high molecular weight (24 kDa) protein substrates for transglutaminases. Inhibition data of the inventive compounds was compared with inhibition of compounds disclosed in U.S. Pat. No. 9,434,763 B2, particularly, compound A8, which is denoted herein as Reference 3. It is noteworthy that the IC₅₀ values of compounds A8 published in U.S. Pat. No. 9,434,763 B2 and E16 from U.S. Pat. No. 11,072,634 B2 cannot be compared to the present data, relying on a fluorogenic isopeptidase assay.

Thus, the inventive compounds of formula (I) rated “A” showed efficacies of about 100-fold higher compared to Ref. 3 (A8). The same argumentation applies to Ref. 6 which is except of the phenylethyl group identical to compound II-111. Ref. 6 is more than 25-times less potent in comparison to compound II-111 as evident from Table 1.

This finding was particularly surprising as a skilled person would not have expected an improved inhibitory activity of the inventive compounds bearing a bridged bicyclic residue over the compounds of the prior art bearing aromatic residues since it is common knowledge that bridged bicyclic groups or bridged cycloalkyl groups are non-classical bioisosters of the phenyl group, such that a skilled person would only expect to obtain a compound having similar physico-chemical and biological properties, including inhibitory activity, when replacing a phenyl group with a bridged bicyclic group. As aromatic moieties showed lower potency, bridged cycloalkyl groups would not be considered improving the physico-chemical and biological properties of the compounds.

The residues bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, and hexamethylenetetraminyl used herein, have the following parent structures respectively:

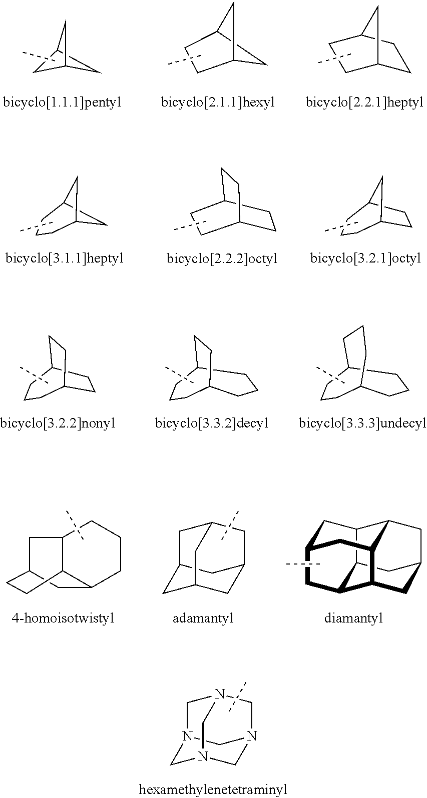

and the afore-mentioned residues optionally contain one or more C═C double bond(s) such as bicyclo[2.2.1]hept-5-enyl (s. II-97) and/or are optionally substituted by one or more of R^{a}, R^{b}, R^{c}, R^{d}, and R^{e}.

The unsubstituted bicyclic residues which can be substituted with 1 to 5 of the substituents R⁹-R¹⁴ and R^{N}; have the following structure and the substituents R⁹-R¹⁴ and R^{N} have the meanings as defined herein:

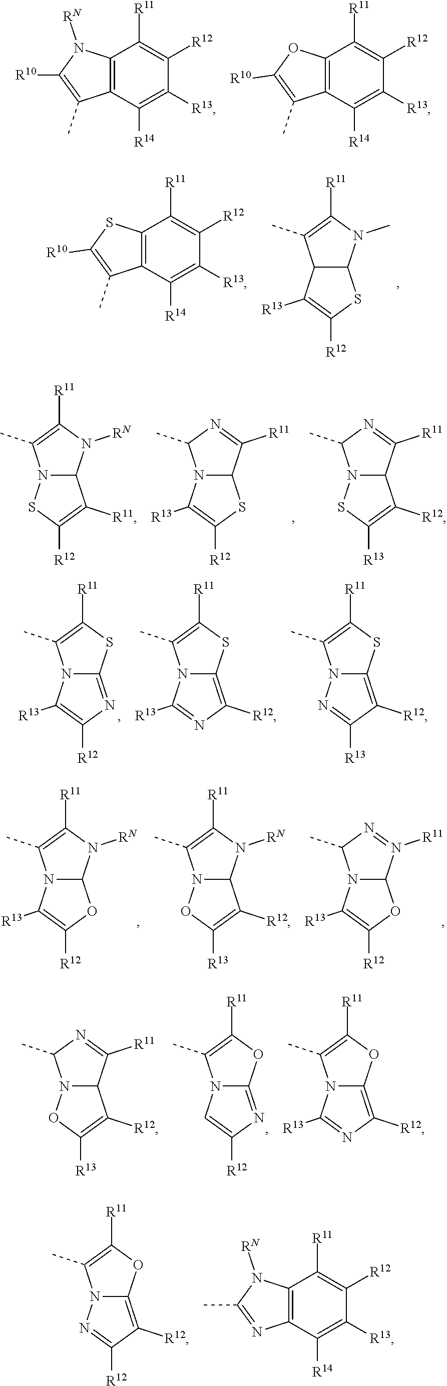

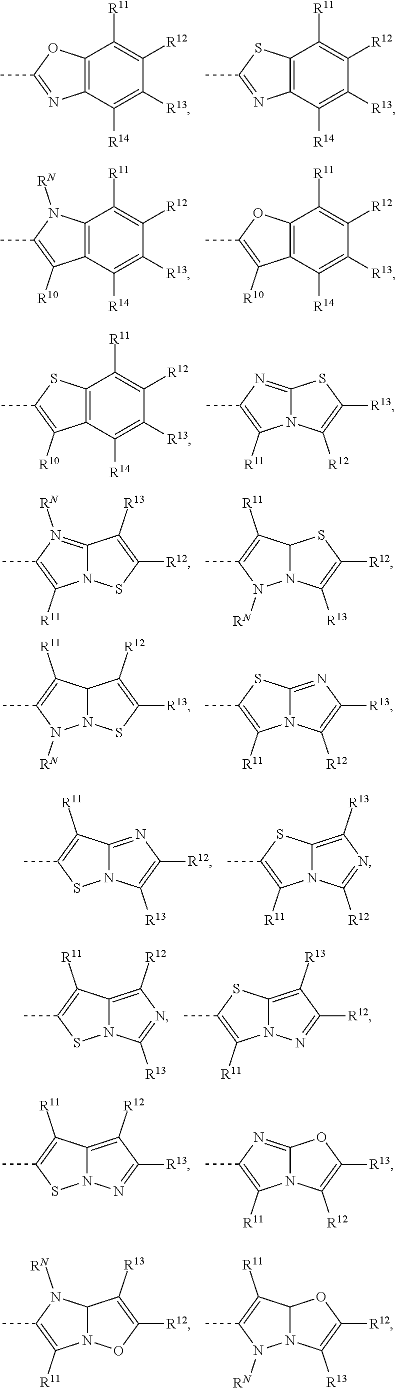

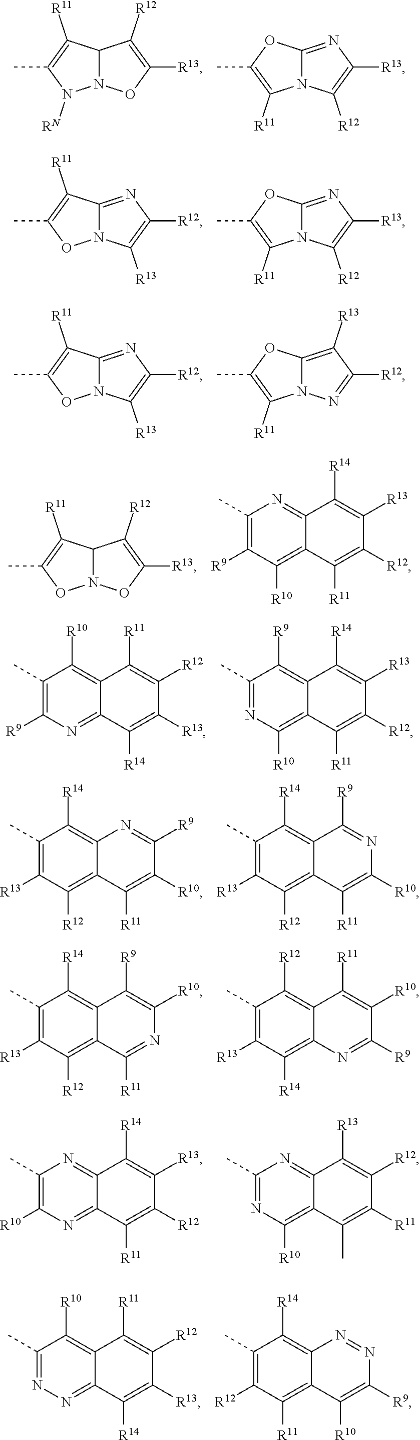

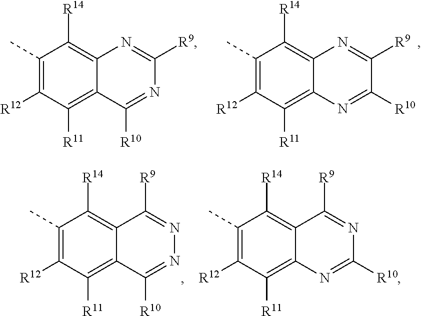

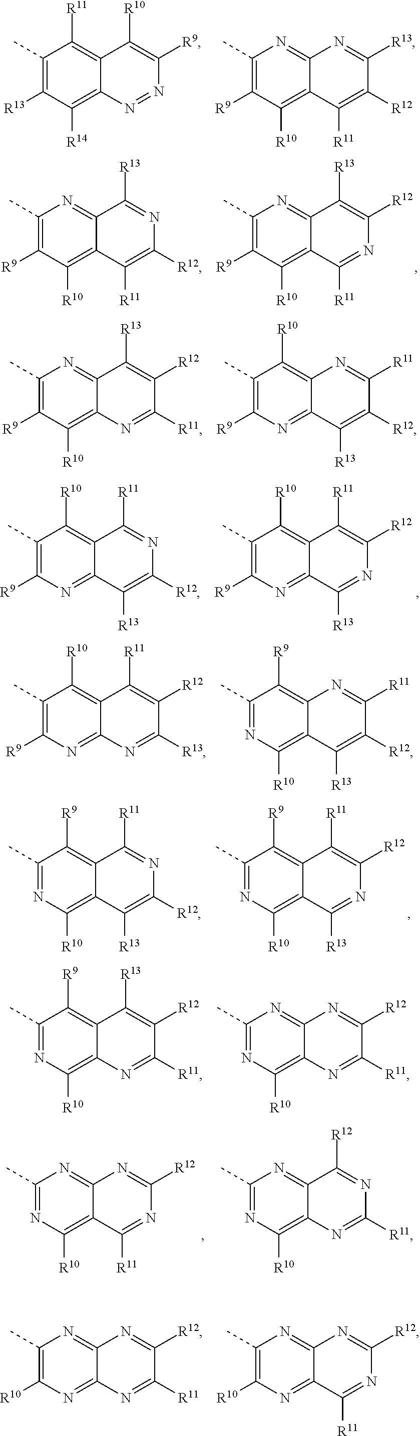

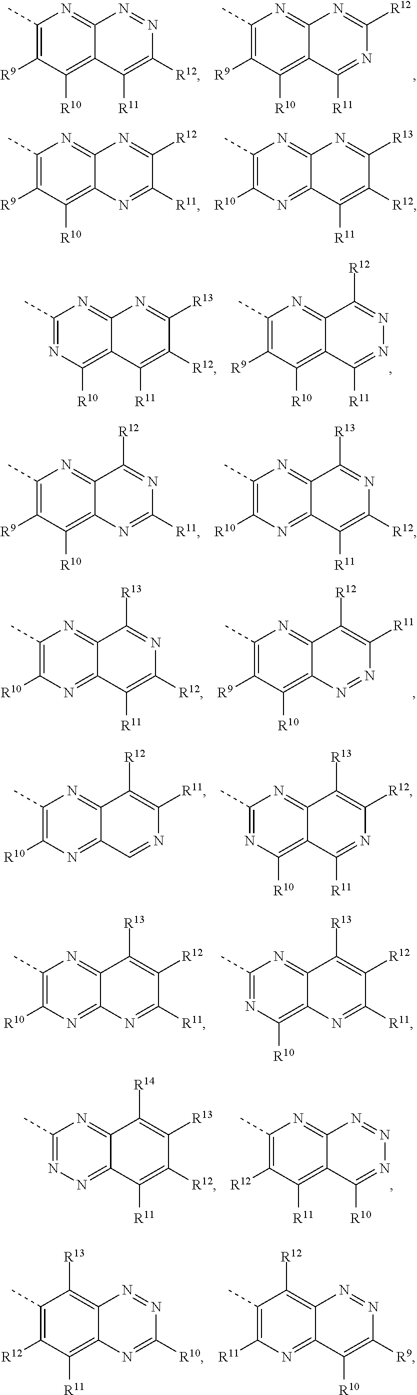

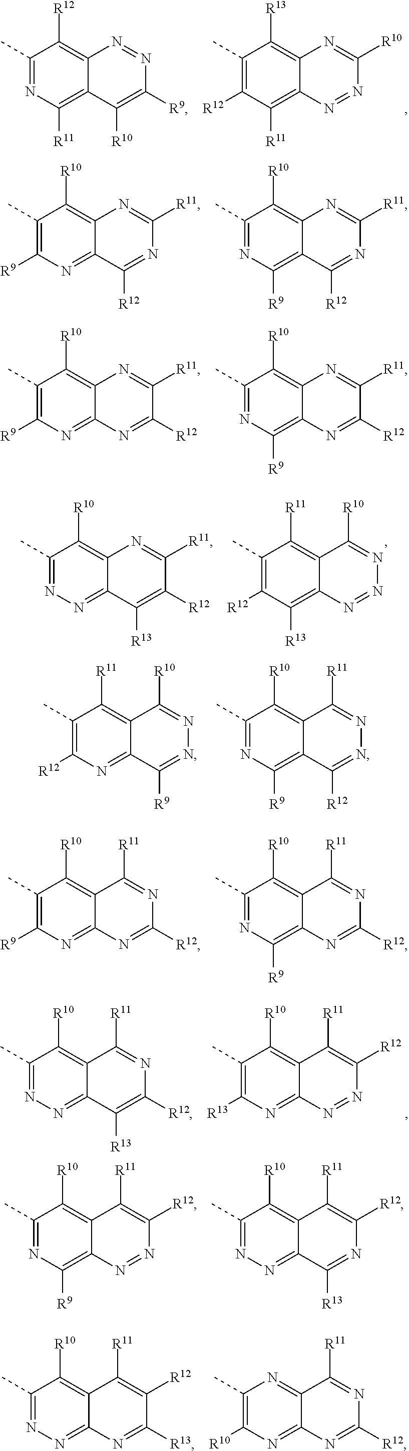

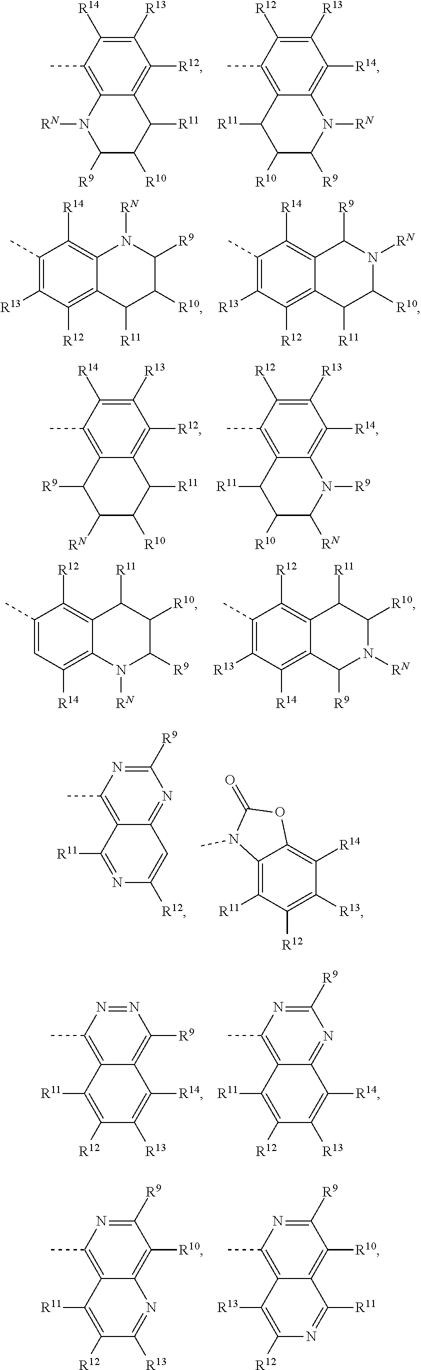

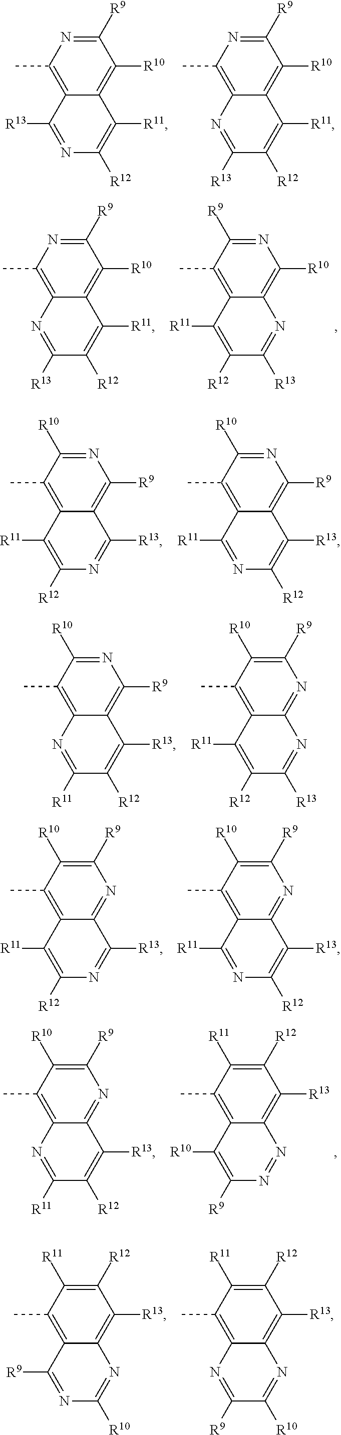

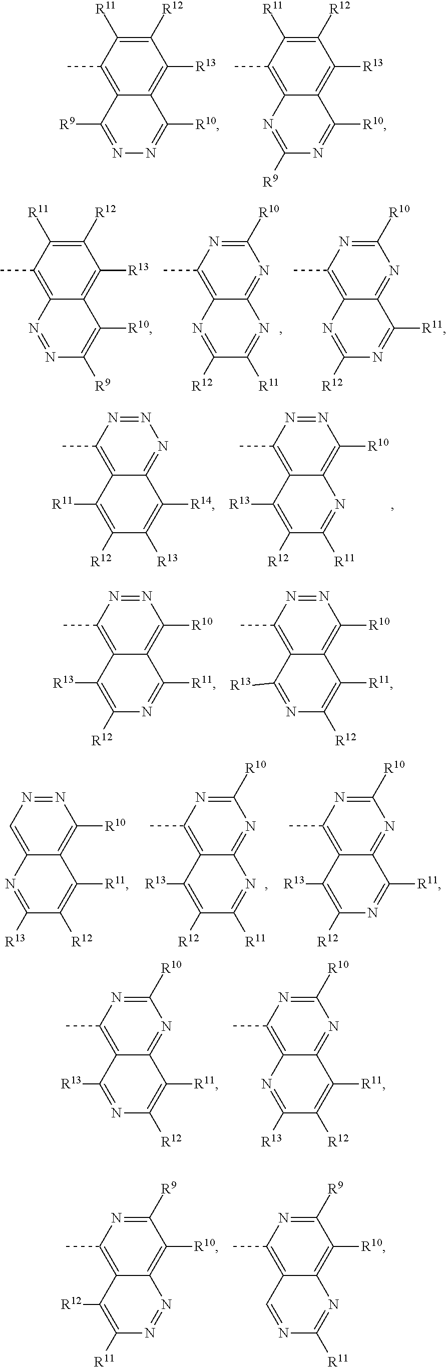

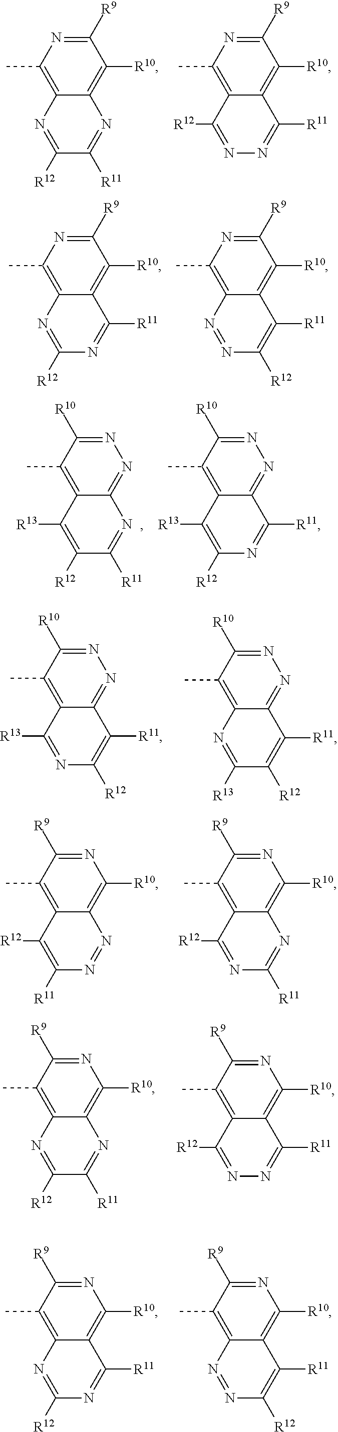

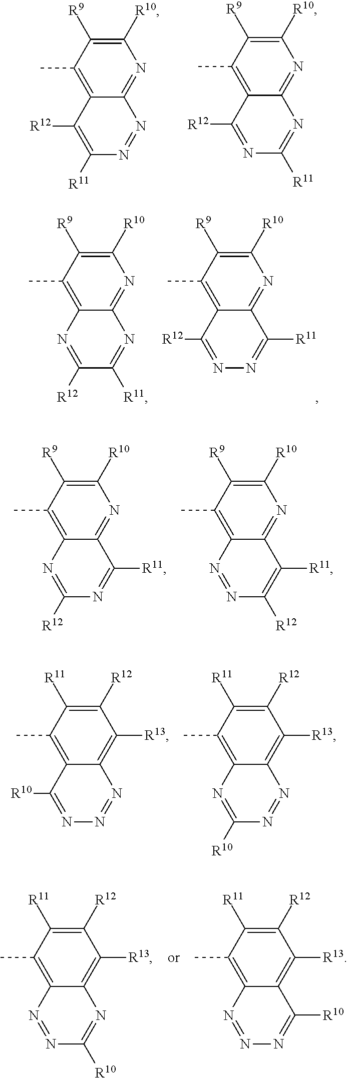

One embodiment is directed to compounds of the general formula (I):

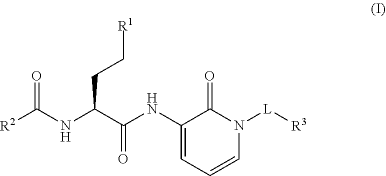

wherein
L represents -L¹- or -L¹-L²-; preferably -L¹-L²-;
L¹ represents —CH₂—, —CH₂CH₂—, —CH₂CH₂CH₂—, —CH₂CO—, or —CH₂CH₂CO—;
R¹ represents

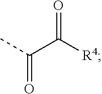

R² represents

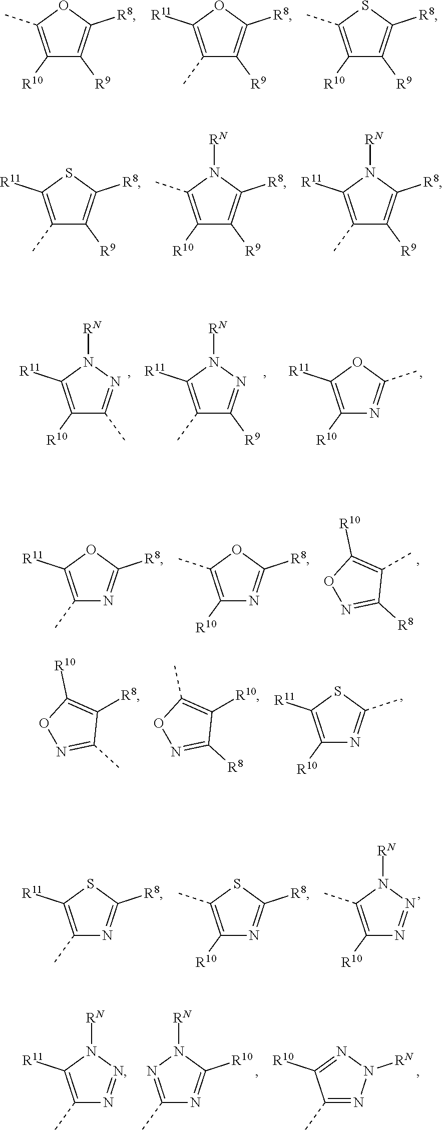

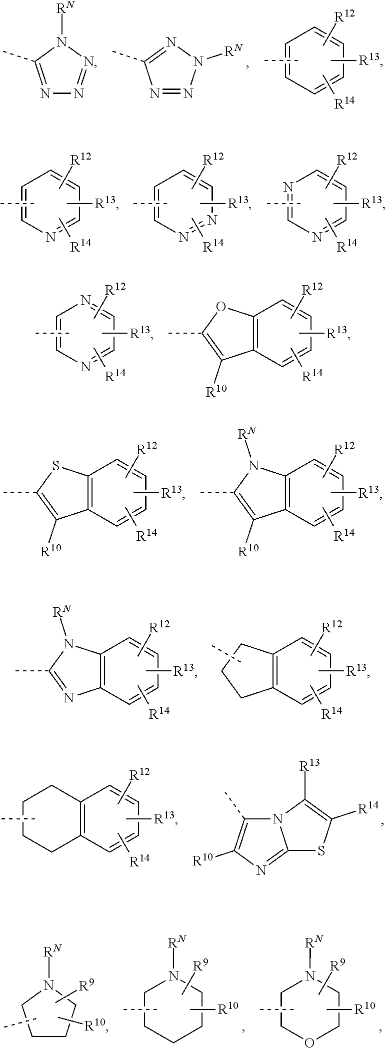

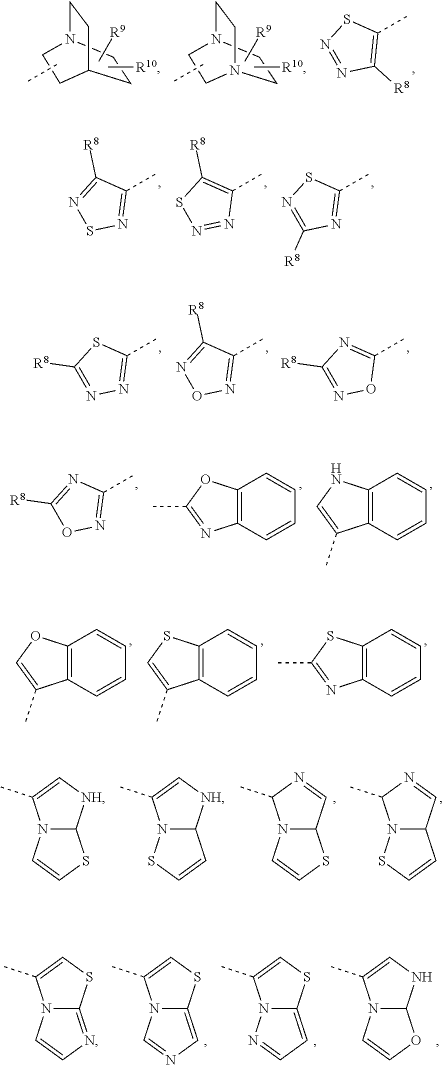

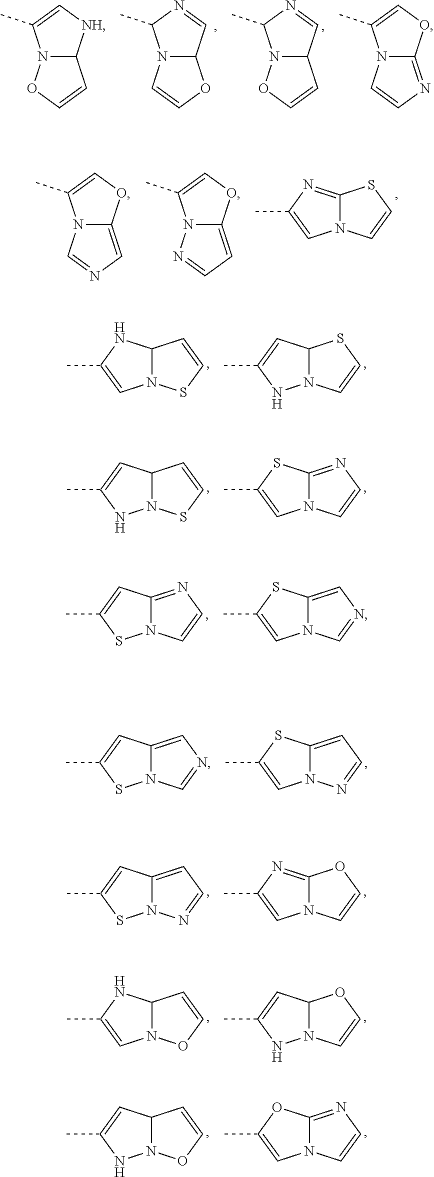

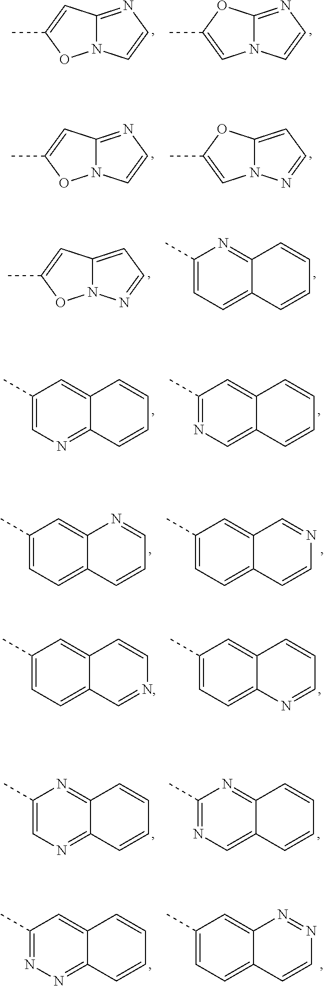

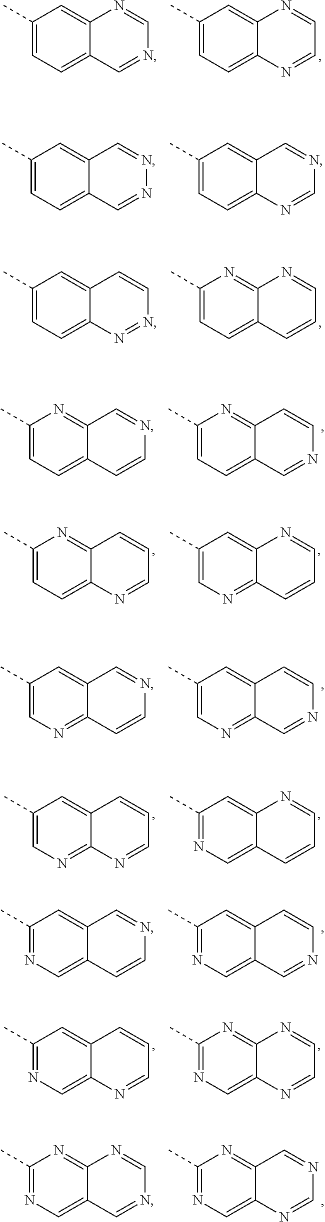

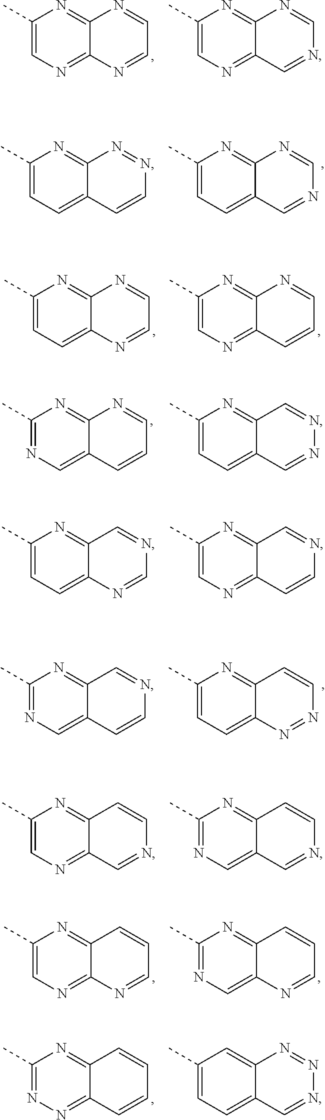

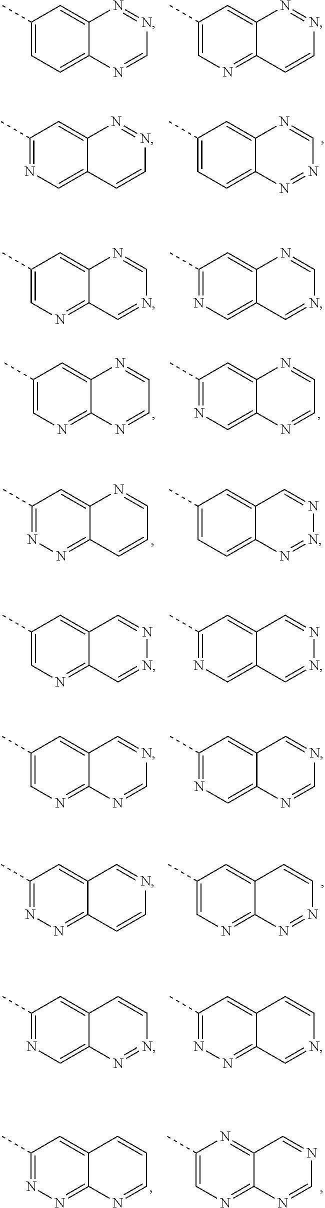

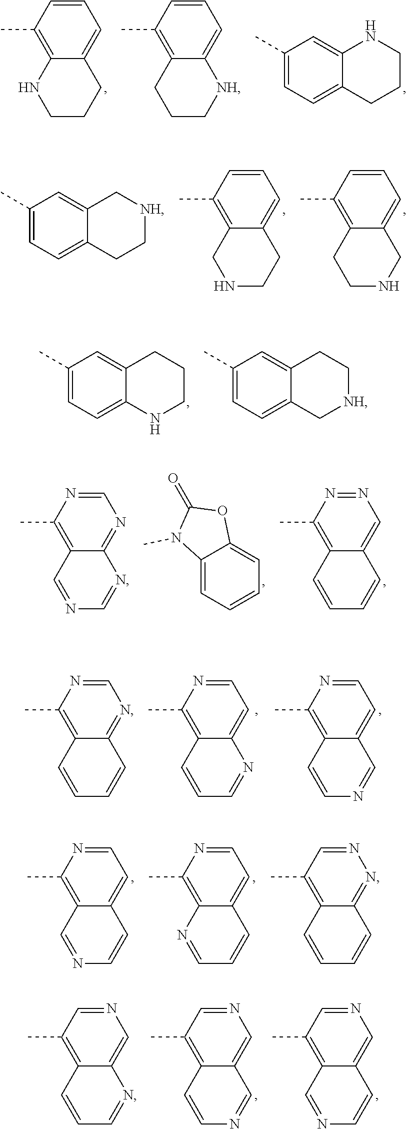

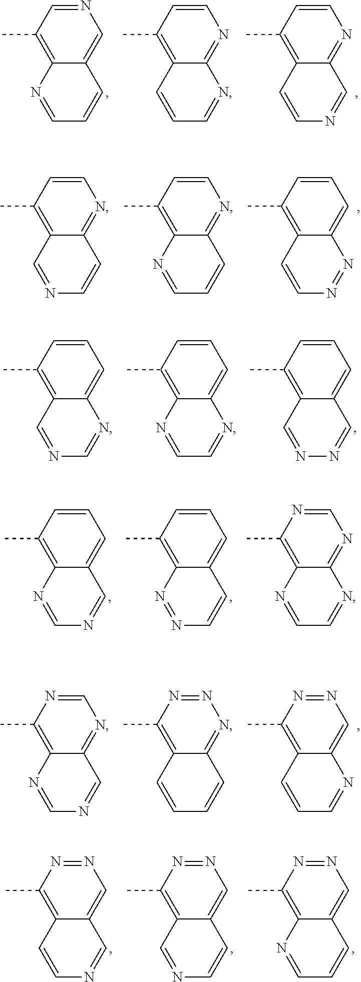

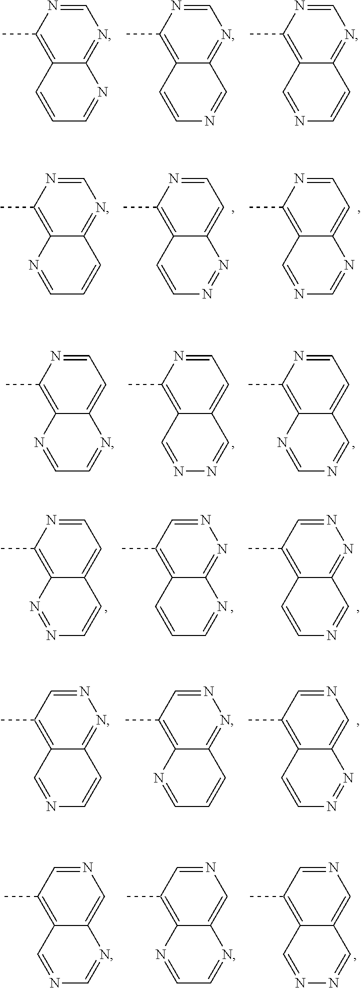

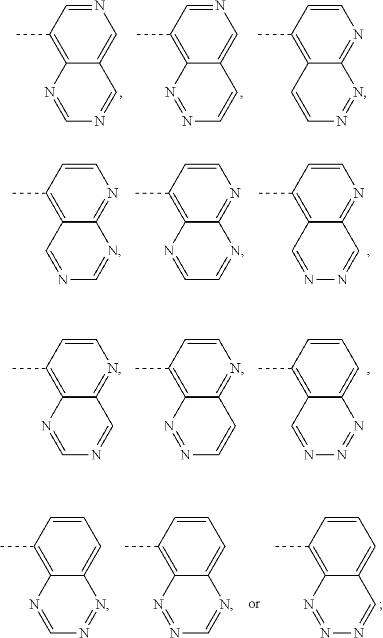

wherein the unsubstituted bicyclic residues can be substituted with 1 to 5 of the substituents R⁹-R¹⁴ and R^{N}; and preferably with 1 to 3 of the substituents R¹¹-R¹³.
wherein
i) L² represents a bond, —NRN₁CH₂—, —NRN₁CH₂CH₂—, or —NR^{N1}CH(CH₃)—; and R³ represents bicyclo[1.1.1]pentyl, bicyclo[2.1.1]hexyl, bicyclo[2.2.1]heptyl, 1-bicyclo[3.1.1]heptyl, 3-bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, diamantyl, hexamethylenetetraminyl and the afore-mentioned residues optionally contain one or more C═C double bond(s) and/or are optionally substituted by one or more of R^{a}, R^{b}, R^{c}, R^{d}, and R^{e}; or
ii) L² represents a bond, —NR^{N1}—, —NR^{N1}CH₂CH₂—, or —NR^{N1}CH(CH₃)—; and R³ represents bicyclo[1.1.1]pentyl, bicyclo[2.1.1]hexyl, bicyclo[2.2.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, 2-adamantyl, diamantyl, hexamethylenetetraminyl and the afore-mentioned residues optionally contain one or more C═C double bond(s) and/or are optionally substituted by one or more of R^{a}, R^{b}, R^{c}, R^{d}, and R^{e},
R^{a}, R^{b}, R^{c}, R^{d}, and R^{e} represents independently of each other —H, —F, —Cl, —Br, —CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, —CONHCH₃, —CON(CH₃)₂, —CONHC₂H₅, —CH₂CO₂H, —CH₂CO₂CH₃, —CH₂CO₂C₂H₅, —CH₂CONH₂, —CH₂CONHCH₃, —CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, —NHSO₂CH₂CF₃;
R⁴ represents —NR⁶R⁷.
R⁶ and R⁷ represent independently of each other —H, —CH₃, —CH₂CH₂CH₃, —CH(CH₃)₂, —CH₂CH₂CH₂CH₃, —CH₂CH₂CH₂CH₂CH₃, —CH₂CH(CH₃)₂, —C(CH₃)₃, —CH₂CH═CH₂, —CH₂CH═CH(CH₃), —CH₂CH═C(CH₃)₂, —CH₂CH═CHCH₂CH₃, -cyclo-C₆H₁₁, —CH₂-cyclo-C₃H₅, -Ph, —CH₂-Ph, —CH₂-cyclo-C₄H₇, —CH₂-cyclo-C₅H₉, —CH₂-cyclo-C₆H₁₁, -cyclo-C₃H₅, -cyclo-C₄H₇, -cyclo-C₅H₉, —CH₂OCH₃, —CH₂OCH₂CH₃, —CH₂CH₂NHCH₃, —CH₂CH₂N(CH₃)₂, —CH₂CH₂OCH₃, —CH₂CH₂OCH₂CH₃, or —NR⁶R⁷ is-N(C₂H₅)₂,

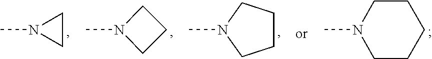

R⁸, R⁹, R¹⁰, R¹¹, R¹², R¹³, and R¹⁴ represent independently of each other —H, —F, —Cl, —Br, —I, —OH, —CN, —NO₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, —CH₂-cyclo-C₃H₅, —CH₂OH, —CH₂F, —CHF₂, —CF₃, —CH₂Cl, —CH₂Br, —CH₂I, —CH₂—CH₂F, —CH₂—CHF 2, —CH₂—CF₃, —CH₂—CH₂Cl, —CH₂—CH₂Br, —CH₂—CH₂I, —OCH₃, —OC₂H₅, —OC₃H₇, —OCH(CH₃)₂, —OC(CH₃)₃, —OCH₉, —OCHF₂, —OCF₃, —OCH₂CF₃, —OC₂F₅, —OCH₂OCH₃, —O-cyclo-C₃H₅, —OCH₂-cyclo-C₃H₅, —O—C₂H₄-cyclo-C₃H₅, —CHO, —COCH₃, —COCF₃, —COC₂H₅, —COC₃H₇, —COCH(CH₃)₂, —COC(CH₃)₃, —COOH, —COOCH₃, —COOC₂H₅, —COOC₃H₇, —COOCH(CH₃)₂, —COOC(CH₃)₃, —OOC—CH₃, —OOC—CF₃, —OOC—C₂H₅, —OOC—C₃H₇, —OOC—CH(CH₃)₂, —OOC—C(CH₃)₃, —NH₂, —NHCH₃, —NHC₂H₅, —NHC₃H₇, —NHCH(CH₃)₂, —NHC(CH₃)₃, —N(CH₃)₂, —N(C₂H₅)₂, —N(C₃H₇)₂, —N[CH(CH₃)₂]₂, —N[C(CH₃)₃]₂, —NHCOCH₃, —NHCOCF₃, —NHCOC₂H₅, —NHCOC₃H₇, —NHCOCH(CH₃)₂, —NHCOC(CH₃)₃, —CONH₂, —CONHCH₃, —CONHC₂H₅, —CONHC₃H₇, —CONHCH(CH₃)₂, —CONH-cyclo-C₃H₅, —CONHC(CH₃)₃, —CON(CH₃)₂, —CON(C₂H₅)₂, —CON (C₃H₇)₂, —CON[CH(CH₃)₂]₂, —CON[C(CH₃)₃]₂, —SO₂NH₂, —SO₂NHCH₃, —SO₂NHC₂H₅, —SO₂NHC₃H₇, —SO₂NHCH(CH₃)₂, —SO₂NH-cyclo-C₃H₅, —SO₂NHC(CH₃)₃, —SO₂N(CH₃)₂, —SO₂N(C₂H₅)₂, —SO₂N(C₃H₇)₂, —SO₂N[CH(CH₃)₂]₂, —SON[C(CH₃)₃]₂, —NHSO₂CH₃, —NHSO₂CF₃, —NHSO₂C₂H₅, —NHSO₂C₃H₇, —NHSO₂CH(CH₃)₂, —NHSO₂C(CH₃)₃, —CH═CH₂, —CH₂—CH═CH₂, —C(CH₃)═CH₂, —CH═CH—CH₃, —C≡CH, —C≡C—CH₃, —CH₂—C≡CH, -Ph, —O-Ph, —O—CH₂-Ph,

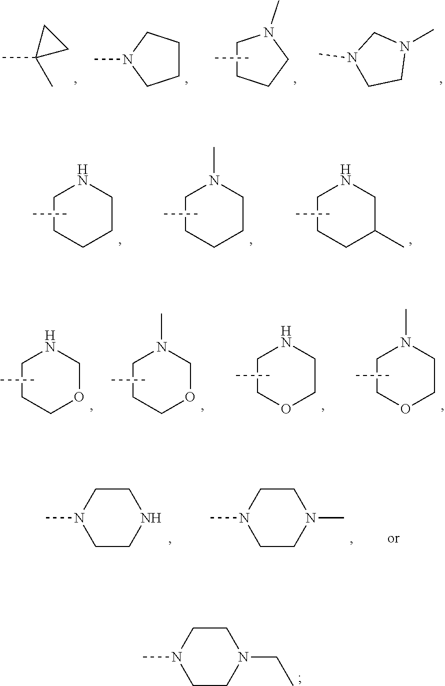

or R⁸ and R⁹ or R³ and R¹⁰ can form together one of the following five-membered or six-membered rings:

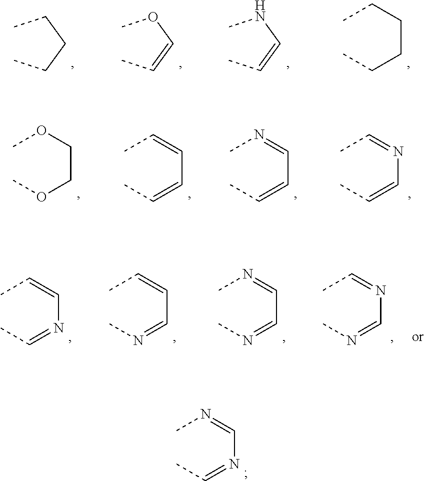

or R¹² and R¹³ or R¹³ and R¹⁴ can form together one of the following five-membered or six-membered rings:

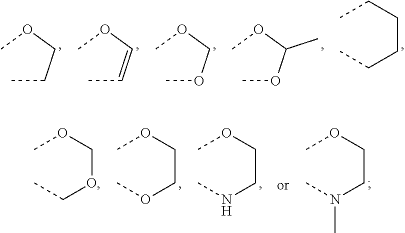

R^{N} represents —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, -cyclo-C₄H₇, -cyclo-C₅H₉, —CH₂-cyclo-C₃H₅, —CH₂-cyclo-C₄H₇, —CH₂-cyclo-C₅H₉, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₂—CH═CH₂, —CH₂—C≡CH, —CHO, —COCH₃, —COC₂H₅, —COC₃H₇, —COCH(CH₃)₂, —COC(CH₃)₃, —CO-cyclo-C₃H₅, —CO-cyclo-C₄H₇, —CO-cyclo-C₅H₉, —COOCH₃, —COOC₂H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, —SO₂-cyclo-C₃H₅, or —SO₂C(CH₃)₃;
R^{N1} represent —H, —CH₃, or —CH₂CH₃;
or a diastereomer, an enantiomer, a mixture of diastereomers, a mixture of enantiomers, a racemate, a solvate, a hydrate, or a pharmaceutically acceptable salt thereof.

In one embodiment the herein disclosed inventive compounds, the moiety -L-R³ is not

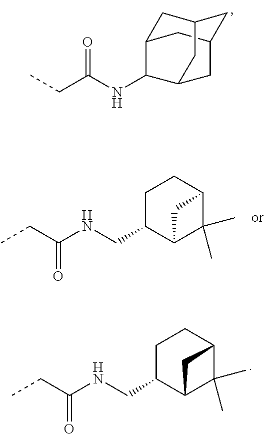

In one embodiment of the herein disclosed inventive compounds, L represents -L¹-L²-;
L¹ represents —CH₂—, or —CH₂CO—; and
L² represents a bond, —NH—, —NHCH₂—, —NHCH₂CH₂—, or —NHCH(CH₃)—.

In one embodiment of the herein disclosed inventive compounds, L represents —CH₂—, —CH₂CO—NH—, —CH₂CO—NH—CH₂—, or —CH₂CO—NH—CH(CH₃)—.

In preferred embodiments of the inventive compound of formula (I), R² represents

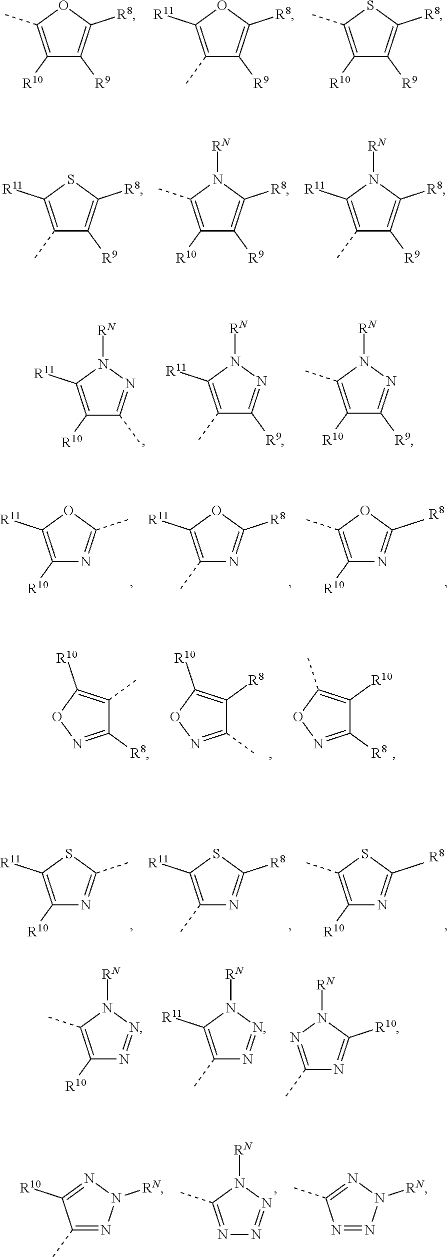

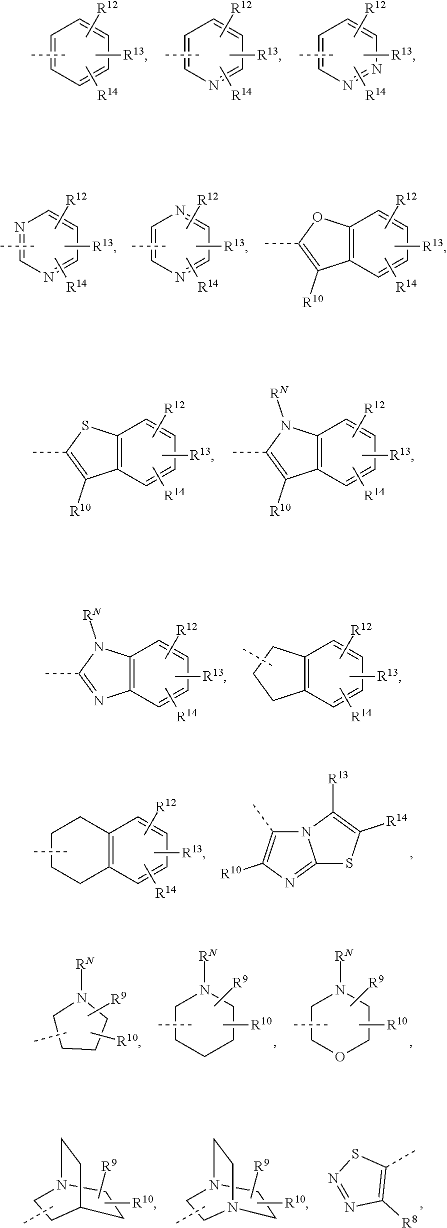

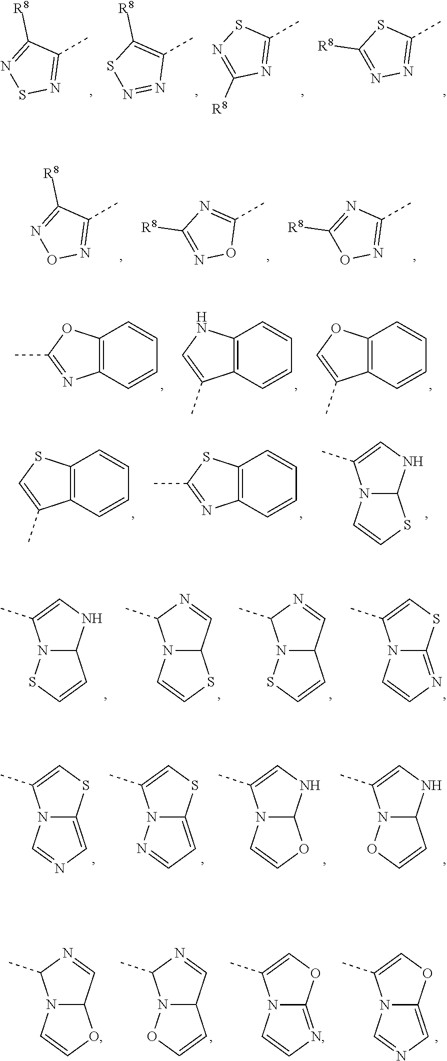

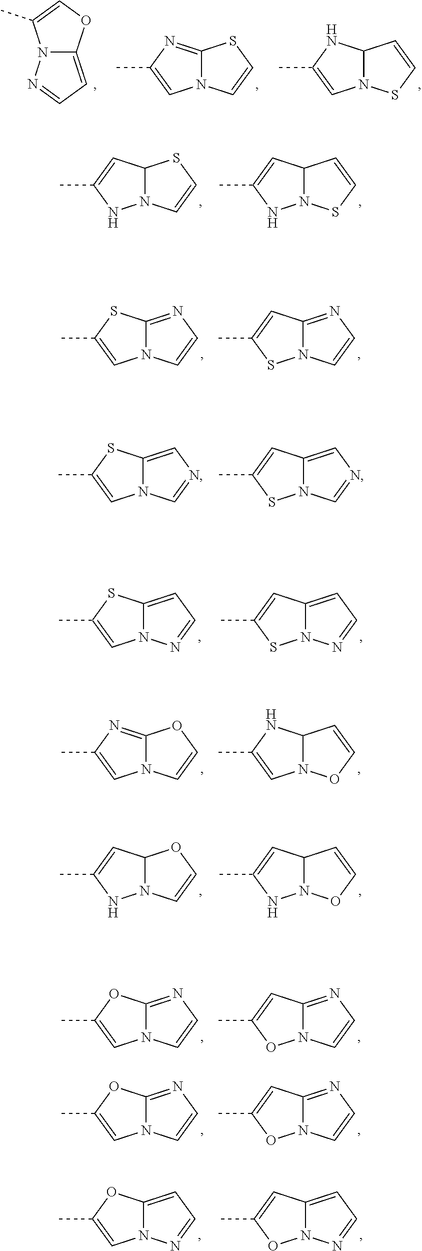

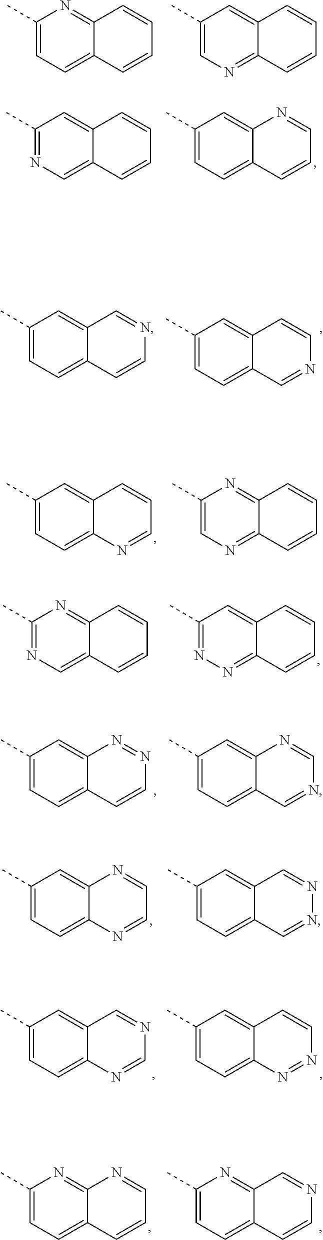

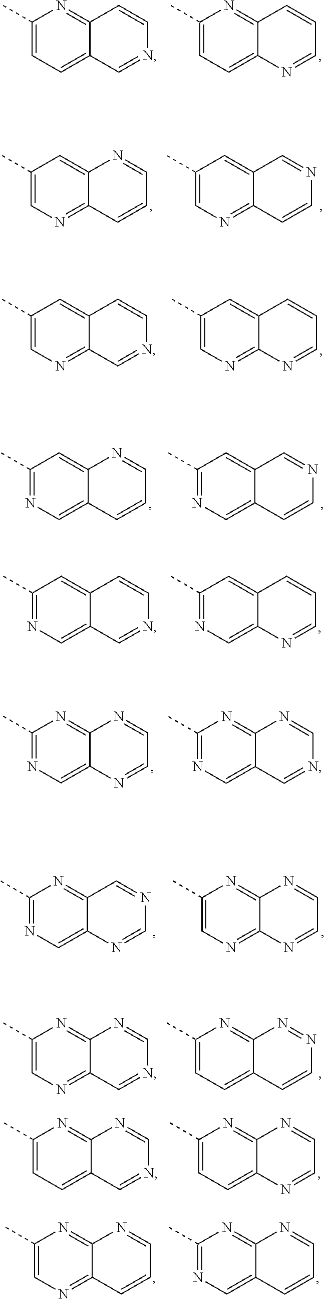

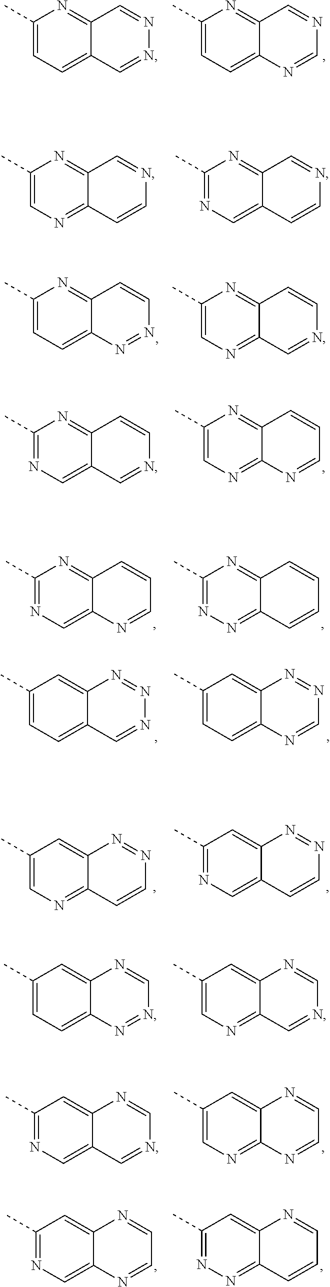

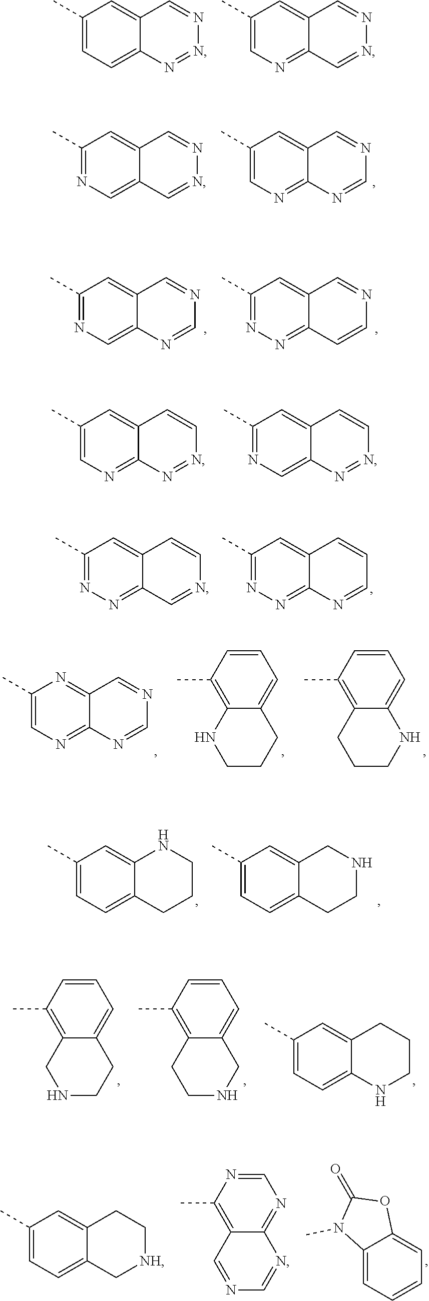

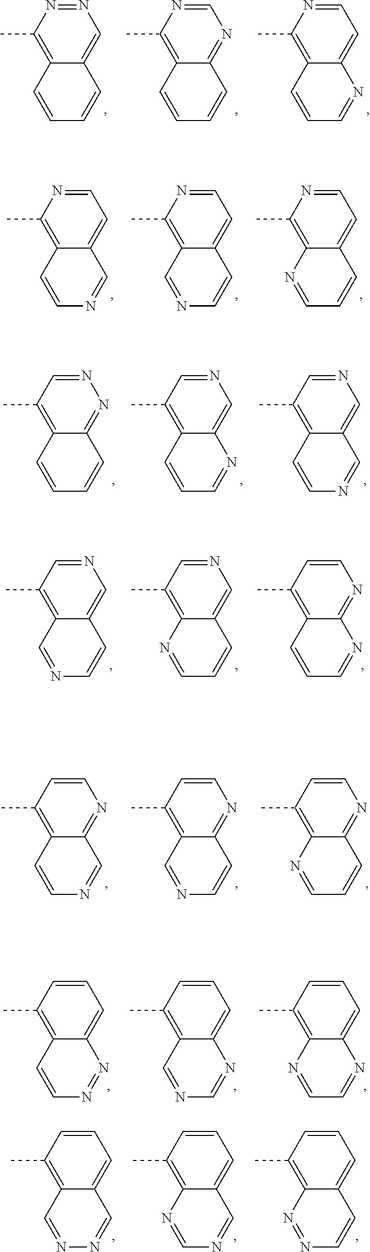

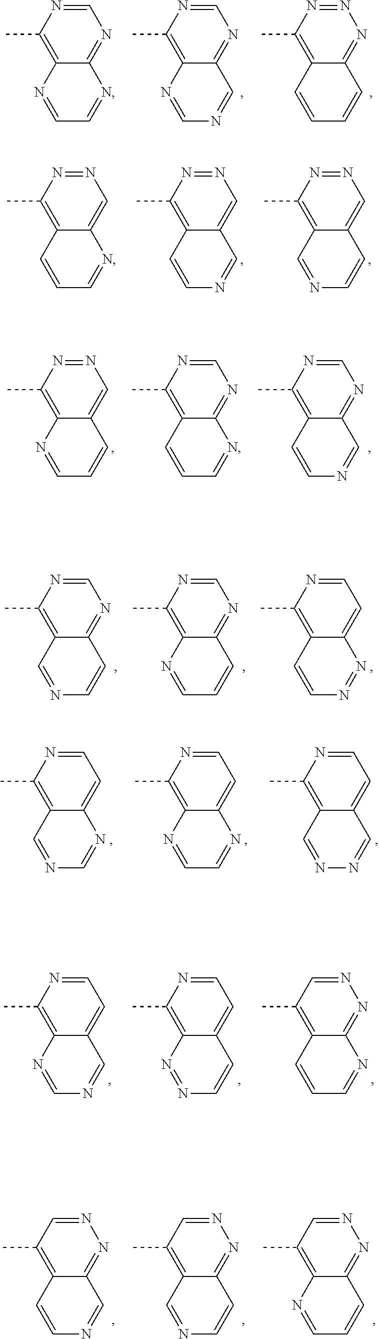

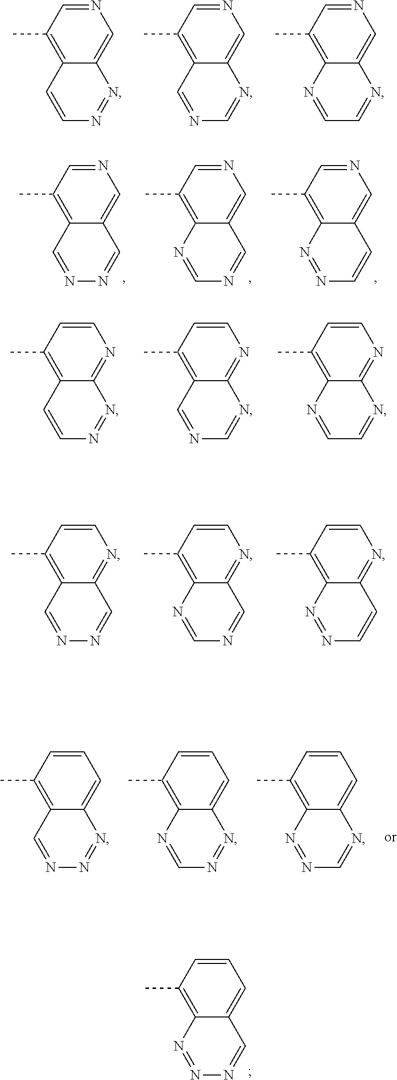

wherein the unsubstituted bicyclic residues can be substituted with 1 to 5 of the substituents R⁹-R¹⁴ and R^{N}; and preferably with 1 to 3 of the substituents R¹¹-R¹³ and the substituents R⁹-R¹⁴ and R^{N3} have the meanings as defined herein.

In even more preferred embodiments, R² represents:

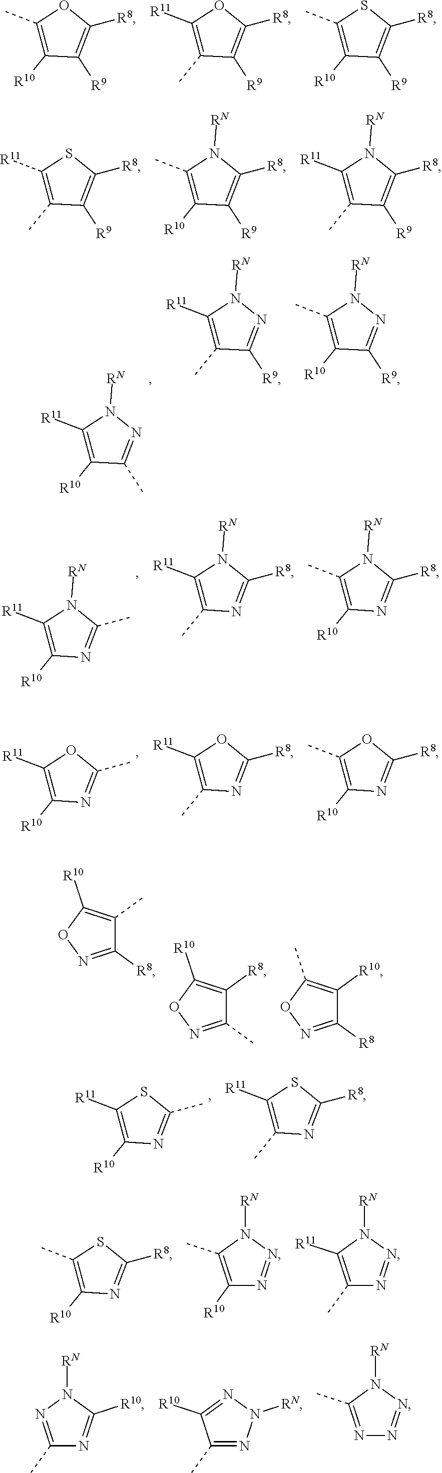

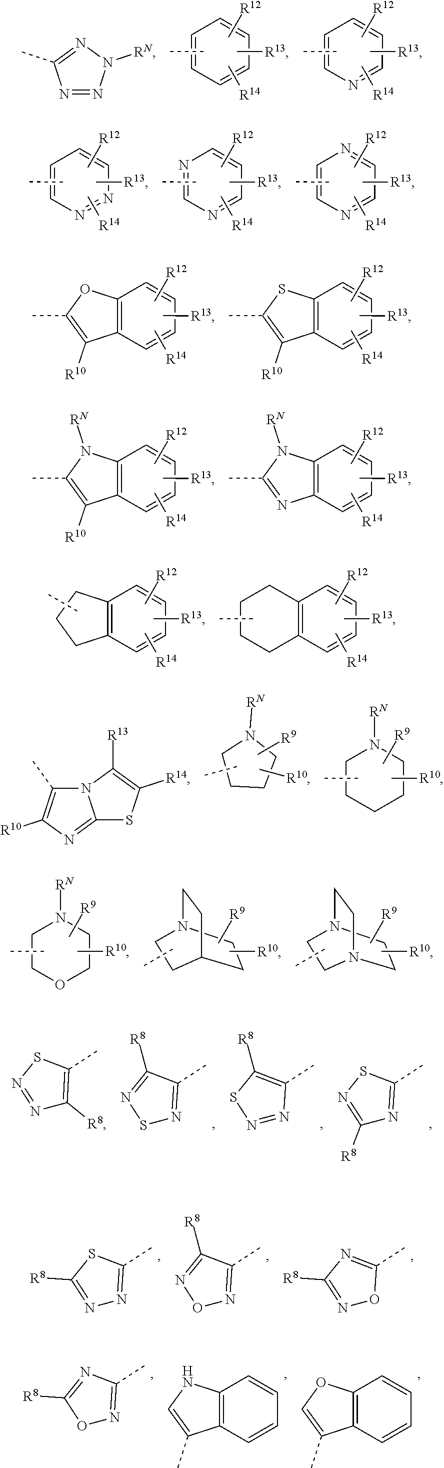

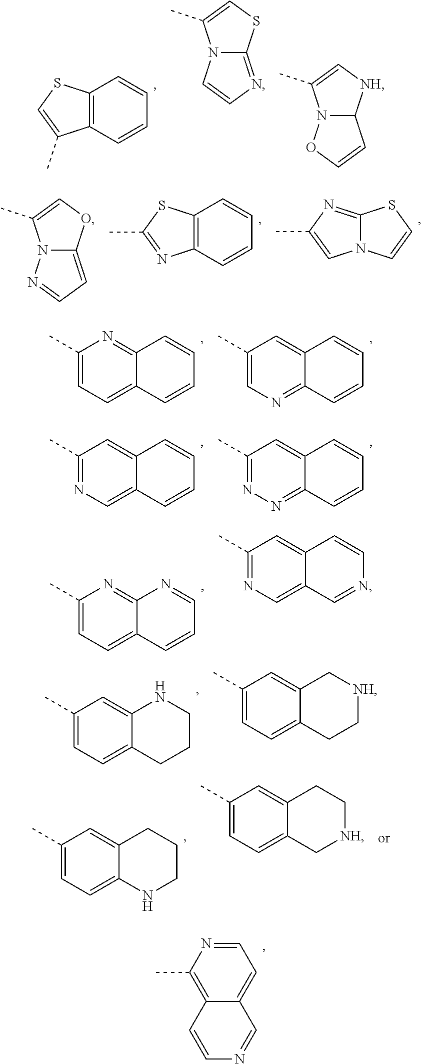

wherein the unsubstituted bicyclic residues can be substituted with 1 to 5 of the substituents R⁹-R¹⁴ and R^{N}; and preferably with 1 to 3 of the substituents R¹¹-R¹³ and the substituents R⁹-R¹⁴ and R^{N} have the meanings as defined herein.

Thus, the present invention relates to compounds of the general formula (I):

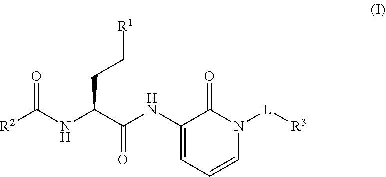

wherein
L represents -L¹- or -L¹-L²-; preferably -L¹-L²;
L¹ represents —CH₂—, or —CH₂CO—;
L² represents a bond, —NH—, —NHCH₂—, —NHCH₂CH₂—, or —NHCH(CH₃)—,
R¹ represents

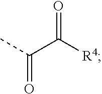

R² represents

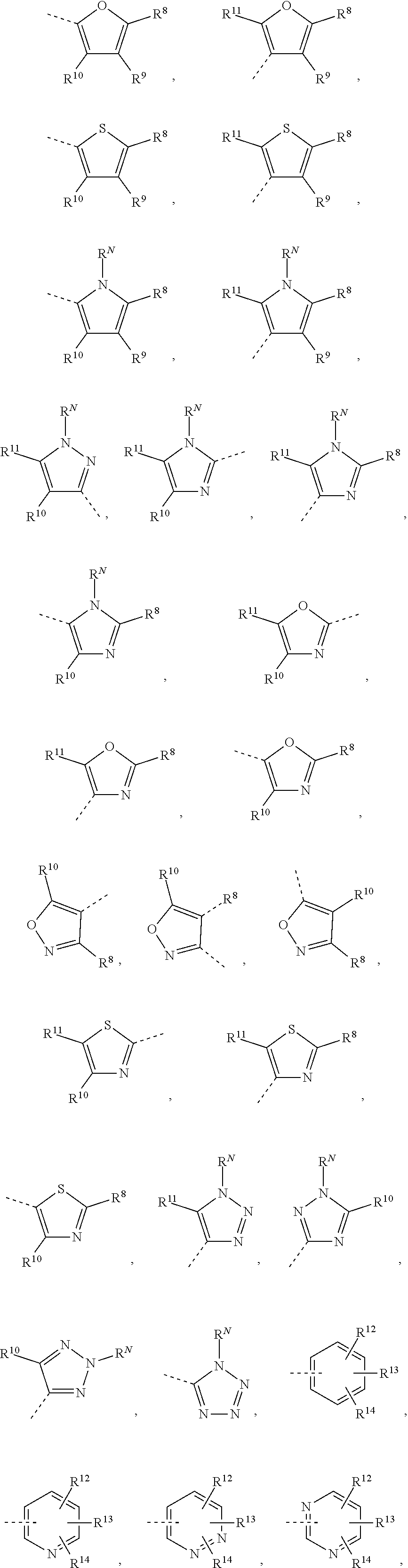

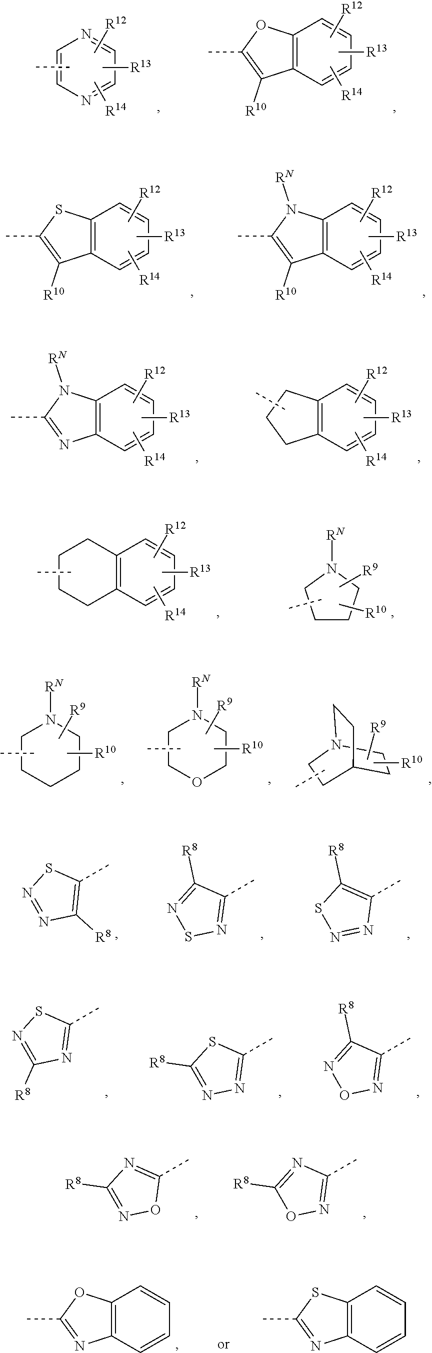

R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl and the afore-mentioned residues optionally contain one or more C═C double bond(s) and/or are optionally substituted by one or more of R^{a}, R^{b}, R^{c}, R^{d}, and R^{e};
R^{a}, R^{b}, R^{c}, R^{d}, and R^{e} represents independently of each other —H, —F, —Cl, —Br, —CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, —CONHCH₃, —CON(CH₃)₂, —CONHC₂H₅, —CH₂CO₂H, —CH₂CO₂CH₃, —CH₂CO₂C₂H₅, —CH₂CONH₂, —CH₂CONHCH₃, —CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, —NHSO₂CH₂CF₃;
R⁴ represents —NR⁶R⁷;
R⁶ and R⁷ represent independently of each other —H, —CH₃, —CH₂CH₂CH₃, —CH(CH₃)₂, —CH₂CH₂CH₂CH₃, —CH₂CH₂CH₂CH₂CH₃, —CH₂CH(CH₃)₂, —CH₂CH═CH₂, —CH₂CH═CH(CH₃), —CH₂CH═C(CH₃)₂, —CH₂CH═CHCH₂CH₃, -cyclo-C₃H₅, -cyclo-C₄H₇, -cyclo-C₅H₉, -cyclo-C₆H₁₁, —CH₂-cyclo-C₃H₅, —CH₂-cyclo-C₄H₇, —CH₂-cyclo-C₅H₉, —CH₂-cyclo-C₆H₁₁, —CH₂OCH₃, —CH₂OCH₂CH₃, —CH₂CH₂OCH₃, —CH₂CH₂OCH₂CH₃, —CH₂CH₂NHCH₃, —CH₂CH₂N(CH₃)₂, or —NR⁶R⁷ is —N(C₂H₅)₂,

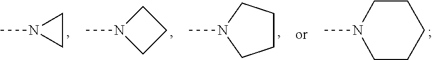

R⁸, R⁹, R¹⁰, R¹¹, R¹², R¹³, and R¹⁴ represent independently of each other —H, —F, —Cl, —Br, —OH, —CN, —NO₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, —CH₂-cyclo-C₃H₅, —CH₂OH, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —OCH₃, —OC₂H₅, —OC₃H₇, —OCH(CH₃)₂, —OC(CH₃)₃, —OC₄H₉, —OCHF₂, —OCF₃, —OCH₂CF₃, —OC₂F₅, —OCH₂OCH₃, —O-cyclo-C₃H₅, —OCH₂-cyclo-C₃H₅, —O—C₂H₄-cyclo-C₃H₅, —CHO, —COCH₃, —COCF₃, —COC₂H₅, —COC₃H₇, —COCH(CH₃)₂, —COC(CH₃)₃, —COOH, —COOCH₃, —COOC₂H₅, —COOC₃H₇, —COOCH(CH₃)₂, —COOC(CH₃)₃, —OOC—CH₃, —OOC—CF₃, —OOC—C₂H₅, —OOC—C₃H₇, —OOC—CH(CH₃)₂, —OOC—C(CH₃)₃, —NH₂, —NHCH₃, —NHC₂H₅, —NHC₃H₇, —NHCH(CH₃)₂, —NHC(CH₃)₃, —N(CH₃)₂, —N(C₂H₅)₂, —N(C₃H₇)₂, —N[CH(CH₃)₂]₂, —N[C(CH₃)₃]₂, —NHCOCH₃, —NHCOCF₃, —NHCOC₂H₅, —NHCOC₃H₇, —NHCOCH(CH₃)₂, —NHCOC(CH₃)₃, —CONH₂, —CONHCH₃, —CONHC₂H₅, —CONHC₃H₇, —CONHCH(CH₃)₂, —CONH-cyclo-C₃H₅, —CONHC(CH₃)₃, —CON(CH₃)₂, —CON (C₂H₅)₂, —CON (C₃H₇)₂, —CON[CH(CH₃)₂]₂, —CON[C(CH₃)₃]₂, —SO₂NH₂, —SO₂NHCH₃, —SO₂NHC₂H₅, —SO₂NHC₃H₇, —SO₂NHCH(CH₃)₂, —SO₂NH-cyclo-C₃H₅, —SO₂NHC(CH₃)₃, —SO₂N(CH₃)₂, —SO₂N(C₂H₅)₂, —SO₂N(C₃H₇)₂, —SO₂N[CH(CH₃)₂]₂, —SO₂N[C(CH₃)₃]₂, —NHSO₂CH₃, —NHSO₂CF₃, —NHSO₂C₂H₅, —NHSO₂C₃H₇, —NHSO₂CH(CH₃)₂, —NHSO₂C(CH₃)₃, —CH═CH₂, —CH₂—CH═CH₂, —C(CH₃)=CH₂, —CH═CH—CH₃, —C≡CH, —C≡C—CH₃, —CH₂—C≡CH, -Ph, —O-Ph, —O—CH₂-Ph,

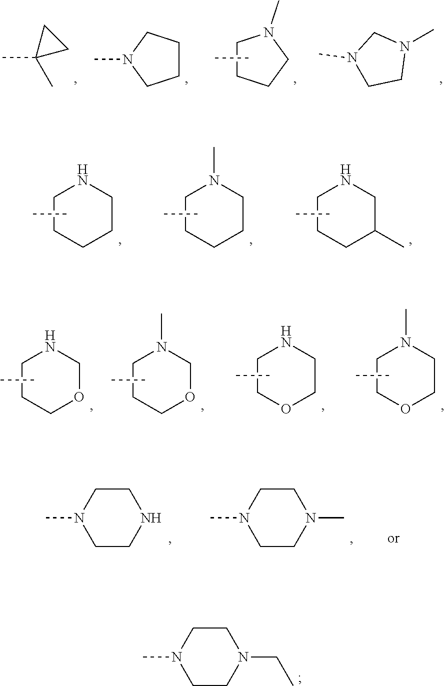

or R⁸ and R⁹ or R⁹ and R¹⁰ can form together one of the following five-membered or six-membered rings:

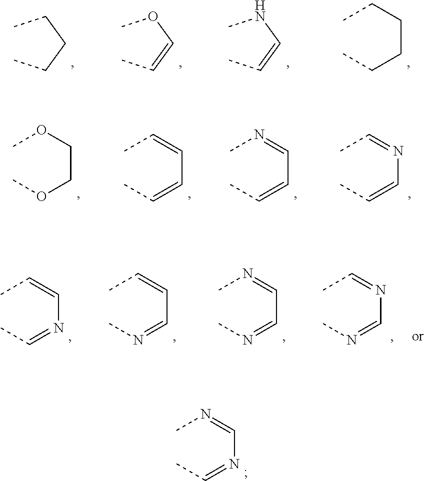

or R¹² and R¹³ or R¹³ and R¹⁴ can form together one of the following five-membered or six-membered rings:

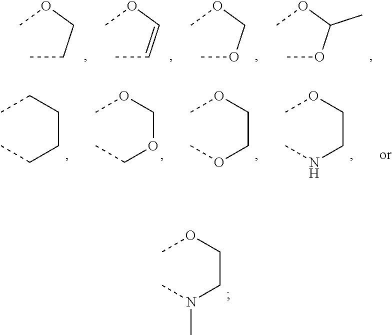

R^{N} represents —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, -cyclo-C₄H₇, -cyclo-C₅H₉, —CH₂-cyclo-C₃H₅, —CH₂-cyclo-C₄H₇, —CH₂-cyclo-C₅Hg, —CH₂F, —CHF₂, —CF₃, —CH₂Cl, —CH₂Br, —CH₂I, —CH₂—CH₂F, —CH₂—CHF₂, —CH₂ CF₃, —CH₂—CH₂Cl, —CH₂—CH₂Br, —CH₂—CH₂I, —CH₂—CH═CH₂, —CH₂—C≡CH, —CHO, —COCH₃, —COC₂H₅, —COC₃H₇, —COCH(CH₃)₂, —COC(CH₃)₃, —CO-cyclo-C₃H₅, —CO-cyclo-C₄H₇, —CO-cyclo-C₅H₉, —COOCH₃, —COOC₂H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, —SO₂-cyclo-C₃H₅, or —SO₂C(CH₃)₃,
or a diastereomer, an enantiomer, a mixture of diastereomers, a mixture of enantiomers, a racemate, a solvate, a hydrate, or a pharmaceutically acceptable salt thereof.

Preferred are the compounds of the formula (Ib):

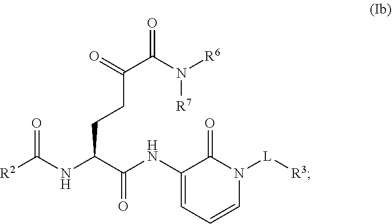

and L, R², R³, R⁶, R⁷ have the same meanings as defined in the formula (I)

Preferred are the compounds of the formula (Ib):

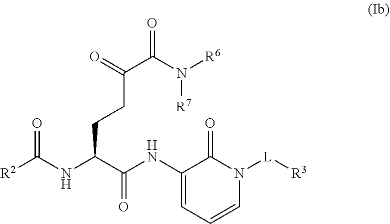

wherein
L represents -L¹- or -L¹-L²-; preferably -L¹-L²-;
L¹ represents —CH₂—, —CH₂CH₂—, —CH₂CH₂CH₂—, —CH₂CO—, or —CH₂CH₂CO—;
L² represents a bond, —NR^{N1}—, —NR^{N1}CH₂—, —NR^{N1}CH₂CH₂—, or —NR^{N1}CH(CH₃)—;
R² represents

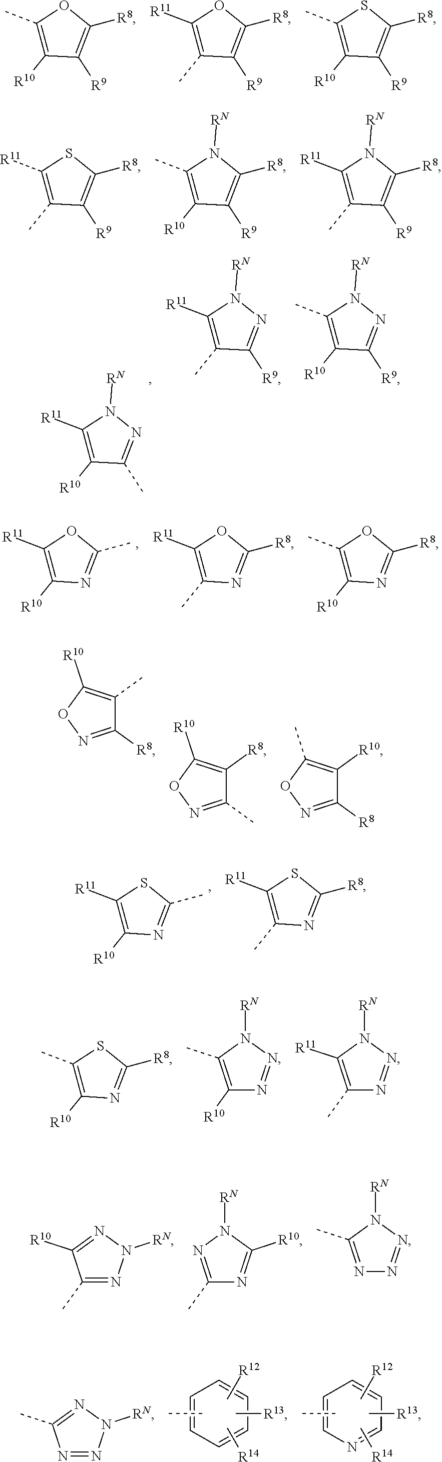

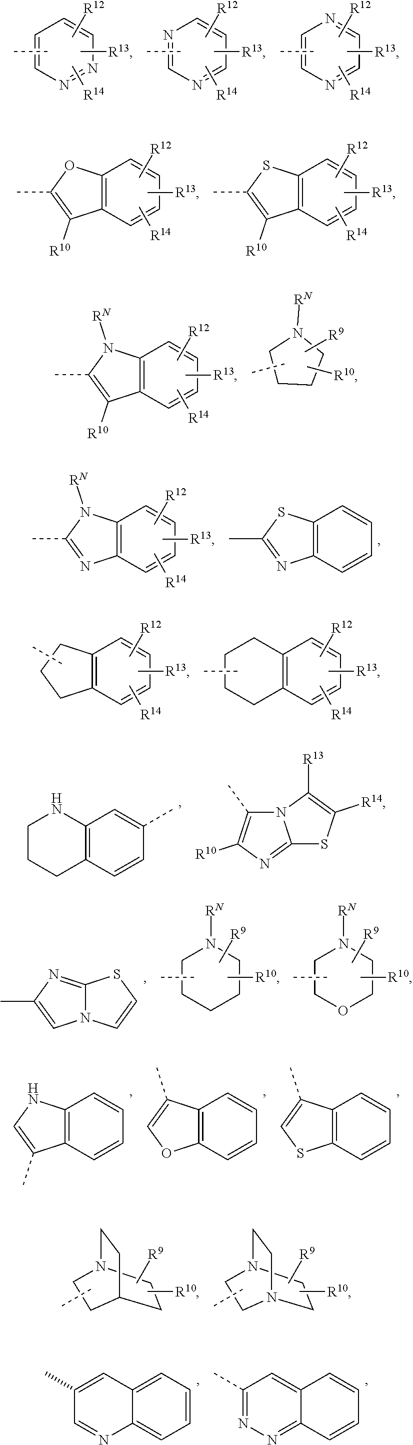

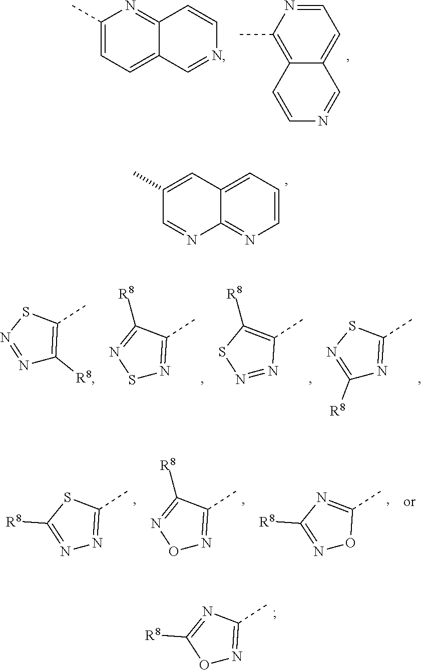

R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl, and the afore-mentioned residues optionally contain one or more C═C double bond(s) and/or are optionally substituted with one or more of R^{a}, R^{b}, R^{c}, R^{d}, and R^{e};
R^{a}, R^{b}, R^{c}, R^{d}, and R^{e} represents independently of each other —H, —F, —Cl, —Br, —CN, —OH, —CH₃, —CH₂CH₃, —CH₂CH₂CH₃, —CH(CH₃)₂, —CHF₂, —CF₃, —COCH₂CH₃, —CO₂H, —CO₂CH₃, —CO₂C₂H₅, —CONH₂, —CH₂CF₃, —COCH₃, —CONHCH₃, —CON(CH₃)₂, —CONHC₂H₅, —CH₂CO₂H, —CH₂CO₂CH₃, —CH₂CO₂C₂H₅, —CH₂CONH₂, —CH₂CONHCH₃, —CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, —NHSO₂CH₂CF₃;
R⁴ represents —NR⁶R⁷;
R⁶ and R⁷ represent independently of each other —H, —CH₃, —CH₂CH₂CH₃, —CH(CH₃)₂, —CH₂CH₂CH₂CH₃, —CH₂CH₂CH₂CH₂CH₃, —CH₂CH(CH₃)₂, —C(CH₃)₃, —CH₂CH═CH₂, —CH₂CH═CH(CH₃), —CH₂CH═C(CH₃)₂, —CH₂CH═CHCH₂CH₃, -cyclo-C₃H₅, -cyclo-C₄H₇, -cyclo-C₅H₉, -cyclo-C₆H₁₁, —CH₂-cyclo-C₃H₅, -Ph, —CH₂-Ph, —CH₂-cyclo-C₄H₇, —CH₂-cyclo-C₅H₉, —CH₂-cyclo-C₆H₁₁, —CH₂OCH₃, —CH₂OCH₂CH₃, —CH₂CH₂OCH₃, —CH₂CH₂OCH₂CH₃, —CH₂CH₂NHCH₃, —CH₂CH₂N(CH₃)₂, or —NR⁶R⁷ is —N(C₂H₅)₂,

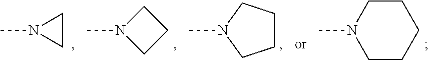

R^{N} represents —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, -cyclo-C₄H₇, -cyclo-C₅H₉, —CH₂-cyclo-C₃H₅, —CH₂F, —CHF₂, —CF 3, —CH₂Cl, —CH₂Br, —CH₂I, —CH₂—CH₂F, —CH₂ CHF₂, —CH₂—CF₃, —CH₂—CH₂Cl, —CH₂—CH₂Br, —CH₂—CH₂I, —CH₂—CH═CH₂, —CH₂—C═CH, —CHO, —COCH₃, —COC₂H₅, —COC₃H₇, —COCH(CH₃)₂, —COC(CH₃)₃, —COOCH₃, —COOC 2H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, or —SO₂C(CH₃)₃,
R^{N1} represent —H, —CH₃, or —CH₂CH₃;
and R⁸-R¹⁴ have the meanings as defined above for formula (I);
or a diastereomer, an enantiomer, a mixture of diastereomers, a mixture of enantiomers, a racemate, a solvate, a hydrate, or a pharmaceutically acceptable salt thereof.

Preferably, R² represents

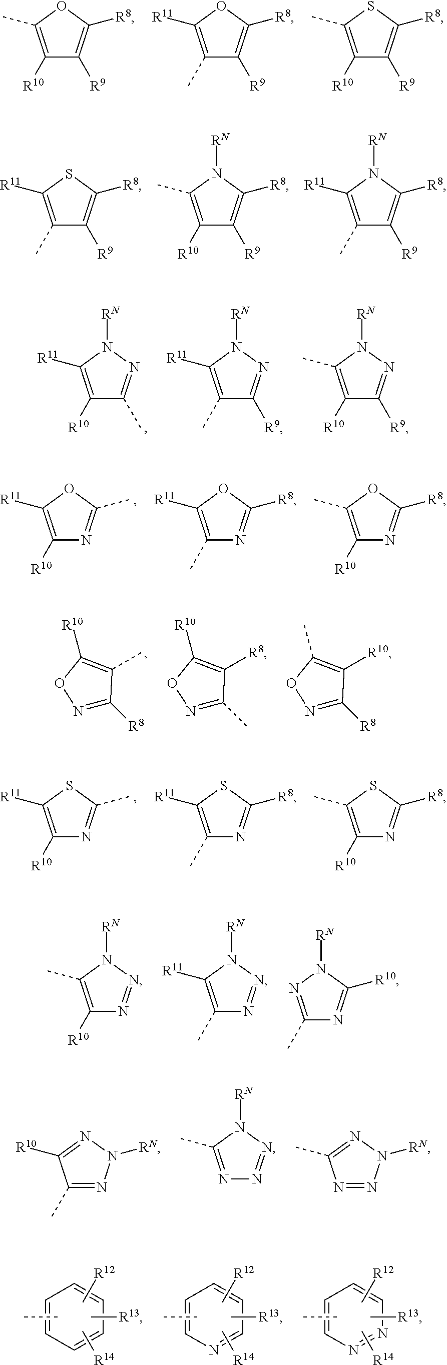

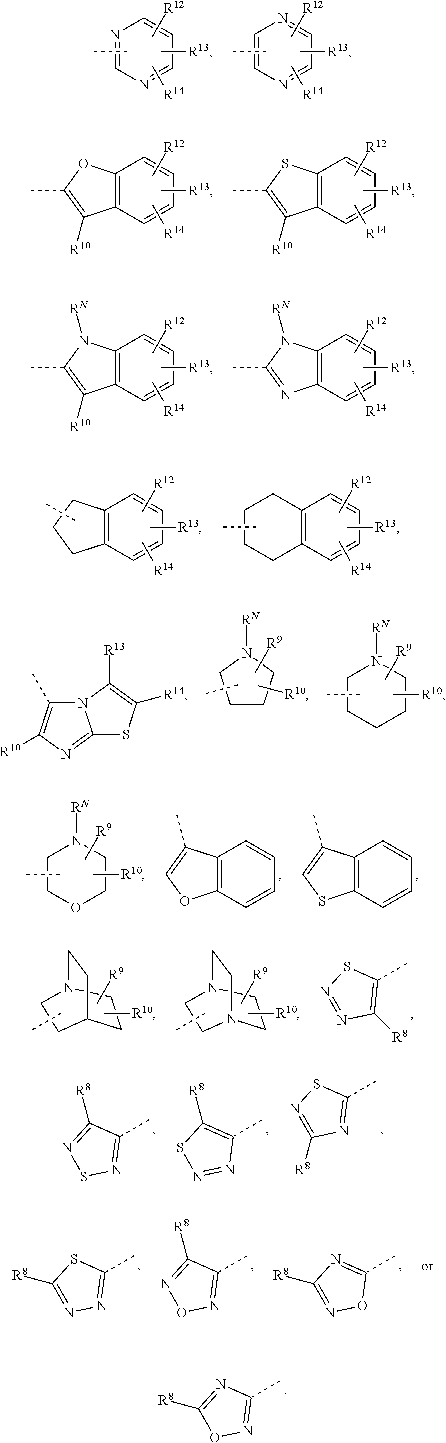

Preferably, —NR⁶R⁷ of the formula (Ib) represents-NH₂, —NHCH₃, —N(CH₃)₂, —NHCH(CH₃)₂, —NHCH₂CH₂CH₃, —NH—CH₂CH═CH₂, —NHCH₂CH₂CH₂CH₃, —NHCH₂CH(CH₃)₂, —NHC(CH₃)₃, —NH-cyclo-C₃H₅, —NHCH₂CH₂CH₂CH₂CH₃, —NH-cyclo-C₄H₇, —NH-cyclo-C₅H₉, —NH-cyclo-C₆H₁₁, —NHCH₂-cyclo-C₃H₅, —NHCH₂-cyclo-C₄H₇, —NHCH₂-cyclo-C₅H₉, —NHCH₂ cyclo-C₆H₁₁, —NHCH₂-Ph, —NHCH₂OCH₃, —NHCH₂OCH₂CH₃, —NHCH₂CH₂OCH₃, —NHCH₂CH₂NHCH₃, —NHCH₂CH₂N(CH₃)₂,

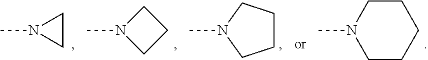

In some embodiments, the present invention relates to the compound of the formula (I),

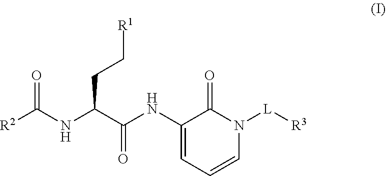

wherein
L represents -L¹- or -L¹-L²-; preferably -L¹-L²-;
L¹ represents —CH₂—, —CH₂CH₂—, —CH₂CH₂CH₂—, —CH₂CO—, or —CH₂CH₂CO—;
L² represents a bond, —NR^{N1}-, —NR^{N1}CH₂—, —NR^{N1}CH₂CH₂—, or —NR^{N1}CH(CH₃)—;
R¹ represents

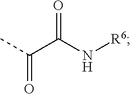

R² represents

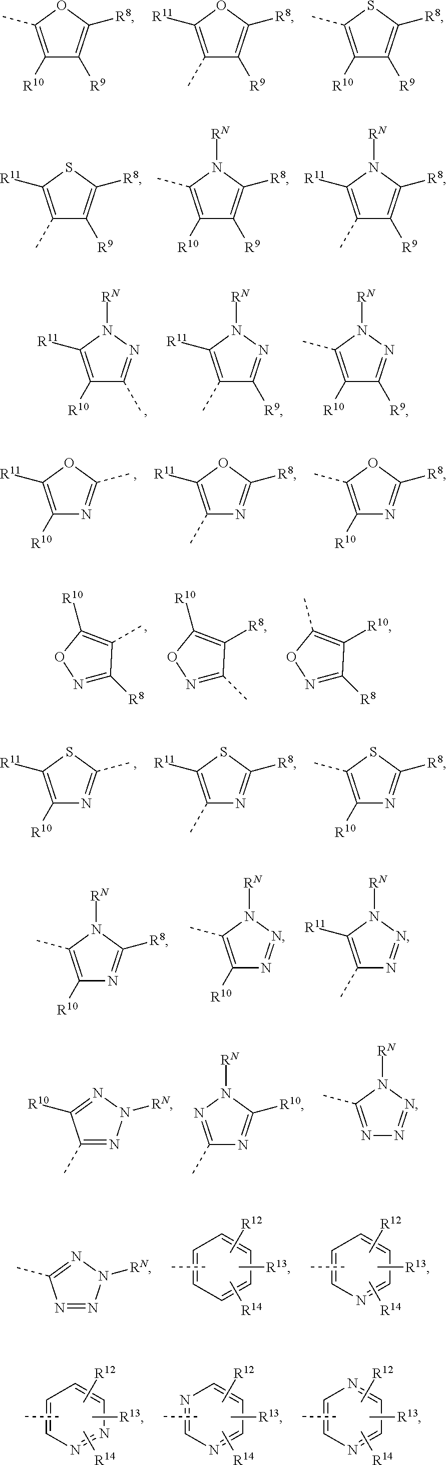

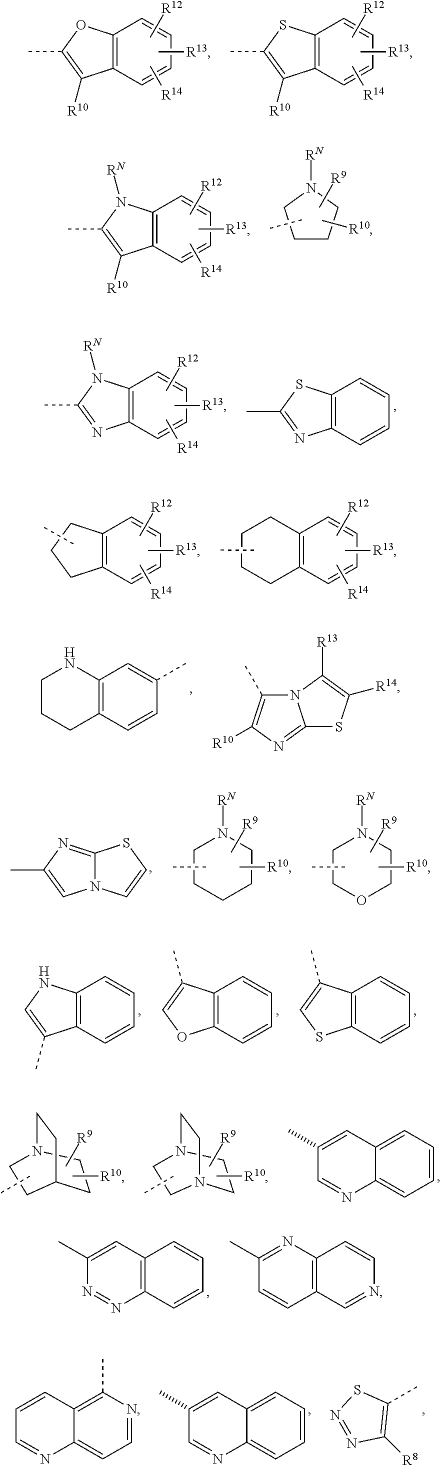

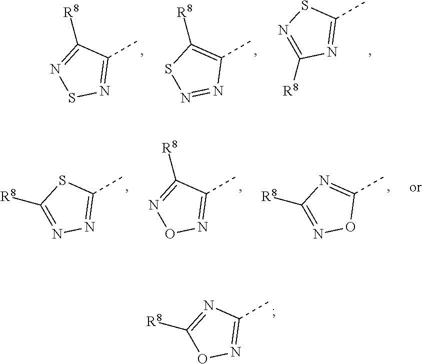

R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl, and the afore-mentioned residues optionally contain one or more C═C double bond(s) and/or are substituted with one or more of R^{a}, R^{b}, R^{c}, R^{d}, and R^{e};
R⁶ represents —H, —CH₃, —CH₂CH═CH₂, -cyclo-C₃H₅, —CH₂CH₂CH₂CH₂CH₃; and R⁸-R¹⁴, R^{a}, R^{b}, R^{c}, R^{d}, R^{e}, R^{N}, and R^{N1} have the meanings and preferred meanings as defined herein.

Also preferred are compounds of the general formula (I),

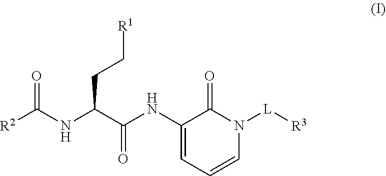

wherein
L represents -L¹- or -L¹-L²-;
L¹ represents —CH₂—, —CH₂CH₂, —CH₂CH₂CH₂—, —CH₂CO—, —CH₂CH₂CO—;
L² is a bond, —NR^{N1}—, —NR^{N1}CH₂—, —NR^{N1}CH₂CH₂—, or —NR^{N1}CH(CH₃)—;
R¹ represents

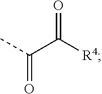

R² represents

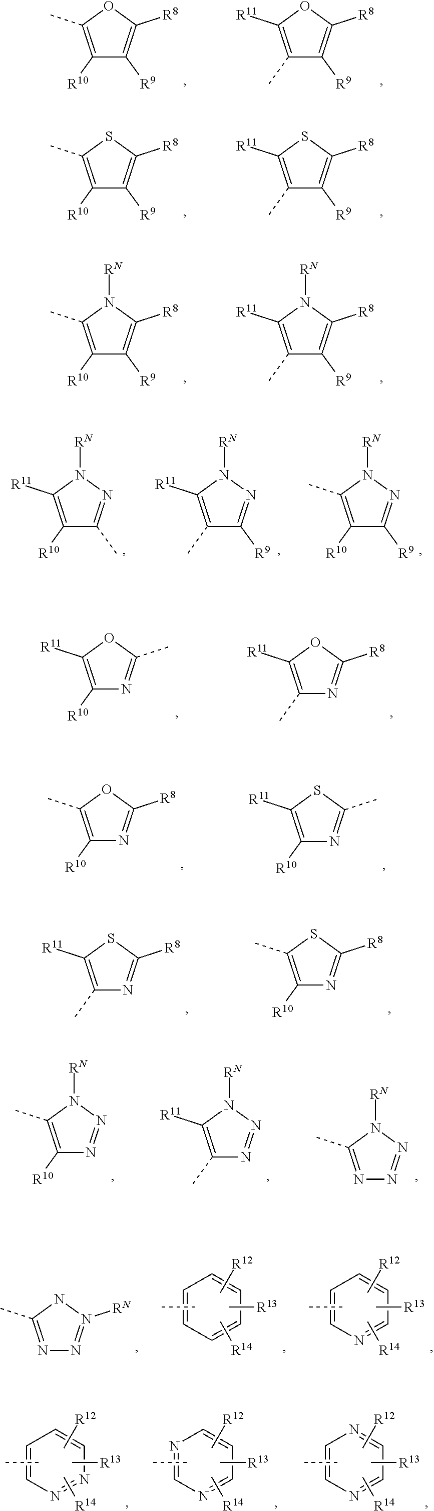

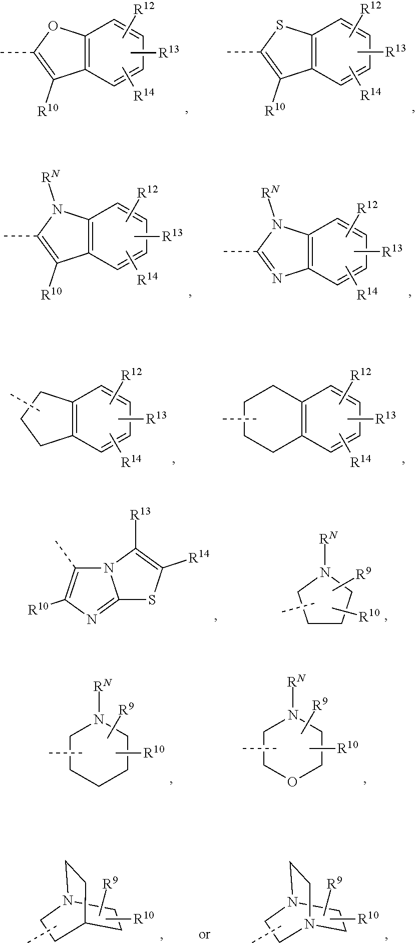

R³ represents bicyclo[1.1.1]pentyl, bicyclo[2.1.1]hexyl, bicyclo[2.2.1]heptyl, bicyclo[3.2.2]nonyl, bicyclo[3.1.1]heptyl, bicyclo[2.2.2]octyl, bicyclo[3.2.1]octyl, bicyclo[3.3.2]decyl, bicyclo[3.3.3]undecyl, 4-homoisotwistyl, adamantyl, diamantyl, hexamethylenetetraminyl, and the afore-mentioned residues optionally contain one or more C═C double bond and/or are substituted one or more of R^{a}, R^{b}, R^{c}, R^{d}, and R^{e};
R^{a}, R^{b}, R^{c}, R^{d}, and R^{e} represents independently of each other —H, —F, —Cl, —Br, —CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, —CONHCH₃, —CON(CH₃)₂, —CONHC₂H₅, —CH₂CO₂H, —CH₂CO₂CH₃, —CH₂CO₂C₂H₅, —CH₂CONH₂, —CH₂CONHCH₃, —CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, —NHSO₂CH₂CF₃;
R⁴ represents —NR⁶R⁷;
R⁶ and R⁷ represent independently of each other —H, —CH₃, —CH₂CH₂CH₃, —CH(CH₃)₂, —CH₂CH₂CH₂CH₃, —CH₂CH₂CH₂CH₂CH₃, —CH₂CH(CH₃)₂, —C(CH₃)₃, —CH₂CH═CH₂, —CH₂CH═CH(CH₃), —CH₂CH═C(CH₃)₂, —CH₂CH═CHCH₂CH₃, -cyclo-C₃H₅, -cyclo-C₄H₇, -cyclo-C₅H₉, -cyclo-C₆H₁₁, —CH₂-cyclo-C₃H₅, —CH₂-cyclo-C₄H₇, —CH₂-cyclo-C₅H₉, —CH₂-cyclo-C₆H₁₁, —CH₂-Ph, —CH₂OCH₃, —CH₂OCH₂CH₃, —CH₂CH₂OCH₃, —CH₂CH₂OCH₂CH₃, —CH₂CH₂NHCH₃, —CH₂CH₂N(CH₃)₂, or —NR⁶R⁷ represents

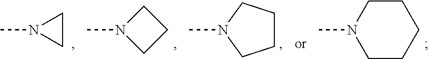

R^{N} represents —H, —CH₃, —C₂H₅, —C₃H₇, —CH(CH₃)₂, —C₄H₉, —CH₂—CH(CH₃)₂, —CH(CH₃)—C₂H₅.—C(CH₃)₃, -cyclo-C₃H₅, -cyclo-C₄H₇, -cyclo-C₅H₉, —CH₂-cyclo-C₃H₅, —CH₂F, —CHF₂, —CF₃, —CH₂Cl, —CH₂Br, —CH₂I, CH₂—CH₂F, CH₂—CHF₂, —CH₂—CF 3, —CH₂—CH₂Cl, CH₂—CH₂Br, —CH₂—CH₂I, —CH₂—CH═CH₂, —CH₂—C═CH, —CHO, —COCH₃, —COC₂H₅, —COC₃H₇, —COCH(CH₃)₂, —COC(CH₃)₃, —COOCH₃, —COOC 2H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, or —SO₂C(CH₃)₃;
R^{N1} represent —H, —CH₃, or —CH₂CH₃;
or a diastereomer, an enantiomer, a mixture of diastereomers, a mixture of enantiomers, a racemate, a solvate, a hydrate, or a pharmaceutically acceptable salt thereof.

Also preferred are compounds of the formula (I) or (Ib), wherein
L¹ represents —CH₂—, or —CH₂CO—;
L² represents a bond, —NR^{N1}—, —NR^{N1}CH₂—, or —NR^{N1}CH(CH₃)—;
R³ represents bicyclo[1.1.1]pentyl, bicyclo[2.1.1]hexyl, bicyclo[2.2.1]heptyl, bicyclo[3.1.1]heptyl, bicyclo[2.2.2]octyl, 4-homoisotwistyl, adamantyl, or diamantyl, and the afore-mentioned residues optionally contain one or more C═C double bond(s) and/or are substituted by one or more of R^{a}, R^{b}, R^{c}, R^{d}, and R^{e}; and R^{a}, R^{b}, R^{c}, R^{d}, R^{e} and R^{N1} have the same meanings as defined herein.

Preferably, R² of the formula (I) or (Ib) represents

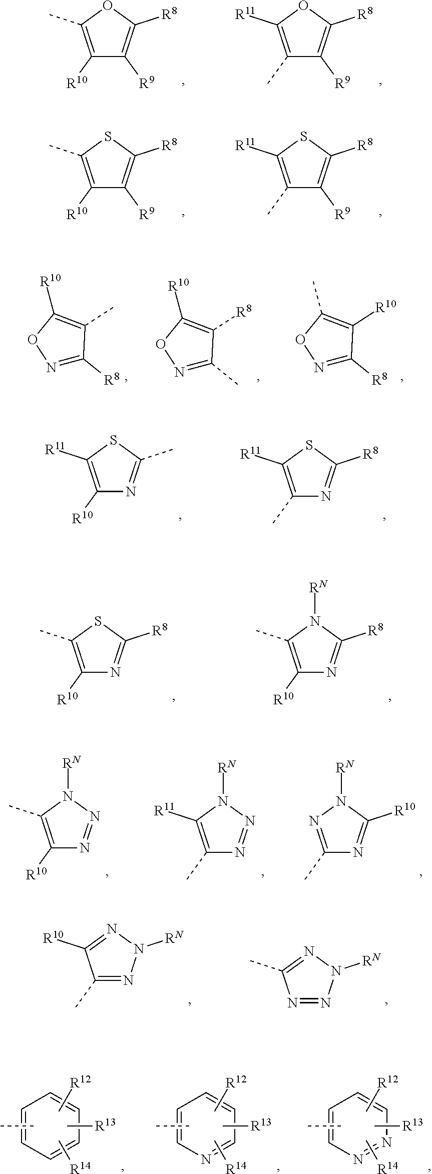

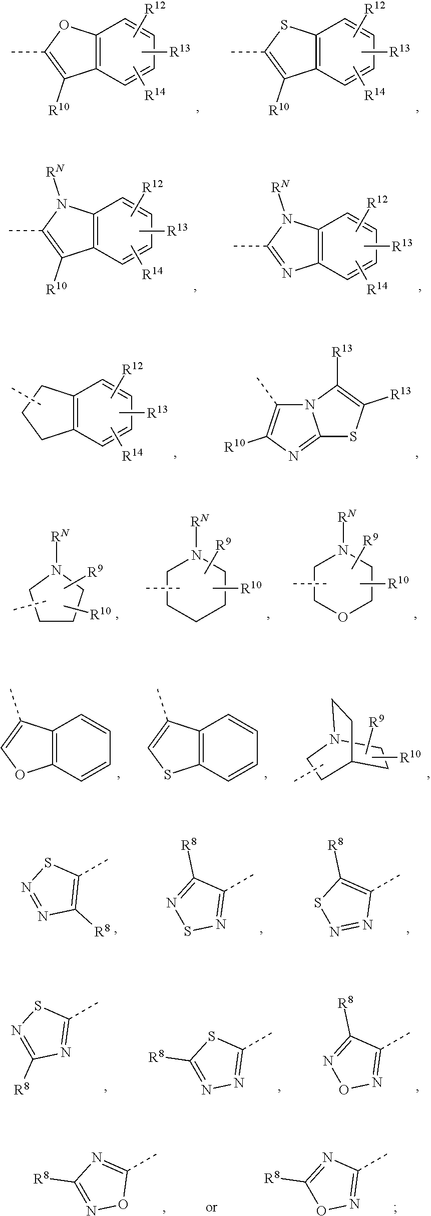

and R⁸-R¹⁴ and R^{N} have the meanings as defined in formula (I) or (Ib).

Preferred are compounds having any one of the formulae (IV-a)-(IV-o) and (V-a)-(V-d):

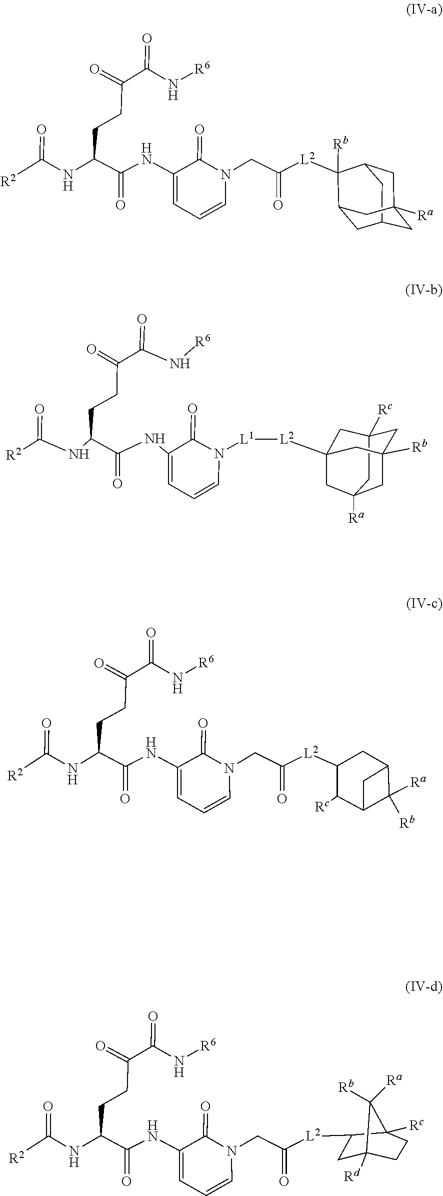

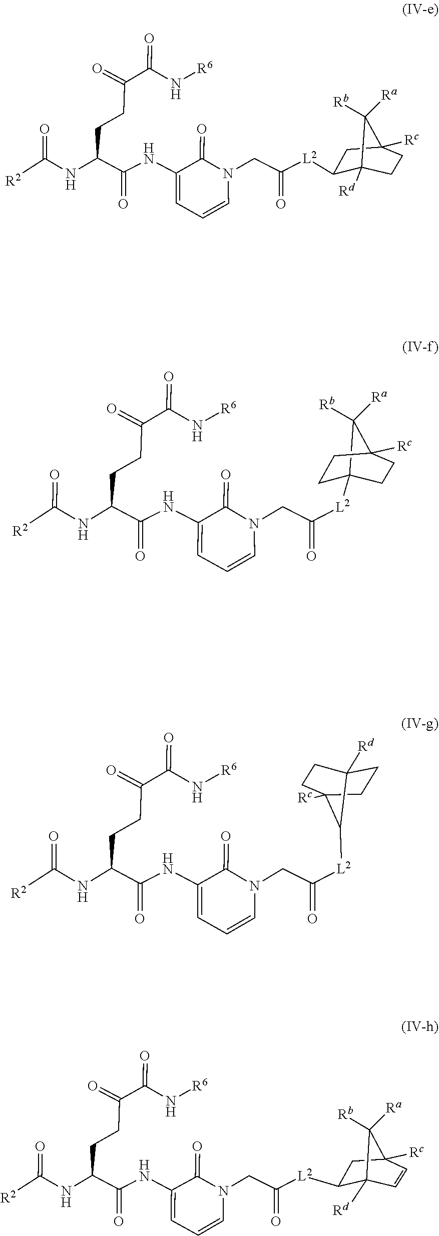

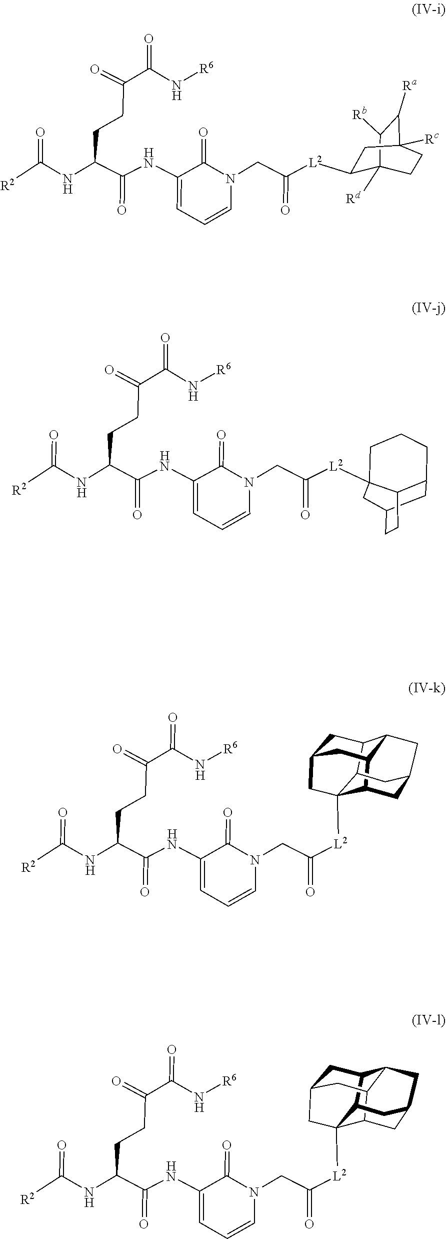

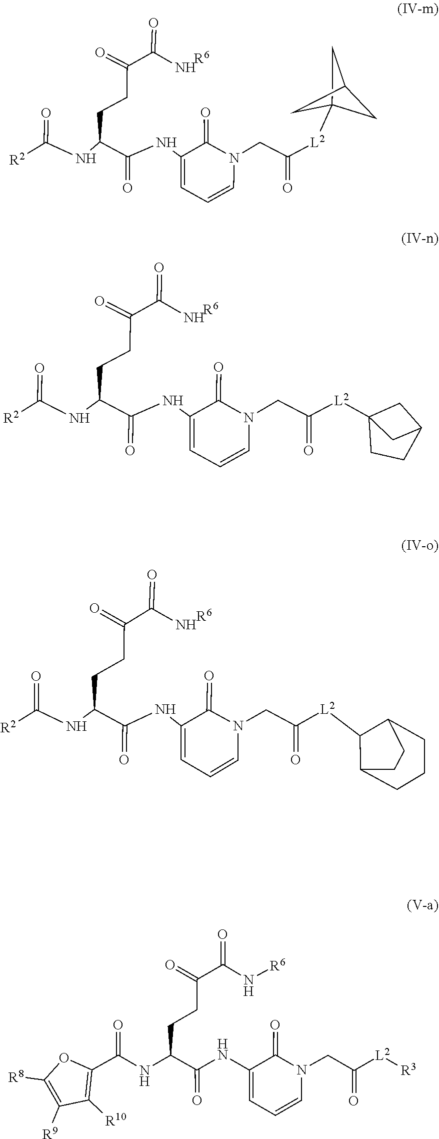

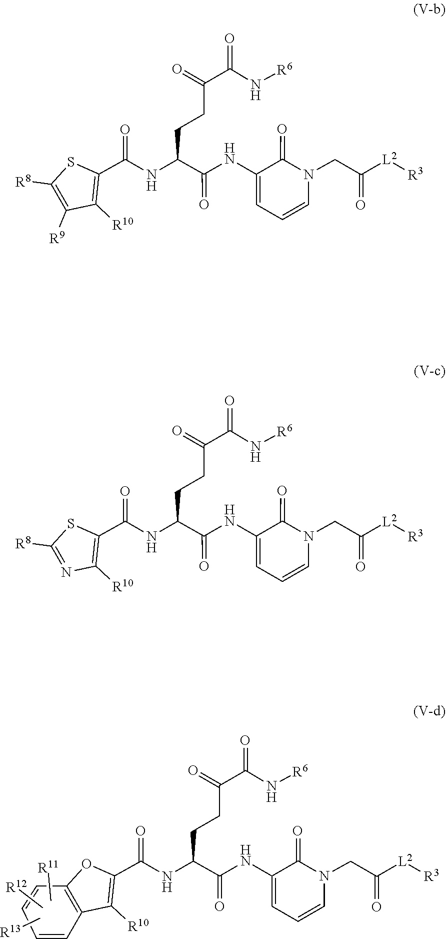

and R², R³, R⁶, R⁸, R⁹, R¹⁰, R¹¹, R¹², R¹³, R^{a}, R^{b}, R^{c}, R^{d} and L² have the same meanings as defined herein, preferably as defined in formula (I) or (Ib).

Also preferred are the compounds of any of the formula (IVa-1),

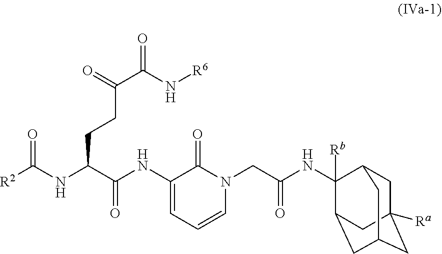

wherein
R⁶ represents —H, —CH₃, —CH(CH₃)₂, —CH₂CH₂CH₃, —CH₂CH═CH₂, —CH₂CH₂CH₂CH₃, —CH₂CH(CH₃)₂, —C(CH₃)₃, —CH₂CH₂CH₂CH₂CH₃, -cyclo-C₃H₅, -cyclo-C₅H₉, -cyclo-C₆H₁₁ or —CH₂ cyclo-C₃H₅.
and R², R^{a}, and R^{b} have the same meanings as defined above.

Preferably, in the compounds of formula (I) or (Ib), R^{a} and R^{b} represent independently of each other —H, —F, —Cl, —Br, —OH, —CN, —CH₃, —C₂H₅, or —CO₂Me.

Preferably, in any of the formula (I), (lb), (IV-a)-(IV-o), or (IVa-1):
R² represents

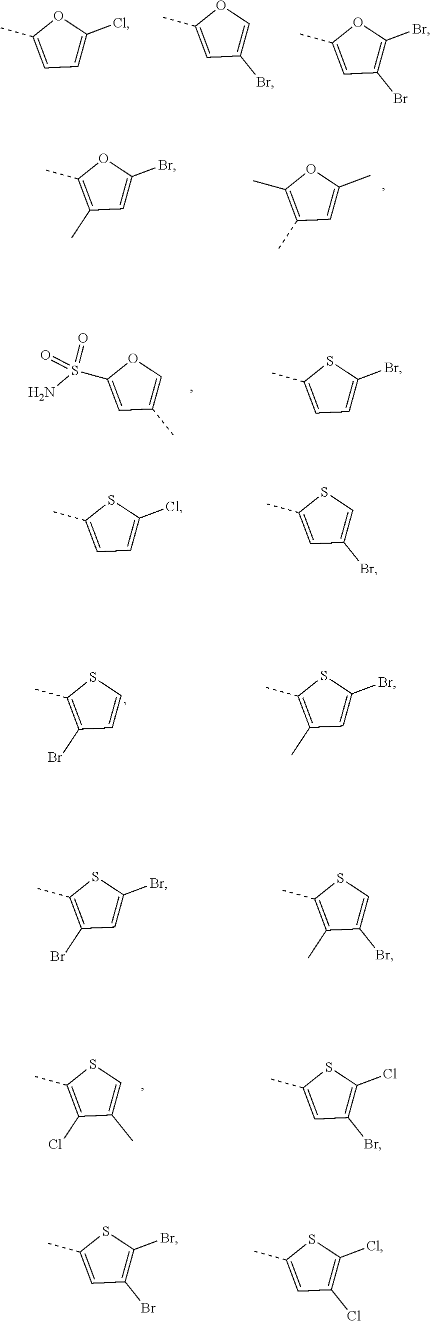

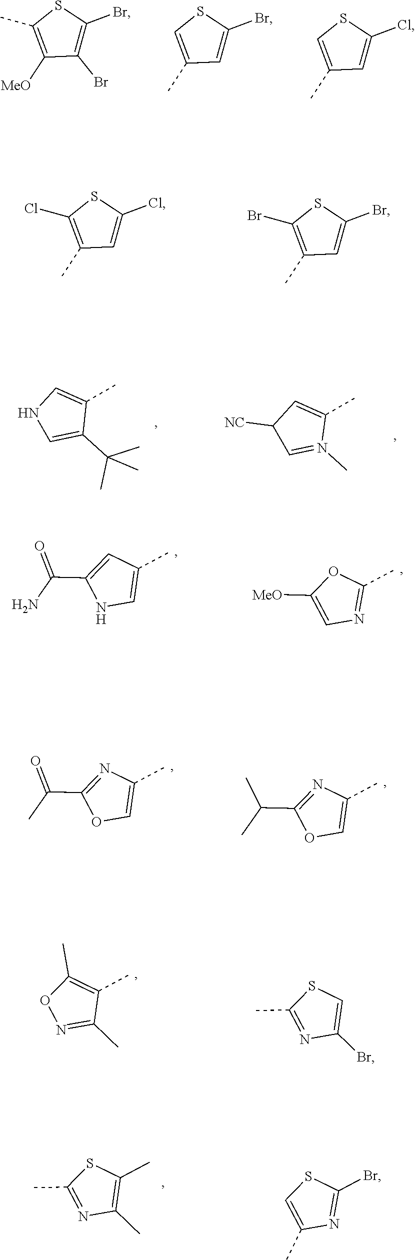

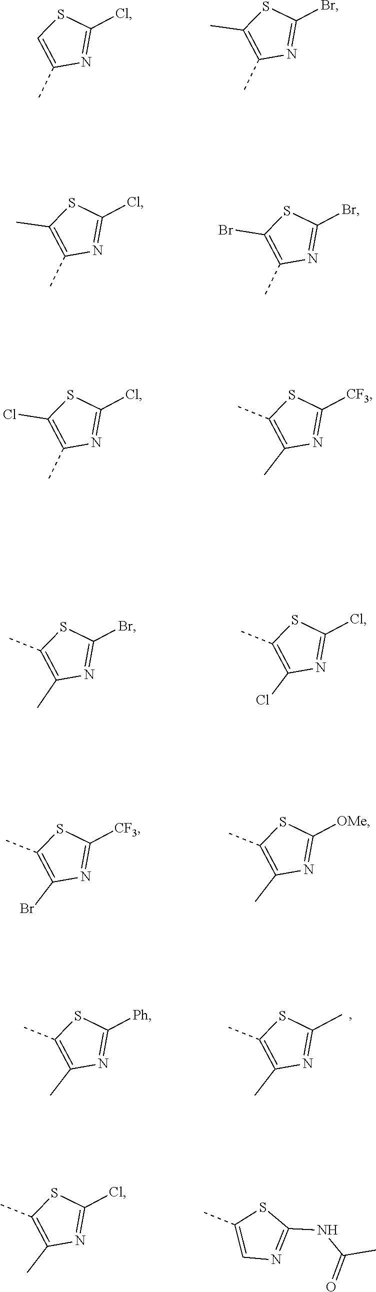

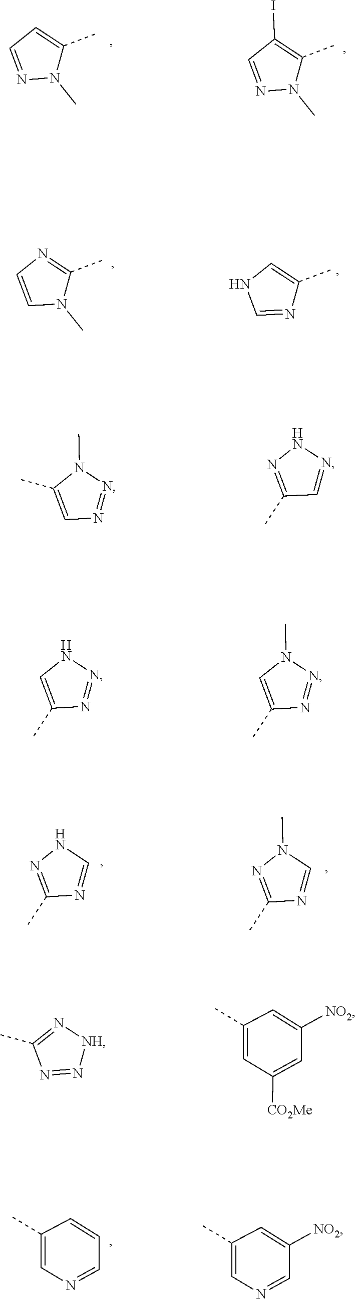

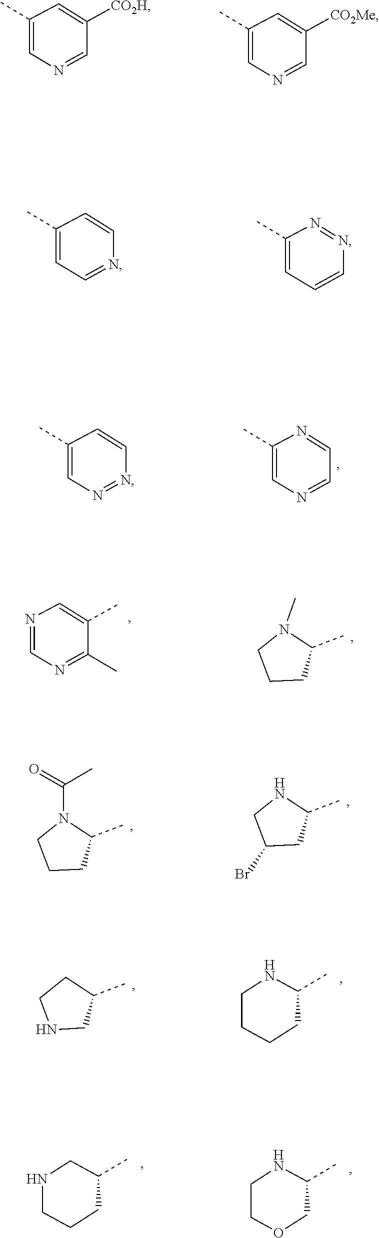

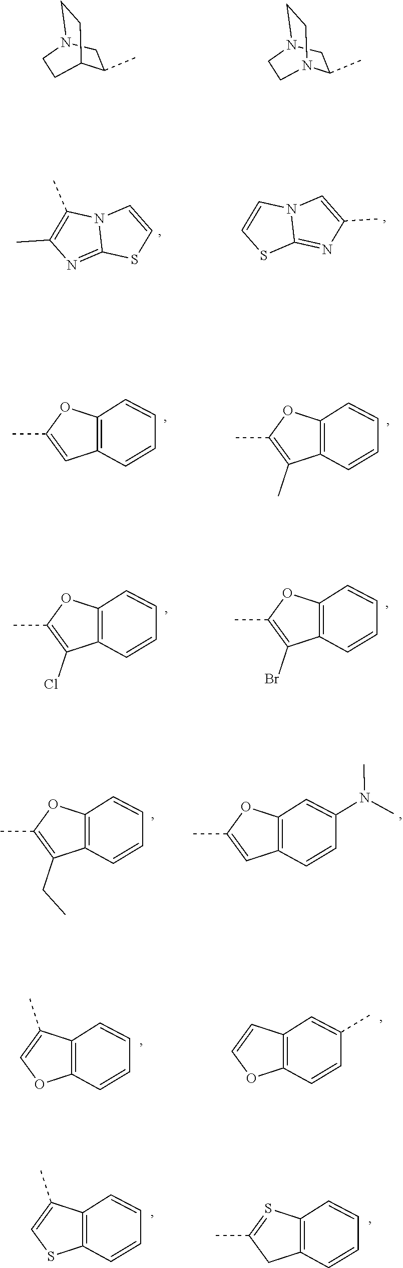

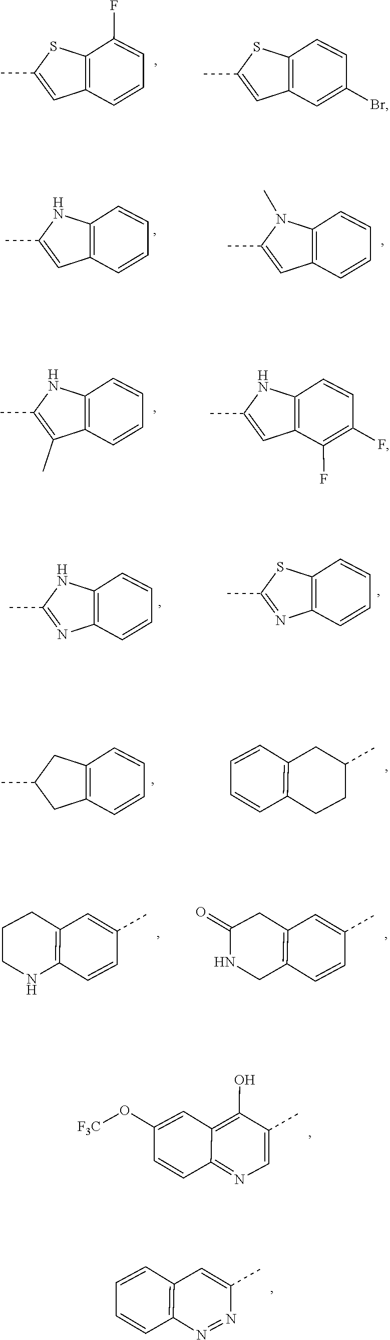

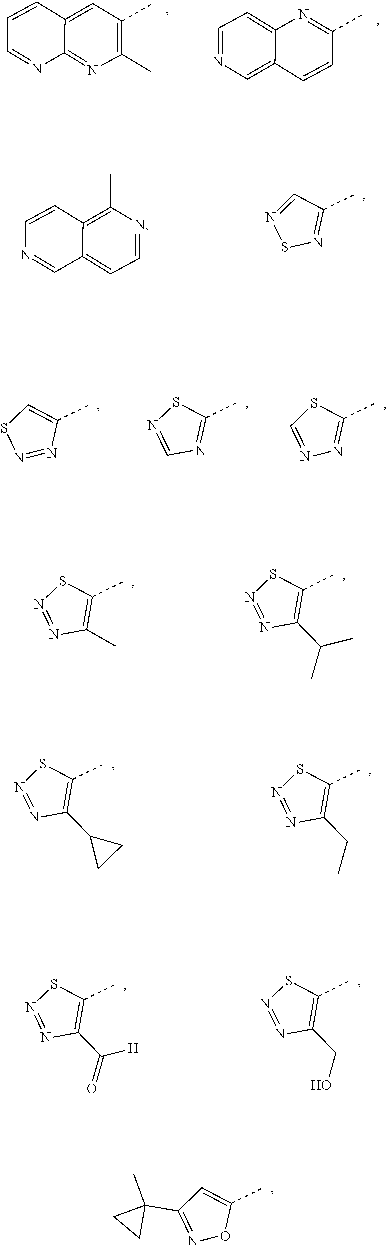

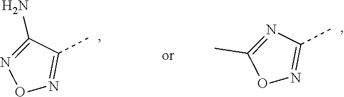

and
R⁶ represents —H, —CH₃, —CH(CH₃)₂, —CH₂CH₂CH₃, —CH₂CH═CH₂, —CH₂CH₂CH₂CH₃, —CH₂CH(CH₃)₂, —C(CH₃)₃, —CH₂CH₂CH₂CH₂CH₃, -cyclo-C₃H₅, -cyclo-C₅H₉, -cyclo-C₆H₁₁ or —CH₂-cyclo-C₃H₅.

Due to the specially selected substituents R² on the N-terminal side and substituents R³ on the C-terminal side and of the inventive compound according to the invention the steric dimension can be adjusted very precisely, so that a binding pocket of a desired target molecule may be addressed with highly matching measurements.

Preferred, are the compound of any of the formulae (I), (Ib), (IV-a)-(IV-o), and (V-a)-(V-d), wherein
R³ represents

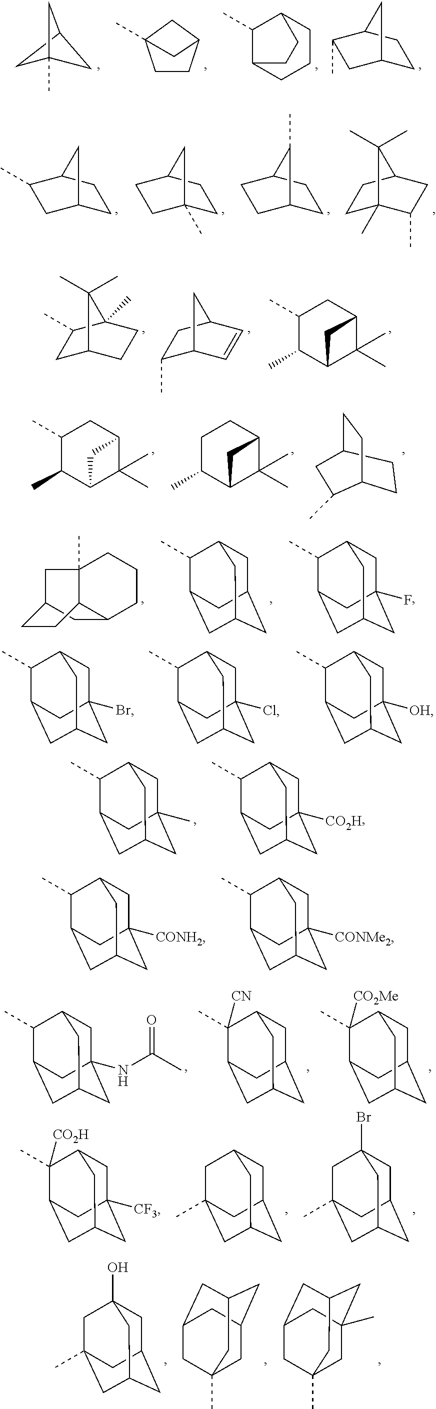

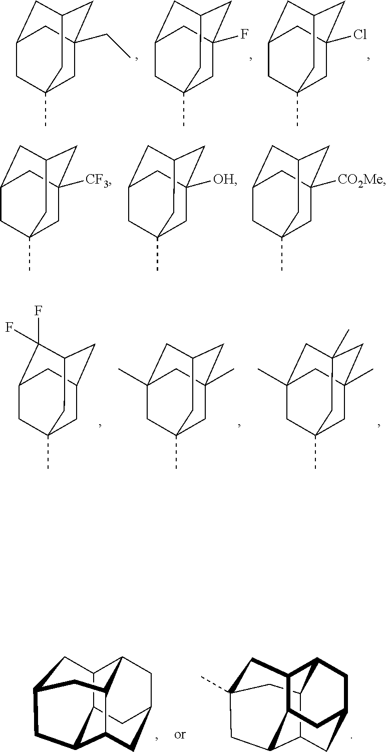

Surprisingly, it was found that the inventive compounds bound to the transglutaminase 2 reversibly and inhibit the transglutaminase effectively. The electrophilic warheads in combination with the preferred embodiment specifically react with highly nucleophilic thiols in the active site of the transglutaminase 2. Accordingly, it was found that potential unspecific reactions with off-targets are reduced.

In one embodiment, the present invention refers to the compound selected from the group consisting of:

| | | |
|---|---|---|
| II-2: | 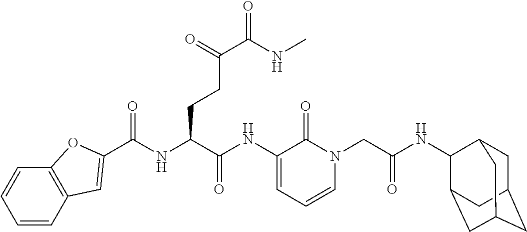 [SMILES: CNC(=O)C(=O)CC[C@H](NC(=O)c1cc2ccccc2o1)C(=O)Nc1cccn(CC(=O)NC2C3CC4CC(C3)CC2C4)c1=O] | (S)-2-(benzofuran-2-carboxamido)-N1-(1- (2-(2-adamantylamino)-2-oxoethyl)-2-oxo- 1,2-dihydropyridin-3-yl)-N6-methyl-5- oxohexanediamide |
| II-3: | 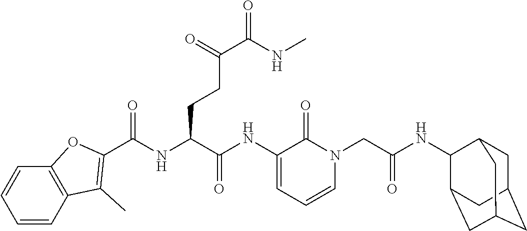 [SMILES: CNC(=O)C(=O)CC[C@H](NC(=O)c1oc2ccccc2c1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(3-methylbenzofuran-2- carboxamido)-5-oxohexanediamide |
| II-4: | 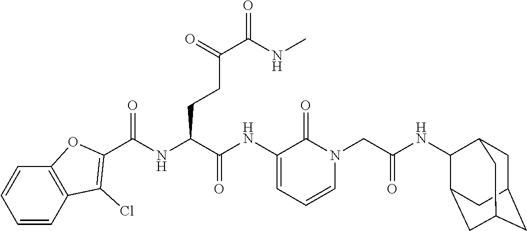 [SMILES: CNC(=O)C(=O)CC[C@H](NC(=O)c1oc2ccccc2c1Cl)C(=O)Nc1cccn(CC(=O)NC2C3CC4CC(C3)CC2C4)c1=O] | (S)-2-(3-chlorobenzofuran-2- carboxamido)-N1-(1-(2-(2- adamantylamino)-2-oxoethyl)-2-oxo-1,2- dihydropyridin-3-yl)-N6-methyl-5- oxohexanediamide |
| II-5: | 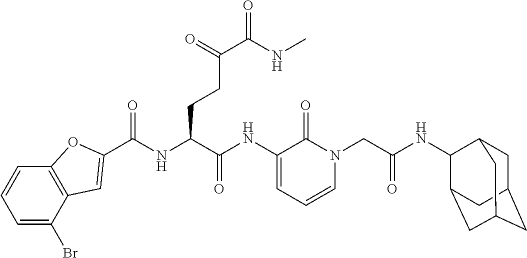 [SMILES: CNC(=O)C(=O)CC[C@H](NC(=O)c1cc2c(Br)cccc2o1)C(=O)Nc1cccn(CC(=O)NC2C3CC4CC(C3)CC2C4)c1=O] | (S)-2-(4-bromobenzofuran-2- carboxamido)-N1-(1-(2-(2- adamantylamino)-2-oxoethyl)-2-oxo-1,2- dihydropyridin-3-yl)-N6-methyl-5- oxohexanediamide |
| II-6: | 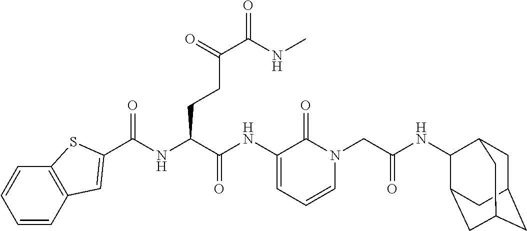 [SMILES: CNC(=O)C(=O)CC[C@H](NC(=O)c1cc2ccccc2s1)C(=O)Nc1cccn(CC(=O)NC2C3CC4CC(C3)CC2C4)c1=O] | (S)-2-(4-bromobenzofuran-2- carboxamido)-N1-(1-(2-(2- adamantylamino)-2-oxoethyl)-2-oxo-1,2- dihydropyridin-3-yl)-N6-methyl-5- oxohexanediamide |
| II-7: | 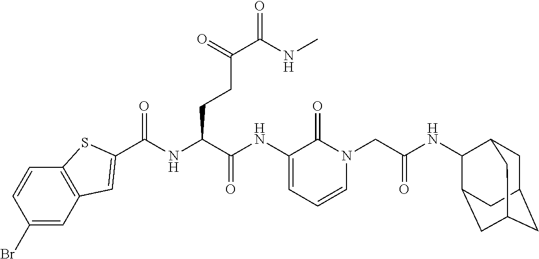 [SMILES: CNC(=O)C(=O)CC[C@H](NC(=O)c1cc2cc(Br)ccc2s1)C(=O)Nc1cccn(CC(=O)NC2C3CC4CC(C3)CC2C4)c1=O] | (S)-2-(benzo[b]thiophene-2-carboxamido)- N1-(1-(2-(2-adamantylamino)-2-oxoethyl)- 2-oxo-1,2-dihydropyridin-3-yl)-N6-methyl- 5-oxohexanediamide |
| II-8: | 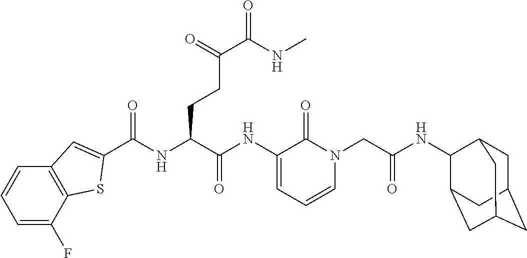 [SMILES: CNC(=O)C(=O)CC[C@H](NC(=O)c1cc2cccc(F)c2s1)C(=O)Nc1cccn(CC(=O)NC2C3CC4CC(C3)CC2C4)c1=O] | (S)-2-(5-bromobenzo[b]thiophene-2- carboxamido)-N1-(1-(2-(2- adamantylamino)-2-oxoethyl)-2-oxo-1,2- dihydropyridin-3-yl)-N6-methyl-5- oxohexanediamide |
| II-9: | 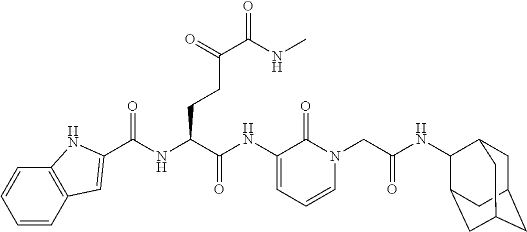 [SMILES: CNC(=O)C(=O)CC[C@H](NC(=O)c1cc2ccccc2[nH]1)C(=O)Nc1cccn(CC(=O)NC2C3CC4CC(C3)CC2C4)c1=O] | (S)-2-(1H-indole-2-carboxamido)-N1-(1-(2- (2-adamantylamino)-2-oxoethyl)-2-oxo- 1,2-dihydropyridin-3-yl)-N6-methyl-5- oxohexanediamide |
| II-10: | 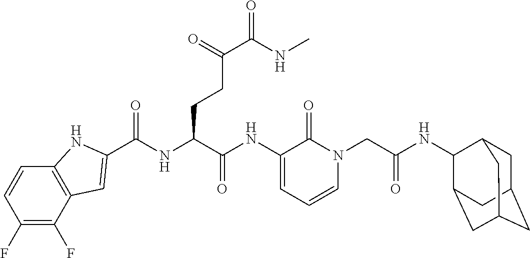 [SMILES: CNC(=O)C(=O)CC[C@H](NC(=O)c1cc2c(F)c(F)ccc2[nH]1)C(=O)Nc1cccn(CC(=O)NC2C3CC4CC(C3)CC2C4)c1=O] | (S)-2-(4,5-difluoro-1H-indole-2- carboxamido)-N1-(1-(2-(2- adamantylamino)-2-oxoethyl)-2-oxo-1,2- dihydropyridin-3-yl)-N6-methyl-5- oxohexanediamide |
| II-11: | 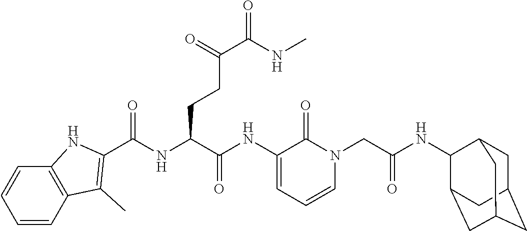 [SMILES: CNC(=O)C(=O)CC[C@H](NC(=O)c1[nH]c2ccccc2c1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(3-methyl-1H-indole-2- carboxamido)-5-oxohexanediamide |
| II-12: | 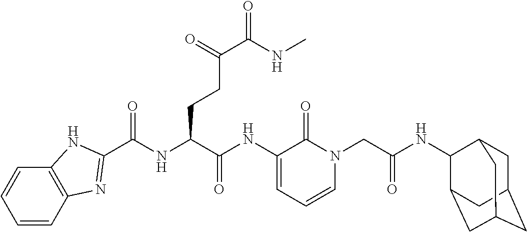 [SMILES: CNC(=O)C(=O)CC[C@H](NC(=O)c1nc2ccccc2[nH]1)C(=O)Nc1cccn(CC(=O)NC2C3CC4CC(C3)CC2C4)c1=O] | (S)-2-(1H-benzo[d]imidazole-2- carboxamido)-N1-(1-(2-(2- adamantylamino)-2-oxoethyl)-2-oxo-1,2- dihydropyridin-3-yl)-N6-methyl-5- oxohexanediamide |
| II-13: | 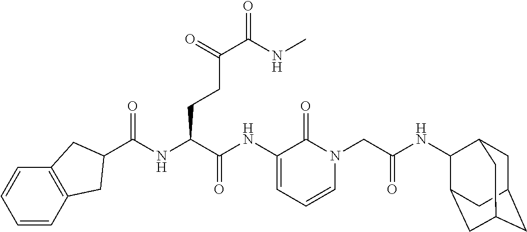 [SMILES: CNC(=O)C(=O)CC[C@H](NC(=O)C1Cc2ccccc2C1)C(=O)Nc1cccn(CC(=O)NC2C3CC4CC(C3)CC2C4)c1=O] | (S)-2-(2,3-dihydro-1H-indene-2- carboxamido)-N1-(1-(2-(2- adamantylamino)-2-oxoethyl)-2-oxo-1,2- dihydropyridin-3-yl)-N6-methyl-5- oxohexanediamide |
| II-14: | 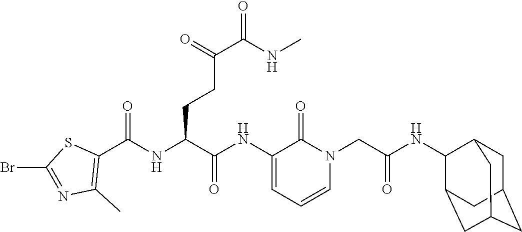 [SMILES: CNC(=O)C(=O)CC[C@H](NC(=O)c1sc(Br)nc1C)C(=O)Nc1cccn(CC(=O)NC2C3CC4CC(C3)CC2C4)c1=O] | (S)--(2-bromo-4-methylthiazole-5- carboxamido)-N1-(1-(2-(2- adamantylamino)-2-oxoethyl)-2-oxo-1,2- dihydropyridin-3-yl)-N6-methyl-5- oxohexanediamide |
| II-15: | 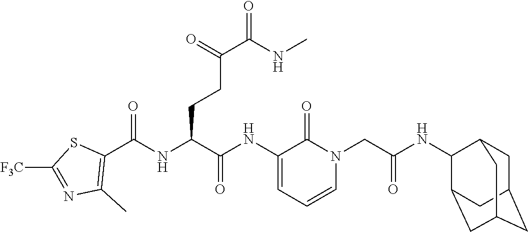 [SMILES: CNC(=O)C(=O)CC[C@H](NC(=O)c1sc(C(F)(F)F)nc1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(4-methyl-2- (trifluoromethyl)thiazole-5-carboxamido)- 5-oxohexanediamide |
| II-16: | 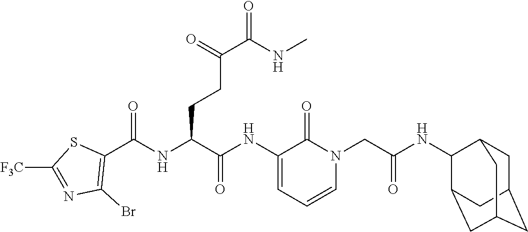 [SMILES: CNC(=O)C(=O)CC[C@H](NC(=O)c1sc(C(F)(F)F)nc1Br)C(=O)Nc1cccn(CC(=O)NC2C3CC4CC(C3)CC2C4)c1=O] | (S)-2-(4-bromo-2-(trifluoromethyl)thiazole- 5-carboxamido)-N1-(1-(2-(2- adamantylamino)-2-oxoethyl)-2-oxo-1,2- dihydropyridin-3-yl)-N6-methyl-5- oxohexanediamide |
| II-17: | 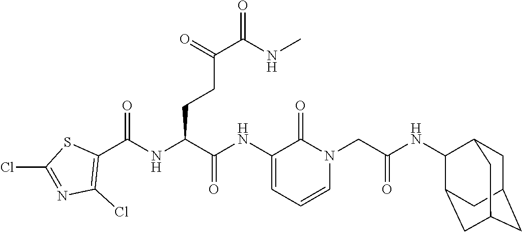 [SMILES: CNC(=O)C(=O)CC[C@H](NC(=O)c1sc(Cl)nc1Cl)C(=O)Nc1cccn(CC(=O)NC2C3CC4CC(C3)CC2C4)c1=O] | (S)-2-(2,4-dichlorothiazole-5- carboxamido)-N1-(1-(2-(2- adamantylamino)-2-oxoethyl)-2-oxo-1,2- dihydropyridin-3-yl)-N6-methyl-5- oxohexanediamide |
| II-18: | 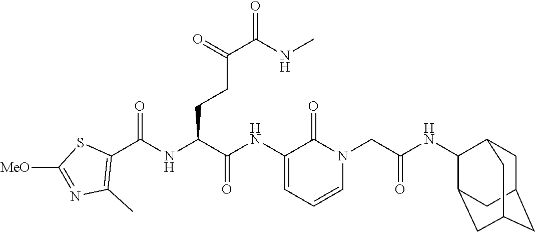 [SMILES: CNC(=O)C(=O)CC[C@H](NC(=O)c1sc(OC)nc1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- 2-(2-methoxy-4-methylthiazole-5- carboxamido)-N6-methyl-5- oxohexanediamide |
| II-19: | 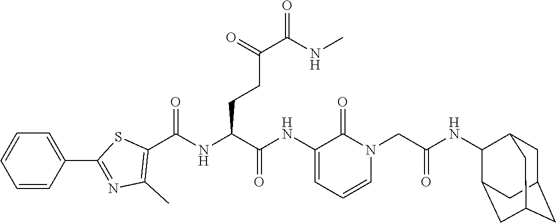 [SMILES: CNC(=O)C(=O)CC[C@H](NC(=O)c1sc(-c2ccccc2)nc1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(4-methyl-2-phenylthiazole-5- carboxamido)-5-oxohexanediamide |
| II-20: | 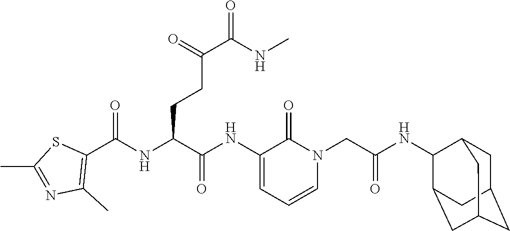 [SMILES: CNC(=O)C(=O)CC[C@H](NC(=O)c1sc(C)nc1C)C(=O)Nc1cccn(CC(=O)NC2C3CC4CC(C3)CC2C4)c1=O] | (S)-2-(2,4-dimethylthiazole-5- carboxamido)-N1-(1-(2-(2- adamantylamino)-2-oxoethyl)-2-oxo-1,2- dihydropyridin-3-yl)-N6-methyl-5- oxohexanediamide |
| II-21: | 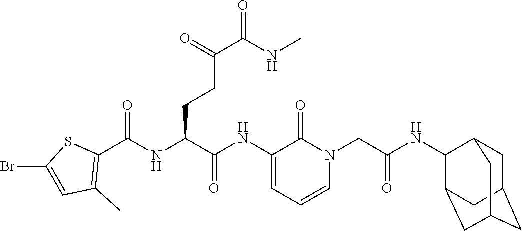 [SMILES: CNC(=O)C(=O)CC[C@H](NC(=O)c1sc(Br)cc1C)C(=O)Nc1cccn(CC(=O)NC2C3CC4CC(C3)CC2C4)c1=O] | (S)-2-(5-bromo-3-methylthiophene-2- carboxamido)-N1-(1-(2-(2- adamantylamino)-2-oxoethyl)-2-oxo-1,2- dihydropyridin-3-yl)-N6-methyl-5- oxohexanediamide |
| II-22: | 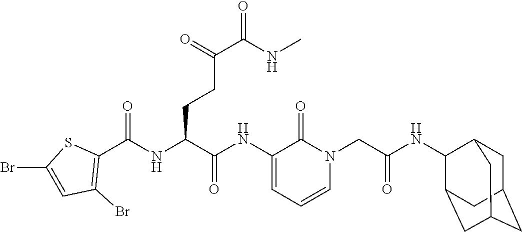 [SMILES: CNC(=O)C(=O)CC[C@H](NC(=O)c1sc(Br)cc1Br)C(=O)Nc1cccn(CC(=O)NC2C3CC4CC(C3)CC2C4)c1=O] | (S)-2-(3,5-dibromothiophene-2- carboxamido)-N1-(1-(2-(2- adamantylamino)-2-oxoethyl)-2-oxo-1,2- dihydropyridin-3-yl)-N6-methyl-5- oxohexanediamide |
| II-23: | 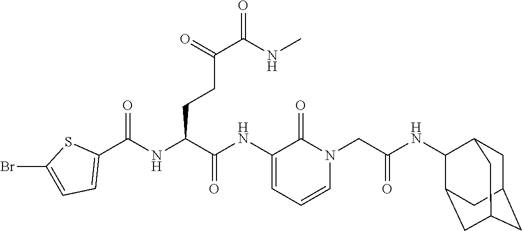 [SMILES: CNC(=O)C(=O)CC[C@H](NC(=O)c1ccc(Br)s1)C(=O)Nc1cccn(CC(=O)NC2C3CC4CC(C3)CC2C4)c1=O] | (S)-2-(5-bromothiophene-2-carboxamido)- N1-(1-(2-(2-adamantylamino)-2-oxoethyl)- 2-oxo-1,2-dihydropyridin-3-yl)-N6-methyl- 5-oxohexanediamide |
| II-24: | 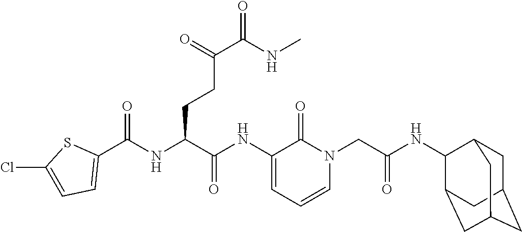 [SMILES: CNC(=O)C(=O)CC[C@H](NC(=O)c1ccc(Cl)s1)C(=O)Nc1cccn(CC(=O)NC2C3CC4CC(C3)CC2C4)c1=O] | (S)-2-(5-chlorothiophene-2-carboxamido)- N1-(1-(2-(2-adamantylamino)-2-oxoethyl)- 2-oxo-1,2-dihydropyridin-3-yl)-N6-methyl- 5-oxohexanediamide |
| II-25: | 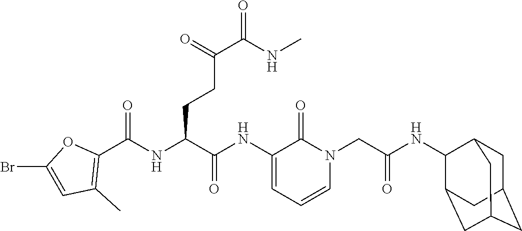 [SMILES: CNC(=O)C(=O)CC[C@H](NC(=O)c1oc(Br)cc1C)C(=O)Nc1cccn(CC(=O)NC2C3CC4CC(C3)CC2C4)c1=O] | (S)-2-(5-bromo-3-methylfuran-2- carboxamido)-N1-(1-(2-(2- adamantylamino)-2-oxoethyl)-2-oxo-1,2- dihydropyridin-3-yl)-N6-methyl-5- oxohexanediamide |
| II-26: | 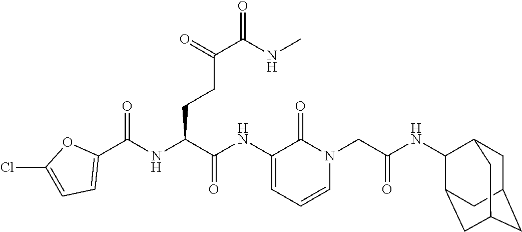 [SMILES: CNC(=O)C(=O)CC[C@H](NC(=O)c1ccc(Cl)o1)C(=O)Nc1cccn(CC(=O)NC2C3CC4CC(C3)CC2C4)c1=O] | (S)-2-(5-chlorofuran-2-carboxamido)-N1- (1-(2-(2-adamantylamino)-2-oxoethyl)-2- oxo-1,2-dihydropyridin-3-yl)-N6-methyl-5- oxohexanediamide |
| II-27: | 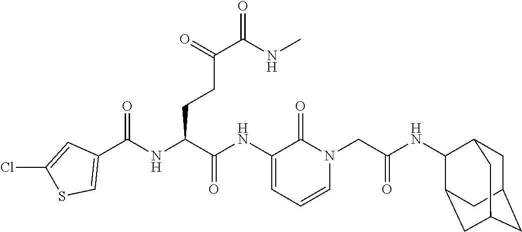 [SMILES: CNC(=O)C(=O)CC[C@H](NC(=O)c1csc(Cl)c1)C(=O)Nc1cccn(CC(=O)NC2C3CC4CC(C3)CC2C4)c1=O] | (S)-2-(5-chlorothiophene-3-carboxamido)- N1-(1-(2-(2-adamantylamino)-2-oxoethyl)- 2-oxo-1,2-dihydropyridin-3-yl)-N6-methyl- 5-oxohexanediamide |
| II-28: | 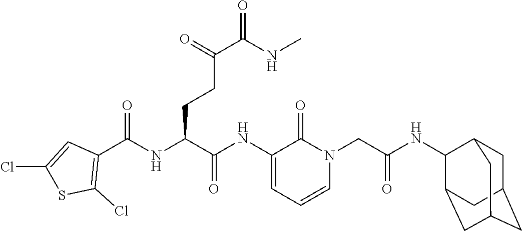 [SMILES: CNC(=O)C(=O)CC[C@H](NC(=O)c1cc(Cl)sc1Cl)C(=O)Nc1cccn(CC(=O)NC2C3CC4CC(C3)CC2C4)c1=O] | (S)-2-(2,5-dichlorothiophene-3- carboxamido)-N1-(1-(2-(2- adamantylamino)-2-oxoethyl)-2-oxo-1,2- dihydropyridin-3-yl)-N6-methyl-5- oxohexanediamide |
| II-29: | 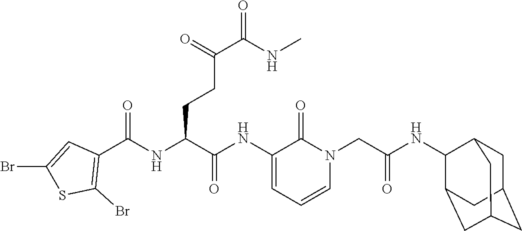 [SMILES: CNC(=O)C(=O)CC[C@H](NC(=O)c1cc(Br)sc1Br)C(=O)Nc1cccn(CC(=O)NC2C3CC4CC(C3)CC2C4)c1=O] | (S)-2-(2,5-dibromothiophene-3- carboxamido)-N1-(1-(2-(2- adamantylamino)-2-oxoethyl)-2-oxo-1,2- dihydropyridin-3-yl)-N6-methyl-5- oxohexanediamide |
| II-30: | 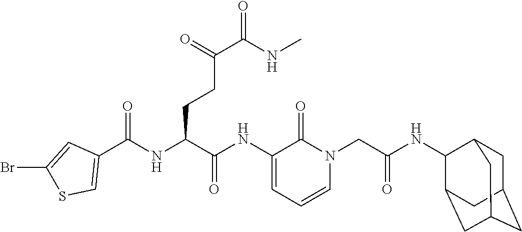 [SMILES: CNC(=O)C(=O)CC[C@H](NC(=O)c1csc(Br)c1)C(=O)Nc1cccn(CC(=O)NC2C3CC4CC(C3)CC2C4)c1=O] | (S)-2-(5-bromothiophene-3-carboxamido)- N1-(1-(2-(2-adamantylamino)-2-oxoethyl)- 2-oxo-1,2-dihydropyridin-3-yl)-N6-methyl- 5-oxohexanediamide |
| II-31: | 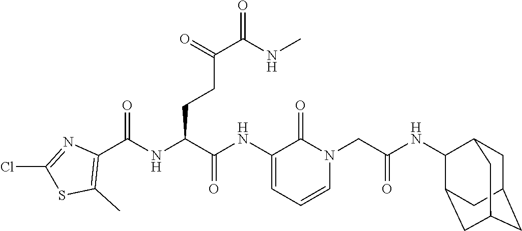 [SMILES: CNC(=O)C(=O)CC[C@H](NC(=O)c1nc(Cl)sc1C)C(=O)Nc1cccn(CC(=O)NC2C3CC4CC(C3)CC2C4)c1=O] | (S)-2-(2-chloro-5-methylthiazole-4- carboxamido)-N1-(1-(2-(2- adamantylamino)-2-oxoethyl)-2-oxo-1,2- dihydropyridin-3-yl)-N6-methyl-5- oxohexanediamide |
| II-32: | 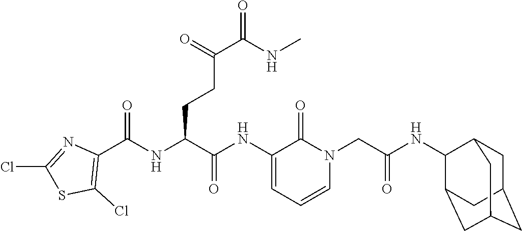 [SMILES: CNC(=O)C(=O)CC[C@H](NC(=O)c1nc(Cl)sc1Cl)C(=O)Nc1cccn(CC(=O)NC2C3CC4CC(C3)CC2C4)c1=O] | (S)-2-(2,5-dichlorothiazole-4- carboxamido)-N1-(1-(2-(2- adamantylamino)-2-oxoethyl)-2-oxo-1,2- dihydropyridin-3-yl)-N6-methyl-5- oxohexanediamide |
| II-33: | 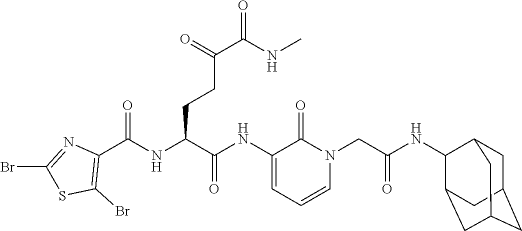 [SMILES: CNC(=O)C(=O)CC[C@H](NC(=O)c1nc(Br)sc1Br)C(=O)Nc1cccn(CC(=O)NC2C3CC4CC(C3)CC2C4)c1=O] | (S)-2-(2,5-dibromothiazole-4- carboxamido)-N1-(1-(2-(2- adamantylamino)-2-oxoethyl)-2-oxo-1,2- dihydropyridin-3-yl)-N6-methyl-5- oxohexanediamide |
| II-34: | 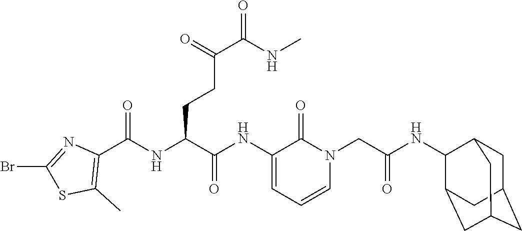 [SMILES: CNC(=O)C(=O)CC[C@H](NC(=O)c1nc(Br)sc1C)C(=O)Nc1cccn(CC(=O)NC2C3CC4CC(C3)CC2C4)c1=O] | (S)-2-(2-bromo-5-methylthiazole-4- carboxamido)-N1-(1-(2-(2- adamantylamino)-2-oxoethyl)-2-oxo-1,2- dihydropyridin-3-yl)-N6-methyl-5- oxohexanediamide |
| II-35: | 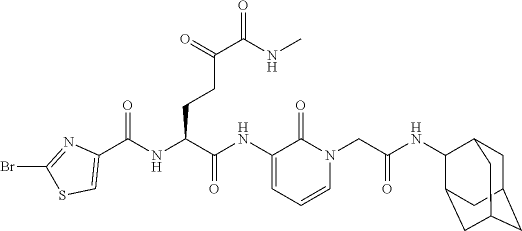 [SMILES: CNC(=O)C(=O)CC[C@H](NC(=O)c1csc(Br)n1)C(=O)Nc1cccn(CC(=O)NC2C3CC4CC(C3)CC2C4)c1=O] | (S)-2-(2-bromothiazole-4-carboxamido)- N1-(1-(2-(2-adamantylamino)-2-oxoethyl)- 2-oxo-1,2-dihydropyridin-3-yl)-N6-methyl- 5-oxohexanediamide |
| II-36: | 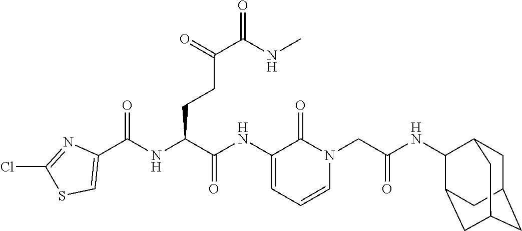 [SMILES: CNC(=O)C(=O)CC[C@H](NC(=O)c1csc(Cl)n1)C(=O)Nc1cccn(CC(=O)NC2C3CC4CC(C3)CC2C4)c1=O] | (S)-2-(2-chlorothiazole-4-carboxamido)- N1-(1-(2-(2-adamantylamino)-2-oxoethyl)- 2-oxo-1,2-dihydropyridin-3-yl)-N6-methyl- 5-oxohexanediamide |
| II-37: | 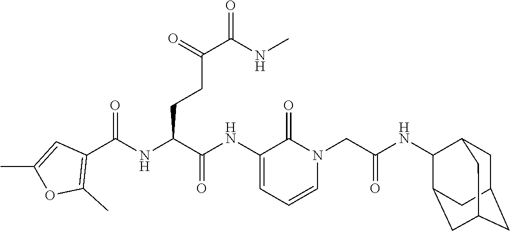 [SMILES: CNC(=O)C(=O)CC[C@H](NC(=O)c1cc(C)oc1C)C(=O)Nc1cccn(CC(=O)NC2C3CC4CC(C3)CC2C4)c1=O] | (S)-2-(2,5-dimethylfuran-3-carboxamido)- N1-(1-(2-(2-adamantylamino)-2-oxoethyl)- 2-oxo-1,2-dihydropyridin-3-yl)-N6-methyl- 5-oxohexanediamide |
| II-38: | 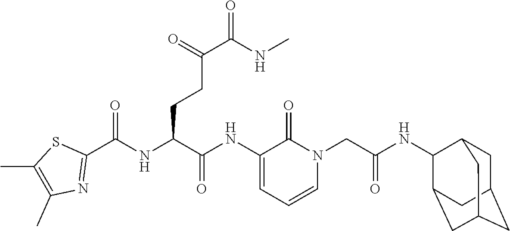 [SMILES: CNC(=O)C(=O)CC[C@H](NC(=O)c1nc(C)c(C)s1)C(=O)Nc1cccn(CC(=O)NC2C3CC4CC(C3)CC2C4)c1=O] | (S)-2-(4,5-dimethylthiazole-2- carboxamido)-N1-(1-(2-(2- adamantylamino)-2-oxoethyl)-2-oxo-1,2- dihydropyridin-3-yl)-N6-methyl-5- oxohexanediamide |
| II-39: | 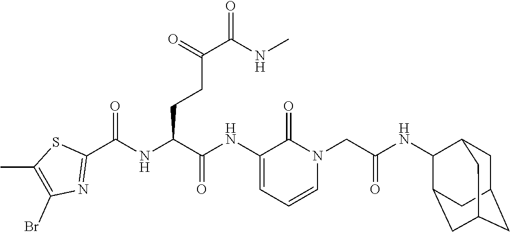 [SMILES: CNC(=O)C(=O)CC[C@H](NC(=O)c1nc(Br)c(C)s1)C(=O)Nc1cccn(CC(=O)NC2C3CC4CC(C3)CC2C4)c1=O] | (S)-2-(4-bromothiazole-2-carboxamido)- N1-(1-(2-(2-adamantylamino)-2-oxoethyl)- 2-oxo-1,2-dihydropyridin-3-yl)-N6-methyl- 5-oxohexanediamide |
| II-40: | 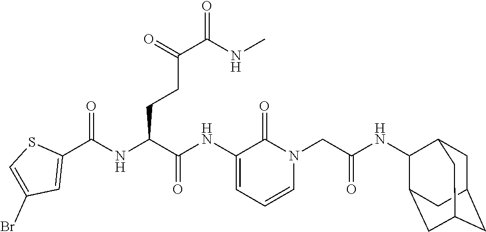 [SMILES: CNC(=O)C(=O)CC[C@H](NC(=O)c1cc(Br)cs1)C(=O)Nc1cccn(CC(=O)NC2C3CC4CC(C3)CC2C4)c1=O] | (S)-2-(4-bromothiophene-2-carboxamido)- N1-(1-(2-(2-adamantylamino)-2-oxoethyl)- 2-oxo-1,2-dihydropyridin-3-yl)-N6-methyl- 5-oxohexanediamide |
| II-41: | 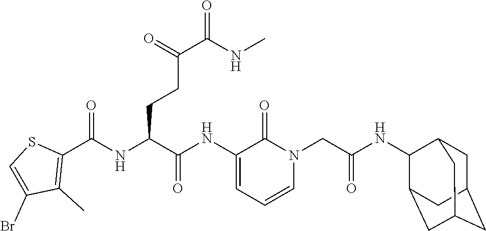 [SMILES: CNC(=O)C(=O)CC[C@H](NC(=O)c1scc(Br)c1C)C(=O)Nc1cccn(CC(=O)NC2C3CC4CC(C3)CC2C4)c1=O] | (S)-2-(4-bromo-3-methylthiophene-2- carboxamido)-N1-(1-(2-(2- adamantylamino)-2-oxoethyl)-2-oxo-1,2- dihydropyridin-3-yl)-N6-methyl-5- oxohexanediamide |
| II-42: | 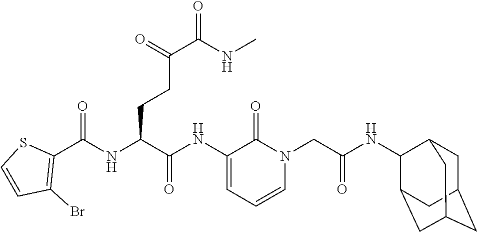 [SMILES: CNC(=O)C(=O)CC[C@H](NC(=O)c1sccc1Br)C(=O)Nc1cccn(CC(=O)NC2C3CC4CC(C3)CC2C4)c1=O] | (S)-2-(3-bromothiophene-2-carboxamido)- N1-(1-(2-(2-adamantylamino)-2-oxoethyl)- 2-oxo-1,2-dihydropyridin-3-yl)-N6-methyl- 5-oxohexanediamide |
| II-43: | 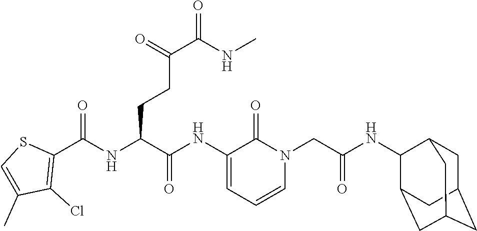 [SMILES: CNC(=O)C(=O)CC[C@H](NC(=O)c1scc(C)c1Cl)C(=O)Nc1cccn(CC(=O)NC2C3CC4CC(C3)CC2C4)c1=O] | (S)-2-(3-chloro-4-methylthiophene-2- carboxamido)-N1-(1-(2-(2- adamantylamino)-2-oxoethyl)-2-oxo-1,2- dihydropyridin-3-yl)-N6-methyl-5- oxohexanediamide |
| II-44: | 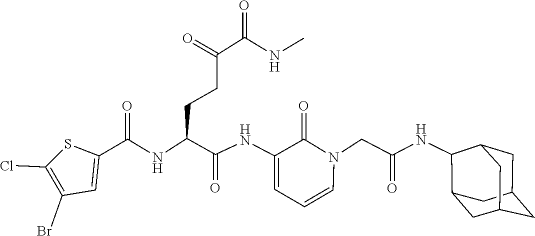 [SMILES: CNC(=O)C(=O)CC[C@H](NC(=O)c1cc(Br)c(Cl)s1)C(=O)Nc1cccn(CC(=O)NC2C3CC4CC(C3)CC2C4)c1=O] | (S)-2-(4-bromo-5-chlorothiophene-2- carboxamido)-N1-(1-(2-(2- adamantylamino)-2-oxoethyl)-2-oxo-1,2- dihydropyridin-3-yl)-N6-methyl-5- oxohexanediamide |
| II-45: | 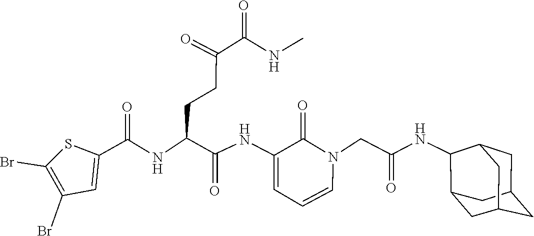 [SMILES: CNC(=O)C(=O)CC[C@H](NC(=O)c1cc(Br)c(Br)s1)C(=O)Nc1cccn(CC(=O)NC2C3CC4CC(C3)CC2C4)c1=O] | (S)-2-(4,5-dibromothiophene-2- carboxamido)-N1-(1-(2-(2- adamantylamino)-2-oxoethyl)-2-oxo-1,2- dihydropyridin-3-yl)-N6-methyl-5- oxohexanediamide |
| II-46: | 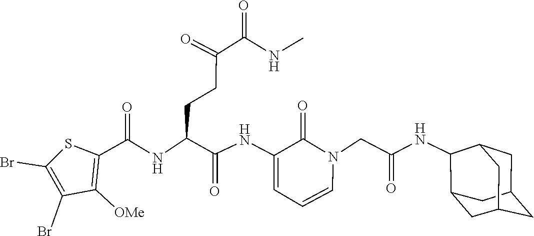 [SMILES: CNC(=O)C(=O)CC[C@H](NC(=O)c1sc(Br)c(Br)c1OC)C(=O)Nc1cccn(CC(=O)NC2C3CC4CC(C3)CC2C4)c1=O] | (S)-2-(4,5-dibromo-3-methoxythiophene-2- carboxamido)-N1-(1-(2-(2- adamantylamino)-2-oxoethyl)-2-oxo-1,2- dihydropyridin-3-yl)-N6-methyl-5- oxohexanediamide |
| II-47: | 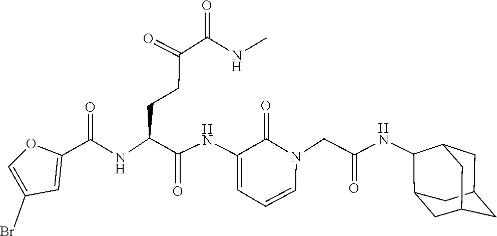 [SMILES: CNC(=O)C(=O)CC[C@H](NC(=O)c1cc(Br)co1)C(=O)Nc1cccn(CC(=O)NC2C3CC4CC(C3)CC2C4)c1=O] | (S)-2-(4-bromofuran-2-carboxamido)-N1- (1-(2-(2-adamantylamino)-2-oxoethyl)-2- oxo-1,2-dihydropyridin-3-yl)-N6-methyl-5- oxohexanediamide |
| II-48: | 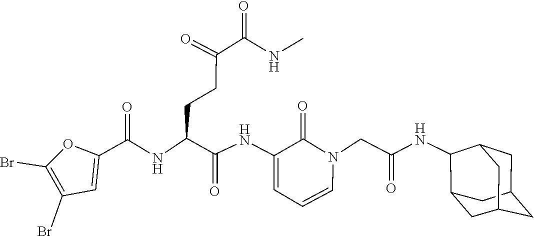 [SMILES: CNC(=O)C(=O)CC[C@H](NC(=O)c1cc(Br)c(Br)o1)C(=O)Nc1cccn(CC(=O)NC2C3CC4CC(C3)CC2C4)c1=O] | (S)-2-(4,5-dibromofuran-2-carboxamido)- N1-(1-(2-(2-adamantylamino)-2-oxoethyl)- 2-oxo-1,2-dihydropyridin-3-yl)-N6-methyl- 5-oxohexanediamide |
| II-49: | 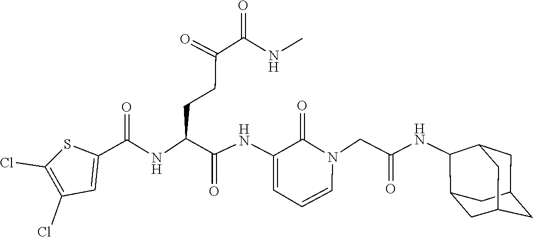 [SMILES: CNC(=O)C(=O)CC[C@H](NC(=O)c1cc(Cl)c(Cl)s1)C(=O)Nc1cccn(CC(=O)NC2C3CC4CC(C3)CC2C4)c1=O] | (S)-2-(4,5-dichlorothiophene-2- carboxamido)-N1-(1-(2-(2- adamantylamino)-2-oxoethyl)-2-oxo-1,2- dihydropyridin-3-yl)-N6-methyl-5- oxohexanediamide |
| II-50: | 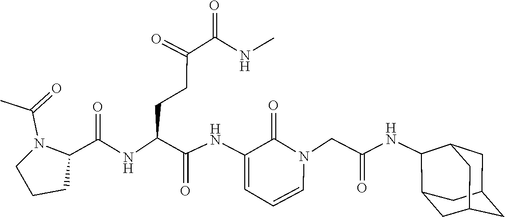 [SMILES: CNC(=O)C(=O)CC[C@H](NC(=O)[C@@H]1CCCN1C(C)=O)C(=O)Nc1cccn(CC(=O)NC2C3CC4CC(C3)CC2C4)c1=O] | (S)-2-((S)-1-acetylpyrrolidine-2- carboxamido)-N1-(1-(2-(2- adamantylamino)-2-oxoethyl)-2-oxo-1,2- dihydropyridin-3-yl)-N6-methyl-5- oxohexanediamide |
| II-51: | 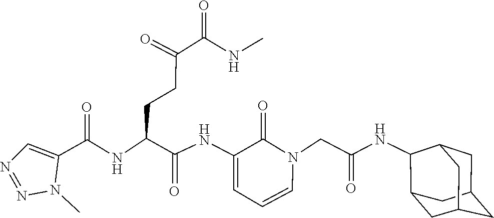 [SMILES: CNC(=O)C(=O)CC[C@H](NC(=O)c1cnnn1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-1,2,3-triazole-5- carboxamido)-5-oxohexanediamide |
| II-52: | 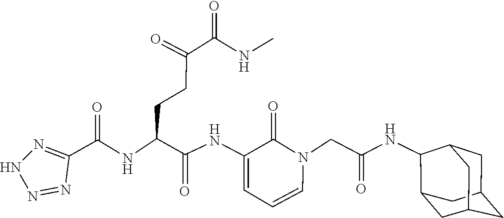 [SMILES: CNC(=O)C(=O)CC[C@H](NC(=O)c1nn[nH]n1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(2H-tetrazole-5- carboxamido)hexanediamide |
| II-53: | 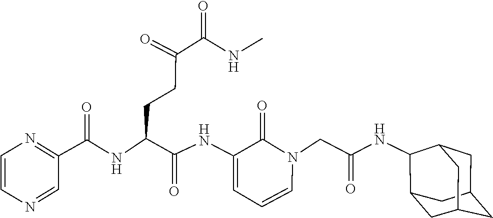 [SMILES: CNC(=O)C(=O)CC[C@H](NC(=O)c1cnccn1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(pyrazine-2- carboxamido)hexanediamide |
| II-54: | 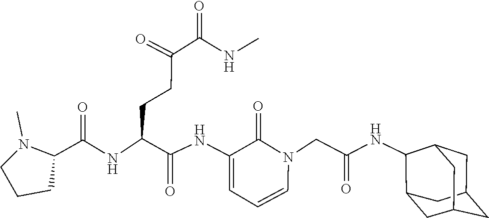 [SMILES: CNC(=O)C(=O)CC[C@H](NC(=O)[C@@H]1CCCN1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-((S)-1-methylpyrrolidine-2- carboxamido)-5-oxohexanediamide |
| II-55: | 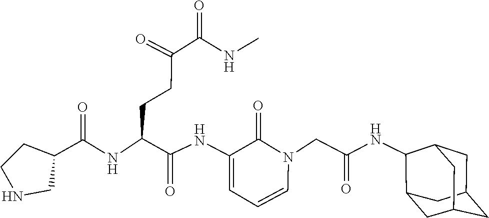 [SMILES: CNC(=O)C(=O)CC[C@H](NC(=O)[C@H]1CCNC1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-((S)-pyrrolidine-3- carboxamido)hexanediamide |
| II-56: | 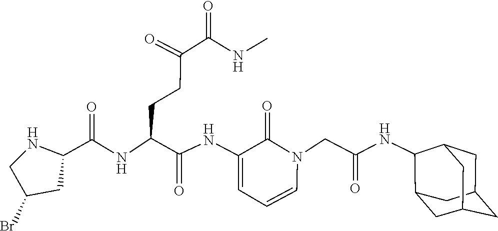 [SMILES: CNC(=O)C(=O)CC[C@H](NC(=O)[C@@H]1C[C@H](Br)CN1)C(=O)Nc1cccn(CC(=O)NC2C3CC4CC(C3)CC2C4)c1=O] | (S)-2-((2S,4S)-4-bromopyrrolidine-2- carboxamido)-N1-(1-(2-(2- adamantylamino)-2-oxoethyl)-2-oxo-1,2- dihydropyridin-3-yl)-N6-methyl-5- oxohexanediamide |
| II-58: | 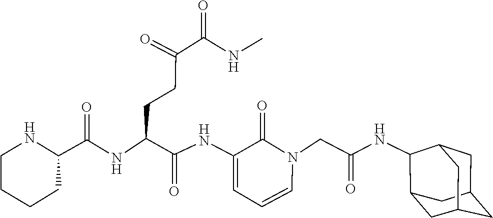 [SMILES: CNC(=O)C(=O)CC[C@H](NC(=O)[C@@H]1CCCCN1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-((S)-piperidine-2- carboxamido)hexanediamide |
| II-59: | 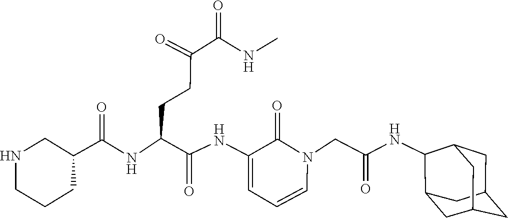 [SMILES: CNC(=O)C(=O)CC[C@H](NC(=O)[C@@H]1CCCNC1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-((R)-piperidine-3- carboxamido)hexanediamide |
| II-60: | 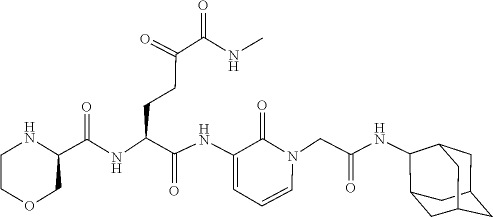 [SMILES: CNC(=O)C(=O)CC[C@H](NC(=O)[C@H]1COCCN1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-((R)-morpholine-3- carboxamido)-5-oxohexanediamide |
| II-61: | 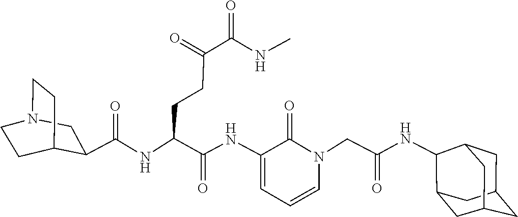 [SMILES: CNC(=O)C(=O)CC[C@H](NC(=O)C1CN2CCC1CC2)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(quinuclidine-3- carboxamido)hexanediamide |
| II-62: | 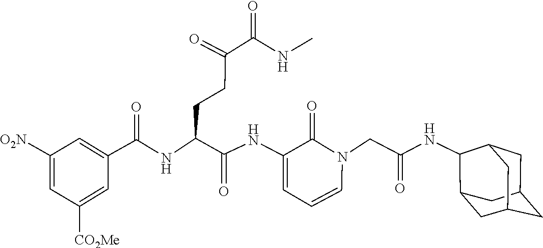 [SMILES: CNC(=O)C(=O)CC[C@H](NC(=O)c1cc(C(=O)OC)cc([N+](=O)[O-])c1)C(=O)Nc1cccn(CC(=O)NC2C3CC4CC(C3)CC2C4)c1=O] | (S)-methyl 3-(1-(1-(2-(2-adamantylamino)- 2-oxoethyl)-2-oxo-1,2-dihydropyridin-3- ylamino)-6-(methylamino)-1,5,6- trioxohexan-2-ylcarbamoyl)-5- nitrobenzoate |
| II-63: | 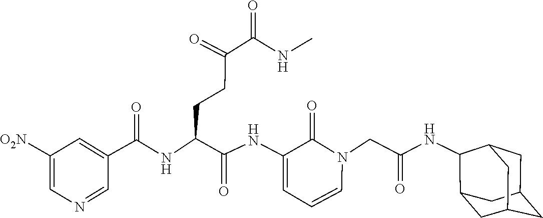 [SMILES: CNC(=O)C(=O)CC[C@H](NC(=O)c1cncc([N+](=O)[O-])c1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(5-nitronicotinamido)-5- oxohexanediamide |
| II-64: | 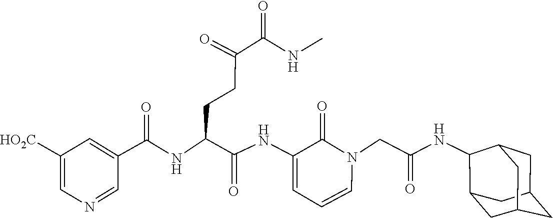 [SMILES: CNC(=O)C(=O)CC[C@H](NC(=O)c1cncc(C(=O)O)c1)C(=O)Nc1cccn(CC(=O)NC2C3CC4CC(C3)CC2C4)c1=O] | (S)-5-(1-(1-(2-(2-adamantylamino)-2- oxoethyl)-2-oxo-1,2-dihydropyridin-3- ylamino)-6-(methylamino)-1,5,6- trioxohexan-2-ylcarbamoyl)nicotinic acid |
| II-65: | 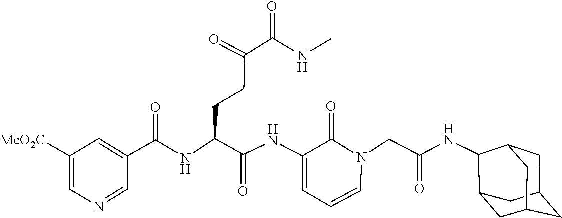 [SMILES: CNC(=O)C(=O)CC[C@H](NC(=O)c1cncc(C(=O)OC)c1)C(=O)Nc1cccn(CC(=O)NC2C3CC4CC(C3)CC2C4)c1=O] | (S)-methyl 5-(1-(1-(2-(2-adamantylamino)- 2-oxoethyl)-2-oxo-1,2-dihydropyridin-3- ylamino)-6-(methylamino)-1,5,6- trioxohexan-2-ylcarbamoyl)nicotinate |
| II-66: | 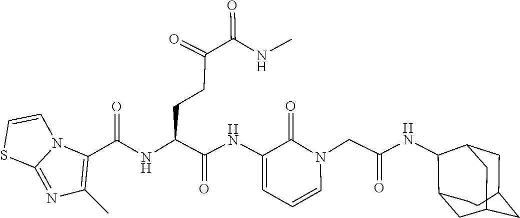 [SMILES: CNC(=O)C(=O)CC[C@H](NC(=O)c1c(C)nc2sccn12)C(=O)Nc1cccn(CC(=O)NC2C3CCCC4CC(C3)CC2C4)c1=O] | (S)-N1-(1-(2-(2-adamantylamino)-2- oxoethyl)-2-oxo-1,2-dihydropyridin-3-yl)- N6-methyl-2-(6-methylimidazo[2,1- b]thiazole-5-carboxamido)-5- oxohexanediamide |
| II-67: | 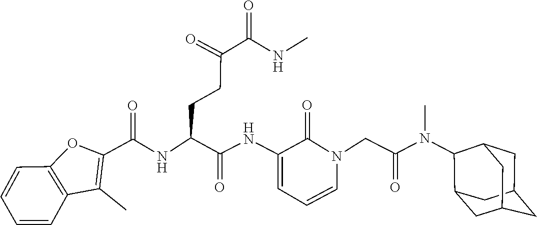 [SMILES: CNC(=O)C(=O)CC[C@H](NC(=O)c1oc2ccccc2c1C)C(=O)Nc1cccn(CC(=O)N(C)C2C3CC4CC(C3)CC2C4)c1=O] | (S)-N1-(1-(2-(2-adamantyl(methyl)amino)- 2-oxoethyl)-2-oxo-1,2-dihydropyridin-3-yl)- N6-methyl-2-(3-methylbenzofuran-2- carboxamido)-5-oxohexanediamide |
| II-68: | 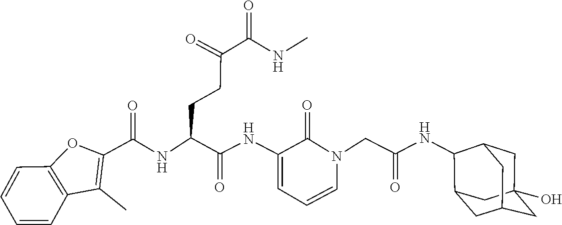 [SMILES: CNC(=O)C(=O)CC[C@H](NC(=O)c1oc2ccccc2c1C)C(=O)Nc1cccn(CC(=O)NC2C3CC4CC2CC(O)(C4)C3)c1=O] | (S)-N1-(1-(2-(5-hydroxyadamantane-2- amino)-2-oxoethyl)-2-oxo-1,2- dihydropyridin-3-yl)-N6-methyl-2-(3- methylbenzofuran-2-carboxamido)-5- oxohexanediamide |
| II-69: | 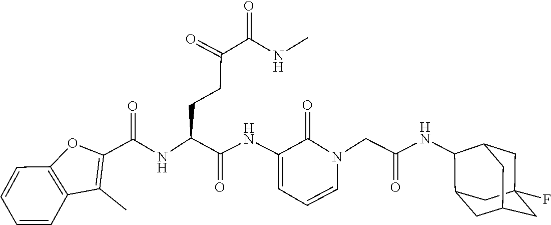 [SMILES: CNC(=O)C(=O)CC[C@H](NC(=O)c1oc2ccccc2c1C)C(=O)Nc1cccn(CC(=O)NC2C3CC4CC2CC(F)(C4)C3)c1=O] | (S)-N1-(1-(2-(5-fluoroadamantane-2- amino)-2-oxoethyl)-2-oxo-1,2- dihydropyridin-3-yl)-N6-methyl-2-(3- methylbenzofuran-2-carboxamido)-5- oxohexanediamide |
| II-70: | 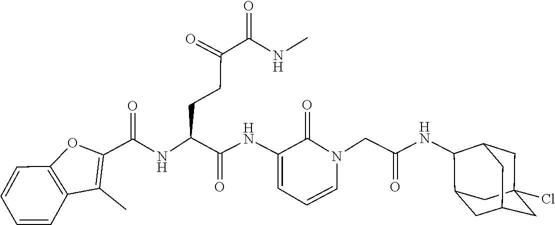 [SMILES: CNC(=O)C(=O)CC[C@H](NC(=O)c1oc2ccccc2c1C)C(=O)Nc1cccn(CC(=O)NC2C3CC4CC2CC(Cl)(C4)C3)c1=O] | (S)-N1-(1-(2-(5-chloroadamantane-2- amino)-2-oxoethyl)-2-oxo-1,2- dihydropyridin-3-yl)-N6-methyl-2-(3- methylbenzofuran-2-carboxamido)-5- oxohexanediamide |
| II-71: | 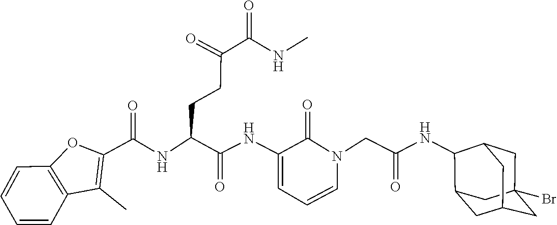 [SMILES: CNC(=O)C(=O)CC[C@H](NC(=O)c1oc2ccccc2c1C)C(=O)Nc1cccn(CC(=O)NC2C3CC4CC2CC(Br)(C4)C3)c1=O] | (S)-N1-(1-(2-(5-bromoadamantane-2- amino)-2-oxoethyl)-2-oxo-1,2- dihydropyridin-3-yl)-N6-methyl-2-(3- methylbenzofuran-2-carboxamido)-5- oxohexanediamide |
| II-72: | 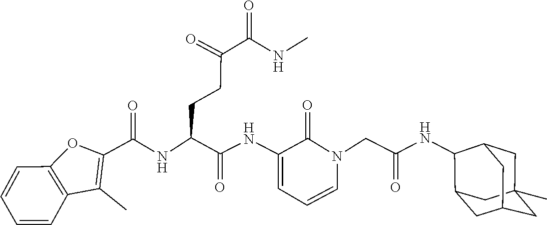 [SMILES: CNC(=O)C(=O)CC[C@H](NC(=O)c1oc2ccccc2c1C)C(=O)Nc1cccn(CC(=O)NC2C3CC4CC2CC(C)(C4)C3)c1=O] | (S)-N1-(1-(2-(5-methyladamantane-2- amino)-2-oxoethyl)-2-oxo-1,2- dihydropyridin-3-yl)-N6-methyl-2-(3- methylbenzofuran-2-carboxamido)-5- oxohexanediamide |
| II-73: | 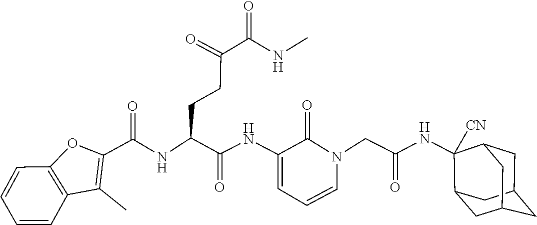 [SMILES: CNC(=O)C(=O)CC[C@H](NC(=O)c1oc2ccccc2c1C)C(=O)Nc1cccn(CC(=O)NC2(C#N)C3CC4CC(C3)CC2C4)c1=O] | (S)-N1-(1-(2-(2-carbonitrileadamantane-2- amino)-2-oxoethyl)-2-oxo-1,2- dihydropyridin-3-yl)-N6-methyl-2-(3- methylbenzofuran-2-carboxamido)-5- oxohexanediamide |
| II-74: | 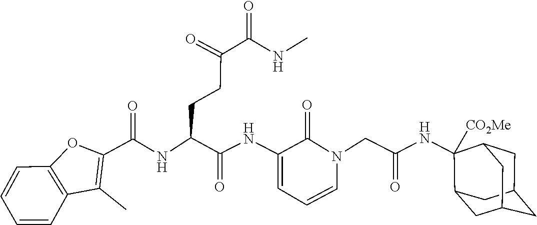 [SMILES: CNC(=O)C(=O)CC[C@H](NC(=O)c1oc2ccccc2c1C)C(=O)Nc1cccn(CC(=O)NC2(C(=O)OC)C3CC4CC(C3)CC2C4)c1=O] | (S)-N1-(1-(2-(2-methyladamantane-2- carboxylate-2-amino)-2-oxoethyl)-2-oxo- 1,2-dihydropyridin-3-yl)-N6-methyl-2-(3- methylbenzofuran-2-carboxamido)-5- oxohexanediamide |
| II-87: | 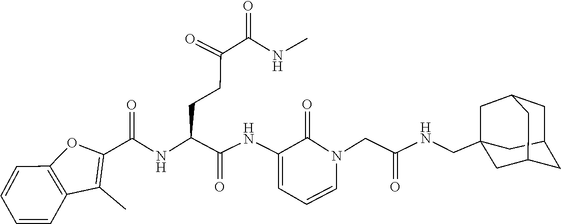 [SMILES: CNC(=O)C(=O)CC[C@H](NC(=O)c1oc2ccccc2c1C)C(=O)Nc1cccn(CC(=O)NCC23CC4CC(CC(C4)C2)C3)c1=O] | (S)-N1-(1-(2-(1-adamantylmethylamino)-2- oxoethyl)-2-oxo-1,2-dihydropyridin-3-yl)- N6-methyl-2-(3-methylbenzofuran-2- carboxamido)-5-oxohexanediamide |
| II-88: | 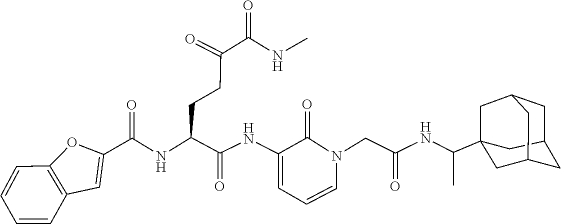 [SMILES: CNC(=O)C(=O)CC[C@H](NC(=O)c1cc2ccccc2o1)C(=O)Nc1cccn(CC(=O)NC(C)C23CC4CC(CC(C4)C2)C3)c1=O] | (S)-N1-(1-(2-(1-(1- adamantyl)ethanamino)-2-oxoethyl)-2-oxo- 1,2-dihydropyridin-3-yl)-N6-methyl-2-(3- methylbenzofuran-2-carboxamido)-5- oxohexanediamide |
| II-90: | 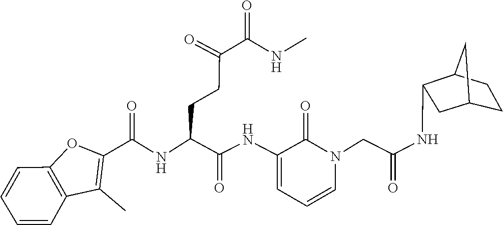 [SMILES: CNC(=O)C(=O)CC[C@H](NC(=O)c1oc2ccccc2c1C)C(=O)Nc1cccn(CC(=O)NC2CC3CCC2C3)c1=O] | (S)-N1-(1-(2-((1R,2S,4S)- bicyclo[2.2.1]heptan-2-ylamino)-2- oxoethyl)-2-oxo-1,2-dihydropyridin-3-yl)- N6-methyl-2-(3-methylbenzofuran-2- carboxamido)-5-oxohexanediamide |
| II-92: | 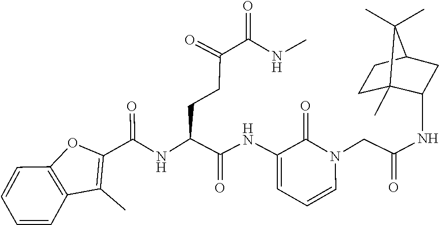 [SMILES: CNC(=O)C(=O)CC[C@H](NC(=O)c1oc2ccccc2c1C)C(=O)Nc1cccn(CC(=O)NC2CC3CCC2(C)C3(C)C)c1=O] | (S)-N1-methyl-5-(3-methylbenzofuran-2- carboxamido)-2-oxo-N6-(2-oxo-1-(2-oxo- 2-((1S,2S,4R)-1,7,7- trimethylbicyclo[2.2.1]heptan-2- ylamino)ethyl)-1,2-dihydropyridin-3- yl)hexanediamide |
| II-94: | 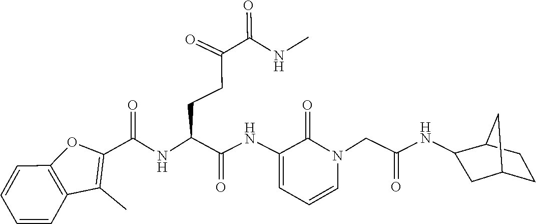 [SMILES: CNC(=O)C(=O)CC[C@H](NC(=O)c1oc2ccccc2c1C)C(=O)Nc1cccn(CC(=O)NC2CC3CCC2C3)c1=O] | (S)-N1-(1-(2-((1R,2R,4S)- bicyclo[2.2.1]heptan-2-ylamino)-2- oxoethyl)-2-oxo-1,2-dihydropyridin-3-yl)- N6-methyl-2-(3-methylbenzofuran-2- carboxamido)-5-oxohexanediamide |
| II-95: | 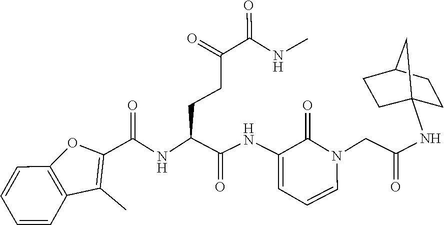 [SMILES: CNC(=O)C(=O)CC[C@H](NC(=O)c1oc2ccccc2c1C)C(=O)Nc1cccn(CC(=O)NC23CCC(CC2)C3)c1=O] | (S)-N1-(1-(2-(bicyclo[2.2.1]heptan-1- ylamino)-2-oxoethyl)-2-oxo-1,2- dihydropyridin-3-yl)-N6-methyl-2-(3- methylbenzofuran-2-carboxamido)-5- oxohexanediamide |
| II-96: | 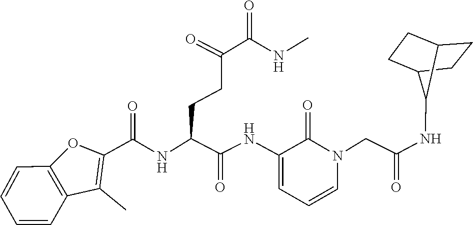 [SMILES: CNC(=O)C(=O)CC[C@H](NC(=O)c1oc2ccccc2c1C)C(=O)Nc1cccn(CC(=O)NC2C3CCC2CC3)c1=O] | (S)-N1-(1-(2-(bicyclo[2.2.1]heptan-7- ylamino)-2-oxoethyl)-2-oxo-1,2- dihydropyridin-3-yl)-N6-methyl-2-(3- methylbenzofuran-2-carboxamido)-5- oxohexanediamide |
| II-97: | 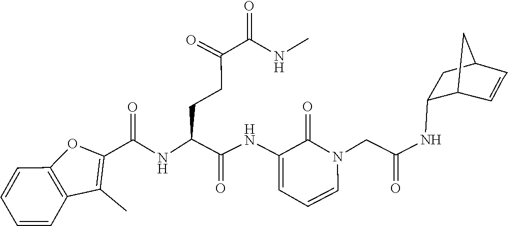 [SMILES: CNC(=O)C(=O)CC[C@H](NC(=O)c1oc2ccccc2c1C)C(=O)Nc1cccn(CC(=O)NC2CC3CC=CC2C3)c1=O] | (S)-N1-(1-(2-(bicyclo[2.2.1]hept-5-en-2- ylamino)-2-oxoethyl)-2-oxo-1,2- dihydropyridin-3-yl)-N6-methyl-2-(3- methylbenzofuran-2-carboxamido)-5- oxohexanediamide |
| II-98: | 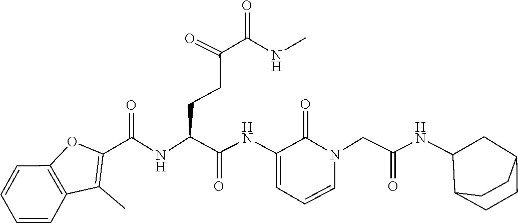 [SMILES: CNC(=O)C(=O)CC[C@H](NC(=O)c1oc2ccccc2c1C)C(=O)Nc1cccn(CC(=O)NC2CC3CCC2CC3)c1=O] | (2S)-N1-(1-(2-(bicyclo[2.2.2]octan-2- ylamino)-2-oxoethyl)-2-oxo-1,2- dihydropyridin-3-yl)-N6-methyl-2-(3- methylbenzofuran-2-carboxamido)-5- oxohexanediamide |
| II-99: | 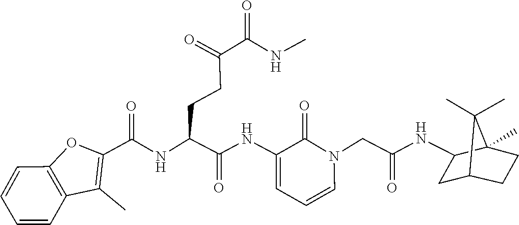 [SMILES: CNC(=O)C(=O)CC[C@H](NC(=O)c1oc2ccccc2c1C)C(=O)Nc1cccn(CC(=O)NC2CC3CC[C@]2(C)C3(C)C)c1=O] | (S)-N1-methyl-5-(3-methylbenzofuran-2- carboxamido)-2-oxo-N6-(2-oxo-1-(2-oxo- 2-((1R,2R,4R)-1,7,7- trimethylbicyclo[2.2.1]heptan-2- ylamino)ethyl)-1,2-dihydropyridin-3- yl)hexanediamide |
| II-100: | 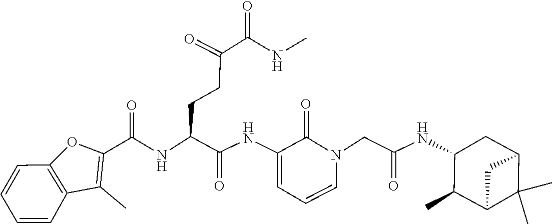 [SMILES: CNC(=O)C(=O)CC[C@H](NC(=O)c1oc2ccccc2c1C)C(=O)Nc1cccn(CC(=O)N[C@@H]2C[C@@H]3C[C@H]([C@H]2C)C3(C)C)c1=O] | (S)-N1-methyl-5-(3-methylbenzofuran-2- carboxamido)-2-oxo-N6-(2-oxo-1-(2-oxo- 2-((1R,2R,3R,5S)-2,6,6- trimethylbicyclo[3.1.1]heptan-3- ylamino)ethyl)-1,2-dihydropyridin-3- yl)hexanediamide |
| II-101: | 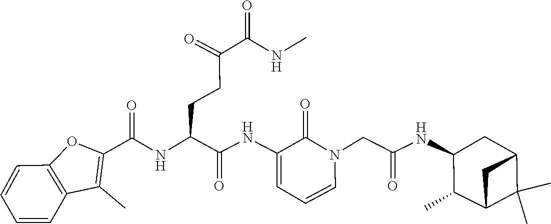 [SMILES: CNC(=O)C(=O)CC[C@H](NC(=O)c1oc2ccccc2c1C)C(=O)Nc1cccn(CC(=O)N[C@H]2C[C@H]3C[C@@H]([C@@H]2C)C3(C)C)c1=O] | (S)-N1-methyl-5-(3-methylbenzofuran-2- carboxamido)-2-oxo-N6-(2-oxo-1-(2-oxo- 2-((1S,2S,3S,5R)-2,6,6- trimethylbicyclo[3.1.1]heptan-3- ylamino)ethyl)-1,2-dihydropyridin-3- yl)hexanediamide |
| II-103: | 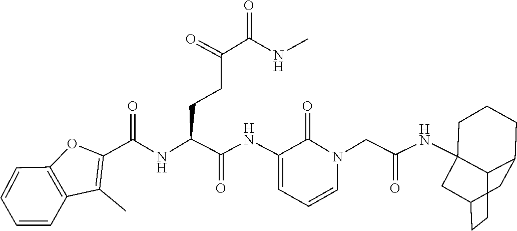 [SMILES: CNC(=O)C(=O)CC[C@H](NC(=O)c1oc2ccccc2c1C)C(=O)Nc1cccn(CC(=O)NC23CCCC4CC(CCC42)C3)c1=O] | (S)-N1-(1-(2-(4-homoisotwistane-3- amino)-2-oxoethyl)-2-oxo-1,2- dihydropyridin-3-yl)-N6-methyl-2-(3- methylbenzofuran-2-carboxamido)-5- oxohexanediamide |
| II-104: | 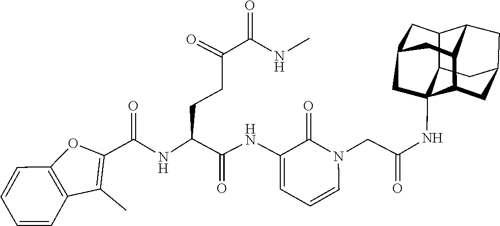 | (S)-N1-(1-(2-(diamantane-1-amino)-2- oxoethyl)-2-oxo-1,2-dihydropyridin-3-yl)- N6-methyl-2-(3-methylbenzofuran-2- carboxamido)-5-oxohexanediamide |
| II-105: | 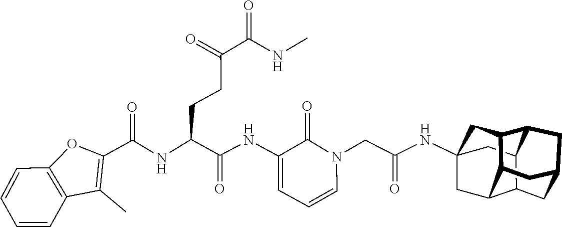 | (S)-N1-(1-(2-(diamantane-4-amino)-2- oxoethyl)-2-oxo-1,2-dihydropyridin-3-yl)- N6-methyl-2-(3-methylbenzofuran-2- carboxamido)-5-oxohexanediamide |
| II-107: | 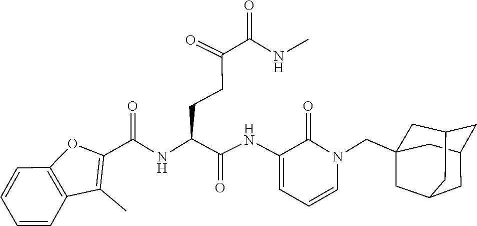 [SMILES: CNC(=O)C(=O)CC[C@H](NC(=O)c1oc2ccccc2c1C)C(=O)Nc1cccn(CC23CC4CC(CC(C4)C2)C3)c1=O] | (S)-N1-(1-(1-adamantylmethyl)-2-oxo-1,2- dihydropyridin-3-yl)-N6-methyl-2-(3- methylbenzofuran-2-carboxamido)-5- oxohexanediamide |
| II-108: | 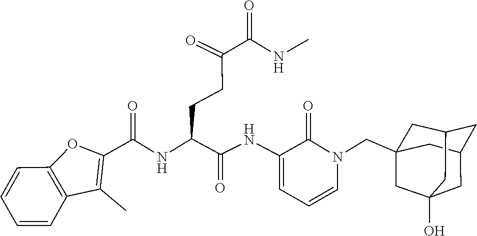 [SMILES: CNC(=O)C(=O)CC[C@H](NC(=O)c1oc2ccccc2c1C)C(=O)Nc1cccn(CC23CC4CC(CC(O)(C4)C2)C3)c1=O] | (2S)-N1-(1-((3-hydroxy-1- adamantyl)methyl)-2-oxo-1,2- dihydropyridin-3-yl)-N6-methyl-2-(3- methylbenzofuran-2-carboxamido)-5- oxohexanediamide |
| II-109: | 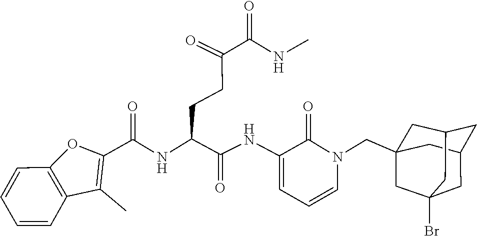 [SMILES: CNC(=O)C(=O)CC[C@H](NC(=O)c1oc2ccccc2c1C)C(=O)Nc1cccn(CC23CC4CC(CC(Br)(C4)C2)C3)c1=O] | (2S)-N1-(1-((3-bromo-1- adamantyl)methyl)-2-oxo-1,2- dihydropyridin-3-yl)-N6-methyl-2-(3- methylbenzofuran-2-carboxamido)-5- oxohexanediamide |
| II-110: | 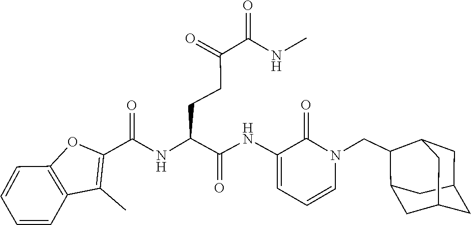 [SMILES: CNC(=O)C(=O)CC[C@H](NC(=O)c1oc2ccccc2c1C)C(=O)Nc1cccn(CC2C3CC4CC(C3)CC2C4)c1=O] | (S)-N1-(1-(2-adamantylmethyl)-2-oxo-1,2- dihydropyridin-3-yl)-N6-methyl-2-(3- methylbenzofuran-2-carboxamido)-5- oxohexanediamide |
| II-111: | 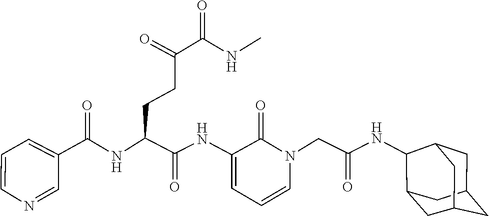 [SMILES: CNC(=O)C(=O)CC[C@H](NC(=O)c1cccnc1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(nicotinamido)-5- oxohexanediamide |
| II-112: | 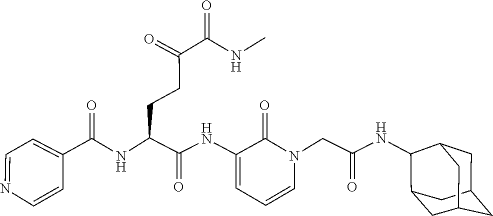 [SMILES: CNC(=O)C(=O)CC[C@H](NC(=O)c1ccncc1)C(=O)Nc1cccn(CC(=O)NC2C3CC4CC(C3)CC2C4)c1=O] | (S)-2-(isonicotinamido)-N1-(1-(2-(2- adamantylamino)-2-oxoethyl)-2-oxo-1,2- dihydropyridin-3-yl)-N6-methyl-5- oxohexanediamide |
| II-113: | 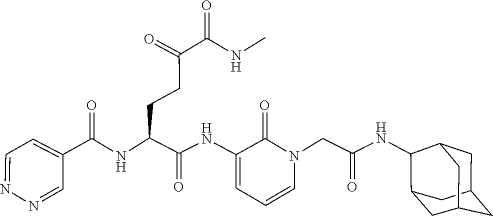 [SMILES: CNC(=O)C(=O)CC[C@H](NC(=O)c1ccnnc1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(pyridazine-4- carboxamido)hexanediamide |
| II-114: | 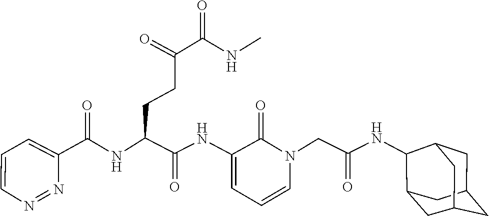 [SMILES: CNC(=O)C(=O)CC[C@H](NC(=O)c1cccnn1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(pyridazine-3- carboxamido)hexanediamide |
| II-115: | 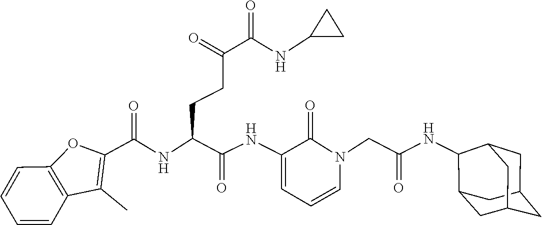 [SMILES: Cc1c(C(=O)N[C@@H](CCC(=O)C(=O)NC2CC2)C(=O)Nc2cccn(CC(=O)NC3C4CC5CC(C4)CC3C5)c2=O)oc2ccccc12] | (S)-N1-cyclopropyl-N6-(1-(2-(2- adamantylamino)-2-oxoethyl)-2-oxo-1,2- dihydropyridin-3-yl)-5-(3- methylbenzofuran-2-carboxamido)-2- oxohexanediamide |
| II-116: | 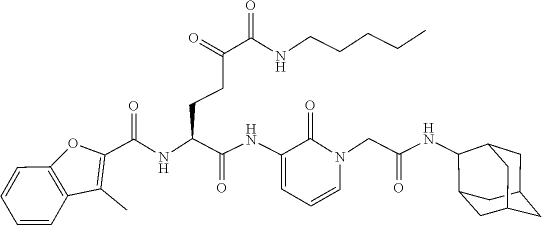 [SMILES: CCCCCNC(=O)C(=O)CC[C@H](NC(=O)c1oc2ccccc2c1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)-2- (3-methylbenzofuran-2-carboxamido)-5- oxo-N6-pentylhexanediamide |
| II-117: | 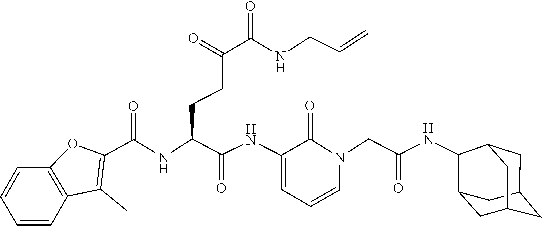 [SMILES: C=CCNC(=O)C(=O)CC[C@H](NC(=O)c1oc2ccccc2c1C)C(=O)Nc1cccn(CC(=O)NC2C3CC4CC(C3)CC2C4)c1=O] | (S)-N1-allyl-N6-(1-(2-(2-adamantylamino)- 2-oxoethyl)-2-oxo-1,2-dihydropyridin-3-yl)- 5-(3-methylbenzofuran-2-carboxamido)-2- oxohexanediamide |
| II-118: | 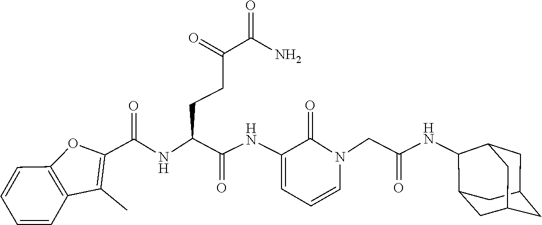 [SMILES: Cc1c(C(=O)N[C@@H](CCC(=O)C(N)=O)C(=O)Nc2cccn(CC(=O)NC3C4CC5CC(C4)CC3C5)c2=O)oc2ccccc12] | (S)-N1-(1-(2-(2-adamantylamino)-2- oxoethyl)-2-oxo-1,2-dihydropyridin-3-yl)-2- (3-methylbenzofuran-2-carboxamido)-5- oxohexanediamide |
| II-119: | 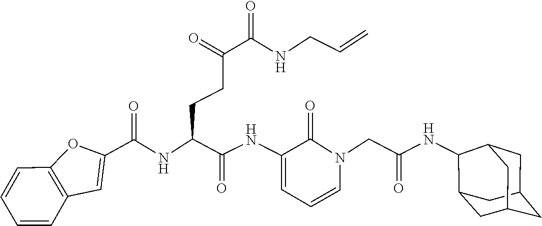 [SMILES: C=CCNC(=O)C(=O)CC[C@H](NC(=O)c1cc2ccccc2o1)C(=O)Nc1cccn(CC(=O)NC2C3CC4CC(C3)CC2C4)c1=O] | (S)-N1-allyl-5-(benzofuran-2- carboxamido)-N6-(1-(2-(2- adamantylamino)-2-oxoethyl)-2-oxo-1,2- dihydropyridin-3-yl)-2-oxohexanediamide |
| II-120: | 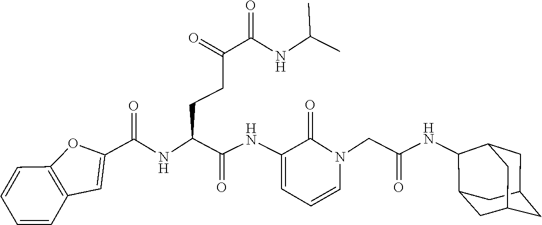 [SMILES: CC(C)NC(=O)C(=O)CC[C@H](NC(=O)c1cc2ccccc2o1)C(=O)Nc1cccn(CC(=O)NC2C3CC4CC(C3)CC2C4)c1=O] | (S)-2-(benzofuran-2-carboxamido)-N6- isopropyl-N1-(1-(2-(2-adamantylamino)-2- oxoethyl)-2-oxo-1,2-dihydropyridin-3-yl)-5- oxohexanediamide |
| II-121: | 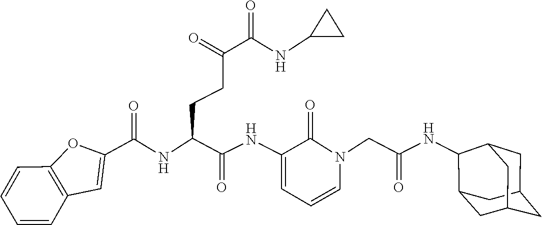 [SMILES: O=C(Cn1cccc(NC(=O)[C@H](CCC(=O)C(=O)NC2CC2)NC(=O)c2cc3ccccc3o2)c1=O)NC1C2CC3CC(C2)CC1C3] | (S)-2-(benzofuran-2-carboxamido)-N6- cyclopropyl-N1-(1-(2-(2-adamantylamino)- 2-oxoethyl)-2-oxo-1,2-dihydropyridin-3-yl)- 5-oxohexanediamide |
| II-122: | 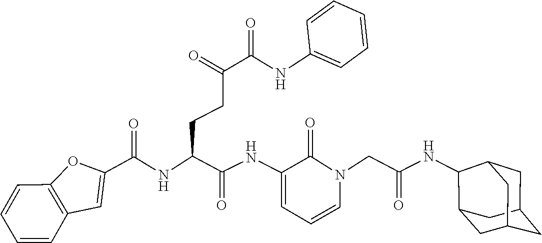 [SMILES: O=C(Cn1cccc(NC(=O)[C@H](CCC(=O)C(=O)Nc2ccccc2)NC(=O)c2cc3ccccc3o2)c1=O)NC1C2CC3CC(C2)CC1C3] | (S)-2-(benzofuran-2-carboxamido)-N1-(1- (2-(2-adamantylamino)-2-oxoethyl)-2-oxo- 1,2-dihydropyridin-3-yl)-5-oxo-N6- phenylhexanediamide |
| II-123: | 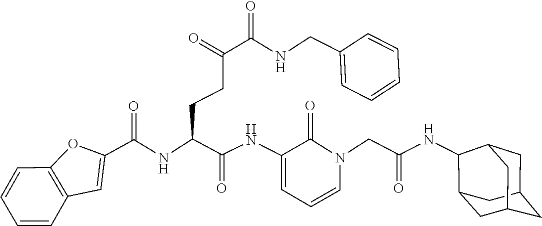 [SMILES: O=C(Cn1cccc(NC(=O)[C@H](CCC(=O)C(=O)NCc2ccccc2)NC(=O)c2cc3ccccc3o2)c1=O)NC1C2CC3CC(C2)CC1C3] | (S)-2-(benzofuran-2-carboxamido)-N6- benzyl-N1-(1-(2-(2-adamantylamino)-2- oxoethyl)-2-oxo-1,2-dihydropyridin-3-yl)-5- oxohexanediamide |
| II-124: | 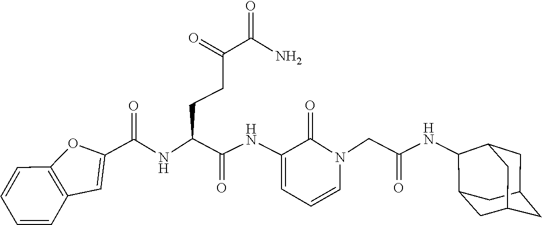 [SMILES: NC(=O)C(=O)CC[C@H](NC(=O)c1cc2ccccc2o1)C(=O)Nc1cccn(CC(=O)NC2C3CC4CC(C3)CC2C4)c1=O] | (S)-2-(benzofuran-2-carboxamido)-N1-(1- (2-(2-adamantylamino)-2-oxoethyl)-2-oxo- 1,2-dihydropyridin-3-yl)-5- oxohexanediamide |
| II-125: | 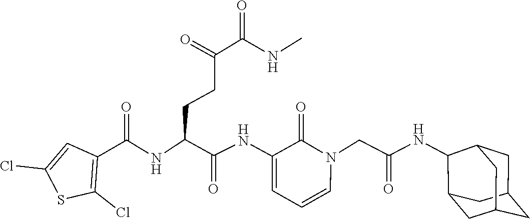 [SMILES: CNC(=O)C(=O)CC[C@H](NC(=O)c1cc(Cl)sc1Cl)C(=O)Nc1cccn(CC(=O)NC2C3CC4CC(C3)CC2C4)c1=O] | (S)-2-(2,5-dichlorothiophene-3- carboxamido)-N1-(1-(2-(2- adamantylamino)-2-oxoethyl)-2-oxo-1,2- dihydropyridin-3-yl)-5-oxohexanediamide |
| II-126: | 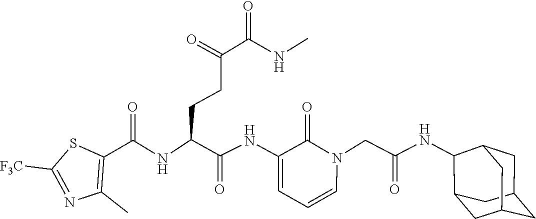 [SMILES: CNC(=O)C(=O)CC[C@H](NC(=O)c1sc(C(F)(F)F)nc1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)-2- (4-methyl-2-(trifluoromethyl)thiazole-5- carboxamido)-5-oxohexanediamide |
| II-127: | 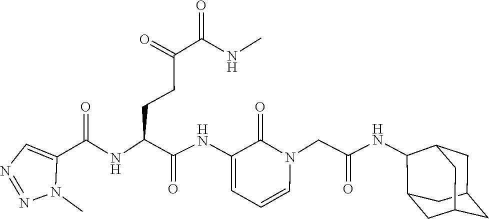 [SMILES: CNC(=O)C(=O)CC[C@H](NC(=O)c1cnnn1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)-2- (1-methyl-1H-1,2,3-triazole-5- carboxamido)-5-oxohexanediamide |
| II-128: | 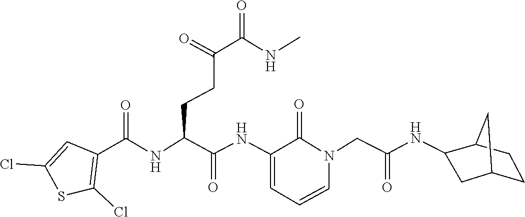 [SMILES: CNC(=O)C(=O)CC[C@H](NC(=O)c1cc(Cl)sc1Cl)C(=O)Nc1cccn(CC(=O)NC2CC3CCC2C3)c1=O] | (2S)-N1-(1-(2-((1R,2R,4S)- bicyclo[2.2.1]heptan-2-ylamino)-2- oxoethyl)-2-oxo-1,2-dihydropyridin-3-yl)-2- (2,5-dichlorothiophene-3-carboxamido)- N6-methyl-5-oxohexanediamide |
| II-129: | 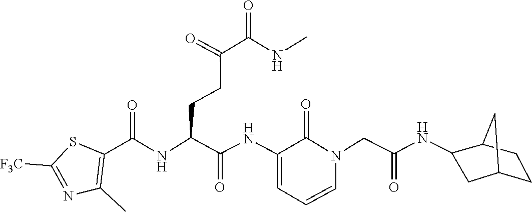 [SMILES: CNC(=O)C(=O)CC[C@H](NC(=O)c1sc(C(F)(F)F)nc1C)C(=O)Nc1cccn(CC(=O)NC2CC3CCC2C3)c1=O] | (2S)-N1-(1-(2-((1R,2R,4S)- bicyclo[2.2.1]heptan-2-ylamino)-2- oxoethyl)-2-oxo-1,2-dihydropyridin-3-yl)- N6-methyl-2-(4-methyl-2- (trifluoromethyl)thiazole-5-carboxamido)-5- oxohexanediamide |
| II-130: | 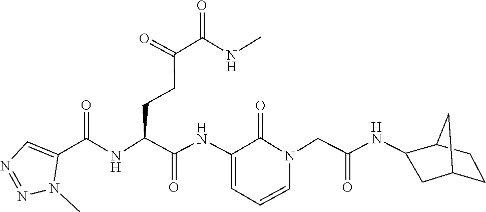 [SMILES: CNC(=O)C(=O)CC[C@H](NC(=O)c1cnnn1C)C(=O)Nc1cccn(CC(=O)NC2CC3CCC2C3)c1=O] | (2S)-N1-(1-(2-((1R,2R,4S)- bicyclo[2.2.1]heptan-2-ylamino)-2- oxoethyl)-2-oxo-1,2-dihydropyridin-3-yl)- N6-methyl-2-(1-methyl-1H-1,2,3-triazole-5- carboxamido)-5-oxohexanediamide |
| II-131: | 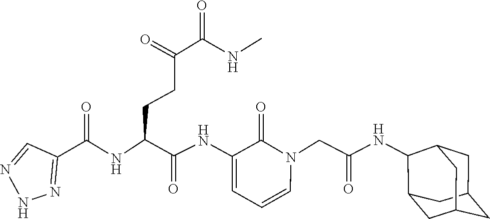 [SMILES: CNC(=O)C(=O)CC[C@H](NC(=O)c1cn[nH]n1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(2H-1,2,3-triazole-4- carboxamido)hexanediamide |
| II-132: | 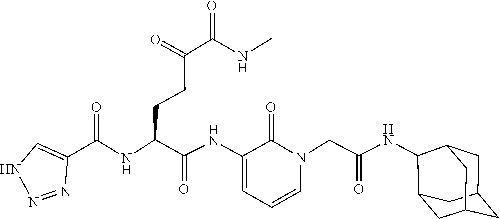 [SMILES: CNC(=O)C(=O)CC[C@H](NC(=O)c1c[nH]nn1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(1H-1,2,3-triazole-4- carboxamido)hexanediamide |
| II-133: | 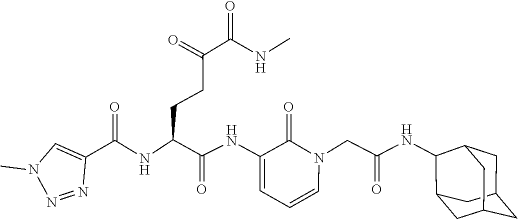 [SMILES: CNC(=O)C(=O)CC[C@H](NC(=O)c1cn(C)nn1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-1,2,3-triazole-4- carboxamido)-5-oxohexanediamide |
| II-134: | 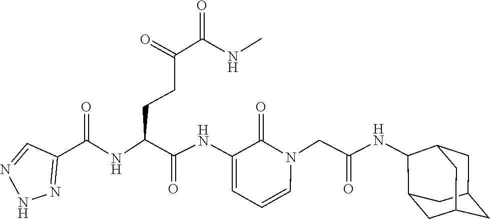 [SMILES: CNC(=O)C(=O)CC[C@H](NC(=O)c1cn[nH]n1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(1H-1,2,4-triazole-3- carboxamido)hexanediamide |
| II-135: | 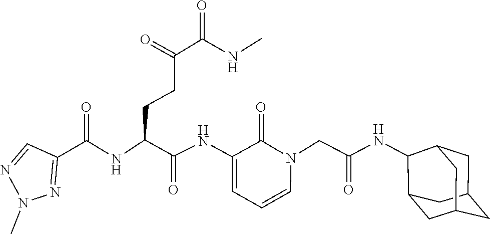 [SMILES: CNC(=O)C(=O)CC[C@H](NC(=O)c1cnn(C)n1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-1,2,4-triazole-3- carboxamido)-5-oxohexanediamide |
| II-136: | 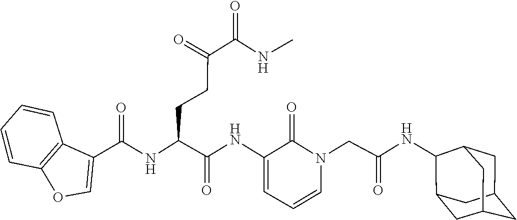 [SMILES: CNC(=O)C(=O)CC[C@H](NC(=O)c1coc2ccccc12)C(=O)Nc1cccn(CC(=O)NC2C3CC4CC(C3)CC2C4)c1=O] | (S)-2-(benzofuran-3-carboxamido)-N1-(1- (2-(2-adamantylamino)-2-oxoethyl)-2-oxo- 1,2-dihydropyridin-3-yl)-N6-methyl-5- oxohexanediamide |
| II-137: | 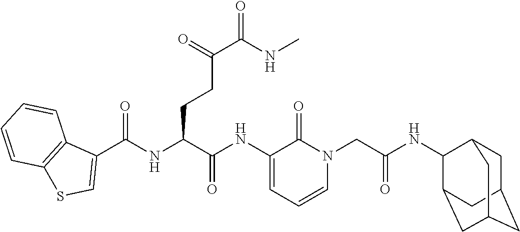 [SMILES: CNC(=O)C(=O)CC[C@H](NC(=O)c1csc2ccccc12)C(=O)Nc1cccn(CC(=O)NC2C3CC4CC(C3)CC2C4)c1=O] | (S)-2-(benzo[b]thiophene-3-carboxamido)- N1-(1-(2-(2-adamantylamino)-2-oxoethyl)- 2-oxo-1,2-dihydropyridin-3-yl)-N6-methyl- 5-oxohexanediamide |
| II-138 | 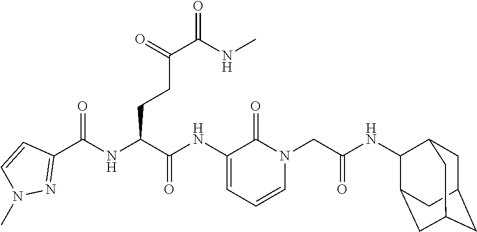 [SMILES: CNC(=O)C(=O)CC[C@H](NC(=O)c1ccn(C)n1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-pyrazole-3- carboxamido)-5-oxohexanediamide |
| II-139 | 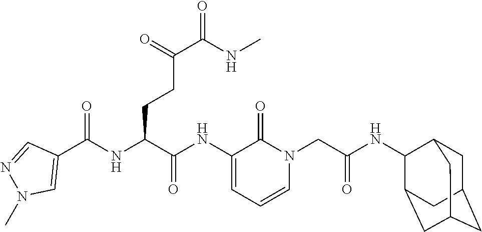 [SMILES: CNC(=O)C(=O)CC[C@H](NC(=O)c1cnn(C)c1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-pyrazole-4- carboxamido)-5-oxohexanediamide |
| II-140 | 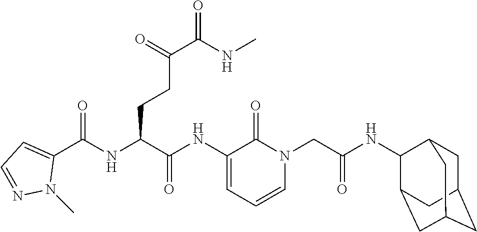 [SMILES: CNC(=O)C(=O)CC[C@H](NC(=O)c1ccnn1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(1-methyl-1H-pyrazole-5- carboxamido)-5-oxohexanediamide |
| II-141 | 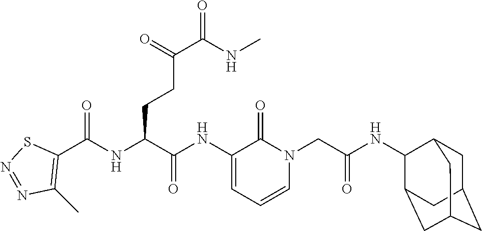 [SMILES: CNC(=O)C(=O)CC[C@H](NC(=O)c1snnc1C)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-2-(4-methyl-1,2,3-thiadiazole-5- carboxamido)-5-oxohexanediamide |
| II-142 | 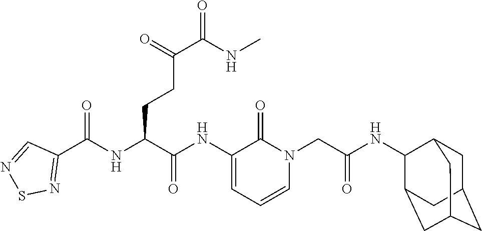 [SMILES: CNC(=O)C(=O)CC[C@H](NC(=O)c1cnsn1)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)- N6-methyl-5-oxo-2-(1,2,5-thiadiazole-3- carboxamido)hexanediamide |
| II-143 | 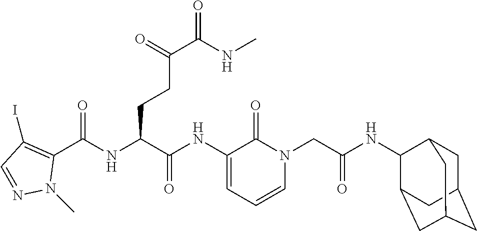 [SMILES: CNC(=O)C(=O)CC[C@H](NC(=O)c1c(I)cnn1C)C(=O)Nc1cccn(CC(=O)NC2C3CC4CC(C3)CC2C4)c1=O] | (S)-2-(4-iodo-1-methyl-1H-pyrazole-5- carboxamido)-N1-(1-(2-(2- adamantylamino)-2-oxoethyl)-2-oxo-1,2- dihydropyridin-3-yl)-N6-methyl-5- oxohexanediamide |
| II-144 | 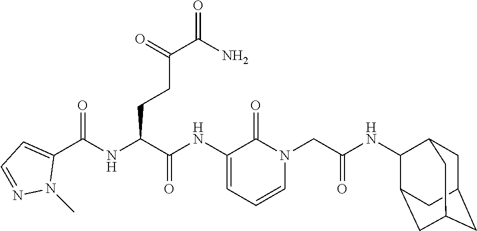 [SMILES: Cn1nccc1C(=O)N[C@@H](CCC(=O)C(N)=O)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)-2- (1-methyl-1H-pyrazole-5-carboxamido)-5- oxohexanediamide |
| II-145 | 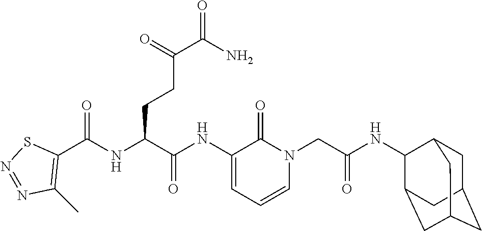 [SMILES: Cc1nnsc1C(=O)N[C@@H](CCC(=O)C(N)=O)C(=O)Nc1cccn(CC(=O)NC2C3CC4CC(C3)CC2C4)c1=O] | (S)-N1-(1-(2-(2-adamantylamino)-2- oxoethyl)-2-oxo-1,2-dihydropyridin-3-yl)-2- (4-methyl-1,2,3-thiadiazole-5- carboxamido)-5-oxohexanediamide |
| II-146 | 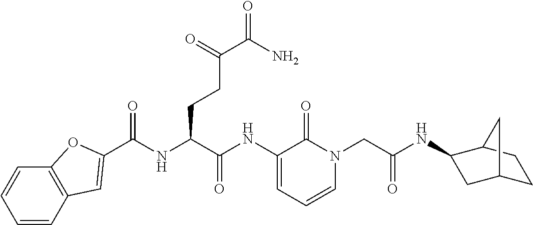 [SMILES: NC(=O)C(=O)CC[C@H](NC(=O)c1cc2ccccc2o1)C(=O)Nc1cccn(CC(=O)N[C@@H]2CC3CCC2C3)c1=O] | (S)-2-(benzofuran-2-carboxamido)-N1-(1- (2-((1R,2R,4S)-bicyclo[2.2.1]heptan-2- ylamino)-2-oxoethyl)-2-oxo-1,2- dihydropyridin-3-yl)-5-oxohexanediamide |
| II-147 | 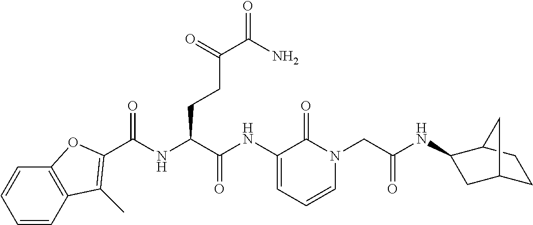 [SMILES: Cc1c(C(=O)N[C@@H](CCC(=O)C(N)=O)C(=O)Nc2cccn(CC(=O)N[C@@H]3CC4CCC3C4)c2=O)oc2ccccc12] | (S)-N1-(1-(2-((1R,2R,4S)- bicyclo[2.2.1]heptan-2-ylamino)-2- oxoethyl)-2-oxo-1,2-dihydropyridin-3-yl)-2- (3-methylbenzofuran-2-carboxamido)-5- oxohexanediamide |
| II-148 | 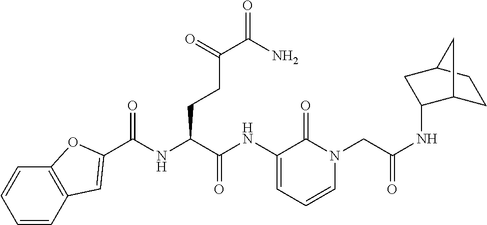 [SMILES: NC(=O)C(=O)CC[C@H](NC(=O)c1cc2ccccc2o1)C(=O)Nc1cccn(CC(=O)NC2CC3CCC2C3)c1=O] | (S)-2-(benzofuran-2-carboxamido)-N1-(1- (2-((1S,2R,4R)-bicyclo[2.2.1]heptan-2- ylamino)-2-oxoethyl)-2-oxo-1,2- dihydropyridin-3-yl)-5-oxohexanediamide |
| II-149 | 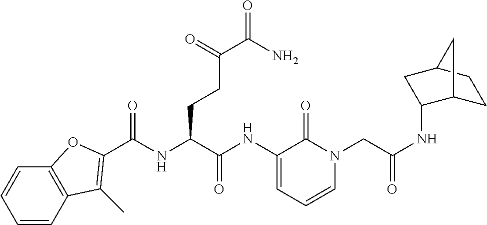 [SMILES: Cc1c(C(=O)N[C@@H](CCC(=O)C(N)=O)C(=O)Nc2cccn(CC(=O)NC3CC4CCC3C4)c2=O)oc2ccccc12] | (S)-N1-(1-(2-((1S,2R,4R)- bicyclo[2.2.1]heptan-2-ylamino)-2- oxoethyl)-2-oxo-1,2-dihydropyridin-3-yl)-2- (3-methylbenzofuran-2-carboxamido)-5- oxohexanediamide |
| II-150 | 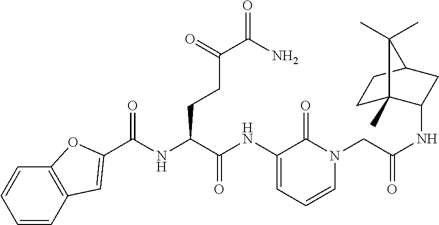 [SMILES: CC1(C)C2CC[C@@]1(C)C(NC(=O)Cn1cccc(NC(=O)[C@H](CCC(=O)C(N)=O)NC(=O)c3cc4ccccc4o3)c1=O)C2] | (S)-2-(benzofuran-2-carboxamido)-5-oxo- N1-(2-oxo-1-(2-oxo-2-((1R,2S,4R)-1,7,7- trimethylbicyclo[2.2.1]heptan-2- ylamino)ethyl)-1,2-dihydropyridin-3- yl)hexanediamide |
| II-151 | 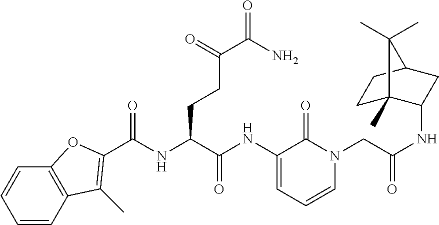 [SMILES: Cc1c(C(=O)N[C@@H](CCC(=O)C(N)=O)C(=O)Nc2cccn(CC(=O)NC3CC4CC[C@]3(C)C4(C)C)c2=O)oc2ccccc12] | (S)-2-(3-methylbenzofuran-2- carboxamido)-5-oxo-N1-(2-oxo-1-(2-oxo-2- ((1R,2S,4R)-1,7,7- trimethylbicyclo[2.2.1]heptan-2- ylamino)ethyl)-1,2-dihydropyridin-3- yl)hexanediamide |
| II-152 | 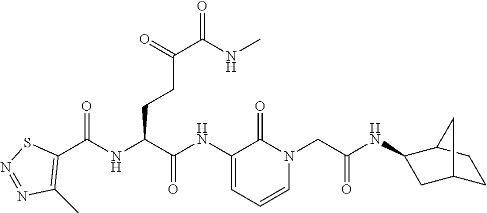 [SMILES: CNC(=O)C(=O)CC[C@H](NC(=O)c1snnc1C)C(=O)Nc1cccn(CC(=O)N[C@@H]2CC3CCC2C3)c1=O] | (S)-N1-(1-(2-((1R,2R,4S)- bicyclo[2.2.1]heptan-2-ylamino)-2- oxoethyl)-2-oxo-1,2-dihydropyridin-3-yl)- N6-methyl-2-(4-methyl-1,2,3-thiadiazole-5- carboxamido)-5-oxohexanediamide |
| II-153 | 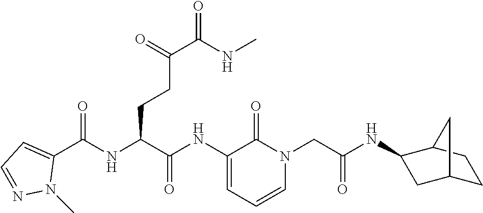 [SMILES: CNC(=O)C(=O)CC[C@H](NC(=O)c1ccnn1C)C(=O)Nc1cccn(CC(=O)N[C@@H]2CC3CCC2C3)c1=O] | (S)-N1-(1-(2-((1R,2R,4S)- bicyclo[2.2.1]heptan-2-ylamino)-2- oxoethyl)-2-oxo-1,2-dihydropyridin-3-yl)- N6-methyl-2-(1-methyl-1H-pyrazole-5- carboxamido)-5-oxohexanediamide |
| II-154 | 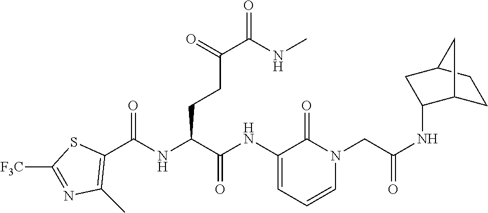 [SMILES: CNC(=O)C(=O)CC[C@H](NC(=O)c1sc(C(F)(F)F)nc1C)C(=O)Nc1cccn(CC(=O)NC2CC3CCC2C3)c1=O] | (S)-N1-(1-(2-((1S,2R,4R)- bicyclo[2.2.1]heptan-2-ylamino)-2- oxoethyl)-2-oxo-1,2-dihydropyridin-3-yl)- N6-methyl-2-(4-methyl-2- (trifluoromethyl)thiazole-5-carboxamido)-5- oxohexanediamide |
| II-155 | 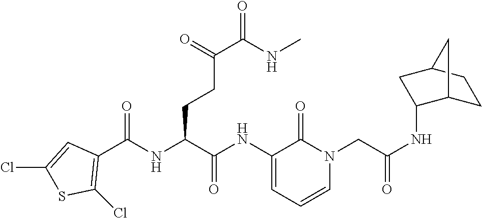 [SMILES: CNC(=O)C(=O)CC[C@H](NC(=O)c1cc(Cl)sc1Cl)C(=O)Nc1cccn(CC(=O)NC2CC3CCC2C3)c1=O] | (S)-N1-(1-(2-((1S,2R,4R)- bicyclo[2.2.1]heptan-2-ylamino)-2- oxoethyl)-2-oxo-1,2-dihydropyridin-3-yl)-2- (2,5-dichlorothiophene-3-carboxamido)- N6-methyl-5-oxohexanediamide |
| II-156 | 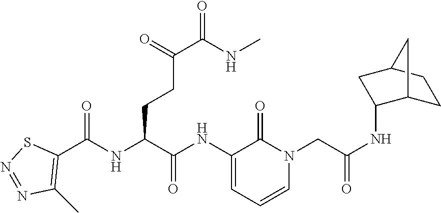 [SMILES: CNC(=O)C(=O)CC[C@H](NC(=O)c1snnc1C)C(=O)Nc1cccn(CC(=O)NC2CC3CCC2C3)c1=O] | (S)-N1-(1-(2-((1S,2R,4R)- bicyclo[2.2.1]heptan-2-ylamino)-2- oxoethyl)-2-oxo-1,2-dihydropyridin-3-yl)- N6-methyl-2-(4-methyl-1,2,3-thiadiazole-5- carboxamido)-5-oxohexanediamide |
| II-157 | 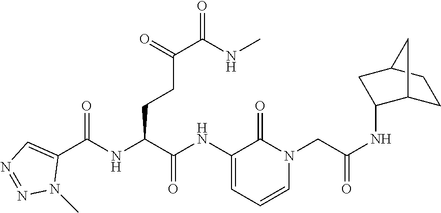 [SMILES: CNC(=O)C(=O)CC[C@H](NC(=O)c1cnnn1C)C(=O)Nc1cccn(CC(=O)NC2CC3CCC2C3)c1=O] | (S)-N1-(1-(2-((1S,2R,4R)- bicyclo[2.2.1]heptan-2-ylamino)-2- oxoethyl)-2-oxo-1,2-dihydropyridin-3-yl)- N6-methyl-2-(1-methyl-1H-1,2,3-triazole-5- carboxamido)-5-oxohexanediamide |
| II-158 | 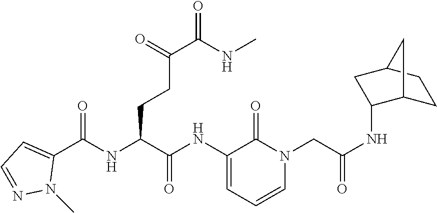 [SMILES: CNC(=O)C(=O)CC[C@H](NC(=O)c1ccnn1C)C(=O)Nc1cccn(CC(=O)NC2CC3CCC2C3)c1=O] | (S)-N1-(1-(2-((1S,2R,4R)- bicyclo[2.2.1]heptan-2-ylamino)-2- oxoethyl)-2-oxo-1,2-dihydropyridin-3-yl)- N6-methyl-2-(1-methyl-1H-pyrazole-5- carboxamido)-5-oxohexanediamide |
| II-159 | 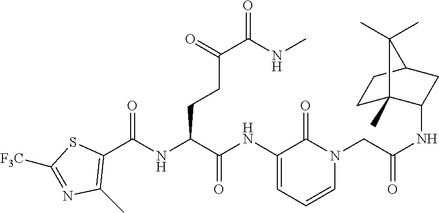 [SMILES: CNC(=O)C(=O)CC[C@H](NC(=O)c1sc(C(F)(F)F)nc1C)C(=O)Nc1cccn(CC(=O)NC2CC3CC[C@]2(C)C3(C)C)c1=O] | (S)-N1-methyl-5-(4-methyl-2- (trifluoromethyl)thiazole-5-carboxamido)-2- OXO-N6-(2-oxo-1-(2-oxo-2-((1R,2S,4R)- 1,7,7-trimethylbicyclo[2.2.1]heptan-2- ylamino)ethyl)-1,2-dihydropyridin-3- yl)hexanediamide |
| II-160 | 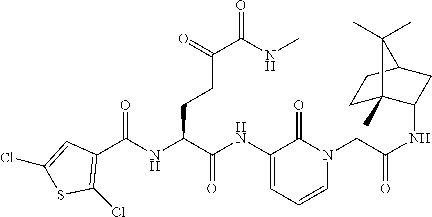 [SMILES: CNC(=O)C(=O)CC[C@H](NC(=O)c1cc(Cl)sc1Cl)C(=O)Nc1cccn(CC(=O)NC2CC3CC[C@]2(C)C3(C)C)c1=O] | (S)-2-(2,5-dichlorothiophene-3- carboxamido)-N6-methyl-5-oxo-N1-(2-oxo- 1-(2-oxo-2-((1R,2S,4R)-1,7,7- trimethylbicyclo[2.2.1]heptan-2- ylamino)ethyl)-1,2-dihydropyridin-3- yl)hexanediamide |
| II-161 | 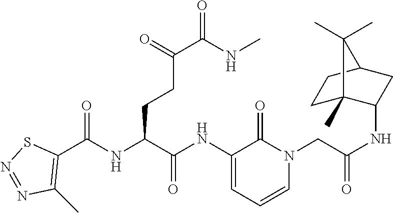 [SMILES: CNC(=O)C(=O)CC[C@H](NC(=O)c1snnc1C)C(=O)Nc1cccn(CC(=O)NC2CC3CC[C@]2(C)C3(C)C)c1=O] | (S)-N1-methyl-5-(4-methyl-1,2,3- thiadiazole-5-carboxamido)-2-oxo-N6-(2- ox0-1-(2-oxo-2-((1R,2S,4R)-1,7,7- trimethylbicyclo[2.2.1]heptan-2- ylamino)ethyl)-1,2-dihydropyridin-3- yl)hexanediamide |
| II-162 | 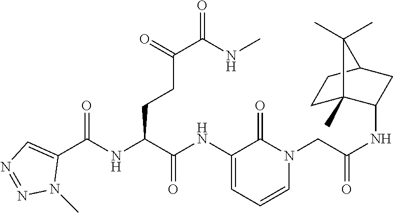 [SMILES: CNC(=O)C(=O)CC[C@H](NC(=O)c1cnnn1C)C(=O)Nc1cccn(CC(=O)NC2CC3CC[C@]2(C)C3(C)C)c1=O] | (S)-N1-methyl-5-(1-methyl-1H-1,2,3- triazole-5-carboxamido)-2-oxo-N6-(2-oxo- 1-(2-oxo-2-((1R,2S,4R)-1,7,7- trimethylbicyclo[2.2.1]heptan-2- ylamino)ethyl)-1,2-dihydropyridin-3- yl)hexanediamide |
| II-163 | 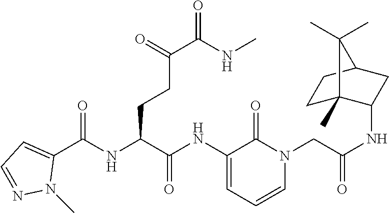 [SMILES: CNC(=O)C(=O)CC[C@H](NC(=O)c1ccnn1C)C(=O)Nc1cccn(CC(=O)NC2CC3CC[C@]2(C)C3(C)C)c1=O] | (S)-N1-methyl-5-(1-methyl-1H-pyrazole-5- carboxamido)-2-oxo-N6-(2-oxo-1-(2-oxo-2- ((1R,2S,4R)-1,7,7- trimethylbicyclo[2.2.1]heptan-2- ylamino)ethyl)-1,2-dihydropyridin-3- yl)hexanediamide |
| II-164 | 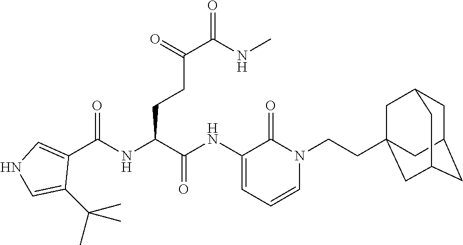 [SMILES: CNC(=O)C(=O)CC[C@H](NC(=O)c1c[nH]cc1C(C)(C)C)C(=O)Nc1cccn(CCC23CC4CC(CC(C4)C2)C3)c1=O] | (2S)-2-(4-tert-butyl-1H-pyrrole-3- carboxamido)-N6-methyl-N1-(1-(2-(1- adamantylamino)ethyl)-2-oxo-1,2- dihydropyridin-3-yl)-5-oxohexanediamide |
| II-165 | 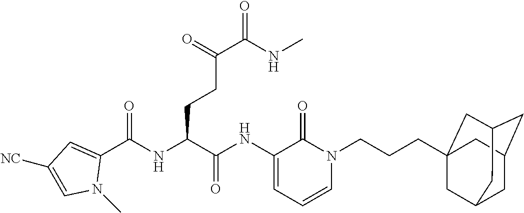 [SMILES: CNC(=O)C(=O)CC[C@H](NC(=O)c1cc(C#N)cn1C)C(=O)Nc1cccn(CCCC23CC4CC(CC(C4)C2)C3)c1=O] | (2S)-2-(4-cyano-1-methyl-1H-pyrrole-2- carboxamido)-N6-methyl-N1-(1-(3-(1- adamantylamino)propyl)-2-oxo-1,2- dihydropyridin-3-yl)-5-oxohexanediamide |
| II-166 | 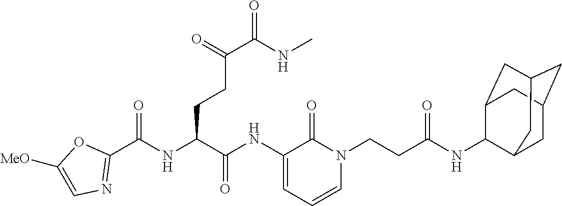 [SMILES: CNC(=O)C(=O)CC[C@H](NC(=O)c1ncc(OC)o1)C(=O)Nc1cccn(CCC(=O)NC2C3CC4CC(C3)CC2C4)c1=O] | (S)-N1-(1-(3-(2-adamantylamino)-3- oxopropyl)-2-oxo-1,2-dihydropyridin-3-yl)- 2-(5-methoxyoxazole-2-carboxamido)-N6- methyl-5-oxohexanediamide |
| II-167 | 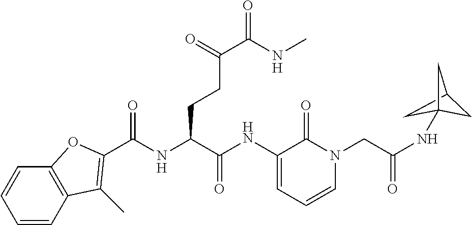 [SMILES: CNC(=O)C(=O)CC[C@H](NC(=O)c1oc2ccccc2c1C)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-methyl-2-(3- methylbenzofuran-2-carboxamido)-5- oxohexanediamide |
| II-168 | 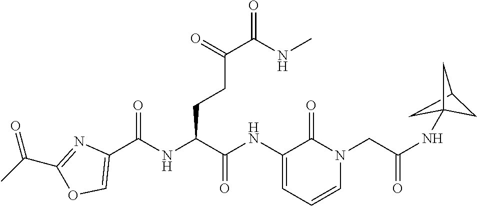 [SMILES: CNC(=O)C(=O)CC[C@H](NC(=O)c1coc(C(C)=O)n1)C(=O)Nc1cccn(CC(=O)NC23CC(C2)C3)c1=O] | (S)-2-(2-acetyloxazole-4-carboxamido)- N1-(1-(2-(bicyclo[1.1.1]pentan-1-ylamino)- 2-oxoethyl)-2-oxo-1,2-dihydropyridin-3-yl)- N6-methyl-5-oxohexanediamide |
| II-169 | 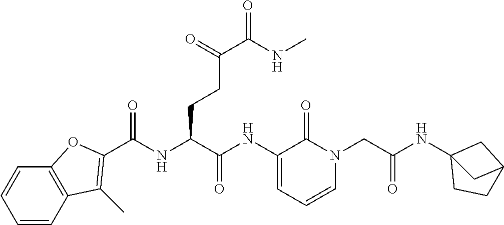 [SMILES: CNC(=O)C(=O)CC[C@H](NC(=O)c1oc2ccccc2c1C)C(=O)Nc1cccn(CC(=O)NC23CCC(C2)C3)c1=O] | (S)-N1-(1-(2-(bicyclo[2.1.1]hexan-1- ylamino)-2-oxoethyl)-2-oxo-1,2- dihydropyridin-3-yl)-N6-methyl-2-(3- methylbenzofuran-2-carboxamido)-5- oxohexanediamide |
| II-170 | 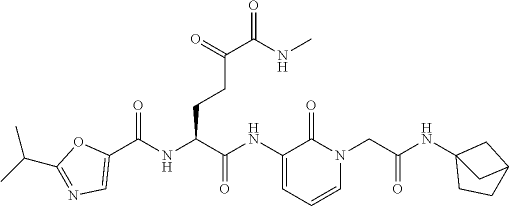 [SMILES: CNC(=O)C(=O)CC[C@H](NC(=O)c1cnc(C(C)C)o1)C(=O)Nc1cccn(CC(=O)NC23CCC(C2)C3)c1=O] | (S)-N1-(1-(2-(bicyclo[2.1.1]hexan-1- ylamino)-2-oxoethyl)-2-oxo-1,2- dihydropyridin-3-yl)-2-(2-isopropyloxazole- 5-carboxamido)-N6-methyl-5- oxohexanediamide |
| II-171 | 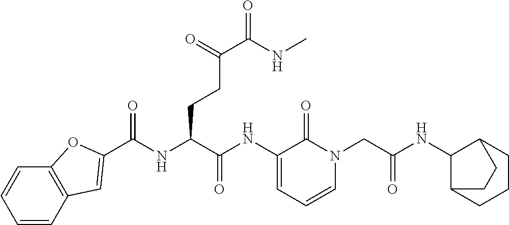 [SMILES: CNC(=O)C(=O)CC[C@H](NC(=O)c1cc2ccccc2o1)C(=O)Nc1cccn(CC(=O)NC2C3CCCC2CC3)c1=O] | (2S)-2-(benzofuran-2-carboxamido)-N1-(1- (2-(bicyclo[3.2.1]octan-8-ylamino)-2- oxoethyl)-2-oxo-1,2-dihydropyridin-3-yl)- N6-methyl-5-oxohexanediamide |
| II-172 | 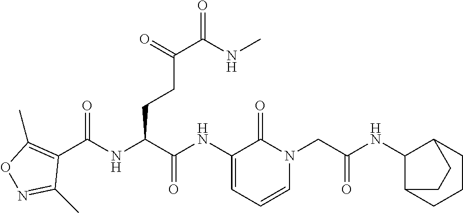 [SMILES: CNC(=O)C(=O)CC[C@H](NC(=O)c1c(C)noc1C)C(=O)Nc1cccn(CC(=O)NC2C3CCCC2CC3)c1=O] | (2S)-N1-(1-(2-(bicyclo[3.2.1]octan-8- ylamino)-2-oxoethyl)-2-oxo-1,2- dihydropyridin-3-yl)-2-(3,5- dimethylisoxazole-4-carboxamido)-N6- methyl-5-oxohexanediamide |
| II-173 | 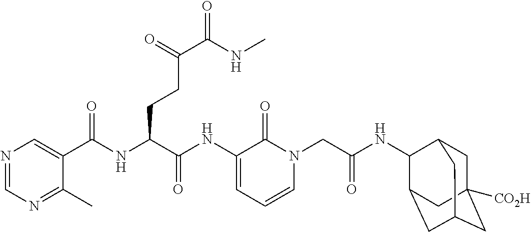 [SMILES: CNC(=O)C(=O)CC[C@H](NC(=O)c1cncnc1C)C(=O)Nc1cccn(CC(=O)NC2C3CC4CC2CC(C(=O)O)(C4)C3)c1=O] | (S)-N1-(1-(2-(5-carboxy-2- aminoadamantane)-2-oxoethyl)-2-oxo-1,2- dihydropyridin-3-yl)-N6-methyl-2-(4- methylpyrimidine-5-carboxamido)-5- oxohexanediamide |
| II-174 | 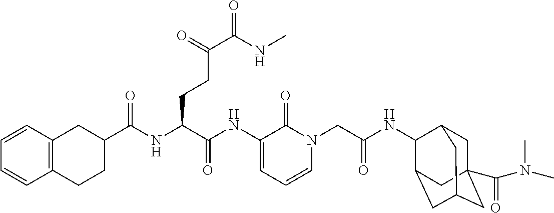 [SMILES: CNC(=O)C(=O)CC[C@H](NC(=O)C1CCc2ccccc2C1)C(=O)Nc1cccn(CC(=O)NC2C3CC4CC2CC(C(=O)N(C)C)(C4)C3)c1=O] | (2S)-N1-(1-(2-(4-aminoadamantane-N,N- dimethyl-1-carboxamide)-2-oxoethyl)-2- oxo-1,2-dihydropyridin-3-yl)-N6-methyl-5- oxo-2-(1,2,3,4-tetrahydronaphthalene-2- carboxamido)hexanediamide |
| II-175 | 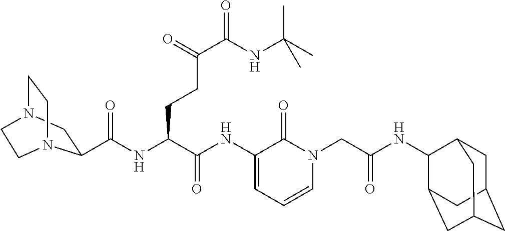 [SMILES: CC(C)(C)NC(=O)C(=O)CC[C@H](NC(=O)C1CN2CCN1CC2)C(=O)Nc1cccn(CC(=O)NC2C3CC4CC(C3)CC2C4)c1=O] | (S)-2-((S)-1,4-diazabicyclo[2.2.2]octane-2- carboxamido)-N6-tert-butyl-N1-(1-(2-(2- adamantylamino)-2-oxoethyl)-2-oxo-1,2- dihydropyridin-3-yl)-5-oxohexanediamide |
| II-176 | 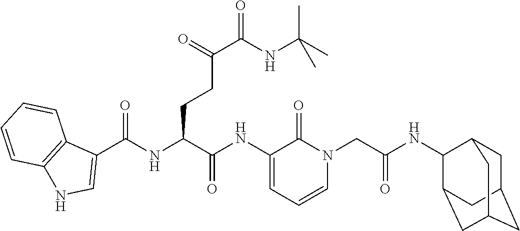 [SMILES: CC(C)(C)NC(=O)C(=O)CC[C@H](NC(=O)c1c[nH]c2ccccc12)C(=O)Nc1cccn(CC(=O)NC2C3CC4CC(C3)CC2C4)c1=O] | (S)-N1-tert-butyl-5-(1H-indole-3- carboxamido)-N6-(1-(2-(2- adamantylamino)-2-oxoethyl)-2-oxo-1,2- dihydropyridin-3-yl)-2-oxohexanediamide |
| II-177 | 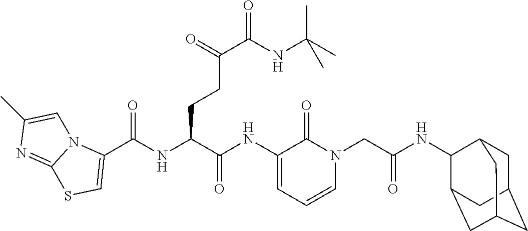 [SMILES: Cc1cn2c(C(=O)N[C@@H](CCC(=O)C(=O)NC(C)(C)C)C(=O)Nc3cccn(CC(=O)NC4C5CC6CC(C5)CC4C6)c3=O)csc2n1] | (S)-N1-tert-butyl-N6-(1-(2-(2- adamantylamino)-2-oxoethyl)-2-oxo-1,2- dihydropyridin-3-yl)-5-(6- methylimidazo[2,1-b]thiazole-3- carboxamido)-2-oxohexanediamide |
| II-178 | 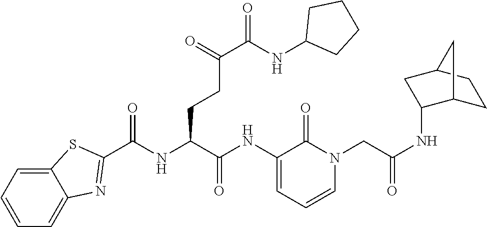 [SMILES: O=C(Cn1cccc(NC(=O)[C@H](CCC(=O)C(=O)NC2CCCC2)NC(=O)c2nc3ccccc3s2)c1=O)NC1CC2CCC1C2] | (S)-2-(benzo[d]thiazole-2-carboxamido)- N1-(1-(2-((1S,2R,4R)-bicyclo[2.2.1]heptan- 2-ylamino)-2-oxoethyl)-2-oxo-1,2- dihydropyridin-3-yl)-N6-cyclopentyl-5- oxohexanediamide |
| II-179 | 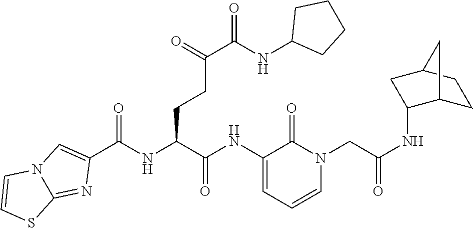 [SMILES: O=C(Cn1cccc(NC(=O)[C@H](CCC(=O)C(=O)NC2CCCC2)NC(=O)c2cn3ccsc3n2)c1=O)NC1CC2CCC1C2] | (S)-N1-(1-(2-((1S,2R,4R)- bicyclo[2.2.1]heptan-2-ylamino)-2- oxoethyl)-2-oxo-1,2-dihydropyridin-3-yl)- N6-cyclopentyl-2-(imidazo[2,1-b]thiazole- 6-carboxamido)-5-oxohexanediamide |
| II-180 | 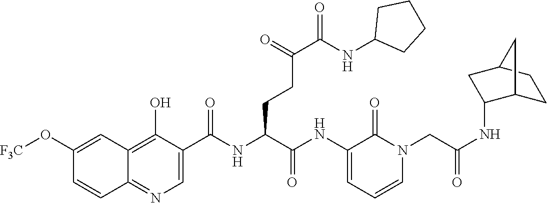 [SMILES: O=C(Cn1cccc(NC(=O)[C@H](CCC(=O)C(=O)NC2CCCC2)NC(=O)c2cnc3ccc(OC(F)(F)F)cc3c2O)c1=O)NC1CC2CCC1C2] | (S)-N1-(1-(2-((1S,2R,4R)- bicyclo[2.2.1]heptan-2-ylamino)-2- oxoethyl)-2-oxo-1,2-dihydropyridin-3-yl)- N6-cyclopentyl-2-(4-hydroxy-6- (trifluoromethoxy)quinoline-3- carboxamido)-5-oxohexanediamide |
| II-181 | 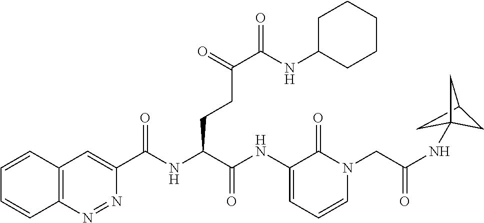 [SMILES: O=C(Cn1cccc(NC(=O)[C@H](CCC(=O)C(=O)NC2CCCCC2)NC(=O)c2cc3ccccc3nn2)c1=O)NC12CC(C1)C2] | (S)-N1-(1-(2-(bicyclo[1.1.1]pentan-1- ylamino)-2-oxoethyl)-2-oxo-1,2- dihydropyridin-3-yl)-2-(cinnoline-3- carboxamido)-N6-cyclohexyl-5- oxohexanediamide |
| II-182 | 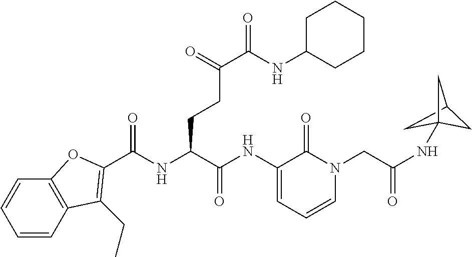 [SMILES: CCc1c(C(=O)N[C@@H](CCC(=O)C(=O)NC2CCCCC2)C(=O)Nc2cccn(CC(=O)NC34CC(C3)C4)c2=O)oc2ccccc12] | (S)-N1-(1-(2-(bicyclo[1.1.1]pentan-1- ylamino)-2-oxoethyl)-2-oxo-1,2- dihydropyridin-3-yl)-N6-cyclohexyl-2-(3- ethylbenzofuran-2-carboxamido)-5- oxohexanediamide |
| II-183 | 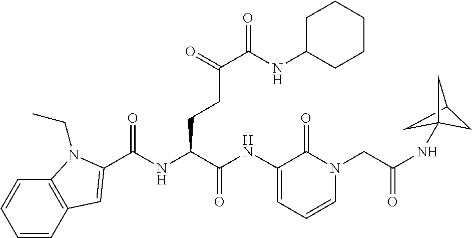 [SMILES: CCn1c(C(=O)N[C@@H](CCC(=O)C(=O)NC2CCCCC2)C(=O)Nc2cccn(CC(=O)NC34CC(C3)C4)c2=O)cc2ccccc21] | (S)-N1-(1-(2-(bicyclo[1.1.1]pentan-1- ylamino)-2-oxoethyl)-2-oxo-1,2- dihydropyridin-3-yl)-N6-cyclohexyl-2-(1- ethyl-1H-indole-2-carboxamido)-5- oxohexanediamide |
| II-184 | 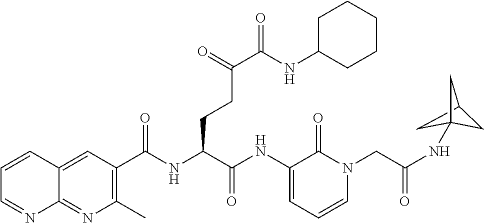 [SMILES: Cc1nc2ncccc2cc1C(=O)N[C@@H](CCC(=O)C(=O)NC1CCCCC1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-cyclohexyl-2-(2- methyl-1,8-naphthyridine-3-carboxamido)- 5-oxohexanediamide |
| II-185 | 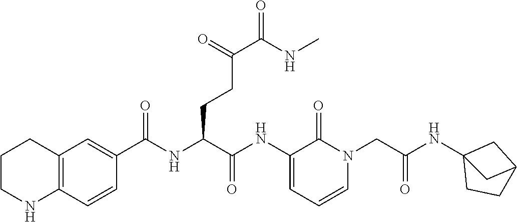 [SMILES: CNC(=O)C(=O)CC[C@H](NC(=O)c1ccc2c(c1)CCCN2)C(=O)Nc1cccn(CC(=O)NC23CCC(C2)C3)c1=O] | (S)-N1-(1-(2-(bicyclo[2.1.1]hexan-1- ylamino)-2-oxoethyl)-2-oxo-1,2- dihydropyridin-3-yl)-N6-methyl-5-oxo-2- (1,2,3,4-tetrahydroquinoline-6- carboxamido)hexanediamide |
| II-186 | 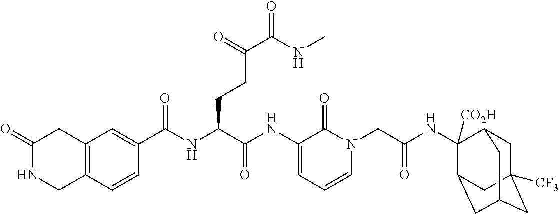 [SMILES: CNC(=O)C(=O)CC[C@H](NC(=O)c1ccc2c(c1)CC(=O)NC2)C(=O)Nc1cccn(CC(=O)NC2(C(=O)O)C3CC4CC2CC(C(F)(F)F)(C4)C3)c1=O] | (S)-N1-(1-(2-(2-carboxy-2-amino-5- (trifluoromethyl)adamantane)-2-oxoethyl)- 2-oxo-1,2-dihydropyridin-3-yl)-N6-methyl- 5-oxo-2-(3-oxo-1,2,3,4- tetrahydroisoquinoline-6- carboxamido)hexanediamide |
| II-187 | 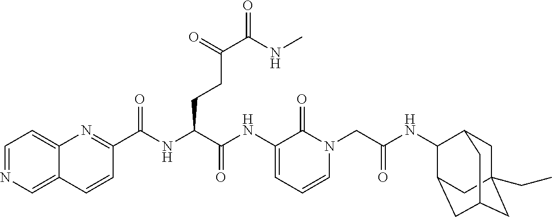 [SMILES: CCC12CC3CC(C1)C(NC(=O)Cn1cccc(NC(=O)[C@H](CCC(=O)C(=O)NC)NC(=O)c4ccc5cnccc5n4)c1=O)C(C3)C2] | (S)-N1-(1-(2-(5-ethyladamantane-2- amino)-2-oxoethyl)-2-oxo-1,2- dihydropyridin-3-yl)-N6-methyl-2-(1,6- naphthyridine-2-carboxamido)-5- oxohexanediamide |
| II-188 | 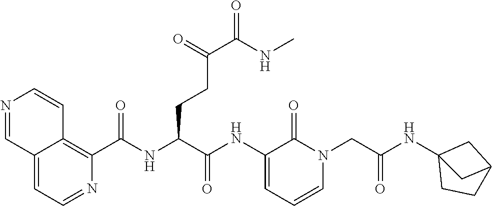 [SMILES: CNC(=O)C(=O)CC[C@H](NC(=O)c1nccc2cnccc12)C(=O)Nc1cccn(CC(=O)NC23CCC(C2)C3)c1=O] | (S)-N1-(1-(2-(bicyclo[2.1.1]hexan-1- ylamino)-2-oxoethyl)-2-oxo-1,2- dihydropyridin-3-yl)-N6-methyl-2-(2,6- naphthyridine-1-carboxamido)-5- oxohexanediamide |
| II-189 | 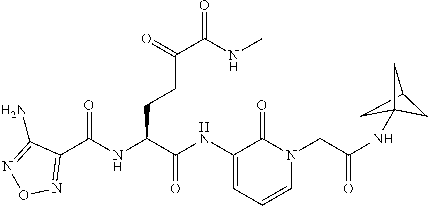 [SMILES: CNC(=O)C(=O)CC[C@H](NC(=O)c1nonc1N)C(=O)Nc1cccn(CC(=O)NC23CC(C2)C3)c1=O] | (S)-2-(4-amino-1,2,5-oxadiazole-3- carboxamido)-N1-(1-(2- (bicyclo[1.1.1]pentan-1-ylamino)-2- oxoethyl)-2-oxo-1,2-dihydropyridin-3-yl)- N6-methyl-5-oxohexanediamide |
| II-190 | 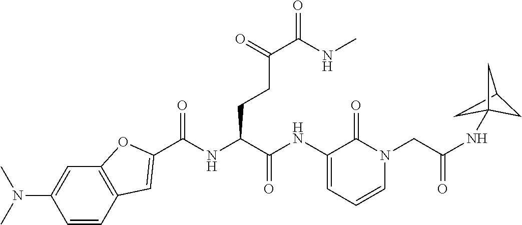 [SMILES: CNC(=O)C(=O)CC[C@H](NC(=O)c1cc2ccc(N(C)C)cc2o1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-2-(6- (dimethylamino)benzofuran-2- carboxamido)-N6-methyl-5- oxohexanediamide |
| II-191 | 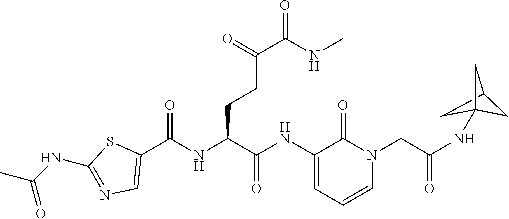 [SMILES: CNC(=O)C(=O)CC[C@H](NC(=O)c1cnc(NC(C)=O)s1)C(=O)Nc1cccn(CC(=O)NC23CC(C2)C3)c1=O] | (S)-2-(2-acetamidothiazole-5- carboxamido)-N1-(1-(2- (bicyclo[1.1.1]pentan-1-ylamino)-2- oxoethyl)-2-oxo-1,2-dihydropyridin-3-yl)- N6-methyl-5-oxohexanediamide |
| II-192 | 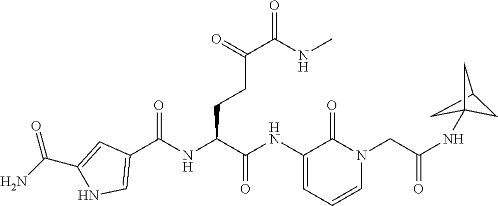 [SMILES: CNC(=O)C(=O)CC[C@H](NC(=O)c1c[nH]c(C(N)=O)c1)C(=O)Nc1cccn(CC(=O)NC23CC(C2)C3)c1=O] | (S)-N4-(1-(1-(2-(bicyclo[1.1.1]pentan-1- ylamino)-2-oxoethyl)-2-oxo-1,2- dihydropyridin-3-ylamino)-6- (methylamino)-1,5,6-trioxohexan-2-yl)-1H- pyrrole-2,4-dicarboxamide |
| II-193 | 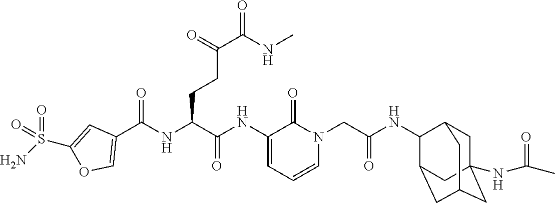 [SMILES: CNC(=O)C(=O)CC[C@H](NC(=O)c1coc(S(N)(=O)=O)c1)C(=O)Nc1cccn(CC(=O)NC2C3CC4CC2CC(NC(C)=O)(C4)C3)c1=O] | (S)-N1-(1-(2-(1-acetylamino-4- aminoadamantane)-2-oxoethyl)-2-oxo-1,2- dihydropyridin-3-yl)-N6-methyl-5-oxo-2-(5- sulfamoylfuran-3- carboxamido)hexanediamide |
| II-194 | 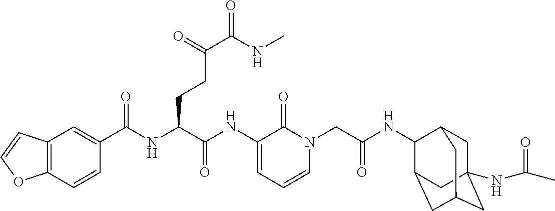 [SMILES: CNC(=O)C(=O)CC[C@H](NC(=O)c1ccc2occc2c1)C(=O)Nc1cccn(CC(=O)NC2C3CC4CC2CC(NC(C)=O)(C4)C3)c1=O] | (S)-2-(benzofuran-5-carboxamido)-N1-(1- (2-(1-acetylamino-4-aminoadamantane)-2- oxoethyl)-2-oxo-1,2-dihydropyridin-3-yl)- N6-methyl-5-oxohexanediamide |
| II-195 | 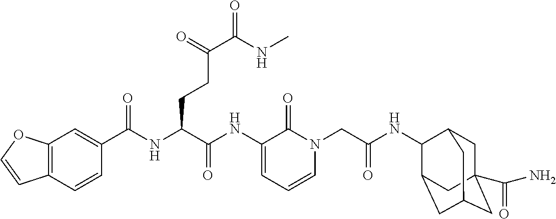 [SMILES: CNC(=O)C(=O)CC[C@H](NC(=O)c1ccc2ccoc2c1)C(=O)Nc1cccn(CC(=O)NC2C3CC4CC2CC(C(N)=O)(C4)C3)c1=O] | (S)-2-(benzofuran-6-carboxamido)-N1-(1- (2-(4-aminoadamantane-1-carboxamide)- 2-oxoethyl)-2-oxo-1,2-dihydropyridin-3-yl)- N6-methyl-5-oxohexanediamide |
| II-196 | 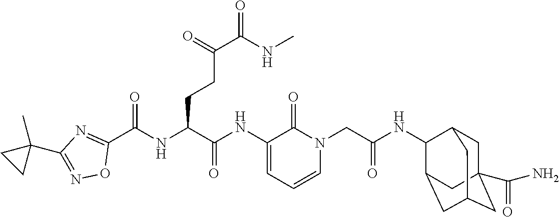 [SMILES: CNC(=O)C(=O)CC[C@H](NC(=O)c1nc(C2(C)CC2)no1)C(=O)Nc1cccn(CC(=O)NC2C3CC4CC2CC(C(N)=O)(C4)C3)c1=O] | (S)-N1-(1-(2-(4-aminoadamantane-1- carboxamide)-2-oxoethyl)-2-oxo-1,2- dihydropyridin-3-yl)-N6-methyl-2-(3-(1- methylcyclopropyl)-1,2,4-oxadiazole-5- carboxamido)-5-oxohexanediamide |
| II-197 | 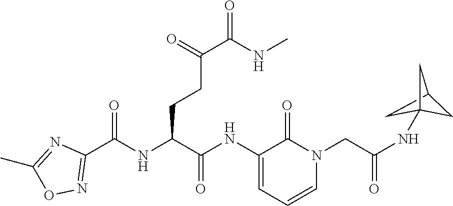 [SMILES: CNC(=O)C(=O)CC[C@H](NC(=O)c1noc(C)n1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-methyl-2-(5- methyl-1,2,4-oxadiazole-3-carboxamido)- 5-oxohexanediamide |
| II-198 | 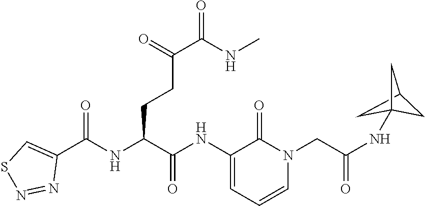 [SMILES: CNC(=O)C(=O)CC[C@H](NC(=O)c1csnn1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-methyl-5-oxo-2- (1,2,3-thiadiazole-4- carboxamido)hexanediamide |
| II-199 | 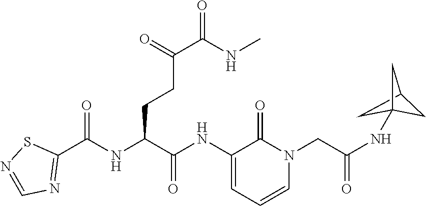 [SMILES: CNC(=O)C(=O)CC[C@H](NC(=O)c1ncns1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-methyl-5-oxo-2- (1,2,4-thiadiazole-5- carboxamido)hexanediamide |
| II-200 | 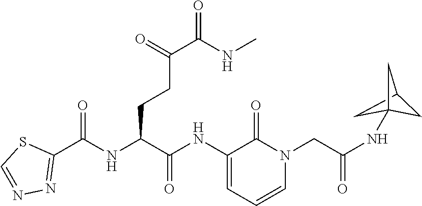 [SMILES: CNC(=O)C(=O)CC[C@H](NC(=O)c1nncs1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-N6-methyl-5-oxo-2- (1,3,4-thiadiazole-2- carboxamido)hexanediamide |
| II-201 | 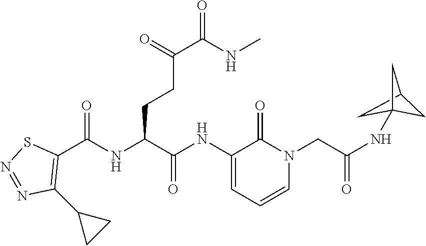 [SMILES: CNC(=O)C(=O)CC[C@H](NC(=O)c1snnc1C1CC1)C(=O)Nc1cccn(CC(=O)NC23CC(C2)C3)c1=O] | (S)-N1-(1-(2-(bicyclo[1.1.1]pentan-1- ylamino)-2-oxoethyl)-2-oxo-1,2- dihydropyridin-3-yl)-2-(4-cyclopropyl-1,2,3- thiadiazole-5-carboxamido)-N6-methyl-5- oxohexanediamide |
| II-202 | 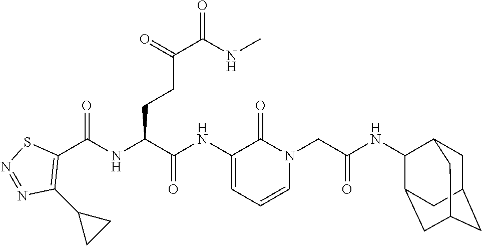 [SMILES: CNC(=O)C(=O)CC[C@H](NC(=O)c1snnc1C1CC1)C(=O)Nc1cccn(CC(=O)NC2C3CC4CC(C3)CC2C4)c1=O] | (S)-2-(4-cyclopropyl-1,2,3-thiadiazole-5- carboxamido)-N1-(1-(2-(2- adamantylamino)-2-oxoethyl)-2-oxo-1,2- dihydropyridin-3-yl)-N6-methyl-5- oxohexanediamide |
| II-203 | 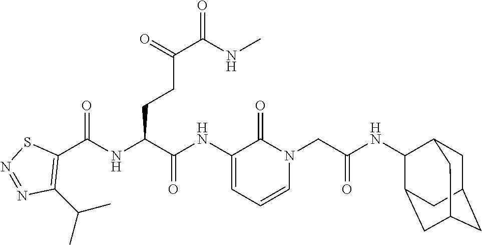 [SMILES: CNC(=O)C(=O)CC[C@H](NC(=O)c1snnc1C(C)C)C(=O)Nc1cccn(CC(=O)NC2C3CC4CC(C3)CC2C4)c1=O] | (S)-2-(4-isopropyl-1,2,3-thiadiazole-5- carboxamido)-N1-(1-(2-(2- adamantylamino)-2-oxoethyl)-2-oxo-1,2- dihydropyridin-3-yl)-N6-methyl-5- oxohexanediamide |
| II-204 | 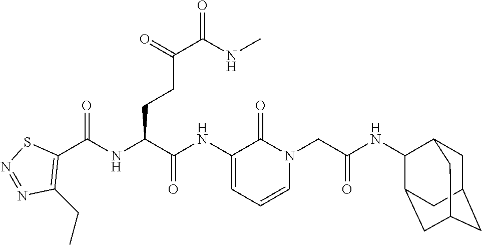 [SMILES: CCc1nnsc1C(=O)N[C@@H](CCC(=O)C(=O)NC)C(=O)Nc1cccn(CC(=O)NC2C3CC4CC(C3)CC2C4)c1=O] | (S)-2-(4-ethyl-1,2,3-thiadiazole-5- carboxamido)-N1-(1-(2-(2- adamantylamino)-2-oxoethyl)-2-oxo-1,2- dihydropyridin-3-yl)-N6-methyl-5- oxohexanediamide |
| II-205 | 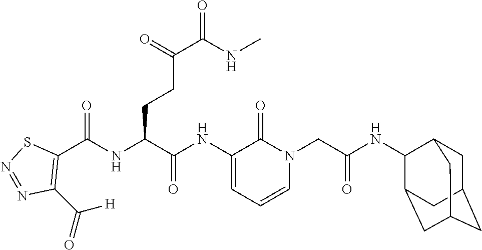 [SMILES: CNC(=O)C(=O)CC[C@H](NC(=O)c1snnc1C=O)C(=O)Nc1cccn(CC(=O)NC2C3CC4CC(C3)CC2C4)c1=O] | (S)-2-(4-formyl-1,2,3-thiadiazole-5- carboxamido)-N1-(1-(2-(2- adamantylamino)-2-oxoethyl)-2-oxo-1,2- dihydropyridin-3-yl)-N6-methyl-5- oxohexanediamide |
| II-206 | 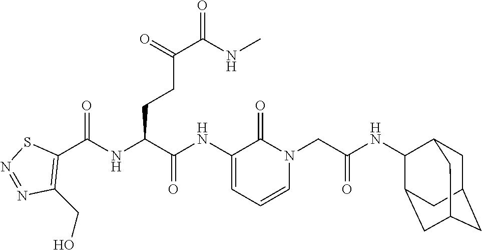 [SMILES: CNC(=O)C(=O)CC[C@H](NC(=O)c1snnc1CO)C(=O)Nc1cccn(CC(=O)NC2C3CC4CC(C3)CC2C4)c1=O] | (S)-2-(4-(hydroxymethyl)-1,2,3-thiadiazole- 5-carboxamido)-N1-(1-(2-(2- adamantylamino)-2-oxoethyl)-2-oxo-1,2- dihydropyridin-3-yl)-N6-methyl-5- oxohexanediamide |
| II-207 | 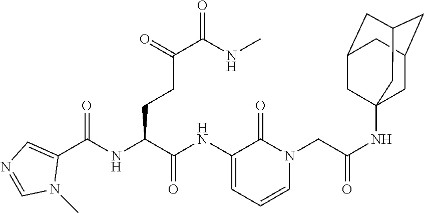 [SMILES: CNC(=O)C(=O)CC[C@H](NC(=O)c1cncn1C)C(=O)Nc1cccn(CC(=O)NC23CC4CC(CC(C4)C2)C3)c1=O] | (S)-N1-(1-(2-(1-adamantylamino)-2- oxoethyl)-2-oxo-1,2-dihydropyridin-3-yl)- N6-methyl-2-(1-methyl-1H-imidazole-5- carboxamido)-5-oxohexanediamide |
| II-208 | 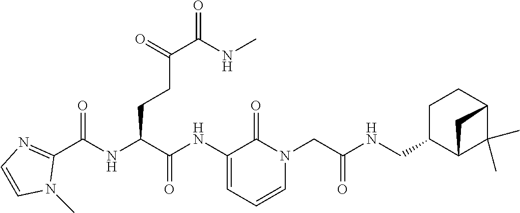 [SMILES: CNC(=O)C(=O)CC[C@H](NC(=O)c1nccn1C)C(=O)Nc1cccn(CC(=O)NC[C@@H]2CC[C@H]3C[C@@H]2C3(C)C)c1=O] | (S)-N1-(1-(2-(((1S,2R,5S)-6,6- dimethylbicyclo[3.1.1]heptan-2- yl)methylamino)-2-oxoethyl)-2-oxo-1,2- dihydropyridin-3-yl)-N6-methyl-2-(1- methyl-1H-imidazole-2-carboxamido)-5- oxohexanediamide |
| II-209 | 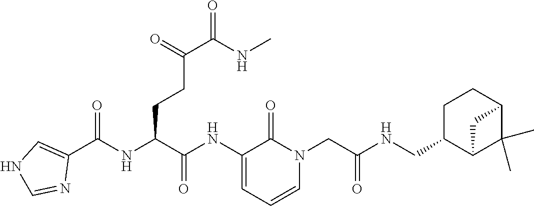 [SMILES: CNC(=O)C(=O)CC[C@H](NC(=O)c1c[nH]cn1)C(=O)Nc1cccn(CC(=O)NC[C@@H]2CC[C@@H]3C[C@H]2C3(C)C)c1=O] | (S)-N1-(1-(2-(((1R,2R,5R)-6,6- dimethylbicyclo[3.1.1]heptan-2- yl)methylamino)-2-oxoethyl)-2-oxo-1,2- dihydropyridin-3-yl)-2-(1H-imidazole-4- carboxamido)-N6-methyl-5- oxohexanediamide |
| II-210 | 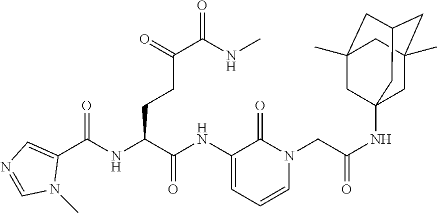 [SMILES: CNC(=O)C(=O)CC[C@H](NC(=O)c1cncn1C)C(=O)Nc1cccn(CC(=O)NC23CC4CC(C)(CC(C)(C4)C2)C3)c1=O] | (S)-N1-(1-(2-(3,5-dimethyladamantane-1- amino)-2-oxoethyl)-2-oxo-1,2- dihydropyridin-3-yl)-N6-methyl-2-(1- methyl-1H-imidazole-5-carboxamido)-5- oxohexanediamide |
| II-211 | 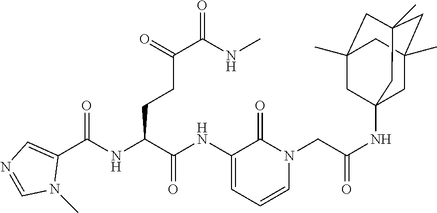 [SMILES: CNC(=O)C(=O)CC[C@H](NC(=O)c1cncn1C)C(=O)Nc1cccn(CC(=O)NC23CC4(C)CC(C)(CC(C)(C4)C2)C3)c1=O] | (S)-N1-(1-(2-(3,5,7-trimethyl-1- adamantylamino)-2-oxoethyl)-2-oxo-1,2- dihydropyridin-3-yl)-N6-methyl-2-(1- methyl-1H-imidazole-5-carboxamido)-5- oxohexanediamide |

or a pharmaceutically acceptable salt thereof.

Especially preferred are the following compounds:

| | |
|---|---|
| II-2: | (S)-2-(benzofuran-2-carboxamido)- |
| | N1-(1-(2-(2-adamantylamino)-2-oxoethyl)-2-oxo- |
| | 1,2-dihydropyridin-3-yl)-N6-methyl- |
| | 5-oxohexanediamide |
| II-3: | (S)-N1-(1-(2-(2-adamantylamino)-2-oxoethyl)- |
| | 2-oxo-1,2-dihydropyridin-3-yl)-N6- |
| | methyl-2-(3-methylbenzofuran-2- |
| | carboxamido)-5-oxohexanediamide |
| II-4: | (S)-2-(3-chlorobenzofuran-2-carboxamido)- |
| | N1-(1-(2-(2-adamantylamino)-2- |
| | oxoethyl)-2-oxo-1,2-dihydropyridin-3- |
| | yl)-N6-methyl-5-oxohexanediamide |
| II-5: | (S)-2-(4-bromobenzofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-6: | (S)-2-(4-bromobenzofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-7: | (S)-2-(benzo[b]thiophene-2-carboxamido)-N1- |
| | (1-(2-(2-adamantylamino)-2-oxoethyl)- |
| | 2-oxo-1,2-dihydropyridin-3-yl)-N6- |
| | methyl-5-oxohexanediamide |
| II-8: | (S)-2-(5-bromobenzo[b]thiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-9: | (S)-2-(1H-indole-2-carboxamido)-N1-(1- |
| | (2-(2-adamantylamino)-2-oxoethyl)-2-oxo- |
| | 1,2-dihydropyridin-3-yl)-N6-methyl- |
| | 5-oxohexanediamide |
| II-10: | (S)-2-(4,5-difluoro-1H-indole-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-11: | (S)-N1-(1-(2-(2-adamantylamino)-2-oxoethyl)- |
| | 2-oxo-1,2-dihydropyridin-3-yl)-N6- |
| | methyl-2-(3-methyl-1H-indole-2- |
| | carboxamido)-5-oxohexanediamide |
| II-12: | (S)-2-(1H-benzo[d]imidazole-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-13: | (S)-2-(2,3-dihydro-1H-indene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-14: | (S)--(2-bromo-4-methylthiazole-5-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-15: | (S)-N1-(1-(2-(2-adamantylamino)-2-oxoethyl)- |
| | 2-oxo-1,2-dihydropyridin-3-yl)-N6- |
| | methyl-2-(4-methyl-2-(trifluoromethyl)thiazole- |
| | 5-carboxamido)-5-oxohexanediamide |
| II-16: | (S)-2-(4-bromo-2-(trifluoromethyl)thiazole- |
| | 5-carboxamido)-N1-(1-(2-(2- |
| | adamantylamino)-2-oxoethyl)-2-oxo- |
| | 1,2-dihydropyridin-3-yl)-N6-methyl-5- |
| | oxohexanediamide |
| II-17: | (S)-2-(2,4-dichlorothiazole-5-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-18: | (S)-N1-(1-(2-(2-adamantylamino)-2-oxoethyl)- |
| | 2-oxo-1,2-dihydropyridin-3-yl)-2-(2- |
| | methoxy-4-methylthiazole-5-carboxamido)- |
| | N6-methyl-5-oxohexanediamide |
| II-19: | (S)-N1-(1-(2-(2-adamantylamino)-2-oxoethyl)- |
| | 2-oxo-1,2-dihydropyridin-3-yl)-N6- |
| | methyl-2-(4-methyl-2-phenylthiazole-5- |
| | carboxamido)-5-oxohexanediamide |
| II-21: | (S)-2-(5-bromo-3-methylthiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-22: | (S)-2-(3,5-dibromothiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-23: | (S)-2-(5-bromothiophene-2-carboxamido)-N1- |
| | (1-(2-(2-adamantylamino)-2-oxoethyl)- |
| | 2-oxo-1,2-dihydropyridin-3-yl)-N6- |
| | methyl-5-oxohexanediamide |
| II-24: | (S)-2-(5-chlorothiophene-2-carboxamido)-N1- |
| | (1-(2-(2-adamantylamino)-2-oxoethyl)- |
| | 2-oxo-1,2-dihydropyridin-3-yl)-N6- |
| | methyl-5-oxohexanediamide |
| II-25: | (S)-2-(5-bromo-3-methylfuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-26: | (S)-2-(5-chlorofuran-2-carboxamido)-N1- |
| | (1-(2-(2-adamantylamino)-2-oxoethyl)-2- |
| | oxo-1,2-dihydropyridin-3-yl)-N6- |
| | methyl-5-oxohexanediamide |
| II-27: | (S)-2-(5-chlorothiophene-3-carboxamido)-N1- |
| | (1-(2-(2-adamantylamino)-2-oxoethyl)- |
| | 2-oxo-1,2-dihydropyridin-3-yl)-N6- |
| | methyl-5-oxohexanediamide |
| II-28: | (S)-2-(2,5-dichlorothiophene-3-carboxamido)-N1- |
| | (1-(2-(2-adamantylamino)-2- |
| | oxoethyl)-2-oxo-1,2-dihydropyridin-3-yl)- |
| | N6-methyl-5-oxohexanediamide |
| II-29: | (S)-2-(2,5-dibromothiophene-3-carboxamido)-N1- |
| | (1-(2-(2-adamantylamino)-2- |
| | oxoethyl)-2-oxo-1,2-dihydropyridin-3-yl)- |
| | N6-methyl-5-oxohexanediamide |
| II-30: | (S)-2-(5-bromothiophene-3-carboxamido)- |
| | N1-(1-(2-(2-adamantylamino)-2-oxoethyl)- |
| | 2-oxo-1,2-dihydropyridin-3-yl)-N6- |
| | methyl-5-oxohexanediamide |
| II-31: | (S)-2-(2-chloro-5-methylthiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-32: | (S)-2-(2,5-dichlorothiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-33: | (S)-2-(2,5-dibromothiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-34: | (S)-2-(2-bromo-5-methylthiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-35: | (S)-2-(2-bromothiazole-4-carboxamido)-N1- |
| | (1-(2-(2-adamantylamino)-2-oxoethyl)-2- |
| | oxo-1,2-dihydropyridin-3-yl)-N6- |
| | methyl-5-oxohexanediamide |
| II-36: | (S)-2-(2-chlorothiazole-4-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-37: | (S)-2-(2,5-dimethylfuran-3-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-39: | (S)-2-(4-bromothiazole-2-carboxamido)-N1-(1- |
| | (2-(2-adamantylamino)-2-oxoethyl)-2-oxo-1,2- |
| | dihydropyridin-3-yl)-N6-methyl-5-oxohexanediamide |
| II-40: | (S)-2-(4-bromothiophene-2-carboxamido)-N1-(1- |
| | (2-(2-adamantylamino)-2-oxoethyl)-2-oxo-1,2- |
| | dihydropyridin-3-yl)-N6-methyl-5-oxohexanediamide |
| II-41: | (S)-2-(4-bromo-3-methylthiophene-2-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-42: | (S)-2-(3-bromothiophene-2-carboxamido)-N1-(1- |
| | (2-(2-adamantylamino)-2-oxoethyl)-2-oxo-1,2- |
| | dihydropyridin-3-yl)-N6-methyl-5-oxohexanediamide |
| II-44: | (S)-2-(4-bromo-5-chlorothiophene-2-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-45: | (S)-2-(4,5-dibromothiophene-2-carboxamido)-N1-(1- |
| | (2-(2-adamantylamino)-2-oxoethyl)-2-oxo-1,2- |
| | dihydropyridin-3-yl)-N6-methyl-5-oxohexanediamide |
| II-46: | (S)-2-(4,5-dibromo-3-methoxythiophene-2- |
| | carboxamido)-N1-(1-(2-(2-adamantylamino)-2- |
| | oxoethyl)-2-oxo-1,2-dihydropyridin-3-yl)-N6- |
| | methyl-5-oxohexanediamide |
| II-47: | (S)-2-(4-bromofuran-2-carboxamido)-N1-(1-(2-(2- |
| | adamantylamino)-2-oxoethyl)-2-oxo-1,2- |
| | dihydropyridin-3-yl)-N6-methyl-5-oxohexanediamide |
| II-48: | (S)-2-(4,5-dibromofuran-2-carboxamido)-N1-(1- |
| | (2-(2-adamantylamino)-2-oxoethyl)-2-oxo-1,2- |
| | dihydropyridin-3-yl)-N6-methyl-5-oxohexanediamide |
| II-49: | (S)-2-(4,5-dichlorothiophene-2-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-50: | (S)-2-((S)-1-acetylpyrrolidine-2-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-51: | (S)-N1-(1-(2-(2-adamantylamino)-2-oxoethyl)-2- |
| | oxo-1,2-dihydropyridin-3-yl)-N6-methyl-2-(1-methyl- |
| | 1H-1,2,3-triazole-5-carboxamido)-5-oxohexanediamide |
| II-53 | (S)-N1-(1-(2-(2-adamantylamino)-2-oxoethyl)-2-oxo- |
| | 1,2-dihydropyridin-3-yl)-N6-methyl-5-oxo-2- |
| | (pyrazine-2-carboxamido)hexanediamide |
| II-54: | (S)-N1-(1-(2-(2-adamantylamino)-2-oxoethyl)-2- |
| | oxo-1,2-dihydropyridin-3-yl)-N6-methyl-2-((S)-1- |
| | methylpyrrolidine-2-carboxamido)-5-oxohexanediamide |
| II-55: | (S)-N1-(1-(2-(2-adamantylamino)-2-oxoethyl)-2-oxo- |
| | 1,2-dihydropyridin-3-yl)-N6-methyl-5-oxo-2-((S)- |
| | pyrrolidine-3-carboxamido)hexanediamide |
| II-56 | (S)-2-((2S,4S)-4-bromopyrrolidine-2-carboxamido)-N1- |
| | (1-(2-(2-adamantylamino)-2-oxoethyl)-2-oxo-1,2- |
| | dihydropyridin-3-yl)-N6-methyl-5-oxohexanediamide |
| II-58: | (S)-N1-(1-(2-(2-adamantylamino)-2-oxoethyl)-2- |
| | oxo-1,2-dihydropyridin-3-yl)-N6- |
| | methyl-5-oxo-2-((S)-piperidine-2- |
| | carboxamido)hexanediamide |
| II-59: | (S)-N1-(1-(2-(2-adamantylamino)-2- |
| | oxoethyl)-2-oxo-1,2-dihydropyridin-3-yl)-N6- |
| | methyl-5-oxo-2-((R)-piperidine-3- |
| | carboxamido)hexanediamide |
| II-60: | (S)-N1-(1-(2-(2-adamantylamino)-2- |
| | oxoethyl)-2-oxo-1,2-dihydropyridin-3-yl)-N6- |
| | methyl-2-((R)-morpholine-3-carboxamido)- |
| | 5-oxohexanediamide |
| II-61: | (S)-N1-(1-(2-(2-adamantylamino)-2-oxoethyl)- |
| | 2-oxo-1,2-dihydropyridin-3-yl)-N6- |
| | methyl-5-oxo-2-(quinuclidine-3- |
| | carboxamido)hexanediamide |
| II-62: | (S)-methyl 3-(1-(1-(2-(2-adamantylamino)- |
| | 2-oxoethyl)-2-oxo-1,2-dihydropyridin-3- |
| | ylamino)-6-(methylamino)-1,5,6-trioxohexan- |
| | 2-ylcarbamoyl)-5-nitrobenzoate |
| II-63: | (S)-N1-(1-(2-(2-adamantylamino)-2- |
| | oxoethyl)-2-oxo-1,2-dihydropyridin-3-yl)-N6- |
| | methyl-2-(5-nitronicotinamido)- |
| | 5-oxohexanediamide |
| II-64: | (S)-5-(1-(1-(2-(2-adamantylamino)-2-oxoethyl)- |
| | 2-oxo-1,2-dihydropyridin-3-ylamino)- |
| | 6-(methylamino)-1,5,6-trioxohexan- |
| | 2-ylcarbamoyl)nicotinic acid |
| II-65: | (S)-methyl 5-(1-(1-(2-(2-adamantylamino)-2- |
| | oxoethyl)-2-oxo-1,2-dihydropyridin-3- |
| | ylamino)-6-(methylamino)-1,5,6-trioxohexan- |
| | 2-ylcarbamoyl)nicotinate |
| II-68: | (S)-N1-(1-(2-(5-hydroxyadamantane-2-amino)- |
| | 2-oxoethyl)-2-oxo-1,2-dihydropyridin- |
| | 3-yl)-N6-methyl-2-(3-methylbenzofuran-2- |
| | carboxamido)-5-oxohexanediamide |
| II-69: | (S)-N1-(1-(2-(5-fluoroadamantane-2-amino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-70: | (S)-N1-(1-(2-(5-chloroadamantane-2-amino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-71: | (S)-N1-(1-(2-(5-bromoadamantane-2-amino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-72: | (S)-N1-(1-(2-(5-methyladamantane-2-amino)-2- |
| | oxoethyl)-2-oxo-1,2-dihydropyridin-3- |
| | yl)-N6-methyl-2-(3-methylbenzofuran-2- |
| | carboxamido)-5-oxohexanediamide |
| II-73: | (S)-N1-(1-(2-(2-carbonitrileadamantane- |
| | 2-amino)-2-oxoethyl)-2-oxo-1,2- |
| | dihydropyridin-3-yl)-N6-methyl-2-(3- |
| | methylbenzofuran-2-carboxamido)-5- |
| | oxohexanediamide |
| II-87: | (S)-N1-(1-(2-(1-adamantylmethylamino)-2- |
| | oxoethyl)-2-oxo-1,2-dihydropyridin-3-yl)- |
| | N6-methyl-2-(3-methylbenzofuran-2- |
| | carboxamido)-5-oxohexanediamide |
| II-88: | (S)-N1-(1-(2-(1-(1-adamantyl)ethanamino)-2- |
| | oxoethyl)-2-oxo-1,2-dihydropyridin-3- |
| | yl)-N6-methyl-2-(3-methylbenzofuran-2- |
| | carboxamido)-5-oxohexanediamide |
| II-90: | (S)-N1-(1-(2-((1R,2S,4S)-bicyclo[2.2.1]heptan- |
| | 2-ylamino)-2-oxoethyl)-2-oxo-1,2- |
| | dihydropyridin-3-yl)-N6-methyl-2-(3- |
| | methylbenzofuran-2-carboxamido)-5- |
| | oxohexanediamide |
| II-92: | (S)-N1-methyl-5-(3-methylbenzofuran-2- |
| | carboxamido)-2-oxo-N6-(2-oxo-1-(2-oxo-2- |
| | ((1S,2S,4R)-1,7,7-trimethylbicyclo[2.2.1]heptan- |
| | 2-ylamino)ethyl)-1,2-dihydropyridin- |
| | 3-yl)hexanediamide |
| II-94: | (S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-N6-methyl-2-(3- |
| | methylbenzofuran-2-carboxamido)-5- |
| | oxohexanediamide |
| II-95: | (S)-N1-(1-(2-(bicyclo[2.2.1]heptan-1-ylamino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-96: | (S)-N1-(1-(2-(bicyclo[2.2.1]heptan-7-ylamino)- |
| | 2-oxoethyl)-2-oxo-1,2-dihydropyridin-3- |
| | yl)-N6-methyl-2-(3-methylbenzofuran- |
| | 2-carboxamido)-5-oxohexanediamide |
| II-97: | (S)-N1-(1-(2-(bicyclo[2.2.1]hept-5-en- |
| | 2-ylamino)-2-oxoethyl)-2-oxo-1,2- |
| | dihydropyridin-3-yl)-N6-methyl-2-(3- |
| | methylbenzofuran-2-carboxamido)-5- |
| | oxohexanediamide |
| II-98: | (2S)-N1-(1-(2-(bicyclo[2.2.2]octan-2-ylamino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-99: | (S)-N1-methyl-5-(3-methylbenzofuran-2- |
| | carboxamido)-2-oxo-N6-(2-oxo-1-(2-oxo-2- |
| | ((1R,2R,4R)-1,7,7-trimethylbicyclo[2.2.1]heptan- |
| | 2-ylamino)ethyl)-1,2-dihydropyridin- |
| | 3-yl)hexanediamide |
| II-100: | (S)-N1-methyl-5-(3-methylbenzofuran-2- |
| | carboxamido)-2-oxo-N6-(2-oxo-1-(2-oxo-2- |
| | ((1R,2R,3R,5S)-2,6,6-trimethylbicyclo[3.1.1]heptan- |
| | 3-ylamino)ethyl)-1,2- |
| | dihydropyridin-3-yl)hexanediamide |
| II-101: | (S)-N1-methyl-5-(3-methylbenzofuran-2- |
| | carboxamido)-2-oxo-N6-(2-oxo-1-(2-oxo-2- |
| | ((1S,2S,3S,5R)-2,6,6-trimethylbicyclo[3.1.1]heptan- |
| | 3-ylamino)ethyl)-1,2- |
| | dihydropyridin-3-yl)hexanediamide |
| II-103: | (S)-N1-(1-(2-(4-homoisotwistane-3-amino)- |
| | 2-oxoethyl)-2-oxo-1,2-dihydropyridin-3-yl)- |
| | N6-methyl-2-(3-methylbenzofuran-2- |
| | carboxamido)-5-oxohexanediamide |
| II-104: | (S)-N1-(1-(2-(diamantane-1-amino)-2-oxoethyl)- |
| | 2-oxo-1,2-dihydropyridin-3-yl)-N6- |
| | methyl-2-(3-methylbenzofuran-2- |
| | carboxamido)-5-oxohexanediamide |
| II-105: | (S)-N1-(1-(2-(diamantane-4-amino)-2-oxoethyl)- |
| | 2-oxo-1,2-dihydropyridin-3-yl)-N6- |
| | methyl-2-(3-methylbenzofuran-2- |
| | carboxamido)-5-oxohexanediamide |
| II-107: | (S)-N1-(1-(1-adamantylmethyl)-2-oxo-1,2- |
| | dihydropyridin-3-yl)-N6-methyl-2-(3- |
| | methylbenzofuran-2-carboxamido)- |
| | 5-oxohexanediamide |
| II-108: | (2S)-N1-(1-((3-hydroxy-1-adamantyl)methyl)-2- |
| | oxo-1,2-dihydropyridin-3-yl)-N6- |
| | methyl-2-(3-methylbenzofuran-2- |
| | carboxamido)-5-oxohexanediamide |
| II-109: | (2S)-N1-(1-((3-bromo-1-adamantyl)methyl)-2- |
| | oxo-1,2-dihydropyridin-3-yl)-N6-methyl- |
| | 2-(3-methylbenzofuran-2-carboxamido)- |
| | 5-oxohexanediamide |
| II-110: | (S)-N1-(1-(2-adamantylmethyl)-2-oxo-1,2- |
| | dihydropyridin-3-yl)-N6-methyl-2-(3- |
| | methylbenzofuran-2-carboxamido)- |
| | 5-oxohexanediamide |
| II-111: | (S)-N1-(1-(2-(2-adamantylamino)-2-oxoethyl)- |
| | 2-oxo-1,2-dihydropyridin-3-yl)-N6- |
| | methyl-2-(nicotinamido)-5-oxohexanediamide |
| II-112: | (S)-2-(isonicotinamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-113: | (S)-N1-(1-(2-(2-adamantylamino)-2-oxoethyl)- |
| | 2-oxo-1,2-dihydropyridin-3-yl)-N6- |
| | methyl-5-oxo-2-(pyridazine-4- |
| | carboxamido)hexanediamide |
| II-114: | (S)-N1-(1-(2-(2-adamantylamino)-2-oxoethyl)- |
| | 2-oxo-1,2-dihydropyridin-3-yl)-N6- |
| | methyl-5-oxo-2-(pyridazine-3- |
| | carboxamido)hexanediamide |
| II-115: | (S)-N1-cyclopropyl-N6-(1-(2-(2- |
| | adamantylamino)-2-oxoethyl)-2-oxo-1,2- |
| | dihydropyridin-3-yl)-5-(3-methylbenzofuran-2- |
| | carboxamido)-2-oxohexanediamide |
| II-116: | (S)-N1-(1-(2-(2-adamantylamino)-2-oxoethyl)- |
| | 2-oxo-1,2-dihydropyridin-3-yl)-2-(3- |
| | methylbenzofuran-2-carboxamido)- |
| | 5-oxo-N6-pentylhexanediamide |
| II-117: | (S)-N1-allyl-N6-(1-(2-(2-adamantylamino)-2- |
| | oxoethyl)-2-oxo-1,2-dihydropyridin-3-yl)- |
| | 5-(3-methylbenzofuran-2-carboxamido)- |
| | 2-oxohexanediamide |
| II-118: | (S)-N1-(1-(2-(2-adamantylamino)-2-oxoethyl)- |
| | 2-oxo-1,2-dihydropyridin-3-yl)-2-(3- |
| | methylbenzofuran-2-carboxamido)- |
| | 5-oxohexanediamide |
| II-119: | (S)-N1-allyl-5-(benzofuran-2-carboxamido)- |
| | N6-(1-(2-(2-adamantylamino)-2- |
| | oxoethyl)-2-oxo-1,2-dihydropyridin- |
| | 3-yl)-2-oxohexanediamide |
| II-120: | (S)-2-(benzofuran-2-carboxamido)-N6-isopropyl- |
| | N1-(1-(2-(2-adamantylamino)-2- |
| | oxoethyl)-2-oxo-1,2-dihydropyridin- |
| | 3-yl)-5-oxohexanediamide |
| II-121: | (S)-2-(benzofuran-2-carboxamido)-N6-cyclopropyl- |
| | N1-(1-(2-(2-adamantylamino)-2- |
| | oxoethyl)-2-oxo-1,2-dihydropyridin- |
| | 3-yl)-5-oxohexanediamide |
| II-124: | (S)-2-(benzofuran-2-carboxamido)-N1-(1-(2- |
| | (2-adamantylamino)-2-oxoethyl)-2-oxo- |
| | 1,2-dihydropyridin-3-yl)-5-oxohexanediamide |
| II-125: | (S)-2-(2,5-dichlorothiophene-3-carboxamido)- |
| | N1-(1-(2-(2-adamantylamino)-2- |
| | oxoethyl)-2-oxo-1,2-dihydropyridin- |
| | 3-yl)-5-oxohexanediamide |
| II-126: | (S)-N1-(1-(2-(2-adamantylamino)-2-oxoethyl)- |
| | 2-oxo-1,2-dihydropyridin-3-yl)-2-(4- |
| | methyl-2-(trifluoromethyl)thiazole-5- |
| | carboxamido)-5-oxohexanediamide |
| II-127: | (S)-N1-(1-(2-(2-adamantylamino)-2-oxoethyl)- |
| | 2-oxo-1,2-dihydropyridin-3-yl)-2-(1- |
| | methyl-1H-1,2,3-triazole-5-carboxamido)- |
| | 5-oxohexanediamide |
| II-128: | (2S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-2-(2,5-dichlorothiophene- |
| | 3-carboxamido)-N6-methyl-5- |
| | oxohexanediamide |
| II-129: | (2S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-N6-methyl-2-(4- |
| | methyl-2-(trifluoromethyl) thiazole-5- |
| | carboxamido)-5-oxohexanediamide |
| II-130: | (2S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-N6-methyl-2-(1-methyl- |
| | 1H-1,2,3-triazole-5-carboxamido)-5- |
| | oxohexanediamide |
| II-131: | (S)-N1-(1-(2-(2-adamantylamino)-2-oxoethyl)- |
| | 2-oxo-1,2-dihydropyridin-3-yl)-N6- |
| | methyl-5-oxo-2-(2H-1,2,3-triazole-4- |
| | carboxamido) hexanediamide |
| II-135: | (S)-N1-(1-(2-(2-adamantylamino)-2-oxoethyl)- |
| | 2-oxo-1,2-dihydropyridin-3-yl)-N6- |
| | methyl-2-(1-methyl-1H-1,2,4-triazole-3- |
| | carboxamido)-5-oxohexanediamide |
| II-136: | (S)-2-(benzofuran-3-carboxamido)-N1-(1-(2- |
| | (2-adamantylamino)-2-oxoethyl)-2-oxo- |
| | 1,2-dihydropyridin-3-yl)-N6-methyl- |
| | 5-oxohexanediamide |
| II-137: | (S)-2-(benzo[b]thiophene-3-carboxamido)-N1- |
| | (1-(2-(2-adamantylamino)-2-oxoethyl)- |
| | 2-oxo-1,2-dihydropyridin-3-yl)-N6- |
| | methyl-5-oxohexanediamide |
| II-138 | (S)-N1-(1-(2-(2-adamantylamino)-2-oxoethyl)- |
| | 2-oxo-1,2-dihydropyridin-3-yl)-N6- |
| | methyl-2-(1-methyl-1H-pyrazole-3- |
| | carboxamido)-5-oxohexanediamide |
| II-139 | (S)-N1-(1-(2-(2-adamantylamino)-2-oxoethyl)- |
| | 2-oxo-1,2-dihydropyridin-3-yl)-N6- |
| | methyl-2-(1-methyl-1H-pyrazole-4- |
| | carboxamido)-5-oxohexanediamide |
| II-140 | (S)-N1-(1-(2-(2-adamantylamino)-2-oxoethyl)- |
| | 2-oxo-1,2-dihydropyridin-3-yl)-N6- |
| | methyl-2-(1-methyl-1H-pyrazole-5- |
| | carboxamido)-5-oxohexanediamide |
| II-141 | (S)-N1-(1-(2-(2-adamantylamino)-2-oxoethyl)- |
| | 2-oxo-1,2-dihydropyridin-3-yl)-N6- |
| | methyl-2-(4-methyl-1,2,3-thiadiazole-5- |
| | carboxamido)-5-oxohexanediamide |
| II-142 | (S)-N1-(1-(2-(2-adamantylamino)-2-oxoethyl)- |
| | 2-oxo-1,2-dihydropyridin-3-yl)-N6- |
| | methyl-5-oxo-2-(1,2,5-thiadiazole-3- |
| | carboxamido)hexanediamide |
| II-145 | (S)-N1-(1-(2-(2-adamantylamino)-2-oxoethyl)- |
| | 2-oxo-1,2-dihydropyridin-3-yl)-2-(4- |
| | methyl-1,2,3-thiadiazole-5-carboxamido)- |
| | 5-oxohexanediamide |
| II-146 | (S)-2-(benzofuran-2-carboxamido)-N1-(1-(2- |
| | ((1R,2R,4S)-bicyclo[2.2.1]heptan-2- |
| | ylamino)-2-oxoethyl)-2-oxo-1,2-dihydropyridin- |
| | 3-yl)-5-oxohexanediamide |
| II-147 | (S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-2-(3-methylbenzofuran- |
| | 2-carboxamido)-5-oxohexanediamide |
| II-148 | (S)-2-(benzofuran-2-carboxamido)-N1-(1-(2- |
| | ((1S,2R,4R)-bicyclo[2.2.1]heptan-2- |
| | ylamino)-2-oxoethyl)-2-oxo-1,2-dihydropyridin- |
| | 3-yl)-5-oxohexanediamide |
| II-149 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-2-(3-methylbenzofuran- |
| | 2-carboxamido)-5-oxohexanediamide |
| II-150 | (S)-2-(benzofuran-2-carboxamido)-5-oxo-N1- |
| | (2-oxo-1-(2-oxo-2-((1R,2S,4R)-1,7,7- |
| | trimethylbicyclo[2.2.1]heptan-2-ylamino)ethyl)- |
| | 1,2-dihydropyridin-3-yl)hexanediamide |
| II-151 | (S)-2-(3-methylbenzofuran-2-carboxamido)- |
| | 5-oxo-N1-(2-oxo-1-(2-oxo-2-((1R,2S,4R)- |
| | 1,7,7-trimethylbicyclo[2.2.1]heptan- |
| | 2-ylamino)ethyl)-1,2-dihydropyridin-3- |
| | yl)hexanediamide |
| II-152 | (S)-N1-(1-(2-((1R,2R,4S)-bicyclo[2.2.1]heptan- |
| | 2-ylamino)-2-oxoethyl)-2-oxo-1,2- |
| | dihydropyridin-3-yl)-N6-methyl-2-(4-methyl- |
| | 1,2,3-thiadiazole-5-carboxamido)-5- |
| | oxohexanediamide |
| II-154 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-N6-methyl-2-(4- |
| | methyl-2-(trifluoromethyl)thiazole-5- |
| | carboxamido)-5-oxohexanediamide |
| II-155 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-2-(2,5-dichlorothiophene- |
| | 3-carboxamido)-N6-methyl-5- |
| | oxohexanediamide |
| II-156 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-N6-methyl-2-(4-methyl- |
| | 1,2,3-thiadiazole-5-carboxamido)-5- |
| | oxohexanediamide |
| II-157 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-N6-methyl-2-(1-methyl- |
| | 1H-1,2,3-triazole-5-carboxamido)-5- |
| | oxohexanediamide |
| II-158 | (S)-N1-(1-(2-((1S,2R,4R)-bicyclo[2.2.1]heptan- |
| | 2-ylamino)-2-oxoethyl)-2-oxo-1,2- |
| | dihydropyridin-3-yl)-N6-methyl-2-(1-methyl- |
| | 1H-pyrazole-5-carboxamido)-5- |
| | oxohexanediamide |
| II-159 | (S)-N1-methyl-5-(4-methyl-2- |
| | (trifluoromethyl)thiazole- |
| | 5-carboxamido)-2-oxo-N6-(2- |
| | oxo-1-(2-oxo-2-((1R,2S,4R)-1,7,7- |
| | trimethylbicyclo[2.2.1]heptan-2-ylamino)ethyl)- |
| | 1,2-dihydropyridin-3-yl)hexanediamide |
| II-160 | (S)-2-(2,5-dichlorothiophene-3-carboxamido)-N6- |
| | methyl-5-oxo-N1-(2-oxo-1-(2-oxo-2- |
| | ((1R,2S,4R)-1,7,7-trimethylbicyclo[2.2.1]heptan- |
| | 2-ylamino)ethyl)-1,2-dihydropyridin- |
| | 3-yl)hexanediamide |
| II-161 | (S)-N1-methyl-5-(4-methyl-1,2,3-thiadiazole-5- |
| | carboxamido)-2-oxo-N6-(2-oxo-1-(2- |
| | oxo-2-((1R,2S,4R)-1,7,7- |
| | trimethylbicyclo[2.2.1]heptan- |
| | 2-ylamino)ethyl)-1,2- |
| | dihydropyridin-3-yl)hexanediamide |
| II-162 | (S)-N1-methyl-5-(1-methyl-1H-1,2,3-triazole- |
| | 5-carboxamido)-2-oxo-N6-(2-oxo-1-(2- |
| | oxo-2-((1R,2S,4R)-1,7,7- |
| | trimethylbicyclo[2.2.1]heptan- |
| | 2-ylamino)ethyl)-1,2- |
| | dihydropyridin-3-yl)hexanediamide |
| II-163 | (S)-N1-methyl-5-(1-methyl-1H- |
| | pyrazole-5-carboxamido)- |
| | 2-oxo-N6-(2-oxo-1-(2-oxo-2- |
| | ((1R,2S,4R)-1,7,7-trimethylbicyclo[2.2.1]heptan- |
| | 2-ylamino)ethyl)-1,2-dihydropyridin- |
| | 3-yl)hexanediamide |
| II-167 | (S)-N1-(1-(2-(bicyclo[1.1.1]pentan-1-ylamino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-168 | (S)-2-(2-acetyloxazole-4-carboxamido)-N1-(1- |
| | (2-(bicyclo[1.1.1]pentan-1-ylamino)-2- |
| | oxoethyl)-2-oxo-1,2-dihydropyridin-3-yl)- |
| | N6-methyl-5-oxohexanediamide |
| II-169 | (S)-N1-(1-(2-(bicyclo[2.1.1]hexan-1-ylamino)- |
| | 2-oxoethyl)-2-oxo-1,2-dihydropyridin-3- |
| | yl)-N6-methyl-2-(3-methylbenzofuran-2- |
| | carboxamido)-5-oxohexanediamide |
| II-170 | (S)-N1-(1-(2-(bicyclo[2.1.1]hexan-1-ylamino)-2- |
| | oxoethyl)-2-oxo-1,2-dihydropyridin-3- |
| | yl)-2-(2-isopropyloxazole-5-carboxamido)- |
| | N6-methyl-5-oxohexanediamide |
| II-171 | (2S)-2-(benzofuran-2-carboxamido)-N1-(1- |
| | (2-(bicyclo[3.2.1]octan-8-ylamino)-2- |
| | oxoethyl)-2-oxo-1,2-dihydropyridin-3-yl)- |
| | N6-methyl-5-oxohexanediamide |
| II-172 | (2S)-N1-(1-(2-(bicyclo[3.2.1]octan-8-ylamino)- |
| | 2-oxoethyl)-2-oxo-1,2-dihydropyridin-3- |
| | yl)-2-(3,5-dimethylisoxazole-4-carboxamido)- |
| | N6-methyl-5-oxohexanediamide |
| II-173 | (S)-N1-(1-(2-(5-carboxy-2-aminoadamantane)-2- |
| | oxoethyl)-2-oxo-1,2-dihydropyridin- |
| | 3-yl)-N6-methyl-2-(4-methylpyrimidine-5- |
| | carboxamido)-5-oxohexanediamide |
| II-174 | (2S)-N1-(1-(2-(4-aminoadamantane-N,N-dimethyl- |
| | 1-carboxamide)-2-oxoethyl)-2-oxo- |
| | 1,2-dihydropyridin-3-yl)-N6-methyl-5-oxo- |
| | 2-(1,2,3,4-tetrahydronaphthalene-2- |
| | carboxamido)hexanediamide |
| II-178 | (S)-2-(benzo[d]thiazole-2-carboxamido)-N1-(1-(2- |
| | ((1S,2R,4R)-bicyclo[2.2.1]heptan-2- |
| | ylamino)-2-oxoethyl)-2-oxo-1,2-dihydropyridin- |
| | 3-yl)-N6-cyclopentyl-5- |
| | oxohexanediamide |
| II-182 | (S)-N1-(1-(2-(bicyclo[1.1.1]pentan-1-ylamino)-2- |
| | oxoethyl)-2-oxo-1,2-dihydropyridin-3- |
| | yl)-N6-cyclohexyl-2-(3-ethylbenzofuran-2- |
| | carboxamido)-5-oxohexanediamide |
| II-183 | (S)-N1-(1-(2-(bicyclo[1.1.1]pentan-1-ylamino)-2- |
| | oxoethyl)-2-oxo-1,2-dihydropyridin-3- |
| | yl)-N6-cyclohexyl-2-(1-ethyl-1H-indole-2- |
| | carboxamido)-5-oxohexanediamide |
| II-194 | (S)-2-(benzofuran-5-carboxamido)-N1-(1-(2- |
| | (1-acetylamino-4-aminoadamantane)-2- |
| | oxoethyl)-2-oxo-1,2-dihydropyridin-3-yl)- |
| | N6-methyl-5-oxohexanediamide |
| II-195 | (S)-2-(benzofuran-6-carboxamido)-N1-(1-(2-(4- |
| | aminoadamantane-1-carboxamide)-2- |
| | oxoethyl)-2-oxo-1,2-dihydropyridin-3-yl)- |
| | N6-methyl-5-oxohexanediamide |
| II-198 | (S)-N1-(1-(2-(bicyclo[1.1.1]pentan-1-ylamino)- |
| | 2-oxoethyl)-2-oxo-1,2-dihydropyridin-3- |
| | yl)-N6-methyl-5-oxo-2-(1,2,3-thiadiazole- |
| | 4-carboxamido)hexanediamide |
| II-199 | (S)-N1-(1-(2-(bicyclo[1.1.1]pentan-1-ylamino)- |
| | 2-oxoethyl)-2-oxo-1,2-dihydropyridin-3- |
| | yl)-N6-methyl-5-oxo-2-(1,2,4-thiadiazole- |
| | 5-carboxamido)hexanediamide |
| II-200 | (S)-N1-(1-(2-(bicyclo[1.1.1]pentan-1-ylamino)- |
| | 2-oxoethyl)-2-oxo-1,2-dihydropyridin-3- |
| | yl)-N6-methyl-5-oxo-2-(1,3,4-thiadiazole- |
| | 2-carboxamido)hexanediamide |
| II-201 | (S)-N1-(1-(2-(bicyclo[1.1.1]pentan-1-ylamino)- |
| | 2-oxoethyl)-2-oxo-1,2-dihydropyridin-3- |
| | yl)-2-(4-cyclopropyl-1,2,3-thiadiazole- |
| | 5-carboxamido)-N6-methyl-5- |
| | oxohexanediamide |
| II-202 | (S)-2-(4-cyclopropyl-1,2,3-thiadiazole-5- |
| | carboxamido)-N1-(1-(2-(2-adamantylamino)- |
| | 2-oxoethyl)-2-oxo-1,2-dihydropyridin-3-yl)- |
| | N6-methyl-5-oxohexanediamide |
| II-203 | (S)-2-(4-isopropyl-1,2,3-thiadiazole-5-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-204 | (S)-2-(4-ethyl-1,2,3-thiadiazole-5-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-205 | (S)-2-(4-formyl-1,2,3-thiadiazole-5-carboxamido)- |
| | N1-(1-(2-(2-adamantylamino)-2- |
| | oxoethyl)-2-oxo-1,2-dihydropyridin-3- |
| | yl)-N6-methyl-5-oxohexanediamide |
| II-206 | (S)-2-(4-(hydroxymethyl)-1,2,3- |
| | thiadiazole-5-carboxamido)-N1-(1-(2-(2- |
| | adamantylamino)-2-oxoethyl)-2-oxo-1,2- |
| | dihydropyridin-3-yl)-N6-methyl-5- |
| | oxohexanediamide |
| II-207 | (S)-N1-(1-(2-(1-adamantylamino)-2-oxoethyl)- |
| | 2-oxo-1,2-dihydropyridin-3-yl)-N6- |
| | methyl-2-(1-methyl-1H-imidazole-5- |
| | carboxamido)-5-oxohexanediamide |
| II-210 | (S)-N1-(1-(2-(3,5-dimethyladamantane- |
| | 1-amino)-2-oxoethyl)-2-oxo-1,2- |
| | dihydropyridin-3-yl)-N6-methyl-2-(1-methyl- |
| | 1H-imidazole-5-carboxamido)-5- |
| | oxohexanediamide |
| II-211 | (S)-N1-(1-(2-(3,5,7-trimethyl-1-adamantylamino)- |
| | 2-oxoethyl)-2-oxo-1,2- |
| | dihydropyridin-3-yl)-N6-methyl-2-(1-methyl- |
| | 1H-imidazole-5-carboxamido)-5- |
| | oxohexanediamide |

or a pharmaceutically acceptable salt thereof.

### Method for Production of Inventive Compounds

In some embodiments, the present invention relates to a method for the synthesis of a compound of formula (I), especially any compound of the formula (Ib):

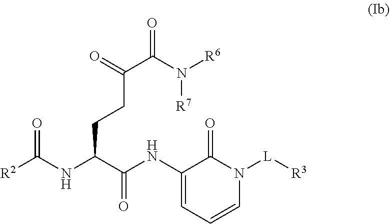

The compound of the formula (Ib) can be produced and thus, the present invention relates to a method for producing the compound of formula (Ib) comprising the following steps in the following order:

Step 1B: providing a compound 4b

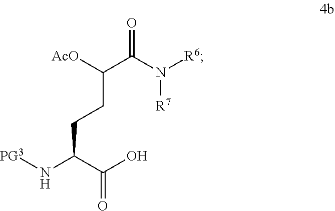

Step 2B: performing coupling reaction of the compound 4b with a compound 5

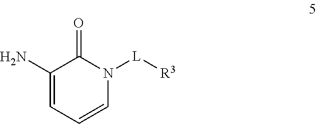

to obtain a compound 6b

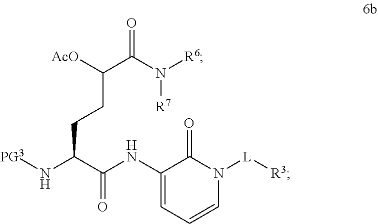

Step 3B: deprotecting an amino protecting group PG³ to obtain a compound 7b

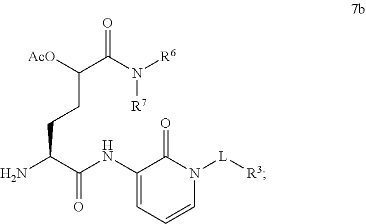

Step 4B: performing coupling reaction of the compound 7b with a carboxylic acid (R²—CO₂H 8) to obtain a compound 9b

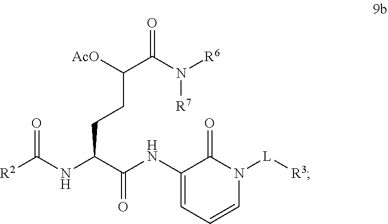

Step 5B: performing oxidation reaction of the compound 9b to produce the compound of the formula (Ib)

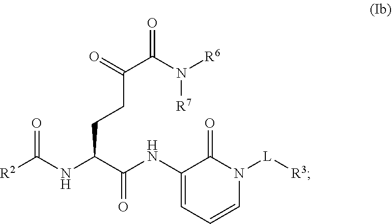

wherein L, R², R³, R⁶ and R⁷ have the same meanings as defined above in the formula (Ib), and PG³ is an amino protecting group.

In the step 5B the chemical warhead precursor

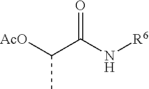

may be firstly converted to

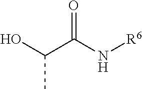

under a basic condition such as treating with K₂CO₃, and then

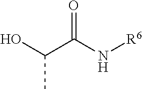

is converted to the corresponding chemical warhead

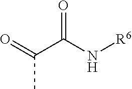

by an oxidation method, preferably by using Dess-Martin periodinane (DMP), iodoxybenzoic acid (IBX), or hypochlorite/TEMPO (2,2,6,6-tetramethylpiperidine-1-oxyl) in a polar solvent, as described in the chemical examples.

In an alternative route first all protecting groups PG¹ and PG² are simultaneously removed and the protecting group PG³ is selectively introduced. Preferably, PG¹ and PG³ are same.

The term “protecting groups” as used herein refers to commonly used protection groups in organic synthesis, preferably for amino and carboxyl groups. PG¹, PG³, and PG⁵ preferably are suitable protecting groups for amino groups. PG² and PG⁴ preferably are suitable protecting groups for carboxyl groups. Preferably, PG¹, PG³, and PG⁵ may be selected from the group consisting of or comprising: acetyl, benzoyl, benzyloxycarbonyl (Cbz), tert-butylcarbonyl, tert-butyloxycarbonyl (Boc), and fluorenylmethylenoxy group (Fmoc). PG² and PG⁴ may be selected from the group consisting of or comprising: methoxy, ethoxy, isobutoxy, tert-butoxy, benzyloxy; preferably, tert-butoxy group.

In Step 2B, to promote the coupling reaction with amino group of intermediate compound, activating reagents are commonly used to activating carboxylic acid (“Peptide Coupling Reagents, More than a Letter Soup”, Ayman El-Faham and Fernando Albericio, Chemical Reviews, 2011, 111 (11), p.6557-6602). The activation may be introduced separate reaction or in situ reaction. Preferably, any of the following coupling reagent can be used to activate carobxylic acid group: BOP (Benzotriazole-1-yl-oxy-tris-(dimethylamino)-phosphonium hexafluorophosphate), PyBOP (Benzotriazole-1-yl-oxy-tris-pyrrolidino-phosphonium hexafluorophosphate), AOP (7-(Azabenzotriazol-1-yl) oxy tris (dimethylamino) phosphonium hexafluorophosphate), PyAOP ((7-Azabenzotriazol-1-yloxy) tripyrrolidinophosphonium hexafluorophosphate), TBTU (2-(1H-Benzotriazole-1-yl)-1,1,3,3-tetramethylaminium tetrafluoroborate), EEDQ (N-Ethoxycarbonyl-2-ethoxy-1,2-dihydroquinoline), Polyphosphoric Acid (PPA), DPPA (Diphenyl phosphoryl azide), HATU (1-[Bis (dimethylamino) methylene]-1H-1,2,3-triazolo[4,5-b]pyridinium 3-oxide hexafluorophosphate), HBTU (O-Benzotriazol-1-yl-N,N,N′,N′-tetramethyluronium hexafluorophosphate), HOBt (1—Hydroxybenzotriazole), HOAt (1—Hydroxy-7-azabenzotriazole), DCC (N,N′-Dicyclohexylcarbodiimide), EDC (or EDAC or EDCl, 1-Ethyl-3-(3-dimethylaminopropyl) carbodiimide), BOP-Cl (Bis (2-oxo-3-oxazolidinyl) phosphinic chloride), TFFH (Tetramethylfluoroformamidinium hexafluorophosphate), BroP (Bromo tris (dimethylamino) phosphonium hexafluorophosphate), PyBroP (Bromo-tris-pyrrolidino-phosphonium hexafluorophosphate) and CIP (2-Chloro-1,3-dimethylimidazolidinium hexafluorophosphate), or further, similar acting reagents, providing an activated intermediate, or a mixture thereof.

### Pharmaceutical Composition & Medical Use

Therefore another aspect of the present invention relates to compounds according to the general formula (I) as medicine as well as their use in medicine. Especially preferred is the use as inhibitors of transglutaminases, in particular transglutaminase 2 (TG2).

Thus the compounds of formula (I) described herein or according to the present invention may be administered themselves or in form of a pharmacologically acceptable salt.

The compounds of the present invention may form of a pharmacologically acceptable salt with organic or inorganic acids or bases. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, trifluoroacetic acid, and other mineral or carboxylic acids well known to those skilled in the art. The salts are prepared by contacting the free base form with a sufficient amount of the desired acid to produce a salt in the conventional manner. Preferred is the mesylate salt, hydrochloride salt and the trifluoroacetate salt and especially preferred is the trifluoroacetate salt and the hydrochloride salt.

In the case the inventive compounds bear acidic groups, salts could also be formed with inorganic or organic bases. Examples for suitable inorganic or organic bases are, for example, NaOH, KOH, NH₄OH, tetraalkylammonium hydroxide, lysine or arginine and the like. Salts may be prepared in a conventional manner using methods well known in the art, for example by treatment of a solution of the compound of the general formula (I) with a solution of an acid, selected out of the group mentioned above.

### Methods of Use

In a further aspect of the present invention, the novel compounds according to the general formula (I) are used as pharmaceutically active agent, i.e. the compound of the formula (I) is used in medicine.

Furthermore, the present invention relates to a pharmaceutical composition comprising at least one compound according to the general formula (I), as an active ingredient or a pharmacologically acceptable salts thereof as an active ingredient, together with at least one pharmacologically acceptable carrier, excipient and/or diluent.

The compounds according to general formula (I) described herein are especially suitable for the treatment and prophylaxis of diseases associated with and/or caused by transglutaminase 2.

Celiac disease, a gluten intolerance is associated with tissue transglutaminase (TG 2). Another very important group of indications for tissue transglutaminase inhibitors are fibrotic disorders. Fibrotic disorders are characterized by the accumulation of cross-linked extracellular matrix proteins. Diabetic nephropathy, cystic fibrosis, idiopathic pulmonary fibrosis, kidney fibrosis as well as liver fibrosis belong to the most important fibrotic disorders to be addressed with the compounds disclosed.

In the biological example B-1, it is proven that the inventive compounds as reversible and irreversible TG inhibitors effectively inhibit the activity of TGs, especially TG2.

As used herein the term “inhibiting” or “inhibition” refers to the ability of a compound to downregulate, decrease, reduce, suppress, inactivate, or inhibit at least partially the activity of an enzyme, or the expression of an enzyme or protein.

Therefore, another aspect of the present invention is the use of the inventive compounds of the general formula (I), or the pharmaceutical composition thereof as described in the treatment or prophylaxis of autoimmune and inflammatory diseases, vascular diseases, fibrotic diseases, liver diseases, cholestatic liver diseases, cancer, neurodegenerative diseases, ocular diseases, and skin disorders.

Further aspects of the present invention relate to the use of the compounds of general formula (I) for the preparation of a pharmaceutical composition useful for prophylaxis and/or treatment of autoimmune and inflammatory diseases, vascular diseases, fibrotic diseases, liver diseases, cholestatic liver diseases, cancer, neurodegenerative diseases, ocular diseases, and skin disorders.

In a further aspect of the present invention, a method for preventing and/or treating autoimmune and inflammatory diseases, vascular diseases, fibrotic diseases, liver diseases, cholestatic liver diseases, cancer, neurodegenerative diseases, ocular diseases, and skin disorders, which method comprises administering to a subject, in particular a human, a pharmaceutically effective amount of at least one compound of the general formula (I), to prevent and/or treat said autoimmune and inflammatory diseases, vascular diseases, fibrotic diseases, liver diseases, cholestatic liver diseases, cancer, neurodegenerative diseases, ocular diseases, and skin disorders.

Preferred, the autoimmune and inflammatory diseases comprises multiple sclerosis, celiac disease, Duhring-Brocq-disease (dermatitis herpetiformis), gluten ataxia, gluten neuropathy, diabetes, rheumatoid arthritis, Graves' disease, inflammatory bowel disease, systemic lupus erythematosus psoriasis, and gingivitis;
the vascular diseases comprise atherosclerosis, thrombosis, vascular stiffness;
the fibrotic diseases affecting the lung, the kidney, the liver, the skin or the gut like cystic fibrosis, kidney fibrosis and diabetic nephropathy, intestinal fibrosis, idiopathic lung fibrosis, liver fibrosis;
the liver diseases like alcoholic hepatitis, alcoholic steatohepatitis, nonalcoholic steatohepatitis, non-alcoholic fatty liver disease, liver cirrhosis, autoimmune hepatitis or liver inflammation;
the cholestatic liver diseases comprise primary biliary cholangitis and primary sclerosing cholangitis;
the cancer comprises glioblastoma, melanoma, pancreatic cancer, renal cell carcinoma, meningioma, and breast cancer,
the neurodegenerative diseases comprise Parkinson's disease, Huntington's disease, or Alzheimer's disease,
the ocular diseases comprise glaucoma, cataracts, macular degeneration, or uveitis;
the skin disorders comprise acne, psoriasis, scarring, and skin aging.

More preferred, the compound of the formula (I), or the pharmaceutical composition thereof is useful in the treatment or prophylaxis of celiac disease.

Furthermore, the compounds of the general formula (I), can be administered in form of their pharmaceutically active salts, optionally using essentially non-toxic pharmaceutically acceptable carriers, adjuvants or extenders. Medications are prepared in a known manner in a conventional solid or fluid carrier or in extenders and a conventional pharmaceutically acceptable adjuvant/expedient in a suitable dose. The preferred preparations are provided in an administrable form suitable for oral application, such as pills, tablets, film tablets, coated tablets, capsules and powders.

Tablets, film tablets, coated tablets, gelatine capsules and opaque capsules are the preferred pharmaceutical formulations. Any pharmaceutical compositions contains at least one compound of the general formula (I), and/or pharmaceutically acceptable salts thereof in an amount of 5 mg to 500 mg, preferably 10 mg to 250 mg and most preferred in an amount of 10 to 100 mg per formulation.

Besides, the object of the present invention also includes pharmaceutical preparations for oral, parenteral, dermal, intradermal, intragastric, intracutaneous, intravascular, intravenous, intramuscular, intraperitoneal, intranasal, intravaginal, intrabuccal, percutaneous, rectal, subcutaneous, sublingual, topic, transdermal or inhalative application, containing, in addition to typical vehicles and extenders, a compound of the general formula (I), and/or a pharmaceutically acceptable salt thereof as active component.

The pharmaceutical compositions of the present invention contain one of the compounds of the formula (I) disclosed herein as active component, typically mixed with suitable carrier materials, selected with respect to the intended form of administration, i.e. tablets to be administered orally, capsules (filled either with a solid, a semi-solid or a liquid), powders, orally administrable gels, elixirs, dispersible granulates, syrups, suspensions and the like in accordance with conventional pharmaceutical practices. For example, the compound of the formula (I) can as active agent component be combined with any oral, non-toxic, pharmaceutically acceptable, inert carrier, such as lactose, starch, sucrose, cellulose, magnesium stearate, dicalcium phosphate, calcium sulfate, talc, mannitol, ethyl alcohol (liquid forms) and the like for the oral administration in form of tablets or capsules. Moreover, suitable binders, lubricants, disintegrants and colorants can be added to the mixture if required. Powders and tablets can consist of said inert carriers to an extent from about 5% per weight to about 95% per weight of the inventive composition.

Suitable binders include starch, gelatine, natural sugars, sweeteners made of corn, natural and gums, such synthetic as acacia gum, sodium alginate, carboxymethylcellulose, polyethylene glycol and waxes. Possible lubricants for the use in said dosage forms include boric acid, sodium benzoate, sodium acetate, sodium chloride and the like. Disintegrants include starch, methylcellulose, cyclodextrins, guar gum and the like. If required, sweeteners and flavor additives and preservatives can also be included. Some of the terms used above, namely disintegrants, extenders, lubricants, binders and the like are discussed in greater detail below.

Additionally, the compositions of the present invention can be formulated in a form with sustained release to provide a controlled release rate of any one or more components or active components, in order to optimize the therapeutic effect, i.e. the inhibitory activity and the like. Suitable dosage forms for sustained release include layered tablets containing layers with varying degradation rates or controlled release polymeric matrices impregnated with the active components and in the form of a tablet or capsule containing such impregnated or encapsulated porous polymeric matrices.

Preparations in fluid form include solutions, suspensions and emulsions. Exemplarily mentioned are water or water propylene glycol solutions for parenteral injections or the addition of sweeteners and opacifiers for oral solutions, suspensions, and emulsions.

Aerosol preparations suitable for inhalation may include solutions and solids in the form of powders which can be combined with a pharmaceutically acceptable carrier, such as a compressed inert gas, e.g. nitrogen.

For the preparation of suppositories a low melting wax, such as a mixture of fatty acid glycerides, e.g. cocoa butter, is melted firstly and the active component is homogenously dispersed therein by stirring or similar mixing operations. The melted homogenous mixture is then poured in fitting forms, cooled and thus hardened.

Further preparations in solid form which are to be converted into preparations in fluid form for either oral or parenteral administration shortly before use are included. Such fluid forms include solutions, suspensions and emulsions.

Furthermore, the compounds of the present invention may be administered via transdermal application. The transdermal compositions can have the form of crèmes, lotions, aerosols and/or emulsions.

The term capsule refers to a special container or casing composed of methylcellulose, polyvinyl alcohols or denatured gelatins or starches, in which the active agents can be enclosed. Typically, hard shell capsules are prepared from mixtures of bones and porcine skin gelatins having comparatively high gel strength. The capsule itself can contain small amounts of colorants, opacifiers, softening agents and preservatives.

Tablet means a compressed or cast solid dosage form containing the active components with suitable extenders. The tablet can be produced by compressing mixtures or granulates obtained by wet granulation, dry granulation or compaction, which are known to the one skilled in the art.

Oral gels refer to the active components dispersed or solubilized in a hydrophilic semi-solid matrix.

Powders for compositions refer to powder mixtures containing the active components and suitable extenders which can be suspended in water or juices.

Suitable extenders are substances which usually form the largest part of the composition or dosage form. Suitable extenders include sugars such as lactose, sucrose, mannitol and sorbitol; starches derived from wheat, corn, rice and potatoes; and celluloses such as microcrystalline cellulose. The amount of extenders in the composition can range from about 5 to about 95% per weight of the total composition, preferably form about 25 to about 75% per weight and further preferred from about 30 to about 60% per weight.

The term disintegrants refers to materials added to the composition in order to support disintegration and release of the medicinal substance. Suitable disintegrants include starches, modified starches which are soluble in cold water, such as sodium carboxymethyl starch; natural and synthetic gums such as locust bean gum, caraya, guar gum, tragacanth and agar; cellulose derivatives such as methylcellulose and sodium carboxymethylcellulose, microcrystalline celluloses and crosslinked microcrystalline celluloses such as croscarmellose sodium; alginates such as alginic acid and sodium alginate; clays such as bentonites and foaming mixtures. The amount of disintegrants used in the composition can range from about 2 to 20% per weight of the composition and further preferred from about 5 to about 10% per weight.

Binders characterize substances binding or “gluing” powders to each other and they consequently serve as “glue” in the formulation. Binders add a cohesion starch which is already available in the extenders or the disintegrant. Suitable binders include sugar, such as sucrose; starches derived from wheat, corn, rice and potatoes; natural gums such as acacia gum, gelatine and tragacanth; derivatives of sea weed such as alginic acid, sodium alginate and ammonium calcium alginate, cellulose materials such as methyl cellulose and sodium carboxymethylcellulose and hydroxypropyl methylcellulose, polyvinylpyrrolidone and inorganic compounds, such as magnesium aluminium silicate. The amount of binders in the composition can range from about 2 to about 20% per weight of the total composition, preferably form about 3 to about 10% per weight and further preferred from about 3 to about 6% per weight.

The term lubricant refers to a substance added to the dosage form in order to allow for the tablet, granulate, etc. to be released from the casting mold or pressing mold, after compression, by reducing the friction. Suitable lubricants include metallic stearates such as magnesium stearate, calcium stearate or potassium stearate; stearic acid; waxes with high melting points and water soluble lubricants such as sodium chloride, sodium benzoate, sodium acetate, sodium oleate, polyethylene glycols and D,L-leucine. Due to the fact that lubricants have to be present on the surface of the granulates as well as between the granulates and parts of the tablet press they are typically added during the last step prior to compression. The amount of lubricants in the composition can range from about 0.2 to about 5% per weight of the total composition, preferably form about 0.5 to about 2% per weight and further preferred from about 0.3 to about 1.5% per weight.

Lubricants are materials preventing caking and improving the flow characteristics of granulates so that the flow is smooth and uniform. Suitable lubricants include silicon dioxide and talc. The amount of lubricants in the composition can range from about 0.1 to about 5% per weight of the total composition, preferably form about 0.5 to about 2% per weight.

Colorants are adjuvants coloring the composition or dosage form. Such adjuvants can include colorants having food quality which are adsorbed on a suitable adsorption means, such as clay or aluminium oxide. The amount of the colorant used can vary from about 0.1 to about 5% per weight of the composition and preferably from about 0.1 to about 1% per weight.

As used herein, a “pharmaceutically effective amount” of a transglutaminase inhibitor is the amount or activity effective for achieving the desired physiological result, either in cells treated in vitro or in a patient treated in vivo. Specifically, a pharmaceutical effective amount is such an amount which is sufficient for inhibiting, for a certain period of time, one or more of the clinically defined pathological processes associated with transglutaminase 2. The effective amount can vary according to the specific compound of the formula (I) and additionally depends on a plurality of factors and conditions related to the subject to be treated and the severity of the disease. If, for example, an inhibitor is to be administered in vivo, factors such as age, weight and health of the patients as well as dose reaction curves and data regarding toxicity obtained from preclinical animal studies are amongst the data to be considered. If the inhibitor in form of the compound of the formula (I) described herein is to be brought in contact with the cells in vivo, a plurality of preclinical in vitro studies would be designed in order to determine parameters such as absorption, half-life, dose, toxicity, etc. Determining a pharmaceutically effective amount for a given pharmaceutically active ingredient is part of the ordinary skills of the one skilled in the art.

### EXAMPLES

Following abbreviations used in the examples have the following meaning.

Boc (tert-butoxycarbonyl), BocOSu (N-tert-butoxycarbonyloxy-succinimide) DCM (dichloromethane), DMAP (4-(Dimethylamino)-pyridine), TEA (triethylamine), DMF (dimethylformamide), DMP (Dess-Martin periodiane), DIPEA (N-Ethyldiisopropylamine), Glu (glutamic acid), EDC (1-ethyl-3-(3′-dimethylaminopropyl) carbodiimide), TFA (trifluoroacetic acid), THF (tetrahydrofuran), EtOAc (ethyl acetate), HATU (1-[Bis (dimethylamino) methylene]-1H-1,2,3-triazolo[4,5-b]pyridinium 3-oxid hexafluorophosphate), HOBt (hydroxybenzotriazole), MTBE (methyl tert-butyl ether), tBu (tert-butyl),

### Chemical Examples

The following examples are intended to illustrate the invention with selected compounds without limiting the protecting scope of the present intellectual property right on these concrete examples. It is clear for a person skilled in the art that analogous compounds and compounds produced according to analogous synthetic ways fall under the protecting scope of the present intellectual property right.

### Example II. Synthetic Method II

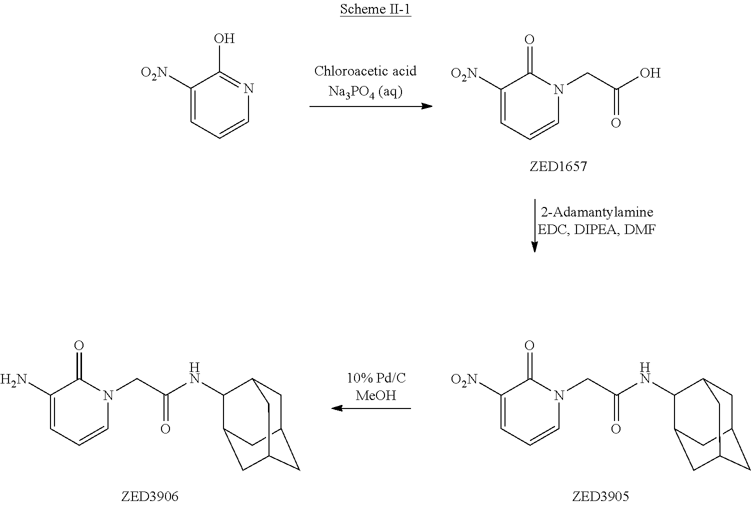 [SMILES: O=[N+]([O-])c1cccnc1O ; O=C(O)Cn1cccc([N+](=O)[O-])c1=O ; Nc1cccn(CC(=O)NC2C3CC4CC(C3)CC2C4)c1=O ; O=C(Cn1cccc([N+](=O)[O-])c1=O)NC1C2CC3CC(C2)CC1C3]

### 1 Preparation of Compound ZED1657

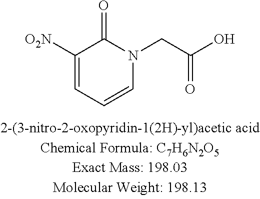 [SMILES: O=C(O)Cn1cccc([N+](=O)[O-])c1=O]

30.0 g (214 mmol) of 2-hydroxy-3-nitropyridine and 40.5 g (2 eq) of chloroacetic acid were suspended in 600 mL water. At 40° C., 245 g (3 eq) trisodium phosphate dodecahydrate were added, and the reaction was stirred at room temperature overnight. 250 mL HCl (32%) were added, and the suspension was stirred for another night at 4° C. The precipitate was filtered and dried. Yield: 41.2 g, 97% ESI-MS: 199.3 [M+H]⁺

### 2 Preparation of Compound ZED3905

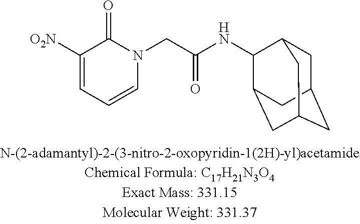 [SMILES: O=C(Cn1cccc([N+](=O)[O-])c1=O)NC1C2CC3CC(C2)CC1C3]

17.0 g (85.8 mmol) of ZED1657, 16.1 g (1 eq) of 2-adamantanamine hydrochloride and 11.6 g (1 eq) of HOBt were dissolved in 200 mL DMF and 17.9 mL (1.2 eq) DIPEA. 18.1 g (1.1 eq) of 1-ethyl-3-(3′-dimethylaminopropyl) carbodiimide hydrochloride were added and the reaction was stirred at room temperature overnight. The solvent was evaporated, and the residue was dissolved in 500 mL DCM. The solution was washed with each 200 mL citric acid solution (10%), NaHCO₃ solution (10%) and brine. The organic phase was dried over Na₂SO₄, filtered and the solvent was evaporated.
Yield: 24.1 g, 85% ESI-MS: 332.4 [M+H]⁺

### 3 Preparation of Compound ZED3906

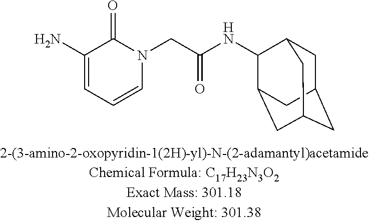 [SMILES: Nc1cccn(CC(=O)NC2C3CC4CC(C3)CC2C4)c1=O]

24.2 g (73.0 mmol) of ZED3905 were suspended in 600 mL MeOH before 2.42 g of palladium (10%) on activated carbon (unreduced) were added. The suspension was stirred overnight at room temperature under an atmosphere of hydrogen. The catalyst was filtered, and the solvent was evaporated. Yield: 15.7 g, 71% ESI-MS: 302.4 [M+H]⁺

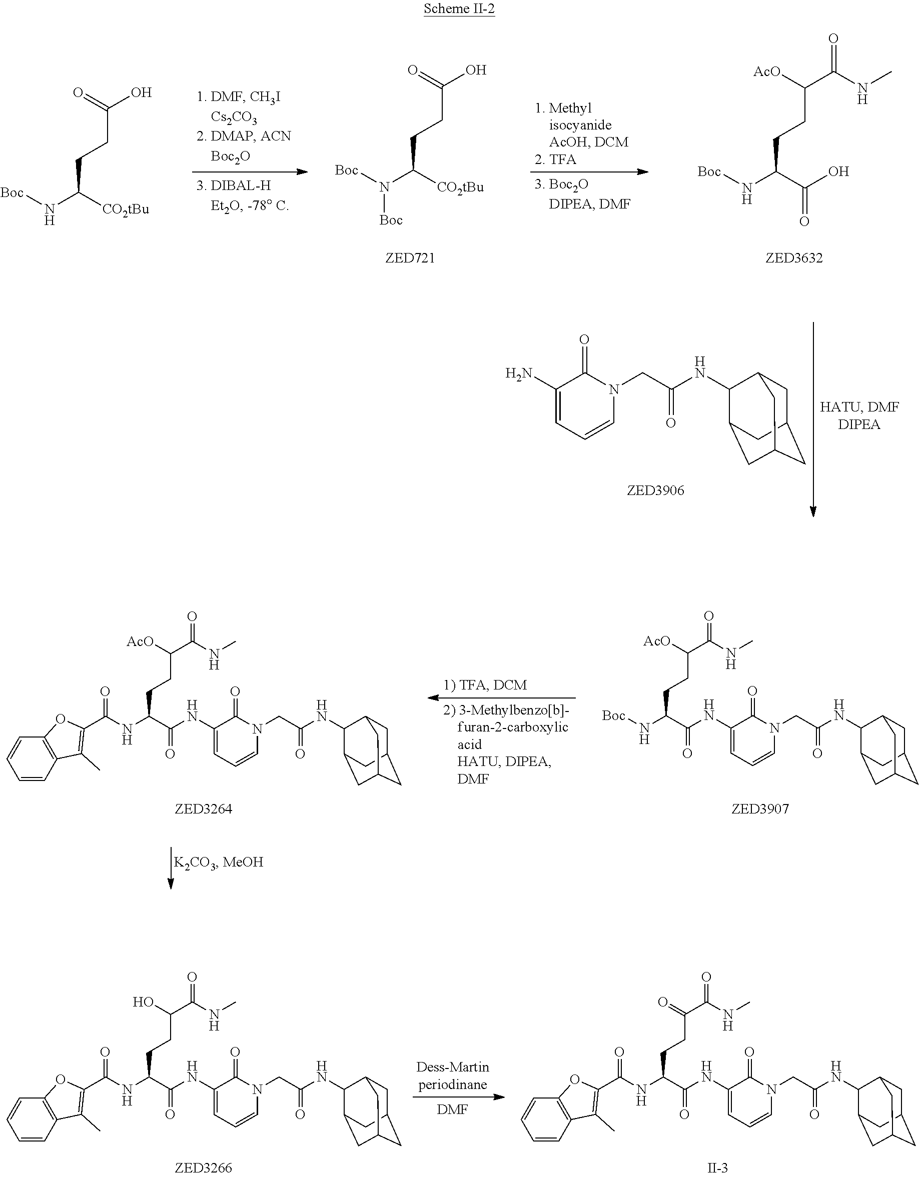

### Preparation of Compound ZED788

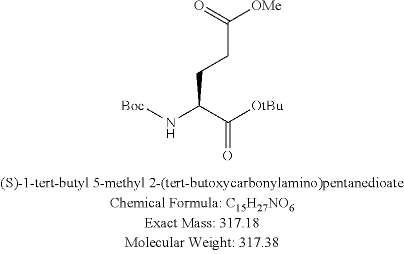 [SMILES: COC(=O)CC[C@H](NC(=O)OC(C)(C)C)C(=O)OC(C)(C)C]

12.0 g of Boc-L-Glu-OtBu (39.6 mmol) and 7.09 g of cesium carbonate (21.8 mmol, 0.55 eq) were suspended in 100 ml of DMF and stirred for 1 h at room temperature. 2.47 ml iodomethane (39.6 mmol) we added, and the mixture was stirred at room temperature overnight. The solvent was evaporated, and the residue was dissolved in ethyl acetate and washed twice with each citric acid solution (10%), NaHCO₃ solution (10%) and 10 brine. The organic phase was dried over Na₂SO₄, filtered and the solvent was evaporated. The raw product was used without further purification.
Yield: 13.4 g, >100%
ESI-MS: 318.3 [M+H]⁺

### Preparation of Compound ZED720

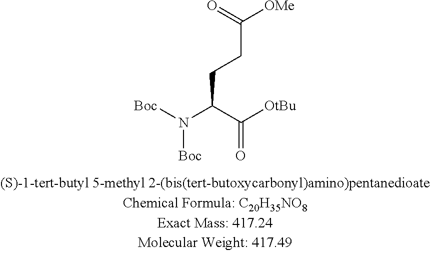 [SMILES: COC(=O)CC[C@@H](C(=O)OC(C)(C)C)N(C(=O)OC(C)(C)C)C(=O)OC(C)(C)C]

13.4 g of ZED788 (˜39.6 mmol) and 986 mg of N, N-dimethyl-4-aminopyridine (DMAP) were dissolved in 30 ml of acetonitrile. 17.6 g of di-tert-butyl bicarbonate (77.1 mmol) in 100 ml of acetonitrile was added and the solution was stirred at room temperature overnight. The solvent was evaporated, and the residue was dissolved in ethyl acetate and washed twice with each citric acid solution (10%), NaHCO₃ solution (10%) and brine. The organic phase was dried over Na₂SO₄, filtered and the solvent was evaporated. The raw product was used without further purification.
Yield: 13.7 g, 83%
ESI-MS: 418.3 [M+H]⁺

### Preparation of Compound ZED721

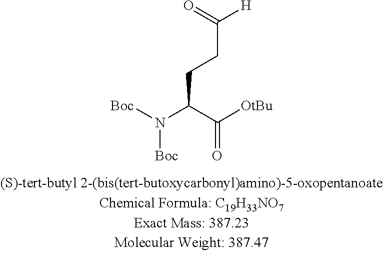 [SMILES: CC(C)(C)OC(=O)[C@H](CCC=O)N(C(=O)OC(C)(C)C)C(=O)OC(C)(C)C]

13.7 g of ZED720 (32.8 mmol) were dissolved in 200 ml of dry diethyl ether and cooled to −78° C. under argon atmosphere. 36.1 ml of diisobutylaluminum hydride (1M in hexane) were added dropwise and the solution was stirred for 30 min at −78° C. before being quenched with potassium sodium tartrate (Rochelle salt) solution. The organic layer was separated, dried over Na₂SO₄, filtered, and concentrated to dryness. The raw product was used without further purification.
Yield: 13.3 g, >100%
ESI-MS: 388.3 [M+H]⁺

### 4 Preparation of Compound ZED3632

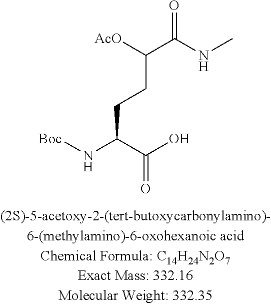 [SMILES: CNC(=O)C(CC[C@H](NC(=O)OC(C)(C)C)C(=O)O)OC(C)=O]

15.0 g (38.7 mmol) of the aldehyde(S)-tert-butyl 2-(bis (tert-butoxycarbonyl) amino)-5-oxopentanoate (ZED721) were dissolved in 60 mL DCM. At 0° C. 2.42 mL (1.05 eq) methyl isocyanide and 2.33 mL (1.05 eq) acetic acid were added, and the reaction was stirred at room temperature overnight. 75 mL TFA were added, and the reaction was stirred for another 3 h. The solvent was evaporated, and the residue was dissolved in 40 mL DMF. 13.2 mL (2 eq) DIPEA and 10.4 g (46.6 mmol) di-tert-butyl dicarbonate in 10 mL DMF were added and the reaction was stirred at room temperature overnight. The solvent was evaporated, and the residue was dissolved in DCM. After extraction with NaHCO₃ solution (1.05 eq in water), 1.5 eq citric acid was added to the aqueous phase, followed by re-extraction with DCM. The organic phase was dried over Na₂SO₄, filtered and the solvent was evaporated. The residue was purified by flash chromatography.
Yield: 12.5 g, 95%
ESI-MS: 333.5 [M+H]⁺

### 5 Preparation of Compound ZED3907

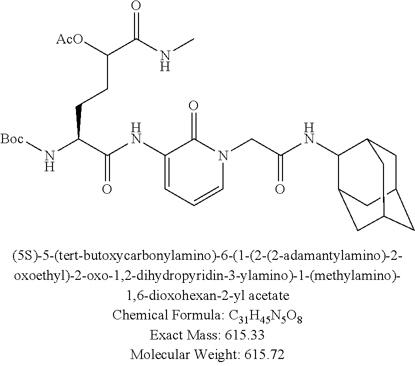 [SMILES: CNC(=O)C(CC[C@H](NC(=O)OC(C)(C)C)C(=O)Nc1cccn(CC(=O)NC2C3CC4CC(C3)CC2C4)c1=O)OC(C)=O]

19.8 g (59.5 mmol) of ZED3632, 22.6 g (1 eq) HATU and 17.9 g (1 eq) ZED3906 were dissolved in 400 mL DMF and 20.8 mL DIPEA (2 eq) and stirred at 45° C. overnight. The solvent was evaporated; the residue was dissolved in 200 mL EtOAc and washed twice with each 150 mL citric acid solution (10%), NaHCO₃ solution (10%) and brine. The organic phase was dried over Na₂SO₄, filtered and the solvent was evaporated.
Yield: 27.4 g, 75%
ESI-MS: 616.4 [M+H]⁺

### 6 Preparation of Compound ZED3264

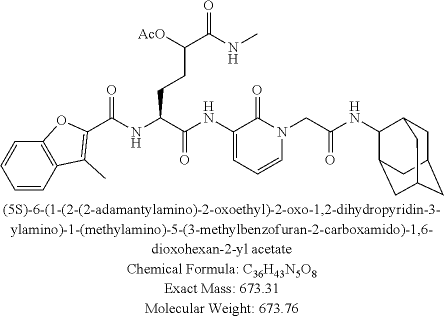 [SMILES: CNC(=O)C(CC[C@H](NC(=O)c1oc2ccccc2c1C)C(=O)Nc1cccn(CC(=O)NC2C3CC4CC(C3)CC2C4)c1=O)OC(C)=O]

480 mg (0.78 mmol) of ZED3907 were dissolved in 4 ml DCM/TFA (1:1) and stirred at room temperature for 1 h. The solvent was evaporated, and the residue was dissolved in 4 ml DMF. 137 mg (1 eq)₃-methylbenzo[b]furan-2-carboxylic acid, 296 mg (1 eq) HATU and 272 ul (2 eq) DIPEA were added, and the reaction was stirred at room temperature overnight. The solvent was evaporated; the residue was dissolved in 20 mL EtOAc and washed with each 10 mL citric acid solution (10%), NaHCO₃ solution (10%) and brine. The organic phase was dried over Na₂SO₄, filtered and the solvent was evaporated.
Yield: 409 mg, 78%
ESI-MS: 674.4 [M+H]⁺

### 7 Preparation of Compound ZED3266

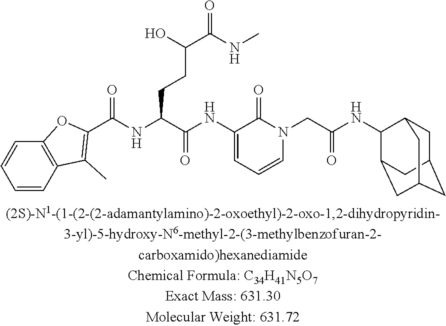 [SMILES: CNC(=O)C(O)CC[C@H](NC(=O)c1oc2ccccc2c1C)C(=O)Nc1cccn(CC(=O)NC2C3CC4CC(C3)CC2C4)c1=O]

409 mg (0.61 mmol) of ZED3264 were dissolved in 5 ml MeOH. 126 mg (1.5 eq) potassium carbonate were added, and the reaction was stirred at room temperature for 1 h. The solution was diluted with DCM and washed with water. The organic phase was dried over Na₂SO₄, filtered and the solvent was evaporated.
Yield: 377 mg, 98%
ESI-MS: 632.4 [M+H]⁺

### 8 Preparation of Compound II-3

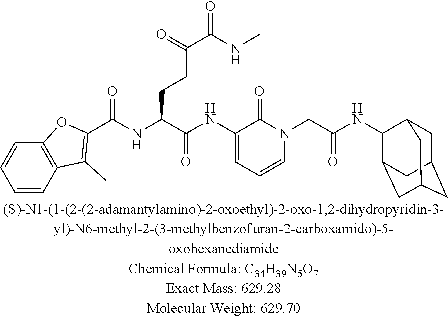 [SMILES: CNC(=O)C(=O)CC[C@H](NC(=O)c1oc2ccccc2c1C)C(=O)Nc1cccn(CC(=O)NC2C3CC4CC(C3)CC2C4)c1=O]

377 mg (0.60 mmol) of ZED3266 were dissolved in 2 ml DMF. 405 mg (1.6 eq) Dess-Martin periodinane (DMP) were added and the reaction was stirred at room temperature over 2 h. The precipitate was filtered off and the filtrate was evaporated. The residue was purified by HPLC.
Yield: 314 mg, 67%
ESI-MS: 630.4 [M+H]⁺

¹H-NMR (DMSO-D₆, 500 MHz, 8 [ppm]: 1.46//1.98 (d//d, 2H//2H, adamantyl-C4-H₂), 1.68//1.78 (m, 4H, adamantyl-C4-H₂), 1.71 (m, 2H, adamantyl-C1-H), 1.75 (m, 2H, adamantyl-C6-H₂), 1.78 (m, 2H, adamantyl-C5-H), 2.05//2.16 (m//m, 1H//1H, β-CH₂), 2.53 (s, 3H, benzofuran-CH₃), 2.64 (d, 3H, amide-N—CH₃), 2.96 (t, 2H, γ-CH₂), 3.82 (m, 1H, adamantyl-C2-H), 4.64 (s, 2H, N—CH₂), 4.70 (ddd, 1H, α-CH₂), 6.25 (t, 1H, pyridinone-C5-H), 7.33 (d, 1H, pyridinone-C6-H), 7.36 (t, 1H, benzofuran-CH), 7.51 (t, 1H, benzofuran-CH), 7.63 (d, 1H, benzofuran-CH), 7.76 (d, 1H, benzofuran-CH), 8.06 (d, 1H, adamantyl-NH), 8.21 (d, 1H, pyridinone-C4-H), 8.54 (q, 1H, methylamide-NH), 8.87 (d, 1H, α-NH), 9.36 (s, 1H, pyridinone-NH).

¹³C-NMR (DMSO-D₆, 500 MHZ, 8 [ppm]: 8.62 (benzofuran-CH₃), 24.50 (β-CH₂), 25.37 (amide-N—CH₃), 26.57//26.62 (adamantyl-C5-H), 30.83 (adamantyl-C4-H₂), 31.35 (adamantyl-C1-H), 33.61 (γ-CH₂), 36.66 (adamantyl-C4′-H₂), 37.01 (adamantyl-C6-H₂), 51.64 (N—CH₂), 52.80 (α-CH₂), 53.24 (adamantyl-C2-H), 104.51 (pyridinone-C5-H), 111.55 (benzofuran-CH), 121.09 (benzofuran-CH), 121.72 (benzofuran-Cq), 122.53 (pyridinone-C4-H), 123.19 (benzofuran-CH), 127.28 (pyridinone-N-Cq), 127.89 (benzofuran-CH), 129.02 (benzofuran-Cq), 133.27 (pyridinone-C6-H), 142.31 (benzofuran-Cq), 152.68 (benzofuran-Cq), 156.55 (pyridinone-C═O), 159.59 (benzofuran-C═O), 161.32 (C═O—NH—CH₃), 165.65 (C═O-adamantylamide), 170.42 (C═O—NH-pyridinone), 198.06 (C═O-methylamide).

### 9 Preparation of Compound II-2

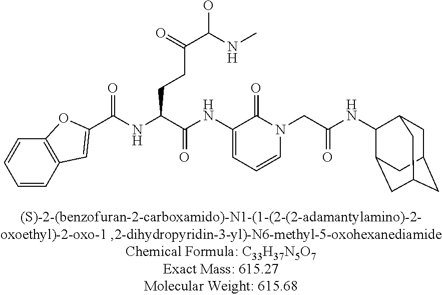 [SMILES: CNC(O)C(=O)CC[C@H](NC(=O)c1cc2ccccc2o1)C(=O)Nc1cccn(CC(=O)NC2C3CC4CC(C3)CC2C4)c1=O]

To the α-hydroxyester precursor of compound II-2 (242 mg, 0.39 mmol, prepared by using benzofuran-2-carboxylic acid in step 6 according to compound ZED3264) in 8 mL of acetonitrile, 1 mg of TEMPO (2,2,6,6-tetramethylpiperidine-1-oxyl, 1 mol %) were added. 56 mg of calcium hypochlorite (1 eq) were added at 0° C. and the reaction mixture was stirred at 25° C. for 2 h. The suspension was filtered, diluted with ethyl acetate and washed with NaHCO₃ solution (10%) and brine. The organic phase was dried over Na₂SO₄, filtered and the solvent was evaporated. The residue was purified by HPLC.
Yield: 102 mg, 42%
ESI-MS: 616.3 [M+H]⁺

### Preparation of Compound II-4

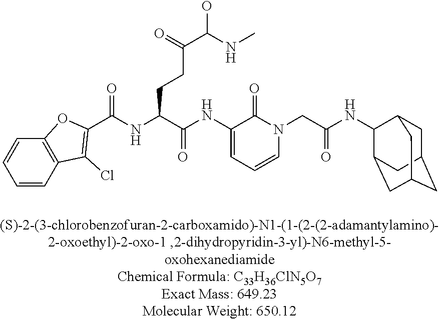 [SMILES: CNC(O)C(=O)CC[C@H](NC(=O)c1oc2ccccc2c1Cl)C(=O)Nc1cccn(CC(=O)NC2C3CC4CC(C3)CC2C4)c1=O]

To the α-hydroxyester precursor of compound II-4 (124 mg, 0.19 mmol, prepared by using 3-chlorobenzofuran-2-carboxylic acid in step 6 according to compound ZED3264) in 4 ml DMSO, 106 mg of 2-iodoxybenzoic acid (IBX, 2 eq) were added and the reaction mixture was stirred at room temperature for 3 h. NaHCO₃ solution (10%) was added and the suspension was extracted with EtOAc. The organic phase was dried over Na₂SO₄, filtered and the solvent was evaporated. The residue was purified by HPLC.
Yield: 37 mg, 30% (last step)
ESI-MS: 650.3/652.3 [M+H]⁺

### 11 Preparation of Compound II-5

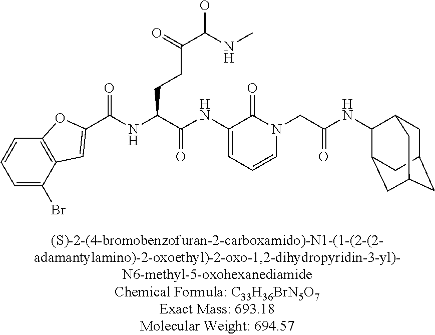 [SMILES: CNC(O)C(=O)CC[C@H](NC(=O)c1cc2c(Br)cccc2o1)C(=O)Nc1cccn(CC(=O)NC2C3CC4CC(C3)CC2C4)c1=O]

The synthesis of compound II-5 was performed according to compound II-3, using 4-bromo-1-benzofuran-2-carboxylic acid instead of 3-methylbenzo[b]furan-2-carboxylic acid in step 6 (according to ZED3264).
Yield: 69 mg, 72% (last step)
ESI-MS: 694.3/696.3 [M+H]⁺

### 12 Preparation of Compound II-6

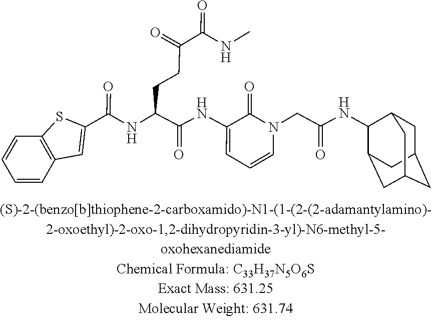 [SMILES: CNC(=O)C(=O)CC[C@H](NC(=O)c1cc2ccccc2s1)C(=O)Nc1cccn(CC(=O)NC2C3CC4CC(C3)CC2C4)c1=O]

The synthesis of compound II-6 was performed according to compound II-3, using benzo[b]thiophene-2-carboxylic acid instead of 3-methylbenzo[b]furan-2-carboxylic acid in step 6 (according to ZED3264).
Yield: 287 mg, 76% (last step)
ESI-MS: 632.3 [M+H]⁺

### 13 Preparation of Compound II-7

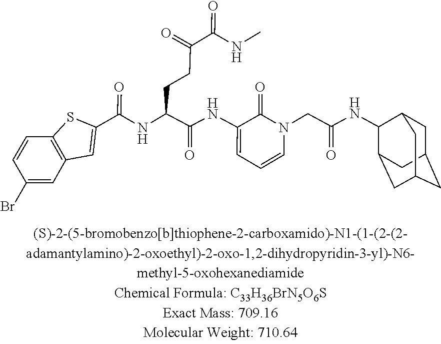 [SMILES: CNC(=O)C(=O)CC[C@H](NC(=O)c1cc2cc(Br)ccc2s1)C(=O)Nc1cccn(CC(=O)NC2C3CC4CC(C3)CC2C4)c1=O]

The synthesis of compound II-7 was performed according to compound II-3, using 5-bromobenzo[b]thiophene-2-carboxylic acid instead of 3-methylbenzo[b]furan-2-carboxylic acid in step 6 (according to ZED3264).
Yield: 145 mg, 59% (last step)
ESI-MS: 710.2/712.2 [M+H]⁺

### 14 Preparation of Compound II-8

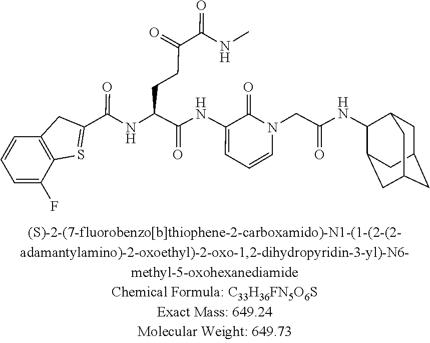

The synthesis of compound II-8 was performed according to compound II-3, using 7-fluorobenzo[b]thiophene-2-carboxylic acid instead of 3-methylbenzo[b]furan-2-carboxylic acid in step 6 (according to ZED3264).
Yield: 78 mg, 71% (last step)
ESI-MS: 650.3 [M+H]⁺

### 15 Preparation of Compound II-9

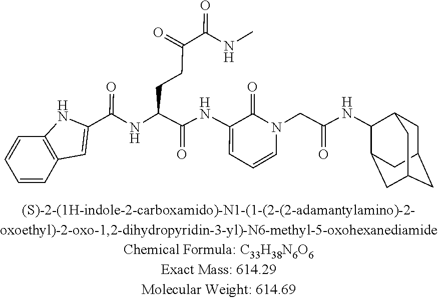 [SMILES: CNC(=O)C(=O)CC[C@H](NC(=O)c1cc2ccccc2[nH]1)C(=O)Nc1cccn(CC(=O)NC2C3CC4CC(C3)CC2C4)c1=O]

The synthesis of compound II-9 was performed according to compound II-3, using 1H-indole-2-carboxylic acid instead of 3-methylbenzo[b]furan-2-carboxylic acid in step 6 (according to ZED3264).
Yield: 57 mg, 69% (last step)
ESI-MS: 615.4 [M+H]⁺

### 16 Preparation of Compound II-10

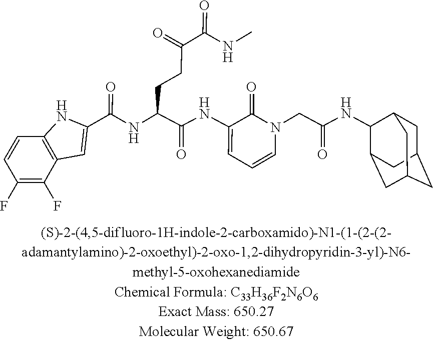 [SMILES: CNC(=O)C(=O)CC[C@H](NC(=O)c1cc2c(F)c(F)ccc2[nH]1)C(=O)Nc1cccn(CC(=O)NC2C3CC4CC(C3)CC2C4)c1=O]

The synthesis of compound II-10 was performed according to compound II-3, using 4,5-difluoro-1H-indole-2-carboxylic acid instead of 3-methylbenzo[b]furan-2-carboxylic acid in step 6 (according to ZED3264).
Yield: 47 mg, 65% (last step)
ESI-MS: 651.3 [M+H]⁺

### 17 Preparation of Compound II-11

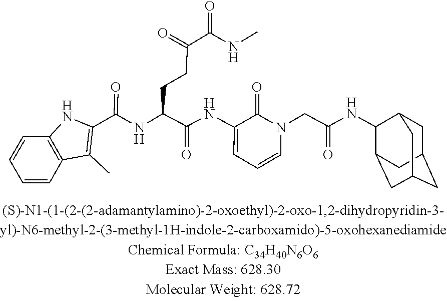 [SMILES: CNC(=O)C(=O)CC[C@H](NC(=O)c1[nH]c2ccccc2c1C)C(=O)Nc1cccn(CC(=O)NC2C3CC4CC(C3)CC2C4)c1=O]

The synthesis of compound II-11 was performed according to compound II-3, using 3-methyl-1H-indole-2-carboxylic acid instead of 3-methylbenzo[b]furan-2-carboxylic acid in step 6 (according to ZED3264).
Yield: 58 mg, 72% (last step)
ESI-MS: 629.4 [M+H]⁺

### 18 Preparation of Compound II-12

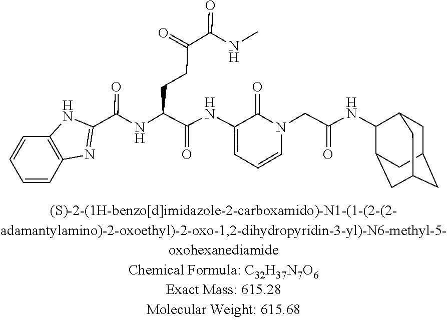 [SMILES: CNC(=O)C(=O)CC[C@H](NC(=O)c1nc2ccccc2[nH]1)C(=O)Nc1cccn(CC(=O)NC2C3CC4CC(C3)CC2C4)c1=O]

The synthesis of compound II-12 was performed according to compound II-3, using 1H-benzo[d]imidazole-2-carboxylic acid instead of 3-methylbenzo[b]furan-2-carboxylic acid in step 6 (according to ZED3264).
Yield: 27 mg, 48% (last step)
ESI-MS: 616.4 [M+H]⁺

### 19 Preparation of Compound II-13

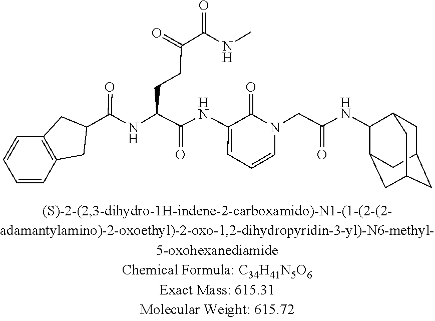 [SMILES: CNC(=O)C(=O)CC[C@H](NC(=O)C1Cc2ccccc2C1)C(=O)Nc1cccn(CC(=O)NC2C3CC4CC(C3)CC2C4)c1=O]

The synthesis of compound II-13 was performed according to compound II-3, using 2,3-dihydro-1H-indene-2-carboxylic acid instead of 3-methylbenzo[b]furan-2-carboxylic acid in step 6 (according to ZED3264).
Yield: 38 mg, 61% (last step)
ESI-MS: 616.4 [M+H]⁺

### 20 Preparation of Compound II-14

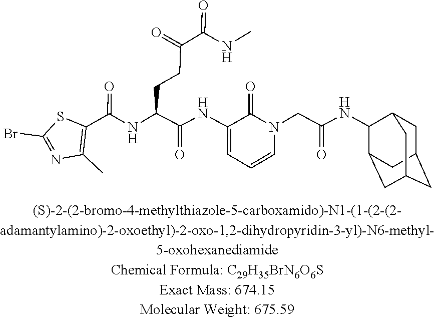 [SMILES: CNC(=O)C(=O)CC[C@H](NC(=O)c1sc(Br)nc1C)C(=O)Nc1cccn(CC(=O)NC2C3CC4CC(C3)CC2C4)c1=O]

The synthesis of compound II-14 was performed according to compound II-3, using 2-bromo-4-methylthiazole-5-carboxylic acid instead of 3-methylbenzo[b]furan-2-carboxylic acid in step 6 (according to ZED3264).
Yield: 98 mg, 70% (last step)
ESI-MS: 675.2/677.2 [M+H]⁺

### 21 Preparation of Compound II-15

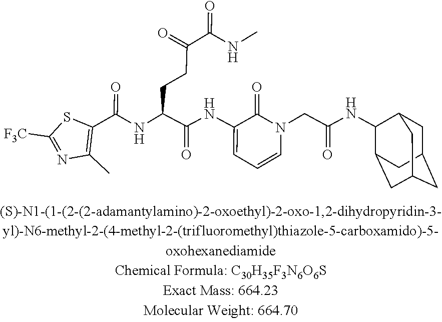 [SMILES: CNC(=O)C(=O)CC[C@H](NC(=O)c1sc(C(F)(F)F)nc1C)C(=O)Nc1cccn(CC(=O)NC2C3CC4CC(C3)CC2C4)c1=O]

The synthesis of compound II-15 was performed according to compound II-3, using 4-methyl-2-(trifluoromethyl) thiazole-5-carboxylic acid instead of 3-methylbenzo[b]furan-2-carboxylic acid in step 6 (according to ZED3264).
Yield: 67 mg, 51% (last step)
ESI-MS: 665.4 [M+H]⁺

### 22 Preparation of Compound II-16

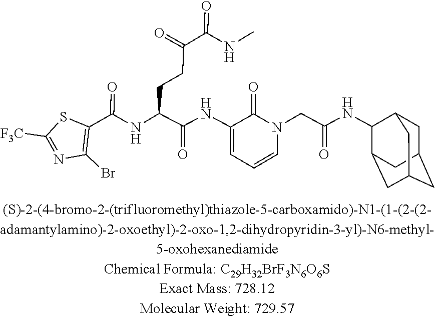 [SMILES: CNC(=O)C(=O)CC[C@H](NC(=O)c1sc(C(F)(F)F)nc1Br)C(=O)Nc1cccn(CC(=O)NC2C3CC4CC(C3)CC2C4)c1=O]

The synthesis of compound II-16 was performed according to compound II-3, using 4-bromo-2-(trifluoromethyl) thiazole-5-carboxylic acid instead of 3-methylbenzo[b]furan-2-carboxylic acid in step 6 (according to ZED3264).
Yield: 136 mg, 63% (last step)
ESI-MS: 729.3/731.3 [M+H]⁺

### 23 Preparation of Compound II-17

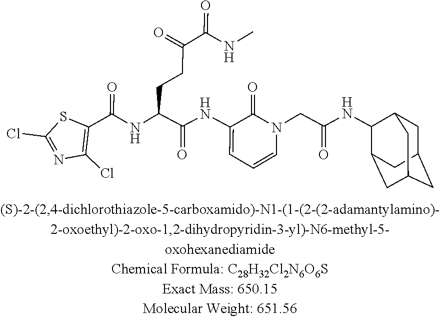 [SMILES: CNC(=O)C(=O)CC[C@H](NC(=O)c1sc(Cl)nc1Cl)C(=O)Nc1cccn(CC(=O)NC2C3CC4CC(C3)CC2C4)c1=O]

The synthesis of compound II-17 was performed according to compound II-3, using 2,4-dichlorothiazole-5-carboxylic acid instead of 3-methylbenzo[b]furan-2-carboxylic acid in step 6 (according to ZED3264).
Yield: 102 mg, 71% (last step)
ESI-MS: 651.2/653.2 [M+H]⁺

### 24 Preparation of Compound II-18

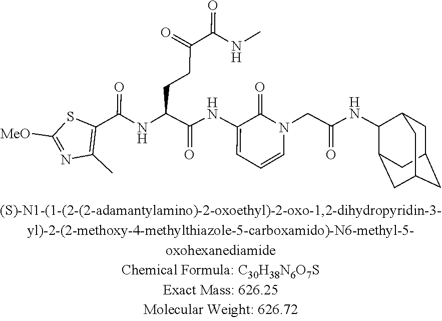 [SMILES: CNC(=O)C(=O)CC[C@H](NC(=O)c1sc(OC)nc1C)C(=O)Nc1cccn(CC(=O)NC2C3CC4CC(C3)CC2C4)c1=O]

The synthesis of compound II-18 was performed according to compound II-3, using 2-methoxy-4-methylthiazole-5-carboxylic acid instead of 3-methylbenzo[b]furan-2-carboxylic acid in step 6 (according to ZED3264).
Yield: 56 mg, 63% (last step)
ESI-MS: 627.3 [M+H]⁺

### 25 Preparation of Compound II-19

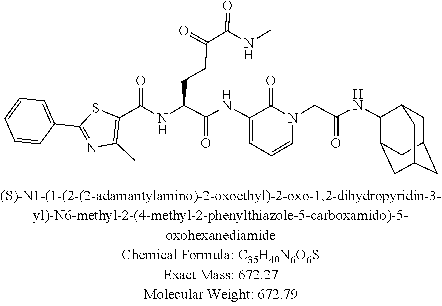 [SMILES: CNC(=O)C(=O)CC[C@H](NC(=O)c1sc(-c2ccccc2)nc1C)C(=O)Nc1cccn(CC(=O)NC2C3CC4CC(C3)CC2C4)c1=O]

The synthesis of compound II-19 was performed according to compound II-3, using 4-methyl-2-phenylthiazole-5-carboxylic acid instead of 3-methylbenzo[b]furan-2-carboxylic acid in step 6 (according to ZED3264).
Yield: 46 mg, 67% (last step)
ESI-MS: 673.4 [M+H]⁺

### 26 Preparation of Compound II-20

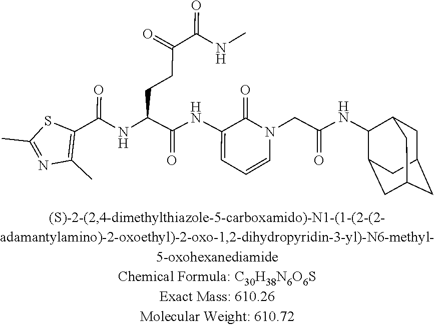 [SMILES: CNC(=O)C(=O)CC[C@H](NC(=O)c1sc(C)nc1C)C(=O)Nc1cccn(CC(=O)NC2C3CC4CC(C3)CC2C4)c1=O]

The synthesis of compound II-20 was performed according to compound II-3, using 2,4-dimethylthiazole-5-carboxylic acid instead of 3-methylbenzo[b]furan-2-carboxylic acid in step 6 (according to ZED3264).
Yield: 216 mg, 77% (last step)
ESI-MS: 611.4 [M+H]⁺

### 27 Preparation of Compound II-21

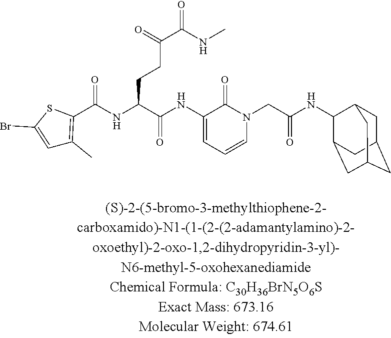 [SMILES: CNC(=O)C(=O)CC[C@H](NC(=O)c1sc(Br)cc1C)C(=O)Nc1cccn(CC(=O)NC2C3CC4CC(C3)CC2C4)c1=O]

The synthesis of compound II-21 was performed according to compound II-3, using 5-bromo-3-methylthiophene-2-carboxylic acid instead of 3-methylbenzo[b]furan-2-carboxylic acid in step 6 (according to ZED3264).
Yield: 178 mg, 79% (last step)
ESI-MS: 674.2/676.2.4 [M+H]⁺

### 28 Preparation of Compound II-22

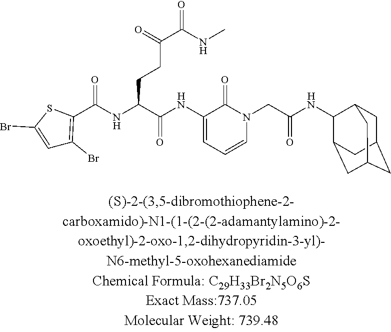 [SMILES: CNC(=O)C(=O)CC[C@H](NC(=O)c1sc(Br)cc1Br)C(=O)Nc1cccn(CC(=O)NC2C3CC4CC(C3)CC2C4)c1=O]

The synthesis of compound II-22 was performed according to compound II-3, using 3,5-dibromothiophene-2-carboxylic acid instead of 3-methylbenzo[b]furan-2-carboxylic acid in step 6 (according to ZED3264).
Yield: 89 mg, 67% (last step)
ESI-MS: 738.2/740.2/742.2 [M+H]⁺

### 29 Preparation of Compound II-23

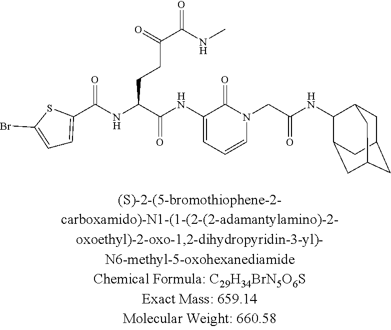 [SMILES: CNC(=O)C(=O)CC[C@H](NC(=O)c1ccc(Br)s1)C(=O)Nc1cccn(CC(=O)NC2C3CC4CC(C3)CC2C4)c1=O]

The synthesis of compound II-23 was performed according to compound II-3, using 5-bromothiophene-2-carboxylic acid instead of 3-methylbenzo[b]furan-2-carboxylic acid in step 6 (according to ZED3264).
Yield: 141 mg, 72% (last step)
ESI-MS: 660.2/662.2 [M+H]⁺

### 30 Preparation of Compound II-24

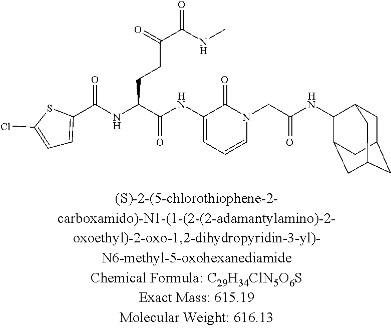 [SMILES: CNC(=O)C(=O)CC[C@H](NC(=O)c1ccc(Cl)s1)C(=O)Nc1cccn(CC(=O)NC2C3CC4CC(C3)CC2C4)c1=O]

The synthesis of compound II-24 was performed according to compound II-3, using 5-chlorothiophene-2-carboxylic acid instead of 3-methylbenzo[b]furan-2-carboxylic acid in step 6 (according to ZED3264).
Yield: 117 mg, 78% (last step)
ESI-MS: 616.3/618.3 [M+H]⁺

### 31 Preparation of Compound II-25

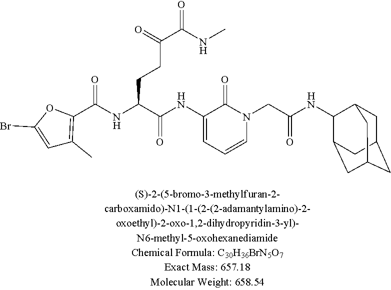 [SMILES: CNC(=O)C(=O)CC[C@H](NC(=O)c1oc(Br)cc1C)C(=O)Nc1cccn(CC(=O)NC2C3CC4CC(C3)CC2C4)c1=O]

The synthesis of compound II-25 was performed according to compound II-3, using 5-bromo-3-methylfuran-2-carboxylic acid instead of 3-methylbenzo[b]furan-2-carboxylic acid in step 6 (according to ZED3264).
Yield: 173 mg, 72% (last step)
ESI-MS: 658.2/660.2 [M+H]⁺

### 32 Preparation of Compound II-26

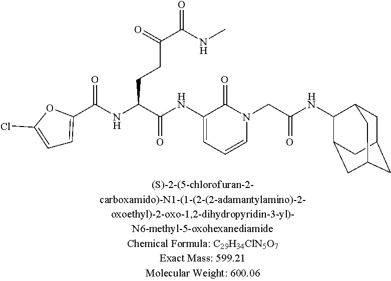 [SMILES: CNC(=O)C(=O)CC[C@H](NC(=O)c1ccc(Cl)o1)C(=O)Nc1cccn(CC(=O)NC2C3CC4CC(C3)CC2C4)c1=O]

The synthesis of compound II-26 was performed according to compound II-3, using 5-chlorofuran-2-carboxylic acid instead of 3-methylbenzo[b]furan-2-carboxylic acid in step 6 (according to ZED3264).
Yield: 127 mg, 56% (last step)
ESI-MS: 600.3/602.3 [M+H]⁺

### 33 Preparation of Compound II-27

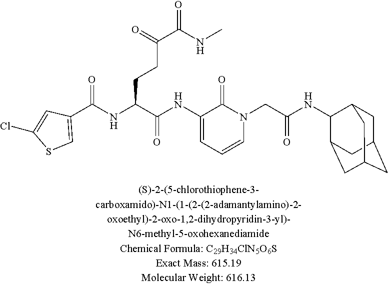 [SMILES: CNC(=O)C(=O)CC[C@H](NC(=O)c1csc(Cl)c1)C(=O)Nc1cccn(CC(=O)NC2C3CC4CC(C3)CC2C4)c1=O]

The synthesis of compound II-27 was performed according to compound II-3, using 5-chlorothiophene-3-carboxylic acid instead of 3-methylbenzo[b]furan-2-carboxylic acid in step 6 (according to ZED3264).
Yield: 112 mg, 65% (last step)
ESI-MS: 616.3/618.3 [M+H]⁺

### 34 Preparation of Compound II-28

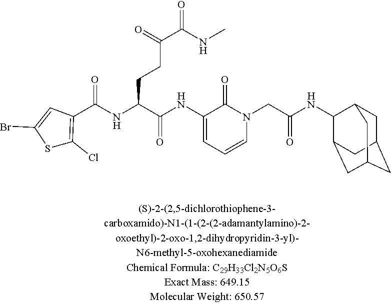 [SMILES: CNC(=O)C(=O)CC[C@H](NC(=O)c1cc(Br)sc1Cl)C(=O)Nc1cccn(CC(=O)NC2C3CC4CC(C3)CC2C4)c1=O]

The synthesis of compound II-28 was performed according to compound II-3, using 2,5-dichlorothiophene-3-carboxylic acid instead of 3-methylbenzo[b]furan-2-carboxylic acid in step 6 (according to ZED3264).
Yield: 319 mg, 77% (last step)
ESI-MS: 650.3/652.3 [M+H]⁺

### 35 Preparation of Compound II-29

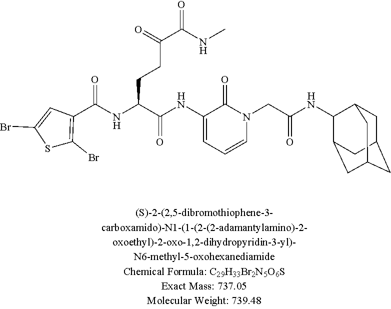 [SMILES: CNC(=O)C(=O)CC[C@H](NC(=O)c1cc(Br)sc1Br)C(=O)Nc1cccn(CC(=O)NC2C3CC4CC(C3)CC2C4)c1=O]

The synthesis of compound II-29 was performed according to compound II-3, using 2,5-dibromothiophene-3-carboxylic acid instead of 3-methylbenzo[b]furan-2-carboxylic acid in step 6 (according to ZED3264).
Yield: 98 mg, 52% (last step)
ESI-MS: 738.2/740.2/742.2 [M+H]⁺

### 36 Preparation of Compound II-30

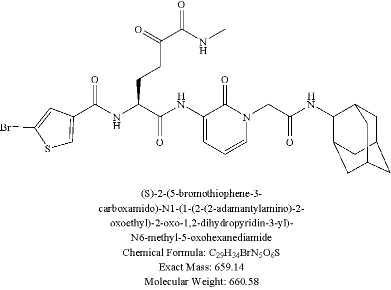 [SMILES: CNC(=O)C(=O)CC[C@H](NC(=O)c1csc(Br)c1)C(=O)Nc1cccn(CC(=O)NC2C3CC4CC(C3)CC2C4)c1=O]

The synthesis of compound II-30 was performed according to compound II-3, using 5-bromothiophene-3-carboxylic acid instead of 3-methylbenzo[b]furan-2-carboxylic acid in step 6 (according to ZED3264).
Yield: 171 mg, 73% (last step)
ESI-MS: 660.2/662.2 [M+H]⁺

### 37 Preparation of Compound II-31

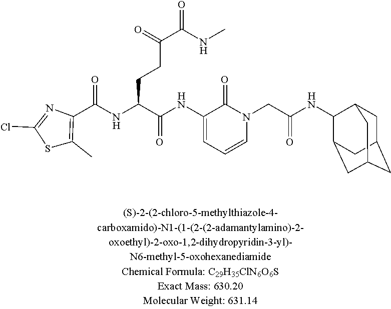 [SMILES: CNC(=O)C(=O)CC[C@H](NC(=O)c1nc(Cl)sc1C)C(=O)Nc1cccn(CC(=O)NC2C3CC4CC(C3)CC2C4)c1=O]

The synthesis of compound II-31 was performed according to compound II-3, using 2-chloro-5-methylthiazole-4-carboxylic acid instead of 3-methylbenzo[b]furan-2-carboxylic acid in step 6 (according to ZED3264).
Yield: 32 mg, 41% (last step)
ESI-MS: 631.3/633.3 [M+H]⁺

### 38 Preparation of Compound II-32

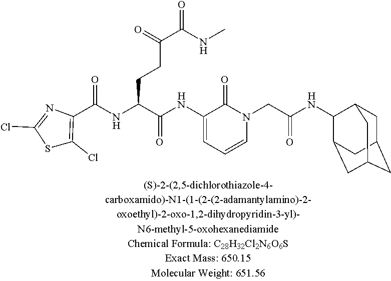 [SMILES: CNC(=O)C(=O)CC[C@H](NC(=O)c1nc(Cl)sc1Cl)C(=O)Nc1cccn(CC(=O)NC2C3CC4CC(C3)CC2C4)c1=O]

The synthesis of compound II-32 was performed according to compound II-3, using 2,5-dichlorothiazole-4-carboxylic acid instead of 3-methylbenzo[b]furan-2-carboxylic acid in step 6 (according to ZED3264).
Yield: 41 mg, 35% (last step)
ESI-MS: 651.2/653.2 [M+H]⁺

### 39 Preparation of Compound II-33

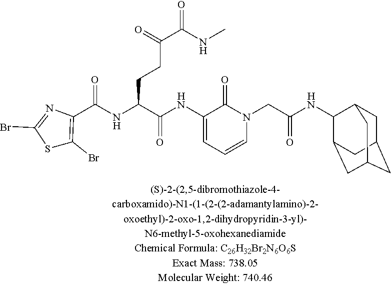 [SMILES: CNC(=O)C(=O)CC[C@H](NC(=O)c1nc(Br)sc1Br)C(=O)Nc1cccn(CC(=O)NC2C3CC4CC(C3)CC2C4)c1=O]

The synthesis of compound II-33 was performed according to compound II-3, using 2,5-dibromothiazole-4-carboxylic acid instead of 3-methylbenzo[b]furan-2-carboxylic acid in step 6 (according to ZED3264).
Yield: 21 mg, 32% (last step)
ESI-MS: 739.2/741.2/743.2 [M+H]⁺

### 40 Preparation of Compound II-34

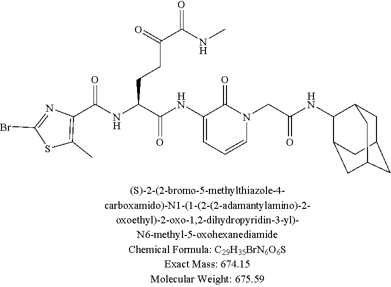 [SMILES: CNC(=O)C(=O)CC[C@H](NC(=O)c1nc(Br)sc1C)C(=O)Nc1cccn(CC(=O)NC2C3CC4CC(C3)CC2C4)c1=O]

The synthesis of compound II-34 was performed according to compound II-3, using 2-bromo-5-methylthiazole-4-carboxylic acid instead of 3-methylbenzo[b]furan-2-carboxylic acid in step 6 (according to ZED3264).
Yield: 42 mg, 57% (last step)
ESI-MS: 675.2/677.2 [M+H]⁺

### 41 Preparation of Compound II-35

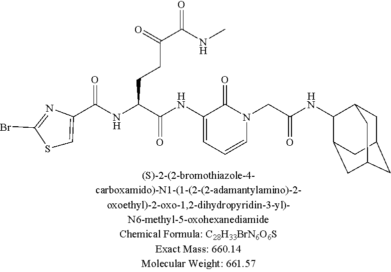 [SMILES: CNC(=O)C(=O)CC[C@H](NC(=O)c1csc(Br)n1)C(=O)Nc1cccn(CC(=O)NC2C3CC4CC(C3)CC2C4)c1=O]

The synthesis of compound II-35 was performed according to compound II-3, using 2-bromothiazole-4-carboxylic acid instead of 3-methylbenzo[b]furan-2-carboxylic acid in step 6 (according to ZED3264).
Yield: 66 mg, 46% (last step)
ESI-MS: 661.2/663.2 [M+H]⁺

### 42 Preparation of Compound II-36

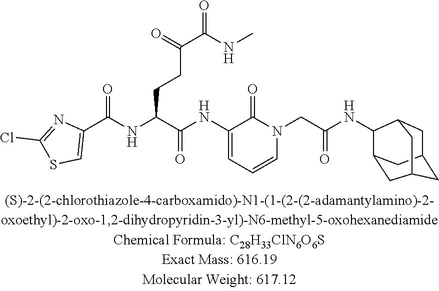 [SMILES: CNC(=O)C(=O)CC[C@H](NC(=O)c1csc(Cl)n1)C(=O)Nc1cccn(CC(=O)NC2C3CC4CC(C3)CC2C4)c1=O]

The synthesis of compound II-36 was performed according to compound II-3, using 2-chlorothiazole-4-carboxylic acid instead of 3-methylbenzo[b]furan-2-carboxylic acid in step 6 (according to ZED3264).
Yield: 74 mg, 58% (last step)
ESI-MS: 617.3/619.3 [M+H]⁺

### 43 Preparation of Compound II-37

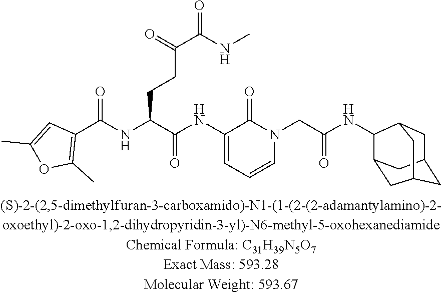 [SMILES: CNC(=O)C(=O)CC[C@H](NC(=O)c1cc(C)oc1C)C(=O)Nc1cccn(CC(=O)NC2C3CC4CC(C3)CC2C4)c1=O]

The synthesis of compound II-37 was performed according to compound II-3, using 2,5-dimethylfuran-3-carboxylic acid instead of 3-methylbenzo[b]furan-2-carboxylic acid in step 6 (according to ZED3264).
Yield: 152 mg, 73% (last step)
ESI-MS: 594.4 [M+H]⁺

### 44 Preparation of Compound II-38

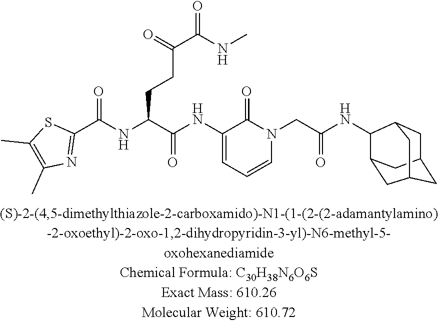 [SMILES: CNC(=O)C(=O)CC[C@H](NC(=O)c1nc(C)c(C)s1)C(=O)Nc1cccn(CC(=O)NC2C3CC4CC(C3)CC2C4)c1=O]

The synthesis of compound II-38 was performed according to compound II-3, using 4,5-dimethylthiazole-2-carboxylic acid instead of 3-methylbenzo[b]furan-2-carboxylic acid in step 6 (according to ZED3264).
Yield: 46 mg, 38% (last step)
ESI-MS: 611.4 [M+H]⁺

### 45 Preparation of Compound II-39

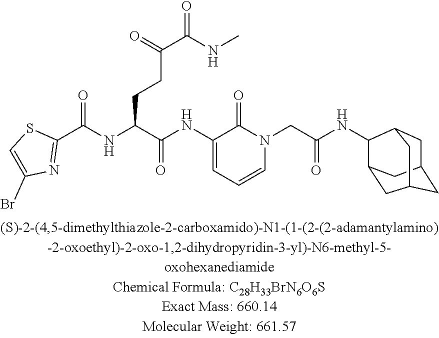 [SMILES: CNC(=O)C(=O)CC[C@H](NC(=O)c1nc(Br)cs1)C(=O)Nc1cccn(CC(=O)NC2C3CC4CC(C3)CC2C4)c1=O]

The synthesis of compound II-39 was performed according to compound II-3, using 4-bromothiazole-2-carboxylic acid instead of 3-methylbenzo[b]furan-2-carboxylic acid in step 6 (according to ZED3264).
Yield: 84 mg, 68% (last step)
ESI-MS: 661.2/663.2 [M+H]⁺

### 46 Preparation of Compound II-40

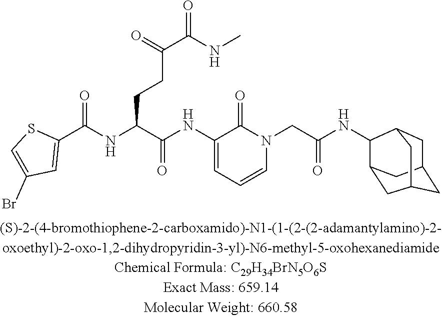 [SMILES: CNC(=O)C(=O)CC[C@H](NC(=O)c1cc(Br)cs1)C(=O)Nc1cccn(CC(=O)NC2C3CC4CC(C3)CC2C4)c1=O]

The synthesis of compound II-40 was performed according to compound II-3, using 4-bromothiophene-2-carboxylic acid instead of 3-methylbenzo[b]furan-2-carboxylic acid in step 6 (according to ZED3264).
Yield: 163 mg, 73% (last step)
ESI-MS: 660.2/662.2 [M+H]⁺

### 47 Preparation of Compound II-41

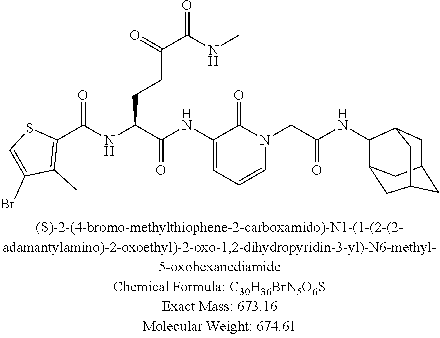 [SMILES: CNC(=O)C(=O)CC[C@H](NC(=O)c1scc(Br)c1C)C(=O)Nc1cccn(CC(=O)NC2C3CC4CC(C3)CC2C4)c1=O]

The synthesis of compound II-41 was performed according to compound II-3, using 4-bromo-3-methylthiophene-2-carboxylic acid instead of 3-methylbenzo[b]furan-2-carboxylic acid in step 6 (according to ZED3264).
Yield: 114 mg, 68% (last step)
ESI-MS: 674.2/676.2 [M+H]⁺

### 48 Preparation of Compound II-42

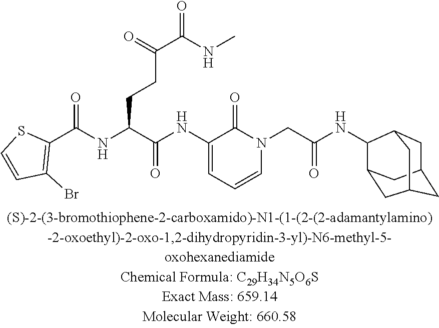 [SMILES: CNC(=O)C(=O)CC[C@H](NC(=O)c1sccc1Br)C(=O)Nc1cccn(CC(=O)NC2C3CC4CC(C3)CC2C4)c1=O]

The synthesis of compound II-42 was performed according to compound II-3, using 3-bromothiophene-2-carboxylic acid instead of 3-methylbenzo[b]furan-2-carboxylic acid in step 6 (according to ZED3264).
Yield: 149 mg, 76% (last step)
ESI-MS: 660.2/662.2 [M+H]⁺

### 49 Preparation of Compound II-43

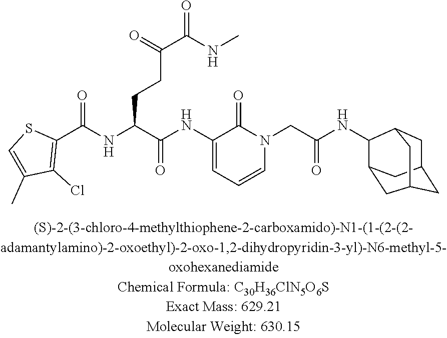 [SMILES: CNC(=O)C(=O)CC[C@H](NC(=O)c1scc(C)c1Cl)C(=O)Nc1cccn(CC(=O)NC2C3CC4CC(C3)CC2C4)c1=O]

The synthesis of compound II-43 was performed according to compound II-3, using 3-chloro-4-methylthiophene-2-carboxylic acid instead of 3-methylbenzo[b]furan-2-carboxylic acid in step 6 (according to ZED3264).
Yield: 110 mg, 64% (last step)
ESI-MS: 630.3/632.3 [M+H]⁺

### 50 Preparation of Compound II-44

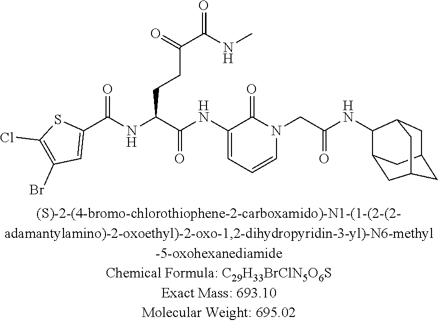 [SMILES: CNC(=O)C(=O)CC[C@H](NC(=O)c1cc(Br)c(Cl)s1)C(=O)Nc1cccn(CC(=O)NC2C3CC4CC(C3)CC2C4)c1=O]

The synthesis of compound II-44 was performed according to compound II-3, using 4-bromo-5-chlorothiophene-2-carboxylic acid instead of 3-methylbenzo[b]furan-2-carboxylic acid in step 6 (according to ZED3264).
Yield: 126 mg, 59% (last step)
ESI-MS: 694.2/696.2/698.2 [M+H]⁺

### 51 Preparation of Compound II-45

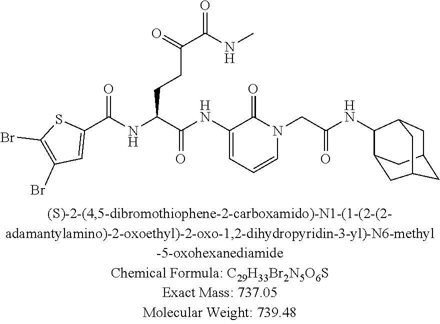 [SMILES: CNC(=O)C(=O)CC[C@H](NC(=O)c1cc(Br)c(Br)s1)C(=O)Nc1cccn(CC(=O)NC2C3CC4CC(C3)CC2C4)c1=O]

The synthesis of compound II-45 was performed according to compound II-3, using 4,5-dibromothiophene-2-carboxylic acid instead of 3-methylbenzo[b]furan-2-carboxylic acid in step 6 (according to ZED3264).
Yield: 89 mg, 51% (last step)
ESI-MS: 738.2/740.2/742.2 [M+H]⁺

### 52 Preparation of Compound II-46

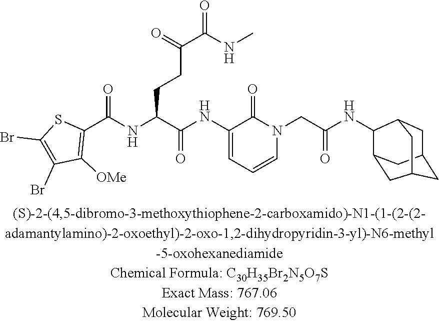 [SMILES: CNC(=O)C(=O)CC[C@H](NC(=O)c1sc(Br)c(Br)c1OC)C(=O)Nc1cccn(CC(=O)NC2C3CC4CC(C3)CC2C4)c1=O]

The synthesis of compound II-46 was performed according to compound II-3, using 4,5-dibromo-3-methoxythiophene-2-carboxylic acid instead of 3-methylbenzo[b]furan-2-carboxylic acid in step 6 (according to ZED3264).
Yield: 59 mg, 40% (last step)
ESI-MS: 768.2/770.2/772.2 [M+H]⁺

### 53 Preparation of Compound II-47

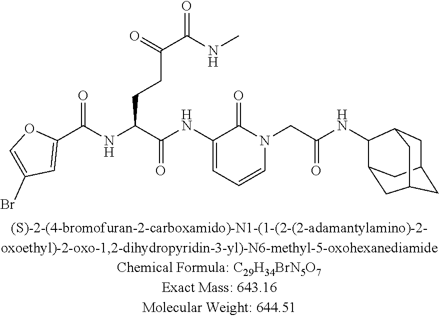 [SMILES: CNC(=O)C(=O)CC[C@H](NC(=O)c1cc(Br)co1)C(=O)Nc1cccn(CC(=O)NC2C3CC4CC(C3)CC2C4)c1=O]

The synthesis of compound II-47 was performed according to compound II-3, using 4-bromofuran-2-carboxylic acid instead of 3-methylbenzo[b]furan-2-carboxylic acid in step 6 (according to ZED3264).
Yield: 90 mg, 69% (last step)
ESI-MS: 644.3/646.3 [M+H]⁺

### 54 Preparation of Compound II-48

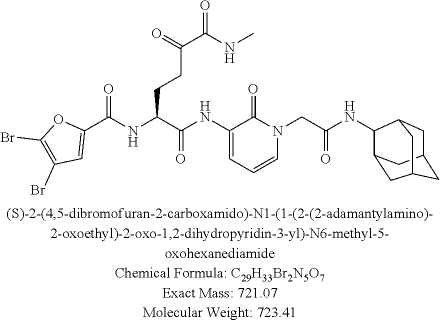 [SMILES: CNC(=O)C(=O)CC[C@H](NC(=O)c1cc(Br)c(Br)o1)C(=O)Nc1cccn(CC(=O)NC2C3CC4CC(C3)CC2C4)c1=O]

The synthesis of compound II-48 was performed according to compound II-3, using 4,5-dibromofuran-2-carboxylic acid instead of 3-methylbenzo[b]furan-2-carboxylic acid in step 6 (according to ZED3264).
Yield: 72 mg, 56% (last step)
ESI-MS: 722.2/724.2/726.2 [M+H]⁺

### 55 Preparation of Compound II-49

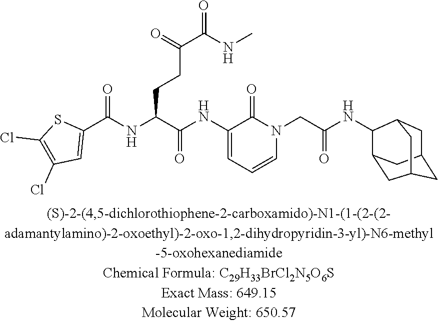 [SMILES: CNC(=O)C(=O)CC[C@H](NC(=O)c1cc(Cl)c(Cl)s1)C(=O)Nc1cccn(CC(=O)NC2C3CC4CC(C3)CC2C4)c1=O]

The synthesis of compound II-49 was performed according to compound II-3, using 4,5-dichlorothiophene-2-carboxylic acid instead of 3-methylbenzo[b]furan-2-carboxylic acid in step 6 (according to ZED3264).
Yield: 55 mg, 48% (last step)
ESI-MS: 650.3/652.3 [M+H]⁺

### 56 Preparation of Compound II-50

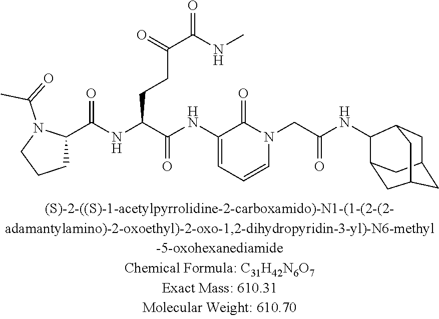 [SMILES: CNC(=O)C(=O)CC[C@H](NC(=O)[C@@H]1CCCN1C(C)=O)C(=O)Nc1cccn(CC(=O)NC2C3CC4CC(C3)CC2C4)c1=O]

The synthesis of compound II-50 was performed according to compound II-3, using(S) -1-acetylpyrrolidine-2-carboxylic acid instead of 3-methylbenzo[b]furan-2-carboxylic acid in step 6 (according to ZED3264).
Yield: 190 mg, 78% (last step)
ESI-MS: 611.4 [M+H]⁺

### 57 Preparation of Compound II-51

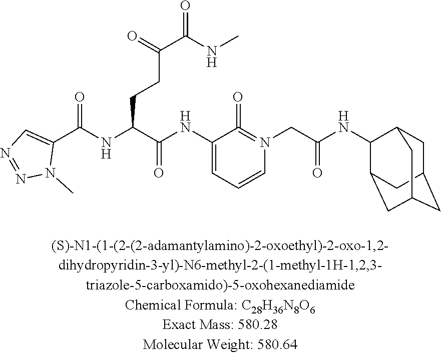

The synthesis of compound II-51 was performed according to compound II-3, using 1-methyl-1H-1,2,3-triazole-5-carboxylic acid instead of 3-methylbenzo[b]furan-2-carboxylic acid in step 6 (according to ZED3264).
Yield: 28 mg, 35% (last step)
ESI-MS: 581.4 [M+H]⁺

### 58 Preparation of Compound II-52

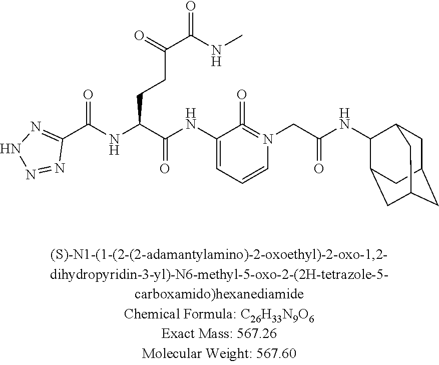

The synthesis of compound II-52 was performed according to compound II-3, using 2H-tetrazole-5-carboxylic acid instead of 3-methylbenzo[b]furan-2-carboxylic acid in step 6 (according to ZED3264).
Yield: 23 mg, 31% (last step)
ESI-MS: 568.4 [M+H]⁺

### 59 Preparation of Compound II-53

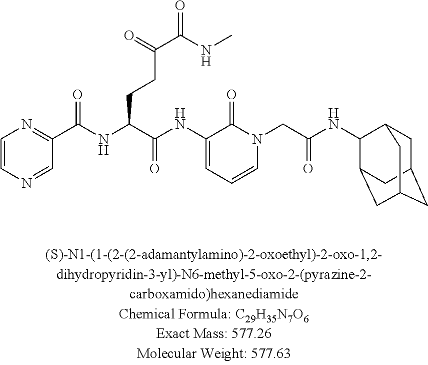 [SMILES: CNC(=O)C(=O)CC[C@H](NC(=O)c1cnccn1)C(=O)Nc1cccn(CC(=O)NC2C3CC4CC(C3)CC2C4)c1=O]

The synthesis of compound II-53 was performed according to compound II-3, using pyrazine-2-carboxylic acid instead of 3-methylbenzo[b]furan-2-carboxylic acid in step 6 (according to ZED3264).
Yield: 79 mg, 74% (last step)
ESI-MS: 578.3 [M+H]⁺

### 60 Preparation of Compound II-54

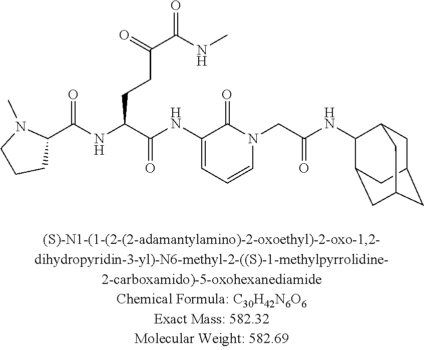 [SMILES: CNC(=O)C(=O)CC[C@H](NC(=O)[C@@H]1CCCN1C)C(=O)Nc1cccn(CC(=O)NC2C3CC4CC(C3)CC2C4)c1=O]

The synthesis of compound II-54 was performed according to compound II-3, using(S) -1-methylpyrrolidine-2-carboxylic acid instead of 3-methylbenzo[b]furan-2-carboxylic acid in step 6 (according to ZED3264).
Yield: 68 mg, 82% (last step)
ESI-MS: 583.4 [M+H]⁺

### 61 Preparation of Compound II-55

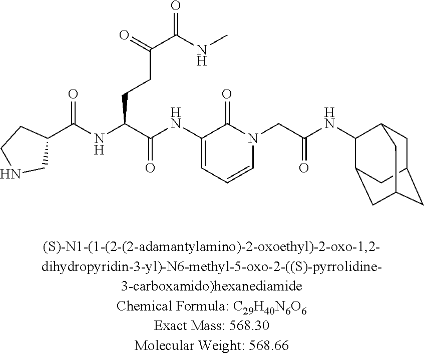 [SMILES: CNC(=O)C(=O)CC[C@H](NC(=O)[C@H]1CCNC1)C(=O)Nc1cccn(CC(=O)NC2C3CC4CC(C3)CC2C4)c1=O]

The synthesis of compound II-55 was performed according to compound II-3, using(S) -1-Boc-pyrrolidine-3-carboxylic acid instead of 3-methylbenzo[b]furan-2-carboxylic acid in step 6 (according to ZED3264). The final product was obtained by deprotection (DCM/TFA) as described above and purified by HPLC.
Yield: 43 mg, 79% (last step)
ESI-MS: 569.4 [M+H]⁺

### 62 Preparation of Compound II-56

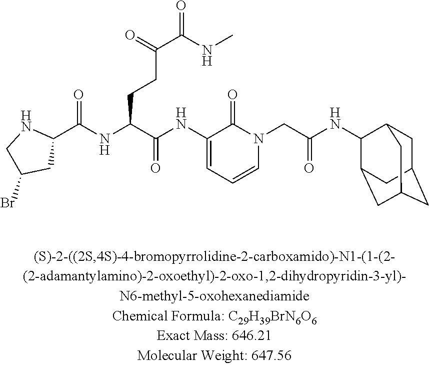 [SMILES: CNC(=O)C(=O)CC[C@H](NC(=O)[C@@H]1C[C@H](Br)CN1)C(=O)Nc1cccn(CC(=O)NC2C3CC4CC(C3)CC2C4)c1=O]

The synthesis of compound II-56 was performed according to compound II-3, using (2S,4S)-1-Boc-4-bromopyrrolidine-2-carboxylic acid instead of 3-methylbenzo[b]furan-2-carboxylic acid in step 6 (according to ZED3264). The final product was obtained by deprotection (DCM/TFA) as described above and purified by HPLC.
Yield: 45 mg, 73% (last step)
ESI-MS: 647.3/649.3 [M+H]⁺

### 63 Preparation of Compound II-58

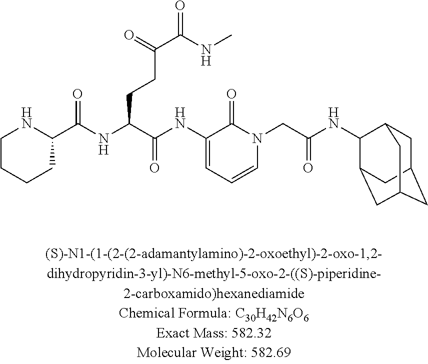 [SMILES: CNC(=O)C(=O)CC[C@H](NC(=O)[C@@H]1CCCCN1)C(=O)Nc1cccn(CC(=O)NC2C3CC4CC(C3)CC2C4)c1=O]

The synthesis of compound II-58 was performed according to compound II-3, using(S)-1-Boc-piperidine-2-carboxylic acid instead of 3-methylbenzo[b]furan-2-carboxylic acid in step 6 (according to ZED3264). The final product was obtained by deprotection (DCM/TFA) as described above and purified by HPLC.
Yield: 53 mg, 86% (last step)
ESI-MS: 583.4 [M+H]⁺

### 64 Preparation of Compound II-59

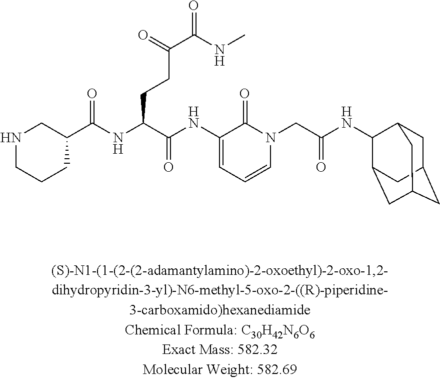 [SMILES: CNC(=O)C(=O)CC[C@H](NC(=O)[C@@H]1CCCNC1)C(=O)Nc1cccn(CC(=O)NC2C3CC4CC(C3)CC2C4)c1=O]

The synthesis of compound II-59 was performed according to compound II-3, using (R)-1-Boc-piperidine-3-carboxylic acid instead of 3-methylbenzo[b]furan-2-carboxylic acid in step 6 (according to ZED3264). The final product was obtained by deprotection (DCM/TFA) as described above and purified by HPLC.
Yield: 43 mg, 77% (last step)
ESI-MS: 583.4 [M+H]⁺

### 65 Preparation of Compound II-60

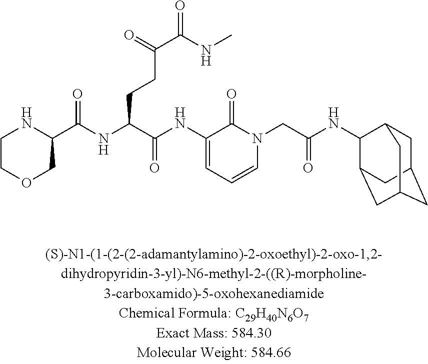 [SMILES: CNC(=O)C(=O)CC[C@H](NC(=O)[C@H]1COCCN1)C(=O)Nc1cccn(CC(=O)NC2C3CC4CC(C3)CC2C4)c1=O]

The synthesis of compound II-60 was performed according to compound II-3, using (R)-4-Boc-morpholine-3-carboxylic acid instead of 3-methylbenzo[b]furan-2-carboxylic acid in step 6 (according to ZED3264). The final product was obtained by deprotection (DCM/TFA) as described above and purified by HPLC.
Yield: 67 mg, 85% (last step)
ESI-MS: 585.4 [M+H]⁺

### 66 Preparation of Compound II-61

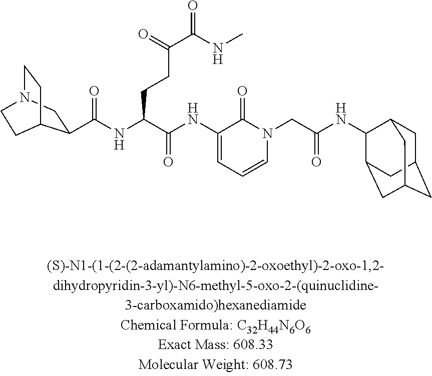 [SMILES: CNC(=O)C(=O)CC[C@H](NC(=O)C1CN2CCC1CC2)C(=O)Nc1cccn(CC(=O)NC2C3CC4CC(C3)CC2C4)c1=O]

The synthesis of compound II-61 was performed according to compound II-3, using quinuclidine-3-carboxylic acid instead of 3-methylbenzo[b]furan-2-carboxylic acid in step 6 (according to ZED3264).
Yield: 24 mg, 54% (last step)
ESI-MS: 609.4 [M+H]⁺

### 67 Preparation of Compound II-62

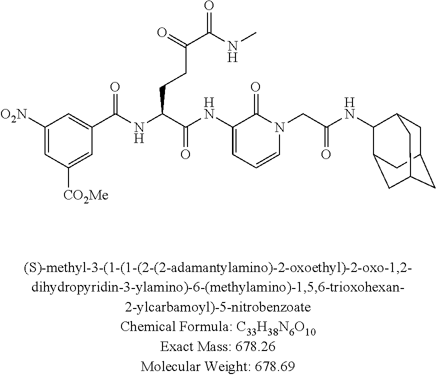 [SMILES: CNC(=O)C(=O)CC[C@H](NC(=O)c1cc(C(=O)OC)cc([N+](=O)[O-])c1)C(=O)Nc1cccn(CC(=O)NC2C3CC4CC(C3)CC2C4)c1=O]

The synthesis of compound II-62 was performed according to compound II-3, using mono-methyl 5-nitroisophthalate instead of 3-methylbenzo[b]furan-2-carboxylic acid in step 6 (according to ZED3264).
Yield: 57 mg, 66% (last step)
ESI-MS: 679.3 [M+H]⁺

### 68 Preparation of Compound II-63

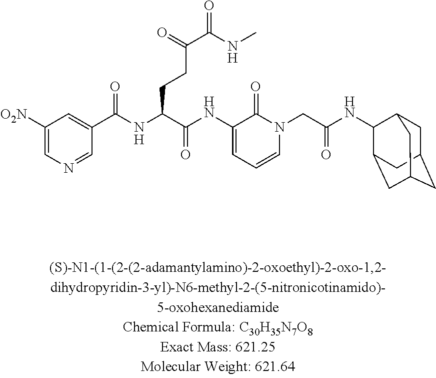 [SMILES: CNC(=O)C(=O)CC[C@H](NC(=O)c1cncc([N+](=O)[O-])c1)C(=O)Nc1cccn(CC(=O)NC2C3CC4CC(C3)CC2C4)c1=O]

The synthesis of compound II-63 was performed according to compound II-3, using 5-nitronicotinic acid instead of 3-methylbenzo[b]furan-2-carboxylic acid in step 6 (according to ZED3264).
Yield: 76 mg, 65% (last step)
ESI-MS: 622.3 [M+H]⁺

### 69 Preparation of Compound II-64

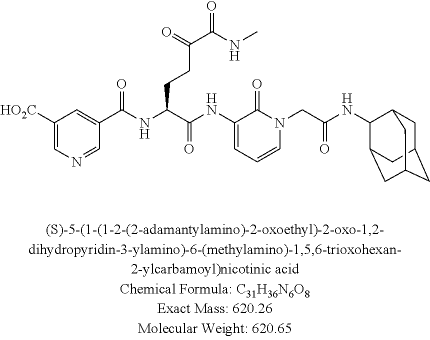 [SMILES: CNC(=O)C(=O)CC[C@H](NC(=O)c1cncc(C(=O)O)c1)C(=O)Nc1cccn(CC(=O)NC2C3CC4CC(C3)CC2C4)c1=O]

The synthesis of compound II-64 was performed according to compound II-3, using 3,5-pyridinedicarboxylic acid instead of 3-methylbenzo[b]furan-2-carboxylic acid in step 6 (according to ZED3264).
Yield: 16 mg, 52% (last step)
ESI-MS: 621.3 [M+H]⁺

### 70 Preparation of Compound II-65

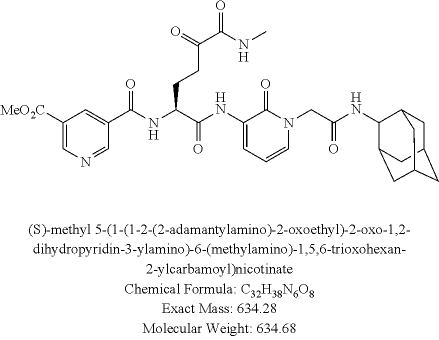 [SMILES: CNC(=O)C(=O)CC[C@H](NC(=O)c1cncc(C(=O)OC)c1)C(=O)Nc1cccn(CC(=O)NC2C3CC4CC(C3)CC2C4)c1=O]

The synthesis of compound II-65 was performed according to compound II-3, using 5-(methoxycarbonyl) nicotinic acid instead of 3-methylbenzo[b]furan-2-carboxylic acid in step 6 (according to ZED3264).
Yield: 34 mg, 62% (last step)
ESI-MS: 635.3 [M+H]⁺

### 71 Preparation of Compound II-66

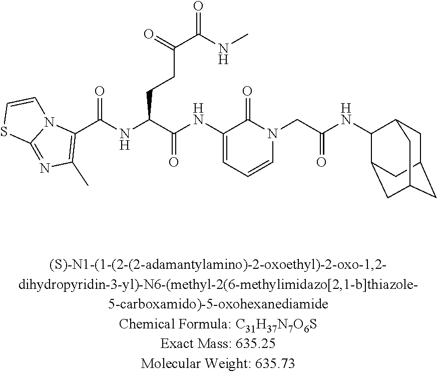 [SMILES: CNC(=O)C(=O)CC[C@H](NC(=O)c1c(C)nc2sccn12)C(=O)Nc1cccn(CC(=O)NC2C3CC4CC(C3)CC2C4)c1=O]

The synthesis of compound II-66 was performed according to compound II-3, using 6-methylimidazo [2,1-b]thiazole-5-carboxylic acid instead of 3-methylbenzo[b]furan-2-carboxylic acid in step 6 (according to ZED3264).
Yield: 45 mg, 37% (last step)
ESI-MS: 636.4 [M+H]⁺

### 72 Preparation of Compound II-67

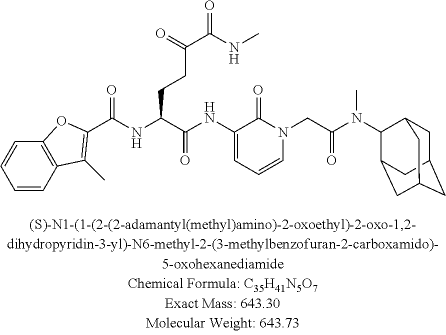 [SMILES: CNC(=O)C(=O)CC[C@H](NC(=O)c1oc2ccccc2c1C)C(=O)Nc1cccn(CC(=O)N(C)C2C3CC4CC(C3)CC2C4)c1=O]

The synthesis of compound II-67 was performed according to compound II-3, using N-methyl-2-adamantanamine instead of 2-adamantanamine in step 2 (according to ZED3905).
Yield: 66 mg, 45% (last step)
ESI-MS: 644.4 [M+H]⁺

### 73 Preparation of Compound II-68

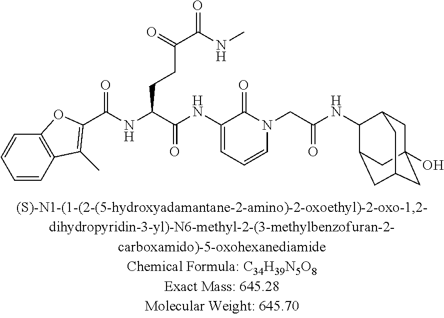 [SMILES: CNC(=O)C(=O)CC[C@H](NC(=O)c1oc2ccccc2c1C)C(=O)Nc1cccn(CC(=O)NC2C3CC4CC2CC(O)(C4)C3)c1=O]

The synthesis of compound II-68 was performed according to compound II-3, using 5-hydroxy-2-adamantanamine instead of 2-adamantanamine in step 2 (according to ZED3905).
Yield: 21 mg, 34% (last step)
ESI-MS: 646.4 [M+H]⁺

### 74 Preparation of Compound II-69

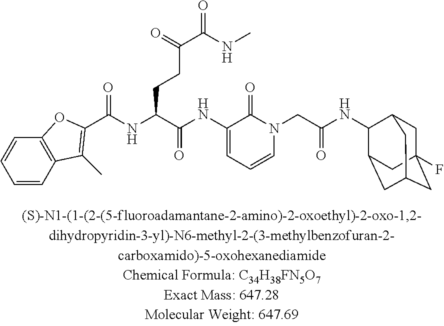 [SMILES: CNC(=O)C(=O)CC[C@H](NC(=O)c1oc2ccccc2c1C)C(=O)Nc1cccn(CC(=O)NC2C3CC4CC2CC(F)(C4)C3)c1=O]

The synthesis of compound II-69 was performed according to compound II-3, using 5-fluoro-2-adamantanamine instead of 2-adamantanamine in step 2 (according to ZED3905).
Yield: 48 mg, 57% (last step)
ESI-MS: 648.4 [M+H]⁺

### 75 Preparation of Compound II-70

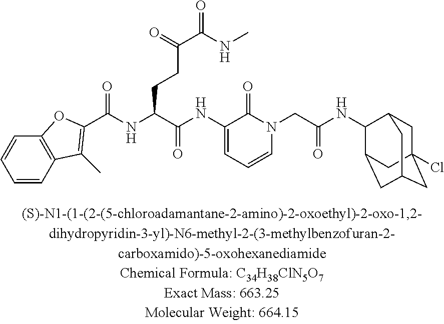 [SMILES: CNC(=O)C(=O)CC[C@H](NC(=O)c1oc2ccccc2c1C)C(=O)Nc1cccn(CC(=O)NC2C3CC4CC2CC(Cl)(C4)C3)c1=O]

The synthesis of compound II-70 was performed according to compound II-3, using 5-chloro-2-adamantanamine instead of 2-adamantanamine in step 2 (according to ZED3905).
Yield: 45 mg, 35% (last step)
ESI-MS: 664.3/666.3 [M+H]⁺

### 76 Preparation of Compound II-71

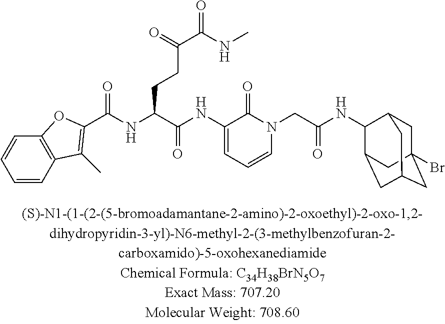 [SMILES: CNC(=O)C(=O)CC[C@H](NC(=O)c1oc2ccccc2c1C)C(=O)Nc1cccn(CC(=O)NC2C3CC4CC2CC(Br)(C4)C3)c1=O]

The synthesis of compound II-71 was performed according to compound II-3, using 5-bromo-2-adamantanamine instead of 2-adamantanamine in step 2 (according to ZED3905).
Yield: 31 mg, 34% (last step)
ESI-MS: 708.3/710.3 [M+H]⁺

### 77 Preparation of Compound II-72

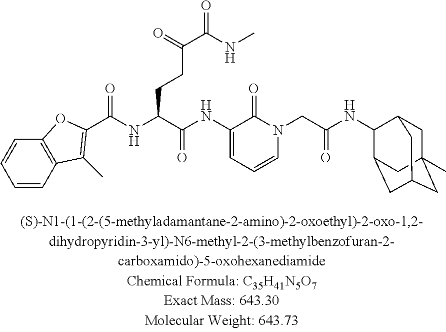 [SMILES: CNC(=O)C(=O)CC[C@H](NC(=O)c1oc2ccccc2c1C)C(=O)Nc1cccn(CC(=O)NC2C3CC4CC2CC(C)(C4)C3)c1=O]

The synthesis of compound II-72 was performed according to compound II-3, using 5-methyl-2-adamantanamine instead of 2-adamantanamine in step 2 (according to ZED3905).
Yield: 68 mg, 54% (last step)
ESI-MS: 644.4 [M+H]⁺

### 78 Preparation of Compound II-73

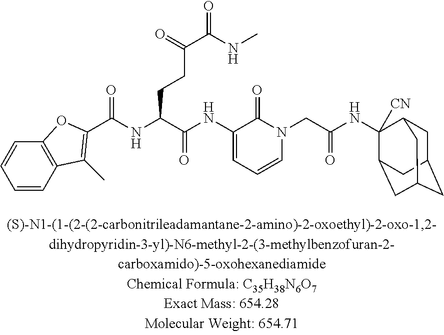 [SMILES: CNC(=O)C(=O)CC[C@H](NC(=O)c1oc2ccccc2c1C)C(=O)Nc1cccn(CC(=O)NC2(C#N)C3CC4CC(C3)CC2C4)c1=O]

The synthesis of compound II-73 was performed according to compound II-3, using 2-aminoadamantane-2-carbonitrile instead of 2-adamantanamine in step 2 (according to ZED3905).
Yield: 26 mg, 46% (last step)
ESI-MS: 655.4 [M+H]⁺

### 79 Preparation of Compound II-74

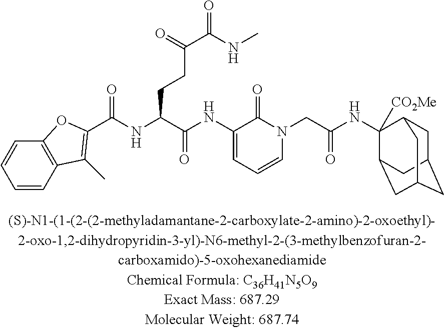 [SMILES: CNC(=O)C(=O)CC[C@H](NC(=O)c1oc2ccccc2c1C)C(=O)Nc1cccn(CC(=O)NC2(C(=O)OC)C3CC4CC(C3)CC2C4)c1=O]

The synthesis of compound II-74 was performed according to compound II-3, using 2-methyl 2-aminoadamantane-2-carboxylate instead of 2-adamantanamine in step 2 (according to ZED3905).
Yield: 38 mg, 61% (last step)
ESI-MS: 688.4 [M+H]⁺

### 80 Preparation of Compound II-87

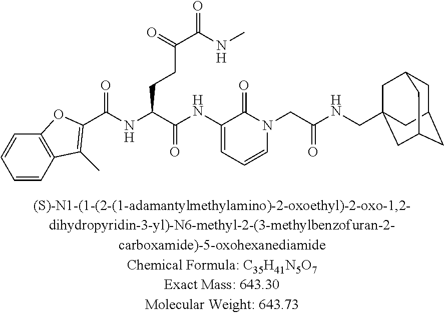 [SMILES: CNC(=O)C(=O)CC[C@H](NC(=O)c1oc2ccccc2c1C)C(=O)Nc1cccn(CC(=O)NCC23CC4CC(CC(C4)C2)C3)c1=O]

The synthesis of compound II-87 was performed according to compound II-3, using 1-adamantanemethylamine instead of 2-adamantanamine in step 2 (according to ZED3905).
Yield: 43 mg, 53% (last step)
ESI-MS: 644.4 [M+H]⁺

### 81 Preparation of Compound II-88

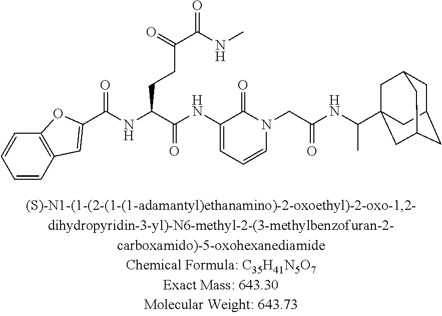 [SMILES: CNC(=O)C(=O)CC[C@H](NC(=O)c1cc2ccccc2o1)C(=O)Nc1cccn(CC(=O)NC(C)C23CC4CC(CC(C4)C2)C3)c1=O]

The synthesis of compound II-88 was performed according to compound II-2, using 1-rimantadine instead of 2-adamantanamine in step 2 (according to ZED3905).
Yield: 31 mg, 41% (last step)
ESI-MS: 644.4 [M+H]⁺

### 82 Preparation of Compound II-90

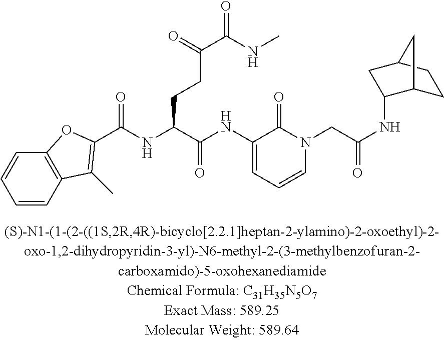 [SMILES: CNC(=O)C(=O)CC[C@H](NC(=O)c1oc2ccccc2c1C)C(=O)Nc1cccn(CC(=O)NC2CC3CCC2C3)c1=O]

The synthesis of compound II-90 was performed according to compound II-3, using (+)-endo-2-norbornylamine instead of 2-adamantanamine in step 2 (according to ZED3905).
Yield: 67 mg, 65% (last step)
ESI-MS: 590.4 [M+H]⁺

### 83 Preparation of Compound II-92

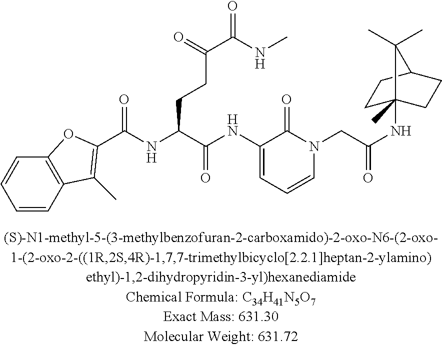

The synthesis of compound II-92 was performed according to compound II-3, using (R)-(+)-bornylamine instead of 2-adamantanamine in step 2 (according to ZED3905).
Yield: 52 mg, 66% (last step) 5
ESI-MS: 632.5 [M+H]⁺

### 84 Preparation of Compound II-94

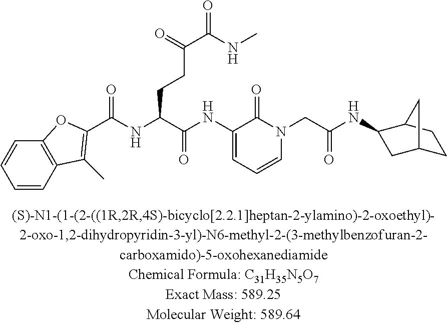 [SMILES: CNC(=O)C(=O)CC[C@H](NC(=O)c1oc2ccccc2c1C)C(=O)Nc1cccn(CC(=O)N[C@@H]2CC3CCC2C3)c1=O]

10 The synthesis of compound II-94 was performed according to compound II-3, using exo-2-aminonorbornane instead of 2-adamantanamine in step 2 (according to ZED3905).
Yield: 62 mg, 68% (last step)
ESI-MS: 590.4 [M+H]⁺

### 85 Preparation of Compound II-95

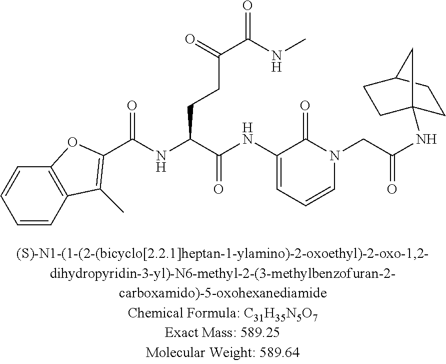 [SMILES: CNC(=O)C(=O)CC[C@H](NC(=O)c1oc2ccccc2c1C)C(=O)Nc1cccn(CC(=O)NC23CCC(CC2)C3)c1=O]

The synthesis of compound II-95 was performed according to compound II-3, using bicyclo[2.2.1]heptan-1-ylamine instead of 2-adamantanamine in step 2 (according to ZED3905).
Yield: 14 mg, 32% (last step)
ESI-MS: 590.4 [M+H]⁺

### 86 Preparation of Compound II-96

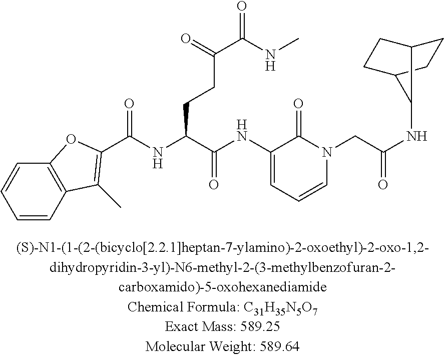 [SMILES: CNC(=O)C(=O)CC[C@H](NC(=O)c1oc2ccccc2c1C)C(=O)Nc1cccn(CC(=O)NC2C3CCC2CC3)c1=O]

The synthesis of compound II-96 was performed according to compound II-3, using bicyclo[2.2.1]heptan-7-ylamine instead of 2-adamantanamine in step 2 (according to ZED3905).
Yield: 36 mg, 53% (last step)
ESI-MS: 590.4 [M+H]⁺

### 87 Preparation of Compound II-97

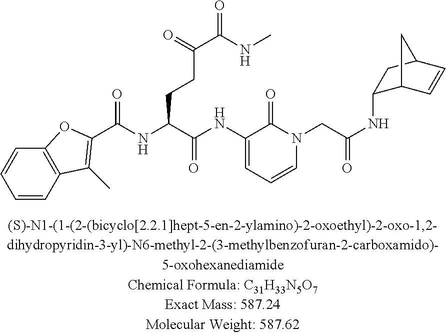 [SMILES: CNC(=O)C(=O)CC[C@H](NC(=O)c1oc2ccccc2c1C)C(=O)Nc1cccn(CC(=O)NC2CC3C=CC2C3)c1=O]

The synthesis of compound II-97 was performed according to compound II-3, using bicyclo[2.2.1]hept-5-en-2-amine instead of 2-adamantanamine in step 2 (according to ZED3905).
Yield: 21 mg, 44% (last step)
ESI-MS: 588.4 [M+H]⁺

### 88 Preparation of Compound II-98

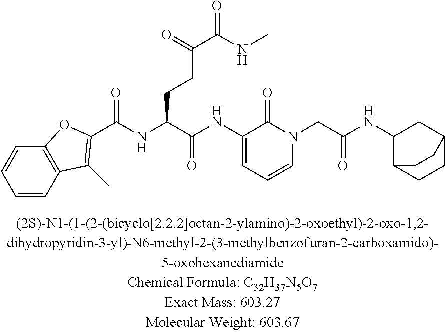 [SMILES: CNC(=O)C(=O)CC[C@H](NC(=O)c1oc2ccccc2c1C)C(=O)Nc1cccn(CC(=O)NC2CC3CCC2CC3)c1=O]

The synthesis of compound II-98 was performed according to compound II-3, using bicyclo[2.2.2]oct-2-ylamine instead of 2-adamantanamine in step 2 (according to ZED3905).
Yield: 25 mg, 41% (last step)
ESI-MS: 604.4 [M+H]⁺

### 89 Preparation of Compound II-99

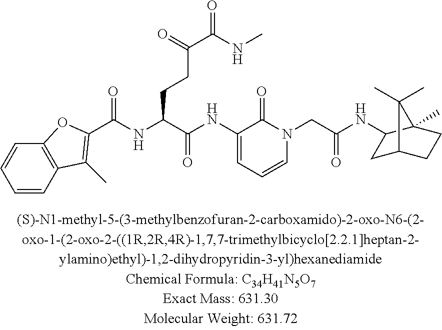 [SMILES: CNC(=O)C(=O)CC[C@H](NC(=O)c1oc2ccccc2c1C)C(=O)Nc1cccn(CC(=O)NC2CC3CC[C@]2(C)C3(C)C)c1=O]

The synthesis of compound II-99 was performed according to compound II-3, using (R)-(−)-isobornylamine instead of 2-adamantanamine in step 2 (according to ZED3905).
Yield: 27 mg, 48% (last step)
ESI-MS: 632.5 [M+H]⁺

### 90 Preparation of Compound II-100

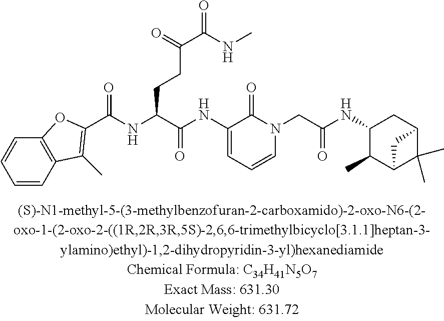 [SMILES: CNC(=O)C(=O)CC[C@H](NC(=O)c1oc2ccccc2c1C)C(=O)Nc1cccn(CC(=O)N[C@@H]2C[C@@H]3C[C@H]([C@H]2C)C3(C)C)c1=O]

The synthesis of compound II-100 was performed according to compound II-3, using (1R,2R,3R,5S)-(−)-isopinocampheylamine instead of 2-adamantanamine in step 2 (according to ZED3905).
Yield: 17 mg, 39% (last step)
ESI-MS: 632.5 [M+H]⁺

### 91 Preparation of Compound II-101

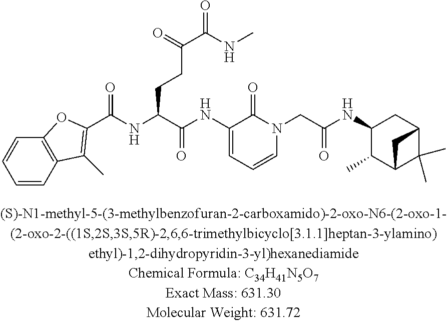 [SMILES: CNC(=O)C(=O)CC[C@H](NC(=O)c1oc2ccccc2c1C)C(=O)Nc1cccn(CC(=O)N[C@H]2C[C@H]3C[C@@H]([C@@H]2C)C3(C)C)c1=O]

The synthesis of compound II-101 was performed according to compound II-3, using (1S,2S,3S,5R)-(+)-isopinocampheylamine instead of 2-adamantanamine in step 2 (according to ZED3905).
Yield: 25 mg, 41% (last step)
ESI-MS: 632.5 [M+H]⁺

### 92 Preparation of Compound II-103

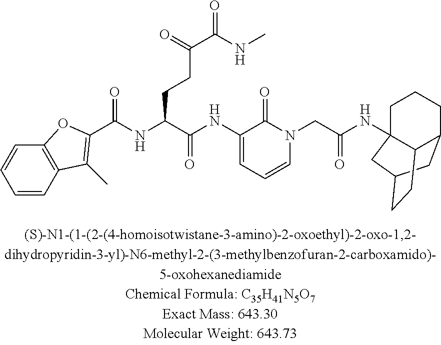 [SMILES: CNC(=O)C(=O)CC[C@H](NC(=O)c1oc2ccccc2c1C)C(=O)Nc1cccn(CC(=O)NC23CCCC4CC(CCC42)C3)c1=O]

The synthesis of compound II-103 was performed according to compound II-3, using 3-amino-4-homoisotwistane instead of 2-adamantanamine in step 2 (according to ZED3905).
Yield: 12 mg, 28% (last step)
ESI-MS: 644.5 [M+H]⁺

### 93 Preparation of Compound II-104

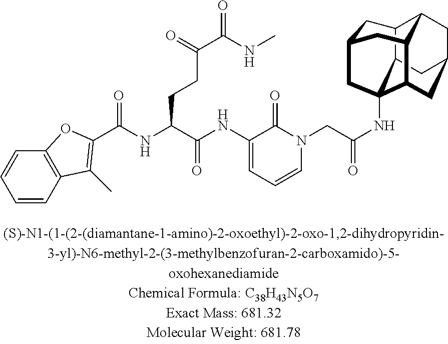

The synthesis of compound II-104 was performed according to compound II-3, using 1-aminodiamantane instead of 2-adamantanamine in step 2 (according to ZED3905).
Yield: 17 mg, 35% (last step)
ESI-MS: 682.5 [M+H]⁺

### 94 Preparation of Compound II-105

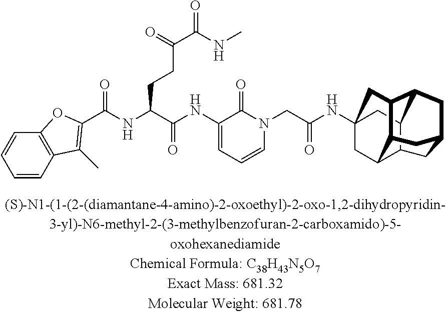

The synthesis of compound II-105 was performed according to compound II-3, using 4-aminodiamantane instead of 2-adamantanamine in step 2 (according to ZED3905).
Yield: 8 mg, 26% (last step)
ESI-MS: 682.5 [M+H]⁺

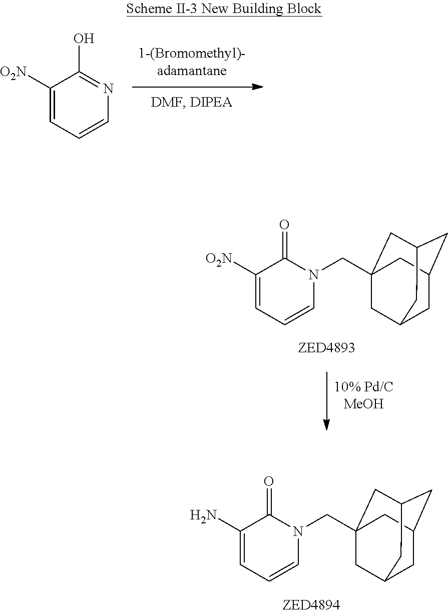 [SMILES: O=[N+]([O-])c1cccnc1O ; O=c1c([N+](=O)[O-])cccn1CC12CC3CC(CC(C3)C1)C2 ; Nc1cccn(CC23CC4CC(CC(C4)C2)C3)c1=O]

95 Preparation of compound ZED4893

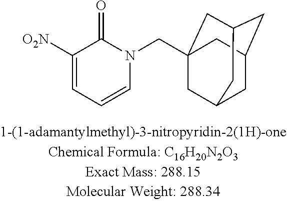 [SMILES: O=c1c([N+](=O)[O-])cccn1CC12CC3CC(CC(C3)C1)C2]

500 mg (3.57 mmol) of 2-hydroxy-3-nitropyridine and 818 mg (1 eq) of 1-(bromomethyl) adamantane were dissolved in 10 mL DMF and 1.24 mL DIPEA (2 eq) and stirred at room temperature overnight. The solvent was evaporated; the residue was dissolved in 30 mL EtOAc and washed twice with each 10 mL citric acid solution (10%), NaHCO₃ solution (10%) and brine. The organic phase was dried over Na₂SO₄, filtered and the solvent was evaporated. The residue was purified by HPLC.
Yield: 484 mg, 47% ESI-MS: 289.3 [M+H]⁺
   96 Preparation of compound ZED4894

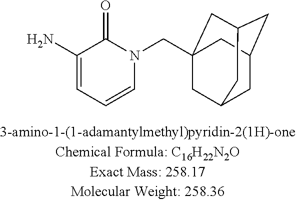 [SMILES: Nc1cccn(CC23CC4CC(CC(C4)C2)C3)c1=O]

484 mg (1.68 mmol) of ZED4893 were suspended in 30 mL MeOH before 50 mg of palladium (10%) on activated carbon (unreduced) were added. The suspension was stirred for 3 h at room temperature under an atmosphere of hydrogen. The catalyst was filtered, and the solvent was evaporated.
Yield: 339 mg, 78%
ESI-MS: 259.4 [M+H]⁺

### 97 Preparation of Compound II-107

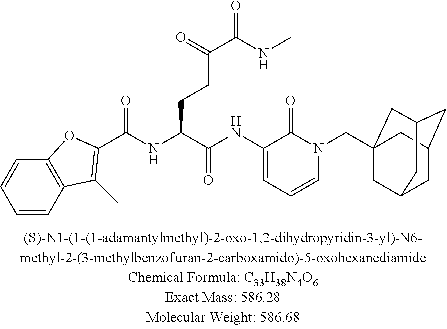 [SMILES: CNC(=O)C(=O)CC[C@H](NC(=O)c1oc2ccccc2c1C)C(=O)Nc1cccn(CC23CC4CC(CC(C4)C2)C3)c1=O]

The synthesis of compound II-107 was performed according to compound II-3, using ZED4894 instead of ZED3906 in step 5 (according to ZED3907).
Yield: 41 mg, 49% (last step)
ESI-MS: 587.4 [M+H]⁺

### 98 Preparation of Compound II-108

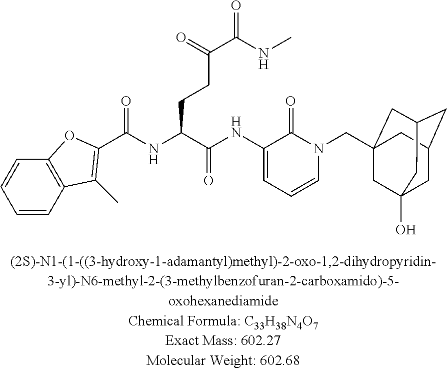 [SMILES: CNC(=O)C(=O)CC[C@H](NC(=O)c1oc2ccccc2c1C)C(=O)Nc1cccn(CC23CC4CC(CC(O)(C4)C2)C3)c1=O]

The synthesis of compound II-108 was performed according to compound II-107, using 3-(bromomethyl)-1-adamantanol instead of 1-(bromomethyl) adamantane (according to ZED4893).
Yield: 16 mg, 36% (last step)
ESI-MS: 603.4 [M+H]⁺

### 99 Preparation of Compound II-109

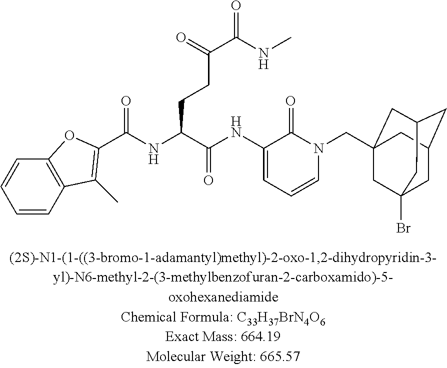 [SMILES: CNC(=O)C(=O)CC[C@H](NC(=O)c1oc2ccccc2c1C)C(=O)Nc1cccn(CC23CC4CC(CC(Br)(C4)C2)C3)c1=O]

The synthesis of compound II-109 was performed according to compound II-107, using 1-bromo-3-(bromomethyl) adamantane instead of 1-(bromomethyl) adamantane (according to ZED4893).
Yield: 24 mg, 41% (last step)
ESI-MS: 665.3/667.3 [M+H]⁺

### 100 Preparation of Compound II-110

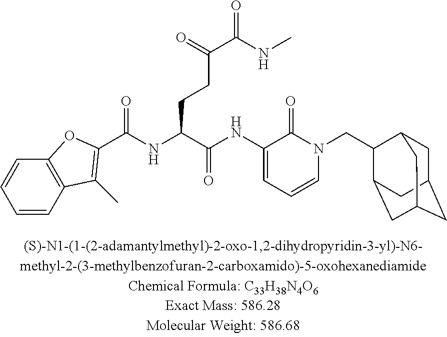 [SMILES: CNC(=O)C(=O)CC[C@H](NC(=O)c1oc2ccccc2c1C)C(=O)Nc1cccn(CC2C3CC4CC(C3)CC2C4)c1=O]

The synthesis of compound II-110 was performed according to compound II-107, using 2-(bromomethyl) adamantane instead of 1-(bromomethyl) adamantane (according to ZED4893).
Yield: 46 mg, 62% (last step)
ESI-MS: 587.4 [M+H]⁺

### 101 Preparation of Compound II-111

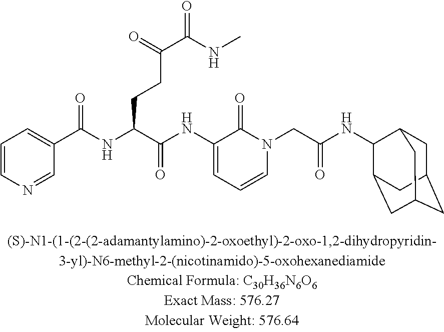 [SMILES: CNC(=O)C(=O)CC[C@H](NC(=O)c1cccnc1)C(=O)Nc1cccn(CC(=O)NC2C3CC4CC(C3)CC2C4)c1=O]

The synthesis of compound II-111 was performed according to compound II-3, using nicotinic acid instead of 3-methylbenzo[b]furan-2-carboxylic acid in step 6 (according to ZED3264).
Yield: 65 mg, 46% (last step)
ESI-MS: 577.4 [M+H]⁺

### 102 Preparation of Compound II-112

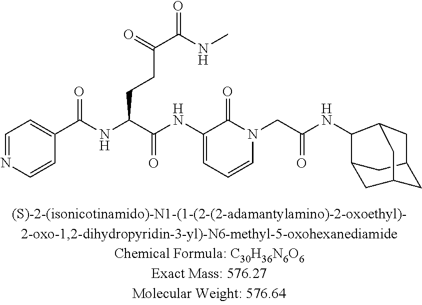 [SMILES: CNC(=O)C(=O)CC[C@H](NC(=O)c1ccncc1)C(=O)Nc1cccn(CC(=O)NC2C3CC4CC(C3)CC2C4)c1=O]

The synthesis of compound II-112 was performed according to compound II-3, using isonicotinic acid instead of 3-methylbenzo[b]furan-2-carboxylic acid in step 6 (according to ZED3264).
Yield: 47 mg, 52% (last step)
ESI-MS: 577.4 [M+H]⁺

### 103 Preparation of Compound II-113

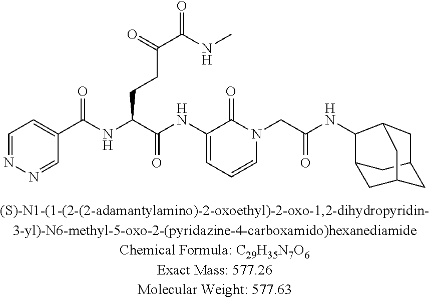 [SMILES: CNC(=O)C(=O)CC[C@H](NC(=O)c1ccnnc1)C(=O)Nc1cccn(CC(=O)NC2C3CC4CC(C3)CC2C4)c1=O]

The synthesis of compound II-113 was performed according to compound II-3, using pyridazine-4-carboxylic acid instead of 3-methylbenzo[b]furan-2-carboxylic acid in step 6 (according to ZED3264).
Yield: 34 mg, 46% (last step)
ESI-MS: 578.4 [M+H]⁺
   104 Preparation of compound II-114

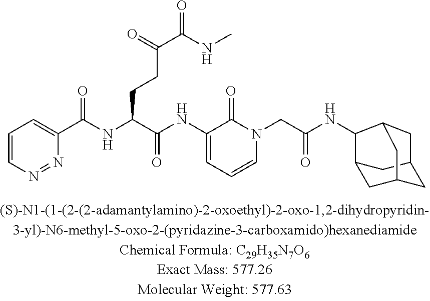 [SMILES: CNC(=O)C(=O)CC[C@H](NC(=O)c1cccnn1)C(=O)Nc1cccn(CC(=O)NC2C3CC4CC(C3)CC2C4)c1=O]

The synthesis of compound II-114 was performed according to compound II-3, using pyridazine-3-carboxylic acid instead of 3-methylbenzo[b]furan-2-carboxylic acid in step 6 (according to ZED3264).
Yield: 43 mg, 56% (last step)
ESI-MS: 578.4 [M+H]⁺
   Preparation of compound II-115

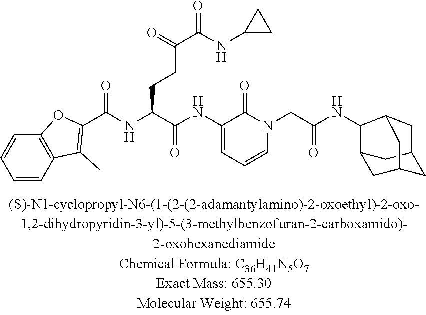 [SMILES: Cc1c(C(=O)N[C@@H](CCC(=O)C(=O)NC2CC2)C(=O)Nc2cccn(CC(=O)NC3C4CC5CC(C4)CC3C5)c2=O)oc2ccccc12]

The synthesis of compound II-115 was performed according to compound II-3, using cyclopropyl isocyanide instead of methyl isocyanide in step 4 (according to ZED3632).
Yield: 47 mg, 64% (last step)
ESI-MS: 656.5 [M+H]⁺
   106 Preparation of compound II-116

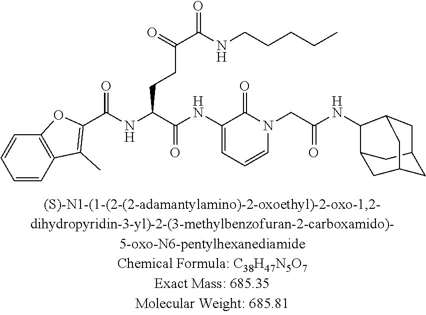 [SMILES: CCCCCNC(=O)C(=O)CC[C@H](NC(=O)c1oc2ccccc2c1C)C(=O)Nc1cccn(CC(=O)NC2C3CC4CC(C3)CC2C4)c1=O]

The synthesis of compound II-116 was performed according to compound II-3, using pentyl isocyanide instead of methyl isocyanide in step 4 (according to ZED3632).
Yield: 87 mg, 71% (last step)
ESI-MS: 686.5 [M+H]⁺

### 107 Preparation of Compound II-117

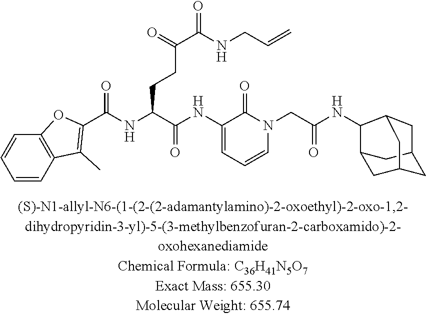 [SMILES: C=CCNC(=O)C(=O)CC[C@H](NC(=O)c1oc2ccccc2c1C)C(=O)Nc1cccn(CC(=O)NC2C3CC4CC(C3)CC2C4)c1=O]

The synthesis of compound MI-117 was performed according to compound II-3, using allyl isocyanide instead of methyl isocyanide in step 4 (according to ZED3632).
Yield: 42 mg, 63% (last step)
ESI-MS: 656.5 [M+H]⁺

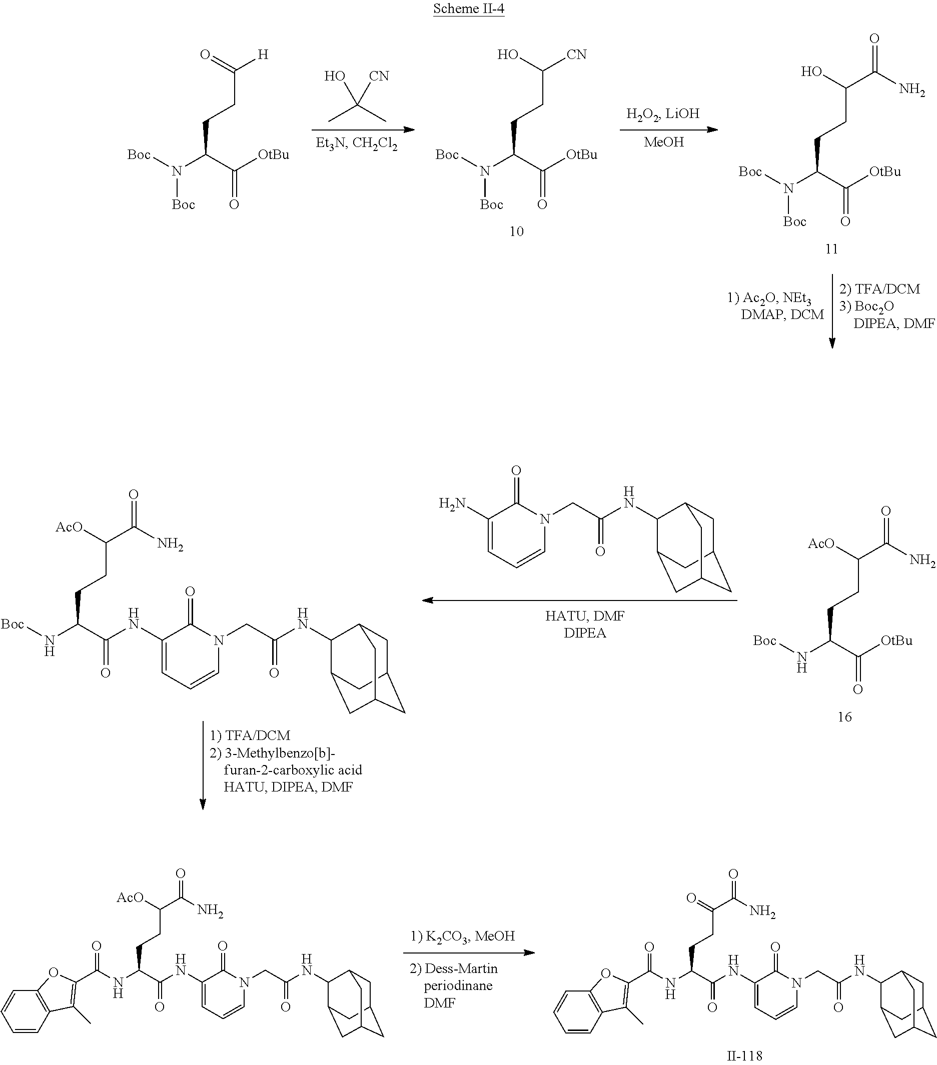

### Preparation of Compound 10

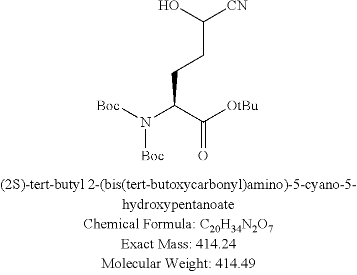 [SMILES: CC(C)(C)OC(=O)[C@H](CCC(O)C#N)N(C(=O)OC(C)(C)C)C(=O)OC(C)(C)C]

15.0 g (38.7 mmol) of the aldehyde(S)-tert-butyl 2-(bis (tert-butoxycarbonyl) amino)-5-oxopentanoate (ZED721) were dissolved in 150 ml DCM. 6.42 ml (46.3 mmol) trimethylamine and 7.37 ml (79.9 mmol) acetone cyanohydrin were added, and the reaction was stirred at room temperature overnight. The solution was washed twice with each citric acid solution (10%) and brine. The organic phase was dried over Na₂SO₄, filtered and the solvent was evaporated. The residue was purified by flash chromatography.
Yield: 16.2 g, >100%
ESI-MS: 437.6 [M+Na]⁺
   Preparation of compound 11

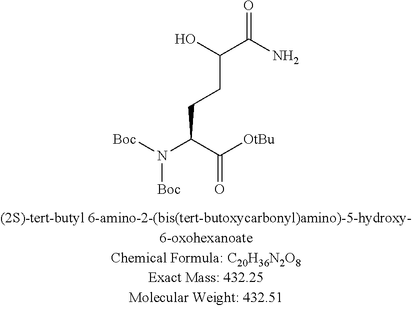 [SMILES: CC(C)(C)OC(=O)[C@H](CCC(O)C(N)=O)N(C(=O)OC(C)(C)C)C(=O)OC(C)(C)C]

16.2 g (˜38.6 mmol) of cyanohydrin 10 were dissolved in 95 ml MeOH at 4° C. and 1.91 g (45.5 mmol) lithium hydroxide monohydrate were added. 18.6 ml hydrogen peroxide (35%) were added dropwise, and the reaction was stirred at room temperature for 1.5 h before quenching with sodium thiosulfate solution (5%). The aqueous phase was extracted with DCM. The combined organic phases were dried over Na₂SO₄, filtered and the solvent was evaporated. The residue was purified by flash chromatography.
Yield: 8.61 g, 52%
ESI-MS: 455.2 [M+Na]⁺

### Preparation of Compound 15

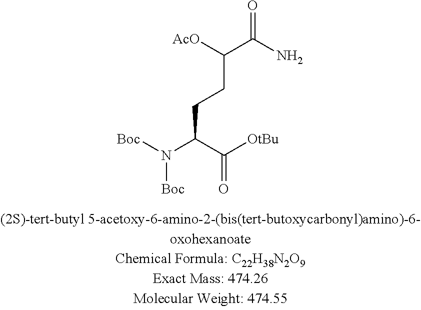 [SMILES: CC(=O)OC(CC[C@@H](C(=O)OC(C)(C)C)N(C(=O)OC(C)(C)C)C(=O)OC(C)(C)C)C(N)=O]

8.61 g (19.9 mmol) of hydroxyamide 10 were dissolved in 55 ml DCM. 3.45 ml (24.9 mmol) 1.91 g (45.5 mmol) trimethylamine, 2.12 ml acetic anhydride and 62 mg (0.50 mmol) DMAP were added, and the reaction was stirred at room temperature for 3 h. After washing with water and brine, the organic phase was dried over Na₂SO₄, filtered and the solvent was evaporated. The product precipitates from MTBE solution by addition of hexane.
Yield: 8.08 g, 86%
ESI-MS: 475.5 [M+H]⁺

### Preparation of Compound 16

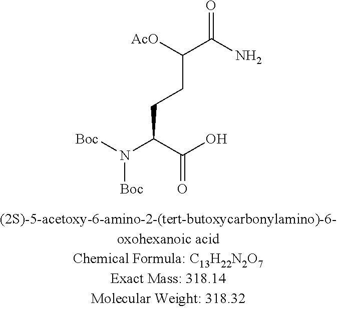 [SMILES: CC(=O)OC(CC[C@@H](C(=O)O)N(C(=O)OC(C)(C)C)C(=O)OC(C)(C)C)C(N)=O]

8.08 g (17.0 mmol) of 15 were dissolved in 140 ml DCM/TFA (1:1) and stirred at room temperature for 3 h. The solvent was evaporated, and the residue was dissolved in 40 ml DMF. 5.80 ml (2 eq) DIPEA and 4.55 g (20.4 mmol) di-tert-butyl dicarbonate in 20 ml DMF were added and the reaction was stirred at room temperature overnight. The solvent was evaporated, and the residue was dissolved in 80 ml EtOAc. After extraction with NaHCO₃ solution (1.05 eq in water), the product precipitates from the aqueous phase by addition of 1.5 eq citric acid.
Yield: 1.64 g, 30%
ESI-MS: 319.4 [M+H]⁺

### 108 Preparation of Compound II-118

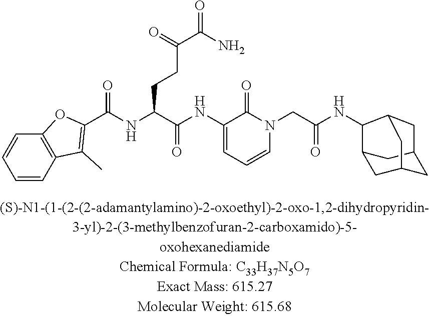 [SMILES: Cc1c(C(=O)N[C@@H](CCC(=O)C(N)=O)C(=O)Nc2cccn(CC(=O)NC3C4CC5CC(C4)CC3C5)c2=O)oc2ccccc12]

The synthesis of compound II-118 was performed according to compound II-3, using compound 16 instead of ZED3632 in step 5 (according to ZED3907).
Yield: 158 mg, 56% (last step)
ESI-MS: 616.4 [M+H]⁺

### 109 Preparation of Compound II-119

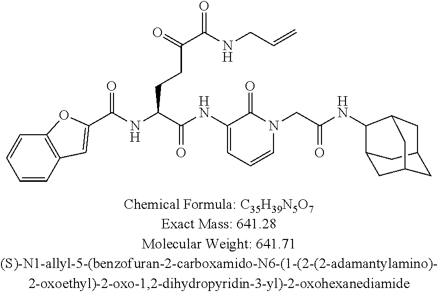 [SMILES: C=CCNC(=O)C(=O)CC[C@H](NC(=O)c1cc2ccccc2o1)C(=O)Nc1cccn(CC(=O)NC2C3CC4CC(C3)CC2C4)c1=O]

10 The synthesis of compound II-119 was performed according to compound II-2, using allyl isocyanide instead of methyl isocyanide in step 4 (according to ZED3632).
Yield: 56 mg, 71% (last step)
ESI-MS: 642.4 [M+H]⁺

### 110 Preparation of Compound II-120

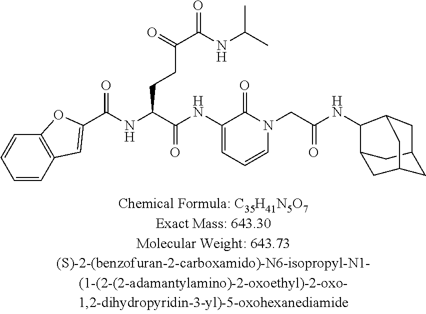 [SMILES: CC(C)NC(=O)C(=O)CC[C@H](NC(=O)c1cc2ccccc2o1)C(=O)Nc1cccn(CC(=O)NC2C3CC4CC(C3)CC2C4)c1=O]

The synthesis of compound II-120 was performed according to compound II-2, using isopropyl isocyanide instead of methyl isocyanide in step 4 (according to ZED3632).
Yield: 62 mg, 65% (last step)
ESI-MS: 644.5 [M+H]⁺
   111 Preparation of compound II-121

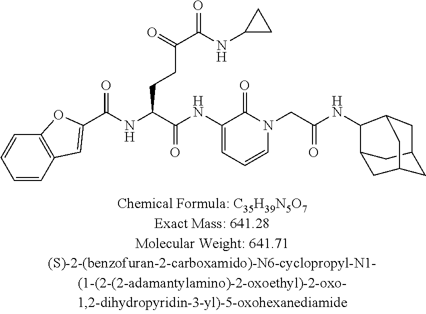 [SMILES: O=C(Cn1cccc(NC(=O)[C@H](CCC(=O)C(=O)NC2CC2)NC(=O)c2cc3ccccc3o2)c1=O)NC1C2CC3CC(C2)CC1C3]

The synthesis of compound II-121 was performed according to compound II-2, using cyclopropyl isocyanide instead of methyl isocyanide in step 4 (according to ZED3632).
Yield: 44 mg, 51% (last step)
ESI-MS: 642.4 [M+H]⁺

### 112 Preparation of Compound II-122

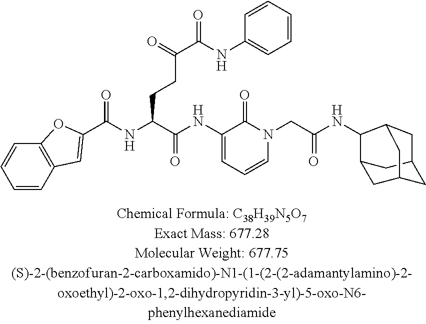 [SMILES: O=C(Cn1cccc(NC(=O)[C@H](CCC(=O)C(=O)Nc2ccccc2)NC(=O)c2cc3ccccc3o2)c1=O)NC1C2CC3CC(C2)CC1C3]

The synthesis of compound II-122 was performed according to compound II-2, using phenyl isocyanide instead of methyl isocyanide in step 4 (according to ZED3632).
Yield: 37 mg, 56% (last step)
ESI-MS: 678.4 [M+H]⁺

### 113 Preparation of Compound II-123

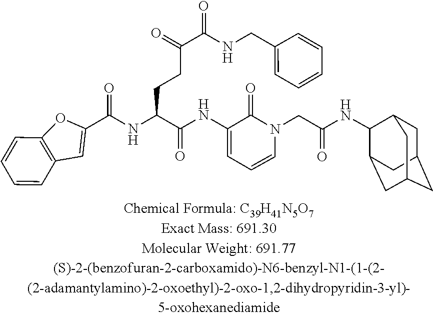 [SMILES: O=C(Cn1cccc(NC(=O)[C@H](CCC(=O)C(=O)NCc2ccccc2)NC(=O)c2cc3ccccc3o2)c1=O)NC1C2CC3CC(C2)CC1C3]

The synthesis of compound II-123 was performed according to compound II-2, using benzyl isocyanide instead of methyl isocyanide in step 4 (according to ZED3632).
Yield: 46 mg, 52% (last step)
ESI-MS: 692.5 [M+H]⁺

### 114 Preparation of Compound II-124

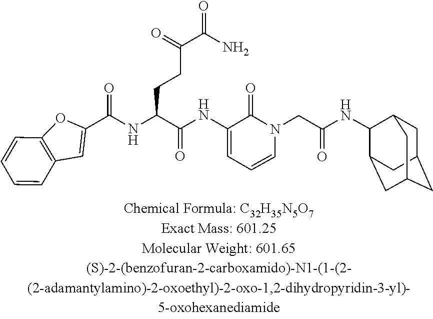 [SMILES: NC(=O)C(=O)CC[C@H](NC(=O)c1cc2ccccc2o1)C(=O)Nc1cccn(CC(=O)NC2C3CC4CC(C3)CC2C4)c1=O]

The synthesis of compound II-124 was performed according to compound II-118, using benzofuran-2-carboxylic acid instead of 3-methylbenzo[b]furan-2-carboxylic acid in step 6 (according to ZED3264).
Yield: 96 mg, 81% (last step)
ESI-MS: 602.4 [M+H]⁺

### 115 Preparation of Compound II-125

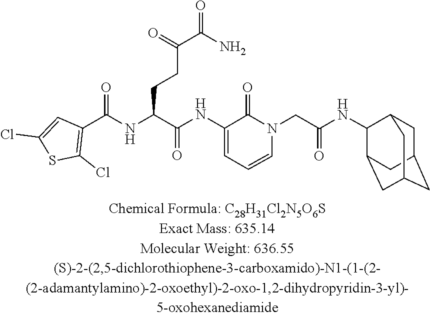 [SMILES: NC(=O)C(=O)CC[C@H](NC(=O)c1cc(Cl)sc1Cl)C(=O)Nc1cccn(CC(=O)NC2C3CC4CC(C3)CC2C4)c1=O]

The synthesis of compound II-125 was performed according to compound II-124, using 2,5-dichlorothiophene-3-carboxylic acid instead of benzofuran-2-carboxylic acid in step 6 (according to ZED3264).
Yield: 78 mg, 71% (last step)
ESI-MS: 636.3/638.3 [M+H]⁺
   116 Preparation of compound II-126

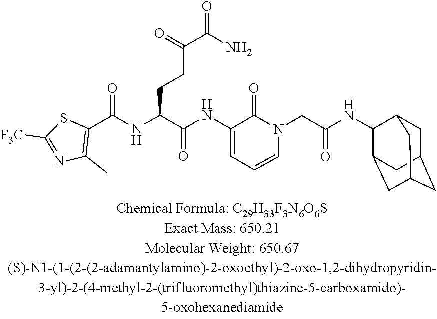 [SMILES: Cc1nc(C(F)(F)F)sc1C(=O)N[C@@H](CCC(=O)C(N)=O)C(=O)Nc1cccn(CC(=O)NC2C3CC4CC(C3)CC2C4)c1=O]

The synthesis of compound II-126 was performed according to compound II-124, using 4-methyl-2-(trifluoromethyl) thiazole-5-carboxylic acid instead of benzofuran-2-carboxylic acid in step 6 (according to ZED3264).
Yield: 53 mg, 67% (last step)
ESI-MS: 651.3 [M+H]⁺

### 117 Preparation of Compound II-127

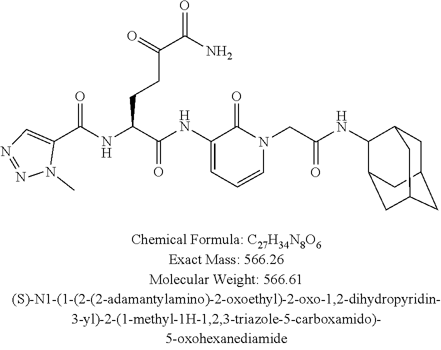 [SMILES: Cn1nncc1C(=O)N[C@@H](CCC(=O)C(N)=O)C(=O)Nc1cccn(CC(=O)NC2C3CC4CC(C3)CC2C4)c1=O]

The synthesis of compound II-127 was performed according to compound II-124, using 1-methyl-1H-1,2,3-triazole-5-carboxylic acid instead of benzofuran-2-carboxylic acid in step 6 (according to ZED3264).
Yield: 26 mg, 49% (last step)
ESI-MS: 567.3 [M+H]⁺

### 118 Preparation of Compound II-128

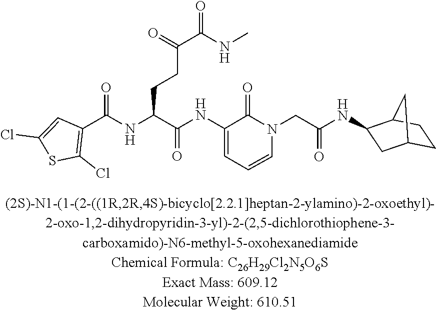 [SMILES: CNC(=O)C(=O)CC[C@H](NC(=O)c1cc(Cl)sc1Cl)C(=O)Nc1cccn(CC(=O)N[C@@H]2CC3CCC2C3)c1=O]

The synthesis of compound II-128 was performed according to compound II-97, using 2,5-dichlorothiophene-3-carboxylic acid instead of 3-methylbenzo[b]furan-2-carboxylic acid in step 6 (according to ZED3264).
Yield: 53 mg, 73% (last step)
ESI-MS: 610.3/612.3 [M+H]⁺
   119 Preparation of compound II-129

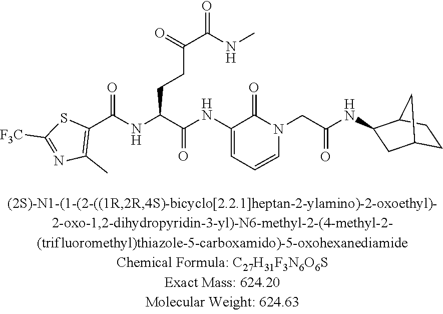 [SMILES: CNC(=O)C(=O)CC[C@H](NC(=O)c1sc(C(F)(F)F)nc1C)C(=O)Nc1cccn(CC(=O)N[C@@H]2CC3CCC2C3)c1=O]

The synthesis of compound II-129 was performed according to compound II-97, using 4-methyl-2-(trifluoromethyl) thiazole-5-carboxylic acid instead of 3-methylbenzo[b]furan-2-carboxylic acid in step 6 (according to ZED3264).
Yield: 42 mg, 60% (last step)
ESI-MS: 625.3 [M+H]⁺

### 120 Preparation of Compound II-130

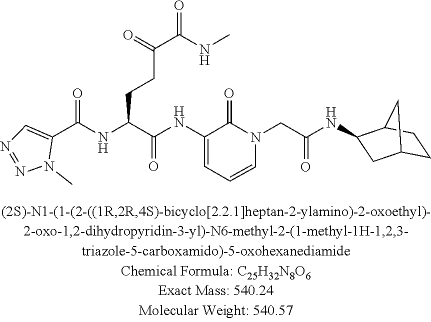 [SMILES: CNC(=O)C(=O)CC[C@H](NC(=O)c1cnnn1C)C(=O)Nc1cccn(CC(=O)N[C@@H]2CC3CCC2C3)c1=O]

The synthesis of compound II-130 was performed according to compound II-97, using 1-methyl-1H-1,2,3-triazole-5-carboxylic acid instead of 3-methylbenzo[b]furan-2-carboxylic acid in step 6 (according to ZED3264).
Yield: 15 mg, 39% (last step)
ESI-MS: 541.3 [M+H]⁺

### 121 Preparation of Compound II-131

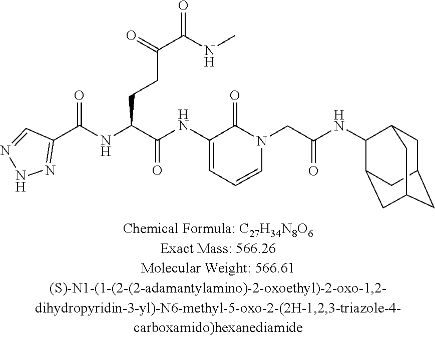 [SMILES: CNC(=O)C(=O)CC[C@H](NC(=O)c1cn[nH]n1)C(=O)Nc1cccn(CC(=O)NC2C3CC4CC(C3)CC2C4)c1=O]

The synthesis of compound II-131 was performed according to compound II-3, using 2H-1,2,3-triazole-4-carboxylic acid instead of 3-methylbenzo[b]furan-2-carboxylic acid in step 6 (according to ZED3264).
Yield: 28 mg, 56% (last step)
ESI-MS: 567.4 [M+H]⁺

### 122 Preparation of Compound II-132

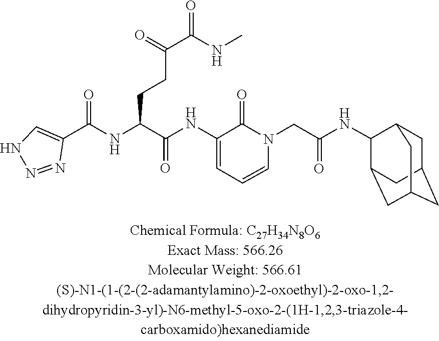 [SMILES: CNC(=O)C(=O)CC[C@H](NC(=O)c1c[nH]nn1)C(=O)Nc1cccn(CC(=O)NC2C3CC4CC(C3)CC2C4)c1=O]

The synthesis of compound II-132 was performed according to compound II-3, using 1H-1,2,3-triazole-4-carboxylic acid instead of 3-methylbenzo[b]furan-2-carboxylic acid in step 6 (according to ZED3264).
Yield: 14 mg, 45% (last step)
ESI-MS: 567.4 [M+H]⁺

### 123 Preparation of Compound II-133

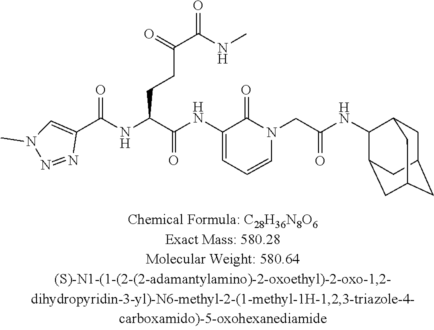 [SMILES: CNC(=O)C(=O)CC[C@H](NC(=O)c1cn(C)nn1)C(=O)Nc1cccn(CC(=O)NC2C3CC4CC(C3)CC2C4)c1=O]

The synthesis of compound II-133 was performed according to compound II-3, using 1-methyl-1H-1,2,3-triazole-4-carboxylic acid instead of 3-methylbenzo[b]furan-2-carboxylic acid in step 6 (according to ZED3264).
Yield: 36 mg, 61% (last step)
ESI-MS: 581.4 [M+H]⁺

### 124 Preparation of Compound II-134

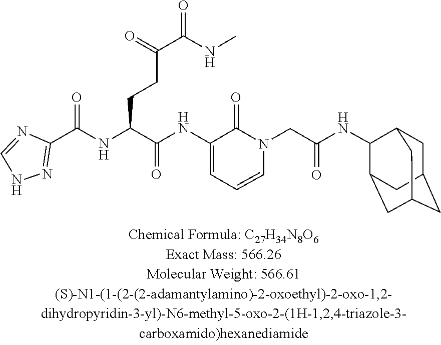 [SMILES: CNC(=O)C(=O)CC[C@H](NC(=O)c1nc[nH]n1)C(=O)Nc1cccn(CC(=O)NC2C3CC4CC(C3)CC2C4)c1=O]

The synthesis of compound II-134 was performed according to compound II-3, using 1H-1,2,4-triazole-3-carboxylic acid instead of 3-methylbenzo[b]furan-2-carboxylic acid in step 6 (according to ZED3264).
Yield: 21 mg, 43% (last step)
ESI-MS: 567.4 [M+H]⁺

### 125 Preparation of Compound II-135

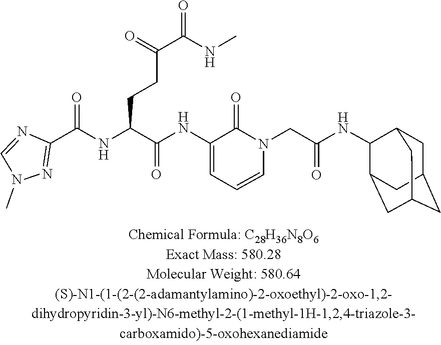 [SMILES: CNC(=O)C(=O)CC[C@H](NC(=O)c1ncn(C)n1)C(=O)Nc1cccn(CC(=O)NC2C3CC4CC(C3)CC2C4)c1=O]

The synthesis of compound II-135 was performed according to compound II-3, using 1-methyl-1H-1,2,4-triazole-3-carboxylic acid instead of 3-methylbenzo[b]furan-2-carboxylic acid in step 6 (according to ZED3264).
Yield: 39 mg, 67% (last step)
ESI-MS: 581.4 [M+H]⁺

### 126 Preparation of Compound II-136

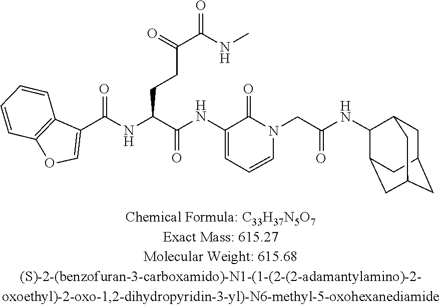 [SMILES: CNC(=O)C(=O)CC[C@H](NC(=O)c1coc2ccccc12)C(=O)Nc1cccn(CC(=O)NC2C3CC4CC(C3)CC2C4)c1=O]

The synthesis of compound II-136 was performed according to compound II-3, using benzofuran-3-carboxylic acid instead of 3-methylbenzo[b]furan-2-carboxylic acid in step 6 (according to ZED3264).
Yield: 56 mg, 71% (last step)
ESI-MS: 616.4 [M+H]⁺

### 127 Preparation of Compound II-137

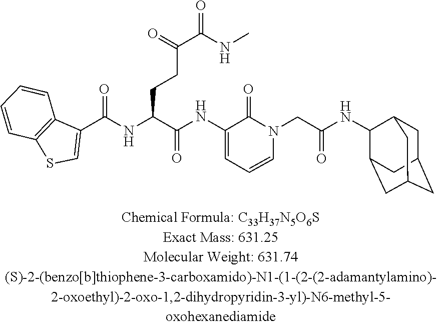 [SMILES: CNC(=O)C(=O)CC[C@H](NC(=O)c1csc2ccccc12)C(=O)Nc1cccn(CC(=O)NC2C3CC4CC(C3)CC2C4)c1=O]

The synthesis of compound II-137 was performed according to compound II-3, using benzo[b]thiophene-3-carboxylic acid instead of 3-methylbenzo[b]furan-2-carboxylic acid in step 6 (according to ZED3264).
Yield: 49 mg, 64% (last step)
ESI-MS: 632.4 [M+H]⁺

### 128 Preparation of Compound II-138

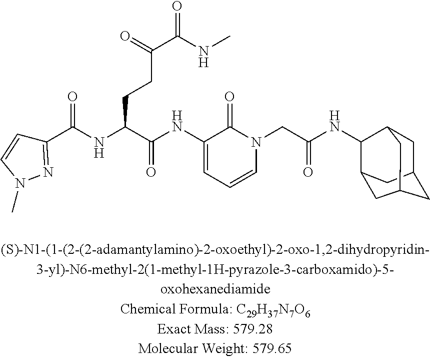 [SMILES: CNC(=O)C(=O)CC[C@H](NC(=O)c1ccn(C)n1)C(=O)Nc1cccn(CC(=O)NC2C3CC4CC(C3)CC2C4)c1=O]

The synthesis of compound II-138 was performed according to compound II-3, using 1-methyl-1H-pyrazole-3-carboxylic acid instead of 3-methylbenzo[b]furan-2-carboxylic acid in step 6 (according to ZED3264).
Yield: 43 mg, 59% (last step)
ESI-MS: 580.4 [M+H]⁺

### 129 Preparation of Compound II-139

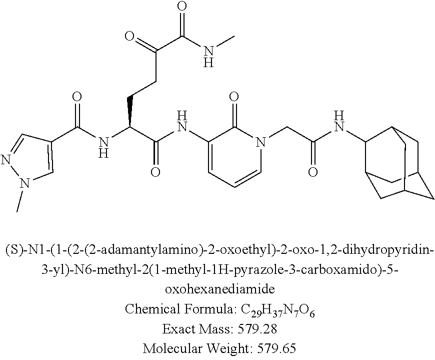 [SMILES: CNC(=O)C(=O)CC[C@H](NC(=O)c1cnn(C)c1)C(=O)Nc1cccn(CC(=O)NC2C3CC4CC(C3)CC2C4)c1=O]

The synthesis of compound II-139 was performed according to compound II-3, using 1-methyl-1H-pyrazole-4-carboxylic acid instead of 3-methylbenzo[b]furan-2-carboxylic acid in step 6 (according to ZED3264).
Yield: 53 mg, 62% (last step)
ESI-MS: 580.4 [M+H]⁺

### 130 Preparation of Compound II-140

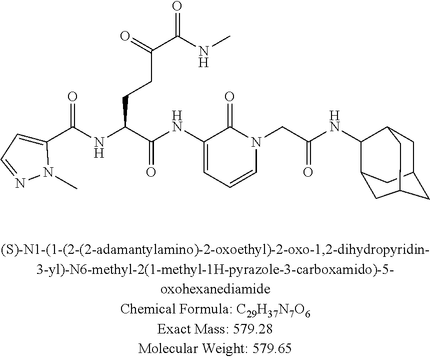 [SMILES: CNC(=O)C(=O)CC[C@H](NC(=O)c1ccnn1C)C(=O)Nc1cccn(CC(=O)NC2C3CC4CC(C3)CC2C4)c1=O]

The synthesis of compound II-140 was performed according to compound II-3, using 1-methyl-1H-pyrazole-5-carboxylic acid instead of 3-methylbenzo[b]furan-2-carboxylic acid in step 6 (according to ZED3264).
Yield: 39 mg, 48% (last step)
ESI-MS: 580.4 [M+H]⁺
   131 Preparation of compound II-141

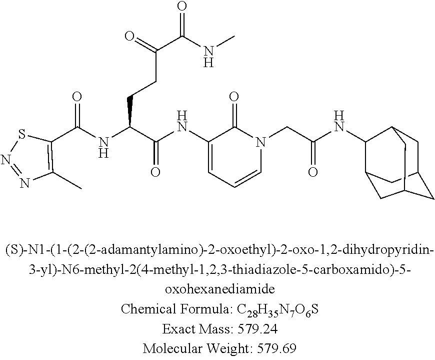 [SMILES: CNC(=O)C(=O)CC[C@H](NC(=O)c1snnc1C)C(=O)Nc1cccn(CC(=O)NC2C3CC4CC(C3)CC2C4)c1=O]

The synthesis of compound II-141 was performed according to compound II-3, using 4-methyl-1,2,3-thiadiazole-5-carboxylic acid instead of 3-methylbenzo[b]furan-2-carboxylic acid in step 6 (according to ZED3264).
Yield: 54 mg, 58% (last step)
ESI-MS: 598.4 [M+H]⁺

### Preparation of Compound II-142

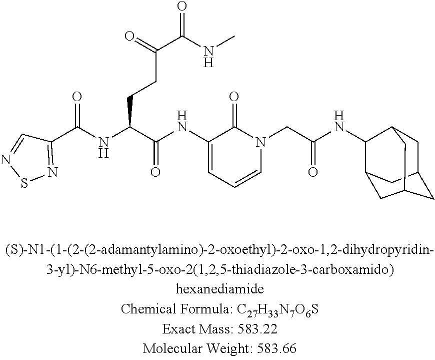 [SMILES: CNC(=O)C(=O)CC[C@H](NC(=O)c1cnsn1)C(=O)Nc1cccn(CC(=O)NC2C3CC4CC(C3)CC2C4)c1=O]

The synthesis of compound II-142 was performed according to compound II-3, using 1,2,5-thiadiazole-3-carboxylic acid instead of 3-methylbenzo[b]furan-2-carboxylic acid in step 6 (according to ZED3264).
Yield: 36 mg, 43% (last step)
ESI-MS: 584.4 [M+H]⁺

### 133 Preparation of Compound II-143

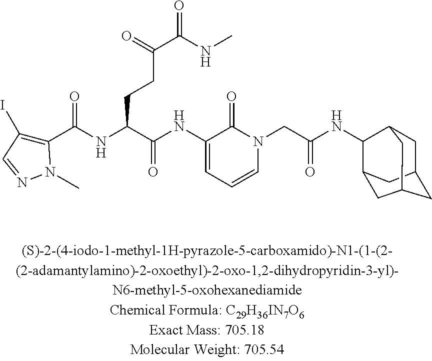 [SMILES: CNC(=O)C(=O)CC[C@H](NC(=O)c1c(I)cnn1C)C(=O)Nc1cccn(CC(=O)NC2C3CC4CC(C3)CC2C4)c1=O]

The synthesis of compound II-143 was performed according to compound II-3, using 4-iodo-1-methyl-1H-pyrazole-5-carboxylic acid instead of 3-methylbenzo[b]furan-2-carboxylic acid in step 6 (according to ZED3264).
Yield: 24 mg, 49% (last step)
ESI-MS: 706.3 [M+H]⁺

### 134 Preparation of Compound II-144

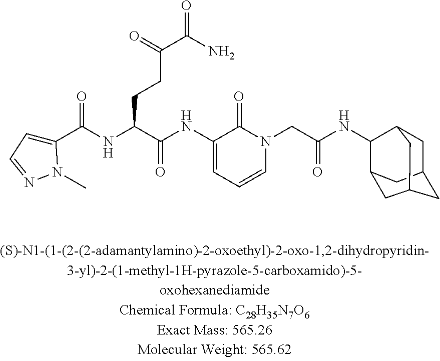 [SMILES: Cn1nccc1C(=O)N[C@@H](CCC(=O)C(N)=O)C(=O)Nc1cccn(CC(=O)NC2C3CC4CC(C3)CC2C4)c1=O]

The synthesis of compound II-144 was performed according to compound II-118, using 1-methyl-1H-pyrazole-5-carboxylic acid instead of 3-methylbenzo[b]furan-2-carboxylic acid in step 6 (according to ZED3264).
Yield: 26 mg, 68% (last step)
ESI-MS: 566.4 [M+H]⁺

### 135 Preparation of Compound II-145

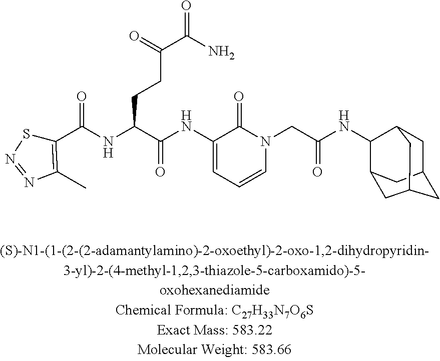 [SMILES: Cc1nnsc1C(=O)N[C@@H](CCC(=O)C(N)=O)C(=O)Nc1cccn(CC(=O)NC2C3CC4CC(C3)CC2C4)c1=O]

The synthesis of compound II-145 was performed according to compound II-118, using 4-methyl-1,2,3-thiadiazole-5-carboxylic acid instead of 3-methylbenzo[b]furan-2-carboxylic acid in step 6 (according to ZED3264).
Yield: 35 mg, 62% (last step)
ESI-MS: 584.4 [M+H]⁺

### 136 Preparation of Compound II-146

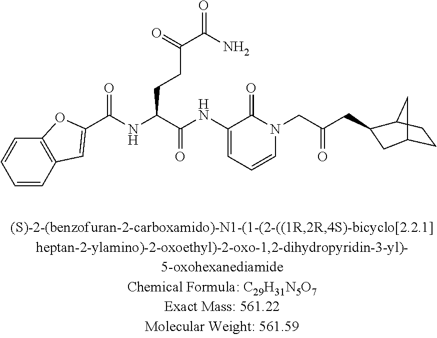 [SMILES: NC(=O)C(=O)CC[C@H](NC(=O)c1cc2ccccc2o1)C(=O)Nc1cccn(CC(=O)C[C@@H]2CC3CCC2C3)c1=O]

The synthesis of compound II-146 was performed according to compound II-118, using benzofuran-2-carboxylic acid instead of 3-methylbenzo[b]furan-2-carboxylic acid in step 6 (according to ZED3264) and using exo-2-aminonorbornane instead of 2-adamantan-amine in step 2 (according to ZED3905).
Yield: 43 mg, 60% (last step)
ESI-MS: 562.4 [M+H]⁺

### 137 Preparation of Compound II-147

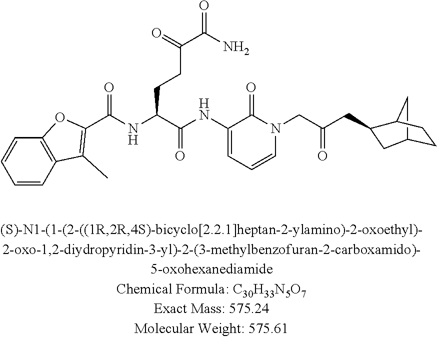 [SMILES: Cc1c(C(=O)N[C@@H](CCC(=O)C(N)=O)C(=O)Nc2cccn(CC(=O)C[C@@H]3CC4CCC3C4)c2=O)oc2ccccc12]

The synthesis of compound II-147 was performed according to compound II-118, using using exo-2-aminonorbornane instead of 2-adamantanamine in step 2 (according to ZED3905).
Yield: 56 mg, 67% (last step)
ESI-MS: 576.4 [M+H]⁺
   138Preparation of compound II-148

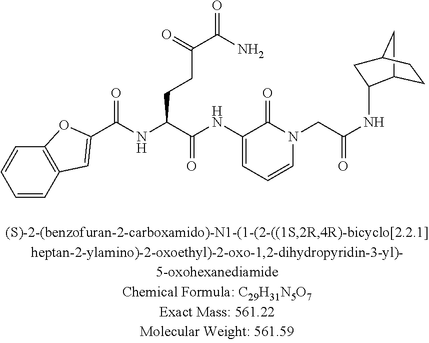 [SMILES: NC(=O)C(=O)CC[C@H](NC(=O)c1cc2ccccc2o1)C(=O)Nc1cccn(CC(=O)NC2CC3CCC2C3)c1=O]

The synthesis of compound II-148 was performed according to compound II-118, using benzofuran-2-carboxylic acid instead of 3-methylbenzo[b]furan-2-carboxylic acid in step 6 (according to ZED3264) and using (+)-endo-2-aminonorbornane instead of 2-adamantanamine in step 2 (according to ZED3905).
Yield: 49 mg, 66% (last step)
ESI-MS: 562.4 [M+H]⁺

### 139 Preparation of Compound II-149

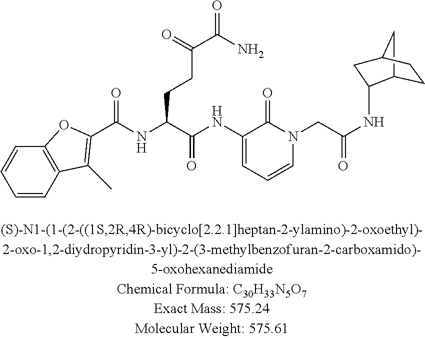 [SMILES: Cc1c(C(=O)N[C@@H](CCC(=O)C(N)=O)C(=O)Nc2cccn(CC(=O)NC3CC4CCC3C4)c2=O)oc2ccccc12]

The synthesis of compound II-149 was performed according to compound II-118, using using (+)-endo-2-aminonorbornane instead of 2-adamantanamine in step 2 (according to ZED3905).
Yield: 64 mg, 75% (last step)
ESI-MS: 576.4 [M+H]⁺

### 140 Preparation of Compound II-150

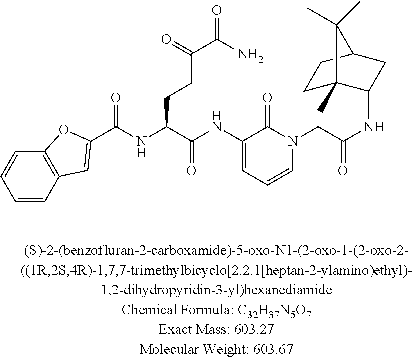 [SMILES: CC1(C)C2CC[C@@]1(C)C(NC(=O)Cn1cccc(NC(=O)[C@H](CCC(=O)C(N)=O)NC(=O)c3cc4ccccc4o3)c1=O)C2]

The synthesis of compound II-150 was performed according to compound II-118, using benzofuran-2-carboxylic acid instead of 3-methylbenzo[b]furan-2-carboxylic acid in step 6 (according to ZED3264) and using (R)-(+)-bornylamine instead of 2-adamantanamine in step 2 (according to ZED3905).
Yield: 38 mg, 53% (last step)
ESI-MS: 604.4 [M+H]⁺

### 141 Preparation of Compound II-151

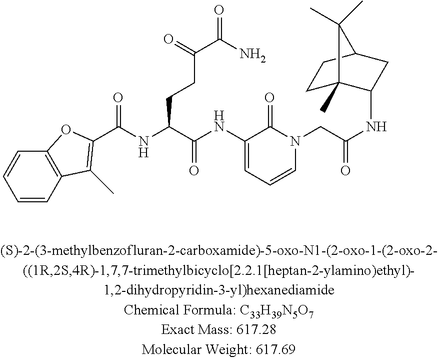 [SMILES: Cc1c(C(=O)N[C@@H](CCC(=O)C(N)=O)C(=O)Nc2cccn(CC(=O)NC3CC4CC[C@]3(C)C4(C)C)c2=O)oc2ccccc12]

The synthesis of compound II-151 was performed according to compound II-118, using using (R)-(+)-bornylamine instead of 2-adamantanamine in step 2 (according to ZED3905).
Yield: 31 mg, 59% (last step)
ESI-MS: 618.5 [M+H]⁺

### Preparation of Compound II-152

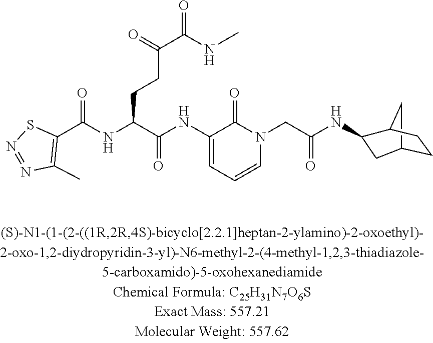 [SMILES: CNC(=O)C(=O)CC[C@H](NC(=O)c1snnc1C)C(=O)Nc1cccn(CC(=O)N[C@@H]2CC3CCC2C3)c1=O]

The synthesis of compound II-152 was performed according to compound II-94, using 4-methyl-1,2,3-thiadiazole-5-carboxylic acid instead of 3-methylbenzo[b]furan-2-carboxylic acid in step 6 (according to ZED3264).
Yield: 53 mg, 68% (last step)
ESI-MS: 558.4 [M+H]⁺

### 143 Preparation of Compound II-153

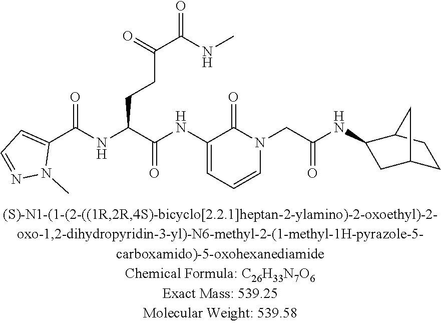 [SMILES: CNC(=O)C(=O)CC[C@H](NC(=O)c1ccnn1C)C(=O)Nc1cccn(CC(=O)N[C@@H]2CC3CCC2C3)c1=O]

The synthesis of compound II-153 was performed according to compound II-94, using 1-methyl-1H-pyrazole-5-carboxylic acid instead of 3-methylbenzo[b]furan-2-carboxylic acid in step 6 (according to ZED3264).
Yield: 60 mg, 67% (last step)
ESI-MS: 540.4 [M+H]⁺

### 144 Preparation of Compound II-154

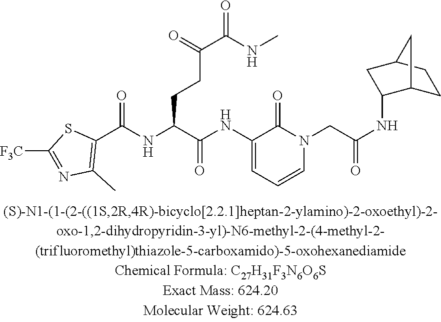 [SMILES: CNC(=O)C(=O)CC[C@H](NC(=O)c1sc(C(F)(F)F)nc1C)C(=O)Nc1cccn(CC(=O)NC2CC3CCC2C3)c1=O]

The synthesis of compound II-154 was performed according to compound II-90, using 4-methyl-2-(trifluoromethyl) thiazole-5-carboxylic acid instead of 3-methylbenzo[b]furan-2-carboxylic acid in step 6 (according to ZED3264).
Yield: 68 mg, 74% (last step)
ESI-MS: 625.3 [M+H]⁺

### 145 Preparation of Compound II-155

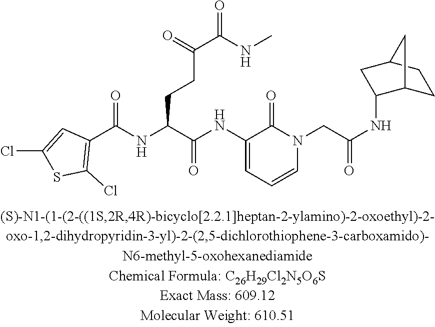 [SMILES: CNC(=O)C(=O)CC[C@H](NC(=O)c1cc(Cl)sc1Cl)C(=O)Nc1cccn(CC(=O)NC2CC3CCC2C3)c1=O]

The synthesis of compound II-155 was performed according to compound II-90, using 2,5-dichlorothiophene-3-carboxylic acid instead of 3-methylbenzo[b]furan-2-carboxylic acid in step 6 (according to ZED3264).
Yield: 74 mg, 68% (last step)
ESI-MS: 610.3/612.3 [M+H]⁺

### 146 Preparation of Compound II-156

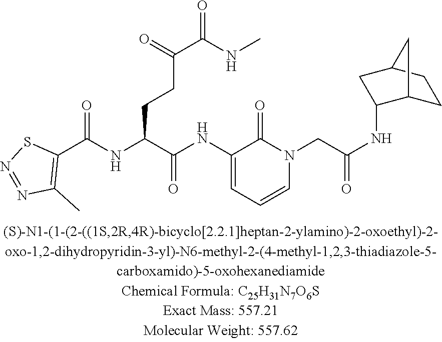 [SMILES: CNC(=O)C(=O)CC[C@H](NC(=O)c1snnc1C)C(=O)Nc1cccn(CC(=O)NC2CC3CCC2C3)c1=O]

The synthesis of compound II-156 was performed according to compound II-90, using 4-methyl-1,2,3-thiadiazole-5-carboxylic acid instead of 3-methylbenzo[b]furan-2-carboxylic acid in step 6 (according to ZED3264).
Yield: 56 mg, 61% (last step)
ESI-MS: 558.4 [M+H]⁺

### 147 Preparation of Compound II-157

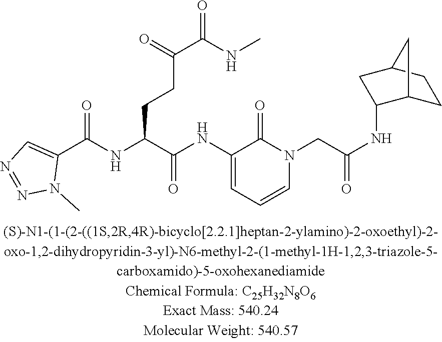 [SMILES: CNC(=O)C(=O)CC[C@H](NC(=O)c1cnnn1C)C(=O)Nc1cccn(CC(=O)NC2CC3CCC2C3)c1=O]

The synthesis of compound II-157 was performed according to compound II-90, using 1-methyl-1H-1,2,3-triazole-5-carboxylic acid instead of 3-methylbenzo[b]furan-2-carboxylic acid in step 6 (according to ZED3264).
Yield: 34 mg, 52% (last step)
ESI-MS: 541.4 [M+H]⁺

### 148 Preparation of Compound II-158

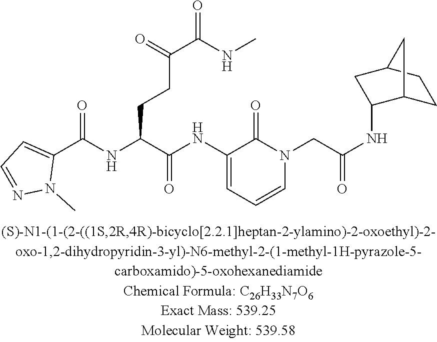 [SMILES: CNC(=O)C(=O)CC[C@H](NC(=O)c1ccnn1C)C(=O)Nc1cccn(CC(=O)NC2CC3CCC2C3)c1=O]

The synthesis of compound II-158 was performed according to compound II-90, using 1-methyl-1H-pyrazole-5-carboxylic acid instead of 3-methylbenzo[b]furan-2-carboxylic acid in step 6 (according to ZED3264).
Yield: 55 mg, 64% (last step)
ESI-MS: 540.4 [M+H]⁺

### 149 Preparation of Compound II-159

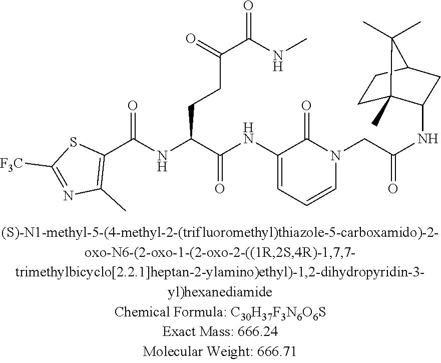 [SMILES: CNC(=O)C(=O)CC[C@H](NC(=O)c1sc(C(F)(F)F)nc1C)C(=O)Nc1cccn(CC(=O)NC2CC3CC[C@]2(C)C3(C)C)c1=O]

The synthesis of compound II-159 was performed according to compound II-92, using 4-methyl-2-(trifluoromethyl) thiazole-5-carboxylic acid instead of 3-methylbenzo[b]furan-2-carboxylic acid in step 6 (according to ZED3264).
Yield: 52 mg, 57% (last step)
ESI-MS: 667.4 [M+H]⁺

### 150 Preparation of Compound II-160

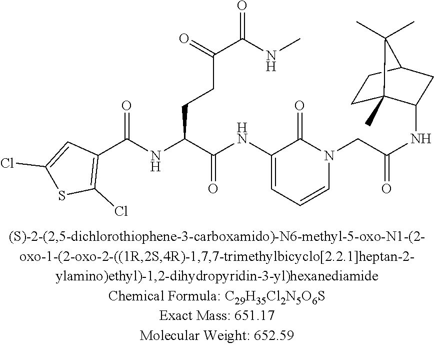 [SMILES: CNC(=O)C(=O)CC[C@H](NC(=O)c1cc(Cl)sc1Cl)C(=O)Nc1cccn(CC(=O)NC2CC3CC[C@]2(C)C3(C)C)c1=O]

The synthesis of compound II-160 was performed according to compound II-92, using 2,5-dichlorothiophene-3-carboxylic acid instead of 3-methylbenzo[b]furan-2-carboxylic acid in step 6 (according to ZED3264).
Yield: 66 mg, 61% (last step)
ESI-MS: 652.3/654.3 [M+H]⁺

### 151 Preparation of Compound II-161

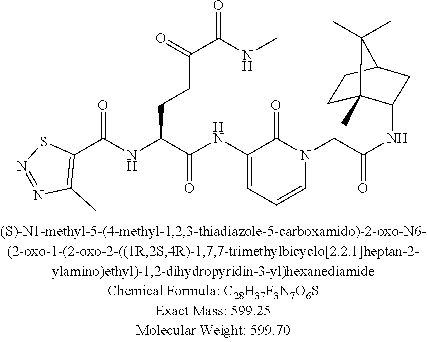 [SMILES: CNC(=O)C(=O)CC[C@H](NC(=O)c1snnc1C)C(=O)Nc1cccn(CC(=O)NC2CC3CC[C@]2(C)C3(C)C)c1=O]

The synthesis of compound II-161 was performed according to compound II-92, using 4-methyl-1,2,3-thiadiazole-5-carboxylic acid instead of 3-methylbenzo[b]furan-2-carboxylic acid in step 6 (according to ZED3264).
Yield: 41 mg, 51% (last step)
ESI-MS: 600.4 [M+H]⁺

### 152 Preparation of Compound II-162

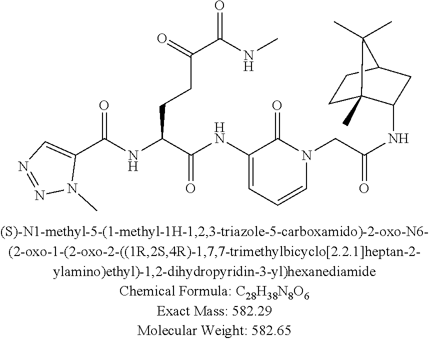 [SMILES: CNC(=O)C(=O)CC[C@H](NC(=O)c1cnnn1C)C(=O)Nc1cccn(CC(=O)NC2CC3CC[C@]2(C)C3(C)C)c1=O]

The synthesis of compound II-162 was performed according to compound II-92, using 1-methyl-1H-1,2,3-triazole-5-carboxylic acid instead of 3-methylbenzo[b]furan-2-carboxylic acid in step 6 (according to ZED3264).
Yield: 28 mg, 46% (last step)
ESI-MS: 583.5 [M+H]⁺

### 153 Preparation of Compound II-163

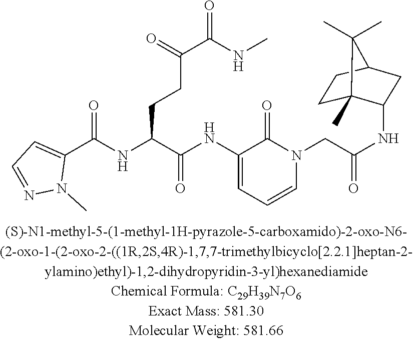 [SMILES: CNC(=O)C(=O)CC[C@H](NC(=O)c1ccnn1C)C(=O)Nc1cccn(CC(=O)NC2CC3CC[C@]2(C)C3(C)C)c1=O]

The synthesis of compound II-163 was performed according to compound II-92, using 1-methyl-1H-pyrazole-5-carboxylic acid instead of 3-methylbenzo[b]furan-2-carboxylic acid in step 6 (according to ZED3264).
Yield: 44 mg, 58% (last step)
ESI-MS: 582.5 [M+H]⁺

### 154 Preparation of Compound II-164

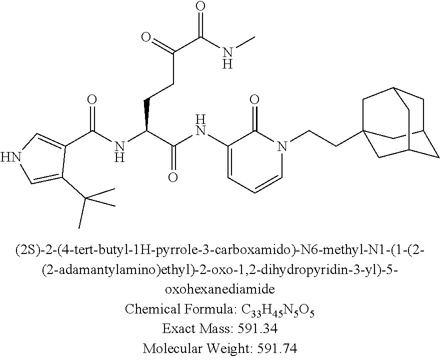 [SMILES: CNC(=O)C(=O)CC[C@H](NC(=O)c1c[nH]cc1C(C)(C)C)C(=O)Nc1cccn(CCC23CC4CC(CC(C4)C2)C3)c1=O]

The synthesis of compound II-164 was performed according to compound II-107, using 1-(2-bromoethyl) adamantane instead of 1-(bromomethyl) adamantane (according to ZED4893) and 5-tert-butyl-1H-pyrrole-3-carboxylic acid instead of 3-methyl-benzo[b]furan-2-carboxylic acid in step 6 (according to ZED3264).
Yield: 32 mg, 56% (last step)
ESI-MS: 592.5 [M+H]⁺

### 155 Preparation of Compound II-165

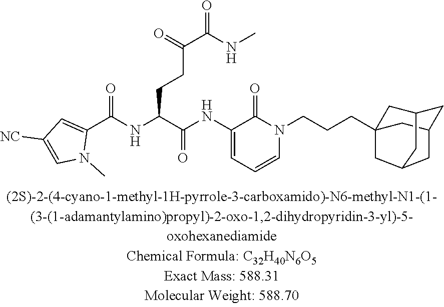 [SMILES: CNC(=O)C(=O)CC[C@H](NC(=O)c1cc(C#N)cn1C)C(=O)Nc1cccn(CCCC23CC4CC(CC(C4)C2)C3)c1=O]

The synthesis of compound II-165 was performed according to compound II-107, using 1-(3-bromopropyl) adamantane instead of 1-(bromomethyl) adamantane (according to ZED4893) and 4-cyano-1-methyl-1H-pyrrole-2-carboxylic acid instead of 3-methyl-benzo[b]furan-2-carboxylic acid in step 6 (according to ZED3264).
Yield: 27 mg, 48% (last step)
ESI-MS: 589.5 [M+H]⁺

### 156 Preparation of Compound II-166

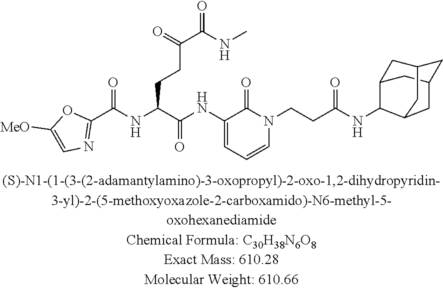 [SMILES: CNC(=O)C(=O)CC[C@H](NC(=O)c1ncc(OC)o1)C(=O)Nc1cccn(CCC(=O)NC2C3CC4CC(C3)CC2C4)c1=O]

The synthesis of compound II-166 was performed according to compound II-3, using 3-chloropropionic acid instead of chloroacetic acid (according to ZED1657) and 5-methoxyoxazole-2-carboxylic acid instead of 3-methylbenzo[b]furan-2-carboxylic acid in step 6 (according to ZED3264).
Yield: 31 mg, 59% (last step)
ESI-MS: 611.4 [M+H]⁺

### 157 Preparation of Compound II-167

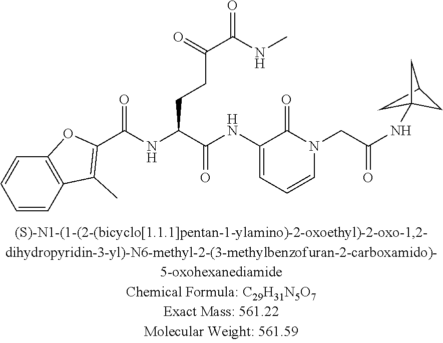 [SMILES: CNC(=O)C(=O)CC[C@H](NC(=O)c1oc2ccccc2c1C)C(=O)Nc1cccn(CC(=O)NC23CC(C2)C3)c1=O]

The synthesis of compound II-167 was performed according to compound II-3, using 1-bicyclo[1.1.1]pentylamine instead of 2-adamantanamine in step 2 (according to ZED3905).
Yield: 45 mg, 67% (last step)
ESI-MS: 562.4 [M+H]⁺

### 158 Preparation of Compound II-168

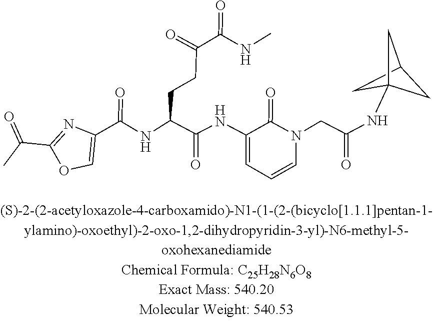 [SMILES: CNC(=O)C(=O)CC[C@H](NC(=O)c1coc(C(C)=O)n1)C(=O)Nc1cccn(CC(=O)NC23CC(C2)C3)c1=O]

The synthesis of compound II-168 was performed according to compound II-167, using 2-acetyloxazole-4-carboxylic acid instead of 3-methylbenzo[b]furan-2-carboxylic acid in step 6 (according to ZED3264).
Yield: 26 mg, 48% (last step)
ESI-MS: 541.4 [M+H]⁺

### 159 Preparation of Compound II-169

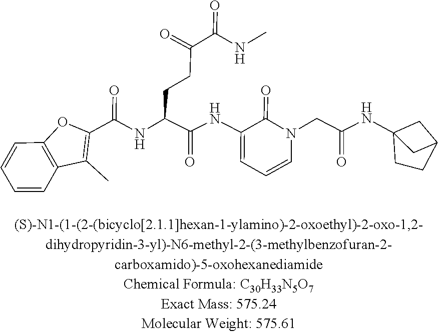 [SMILES: CNC(=O)C(=O)CC[C@H](NC(=O)c1oc2ccccc2c1C)C(=O)Nc1cccn(CC(=O)NC23CCC(C2)C3)c1=O]

The synthesis of compound II-169 was performed according to compound II-3, using bicyclo[2.1.1]hexan-1-amine instead of 2-adamantanamine in step 2 (according to ZED3905).
Yield: 33 mg, 61% (last step)
ESI-MS: 576.4 [M+H]⁺

### 160 Preparation of Compound II-170

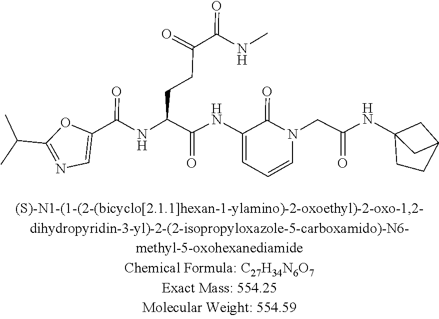 [SMILES: CNC(=O)C(=O)CC[C@H](NC(=O)c1cnc(C(C)C)o1)C(=O)Nc1cccn(CC(=O)NC23CCC(C2)C3)c1=O]

The synthesis of compound II-170 was performed according to compound II-169, using 2-isopropyloxazole-5-carboxylic acid instead of 3-methylbenzo[b]furan-2-carboxylic acid in step 6 (according to ZED3264).
Yield: 32 mg, 54% (last step)
ESI-MS: 555.4 [M+H]⁺

### 161 Preparation of Compound II-171

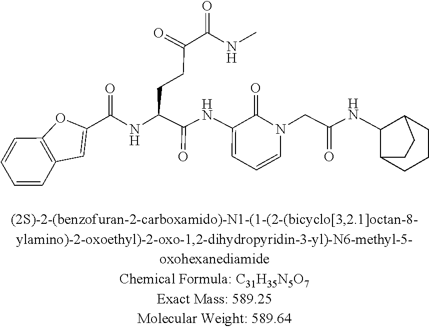 [SMILES: CNC(=O)C(=O)CC[C@H](NC(=O)c1cc2ccccc2o1)C(=O)Nc1cccn(CC(=O)NC2C3CCCC2CC3)c1=O]

The synthesis of compound II-171 was performed according to compound II-2, using bicyclo[3.2.1]octan-8-amine instead of 2-adamantanamine in step 2 (according to ZED3905).
Yield: 42 mg, 60% (last step)
ESI-MS: 590.4 [M+H]⁺

### 162 Preparation of Compound II-172

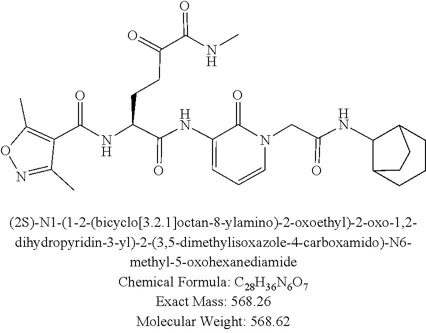 [SMILES: CNC(=O)C(=O)CC[C@H](NC(=O)c1c(C)noc1C)C(=O)Nc1cccn(CC(=O)NC2C3CCCC2CC3)c1=O]

The synthesis of compound II-172 was performed according to compound II-171, using 3,5-dimethylisoxazole-4-carboxylic acid instead of benzofuran-2-carboxylic acid in step 6 (according to ZED3264).
Yield: 35 mg, 58% (last step)
ESI-MS: 569.4 [M+H]⁺

### 163 Preparation of Compound II-173

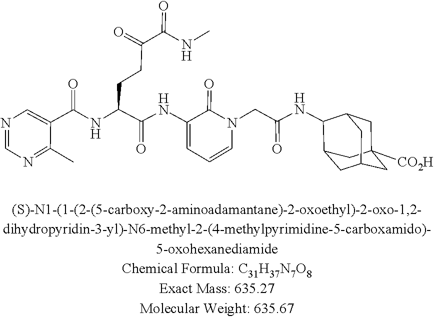 [SMILES: CNC(=O)C(=O)CC[C@H](NC(=O)c1cncnc1C)C(=O)Nc1cccn(CC(=O)NC2C3CC4CC2CC(C(=O)O)(C4)C3)c1=O]

The synthesis of compound II-173 was performed according to compound II-3, using 4-aminoadamantane-1-carboxylic acid instead of 2-adamantanamine in step 2 (according to ZED3905) and 4-methylpyrimidine-5-carboxylic acid instead of 3-methyl-benzo[b]furan-2-carboxylic acid in step 6 (according to ZED3264).
Yield: 25 mg, 49% (last step)
ESI-MS: 636.4 [M+H]⁺

### 164 Preparation of Compound II-174

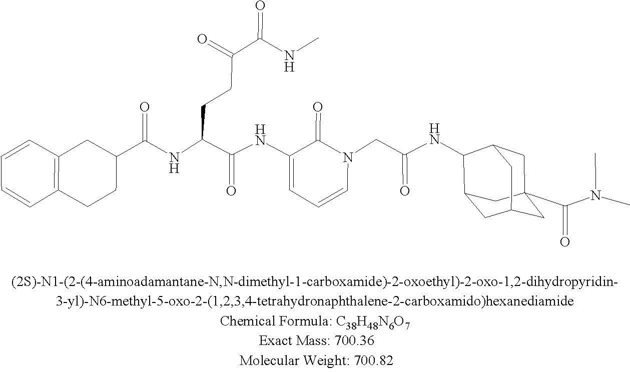 [SMILES: CNC(=O)C(=O)CC[C@H](NC(=O)C1CCc2ccccc2C1)C(=O)Nc1cccn(CC(=O)NC2C3CC4CC2CC(C(=O)N(C)C)(C4)C3)c1=O]

The synthesis of compound II-174 was performed according to compound II-3, using 4-aminoadamantane-N, N-dimethyl-1-carboxamide instead of 2-adamantanamine in step 2 (according to ZED3905) and 1,2,3,4-tetrahydronaphthalene-2-carboxylic acid instead of 3-methylbenzo[b]furan-2-carboxylic acid in step 6 (according to ZED3264).
Yield: 33 mg, 56% (last step)
ESI-MS: 701.5 [M+H]⁺

### 165 Preparation of Compound II-175

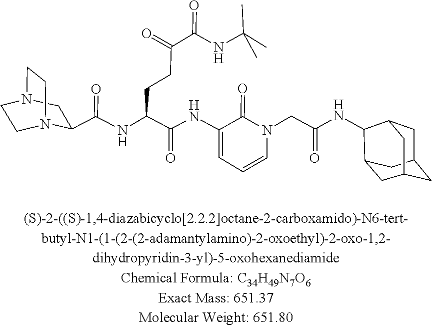 [SMILES: CC(C)(C)NC(=O)C(=O)CC[C@H](NC(=O)C1CN2CCN1CC2)C(=O)Nc1cccn(CC(=O)NC2C3CC4CC(C3)CC2C4)c1=O]

The synthesis of compound II-175 was performed according to compound II-3, using tert-butyl isocyanide instead of methyl isocyanide in step 4 (according to ZED3632) and 1,4-diazabicyclo[2.2.2]octane-2-carboxylic acid instead of 3-methylbenzo[b]furan-2-carboxylic acid in step 6 (according to ZED3264).
Yield: 39 mg, 53% (last step)
ESI-MS: 652.5 [M+H]⁺
   166 Preparation of compound II-176

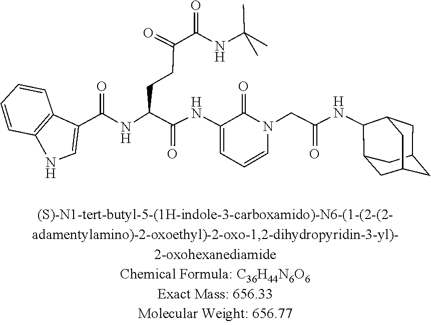 [SMILES: CC(C)(C)NC(=O)C(=O)CC[C@H](NC(=O)c1c[nH]c2ccccc12)C(=O)Nc1cccn(CC(=O)NC2C3CC4CC(C3)CC2C4)c1=O]

The synthesis of compound II-176 was performed according to compound II-3, using tert-butyl isocyanide instead of methyl isocyanide in step 4 (according to ZED3632) and 1H-indole-3-carboxylic acid instead of 3-methylbenzo[b]furan-2-carboxylic acid in step 6 (according to ZED3264).
Yield: 52 mg, 62% (last step)
ESI-MS: 657.5 [M+H]⁺

### 167 Preparation of Compound II-177

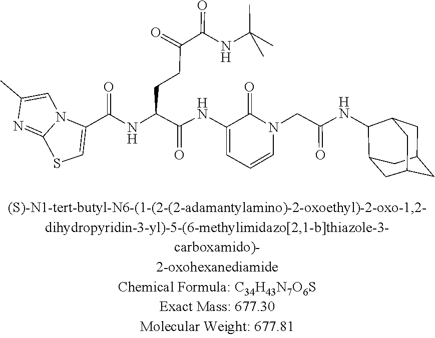 [SMILES: Cc1cn2c(C(=O)N[C@@H](CCC(=O)C(=O)NC(C)(C)C)C(=O)Nc3cccn(CC(=O)NC4C5CC6CC(C5)CC4C6)c3=O)csc2n1]

The synthesis of compound II-177 was performed according to compound II-3, using tert-butyl isocyanide instead of methyl isocyanide in step 4 (according to ZED3632) and 6-methylimidazo [2,1-b][1,3]thiazole-3-carboxylic acid instead of 3-methylbenzo[b]furan-2-carboxylic acid in step 6 (according to ZED3264).
Yield: 42 mg, 53% (last step)
ESI-MS: 678.5 [M+H]⁺

### 168 Preparation of Compound II-178

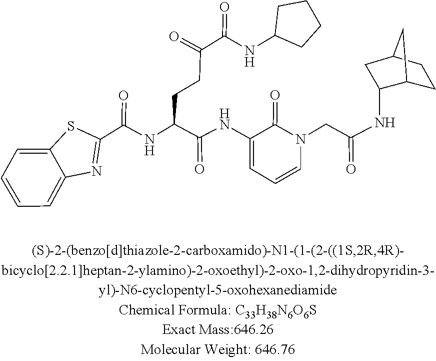 [SMILES: O=C(Cn1cccc(NC(=O)[C@H](CCC(=O)C(=O)NC2CCCC2)NC(=O)c2nc3ccccc3s2)c1=O)NC1CC2CCC1C2]

The synthesis of compound II-178 was performed according to compound II-90, using cyclopentyl isocyanide instead of methyl isocyanide in step 4 (according to ZED3632) and 1,3-benzothiazole-2-carboxylic acid instead of 3-methylbenzo[b]furan-2-carboxylic acid in step 6 (according to ZED3264).
Yield: 54 mg, 61% (last step)
ESI-MS: 647.4 [M+H]⁺

### 169 Preparation of Compound II-179

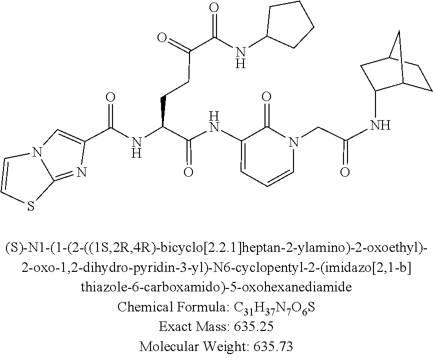 [SMILES: O=C(Cn1cccc(NC(=O)[C@H](CCC(=O)C(=O)NC2CCCC2)NC(=O)c2cn3ccsc3n2)c1=O)NC1CC2CCC1C2]

The synthesis of compound II-179 was performed according to compound II-90, using cyclopentyl isocyanide instead of methyl isocyanide in step 4 (according to ZED3632) and imidazo [2,1-b][1,3]thiazole-6-carboxylic acid instead of 3-methylbenzo[b]furan-2-carboxylic acid in step 6 (according to ZED3264).
Yield: 36 mg, 51% (last step)
ESI-MS: 636.4 [M+H]⁺
   170 Preparation of compound II-180

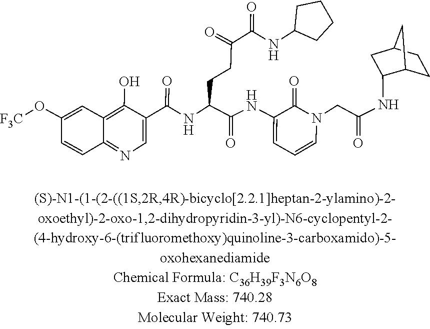 [SMILES: O=C(Cn1cccc(NC(=O)[C@H](CCC(=O)C(=O)NC2CCCC2)NC(=O)c2cnc3ccc(OC(F)(F)F)cc3c2O)c1=O)NC1CC2CCC1C2]

The synthesis of compound II-180 was performed according to compound II-90, using cyclopentyl isocyanide instead of methyl isocyanide in step 4 (according to ZED3632) and 4-hydroxy-6-(trifluoromethoxy) quinoline-3-carboxylic acid instead of 3-methyl-benzo[b]furan-2-carboxylic acid in step 6 (according to ZED3264).
Yield: 19 mg, 41% (last step)
ESI-MS: 741.5 [M+H]⁺

### 171 Preparation of Compound II-181

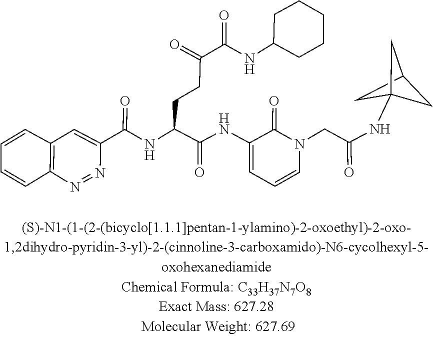 [SMILES: O=C(Cn1cccc(NC(=O)[C@H](CCC(=O)C(=O)NC2CCCCC2)NC(=O)c2cc3ccccc3nn2)c1=O)NC12CC(C1)C2]

The synthesis of compound II-181 was performed according to compound II-167, using cyclohexyl isocyanide instead of methyl isocyanide in step 4 (according to ZED3632) and 3-cinnolinecarboxylic acid instead of 3-methylbenzo[b]furan-2-carboxylic acid in step 6 (according to ZED3264).
Yield: 37 mg, 56% (last step)
ESI-MS: 628.5 [M+H]⁺

### 172 Preparation of Compound II-182

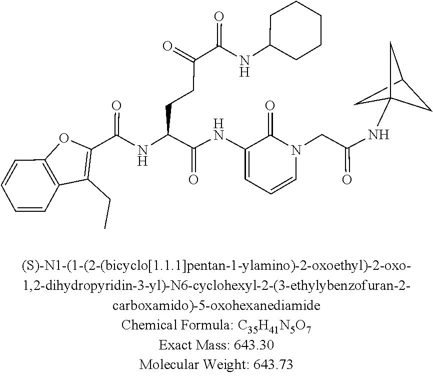 [SMILES: CCc1c(C(=O)N[C@@H](CCC(=O)C(=O)NC2CCCCC2)C(=O)Nc2cccn(CC(=O)NC34CC(C3)C4)c2=O)oc2ccccc12]

The synthesis of compound II-182 was performed according to compound II-167, using cyclohexyl isocyanide instead of methyl isocyanide in step 4 (according to ZED3632) and 3-ethylbenzofuran-2-carboxylic acid instead of 3-methylbenzo[b]furan-2-carboxylic acid in step 6 (according to ZED3264).
Yield: 53 mg, 68% (last step)
ESI-MS: 644.5 [M+H]⁺

### 173 Preparation of Compound II-183

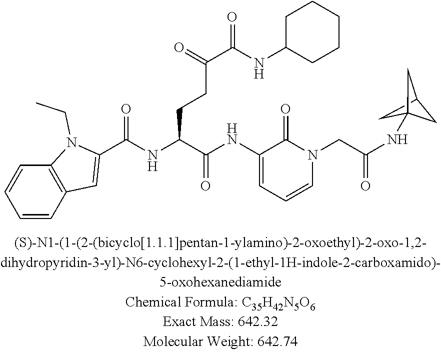 [SMILES: CCn1c(C(=O)N[C@@H](CCC(=O)C(=O)NC2CCCCC2)C(=O)Nc2cccn(CC(=O)NC34CC(C3)C4)c2=O)cc2ccccc21]

The synthesis of compound II-183 was performed according to compound II-167, using cyclohexyl isocyanide instead of methyl isocyanide in step 4 (according to ZED3632) and 1-ethyl-1H-indole-2-carboxylic acid instead of 3-methylbenzo[b]furan-2-carboxylic acid in step 6 (according to ZED3264).
Yield: 41 mg, 57% (last step)
ESI-MS: 643.5 [M+H]⁺

### 174 Preparation of Compound II-184

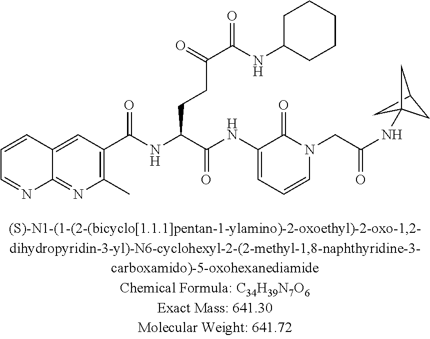 [SMILES: Cc1nc2ncccc2cc1C(=O)N[C@@H](CCC(=O)C(=O)NC1CCCCC1)C(=O)Nc1cccn(CC(=O)NC23CC(C2)C3)c1=O]

The synthesis of compound II-184 was performed according to compound II-167, using cyclohexyl isocyanide instead of methyl isocyanide in step 4 (according to ZED3632) and 2-methyl-1,8-naphthyridine-3-carboxylic acid instead of 3-methylbenzo[b]furan-2-carboxylic acid in step 6 (according to ZED3264).
Yield: 26 mg, 54% (last step)
ESI-MS: 642.5 [M+H]⁺

### 175 Preparation of Compound II-185

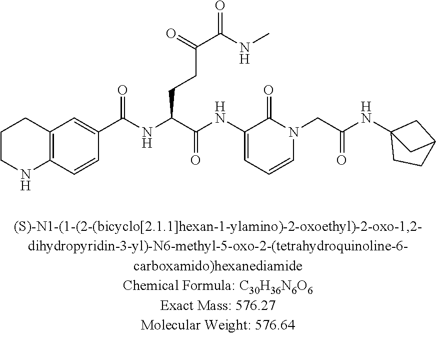 [SMILES: CNC(=O)C(=O)CC[C@H](NC(=O)c1ccc2c(c1)CCCN2)C(=O)Nc1cccn(CC(=O)NC23CCC(C2)C3)c1=O]

The synthesis of compound II-185 was performed according to compound II-169, using N-Boc-1,2,3,4-tetrahydroquinoline-6-carboxylic acid instead of 3-methylbenzo[b]furan-2-carboxylic acid in step 6 (according to ZED3264). The final product was obtained by deprotection (DCM/TFA) as described above and purified by HPLC.
Yield: 39 mg, 61% (last step)
ESI-MS: 577.4 [M+H]⁺

### 176 Preparation of Compound II-186

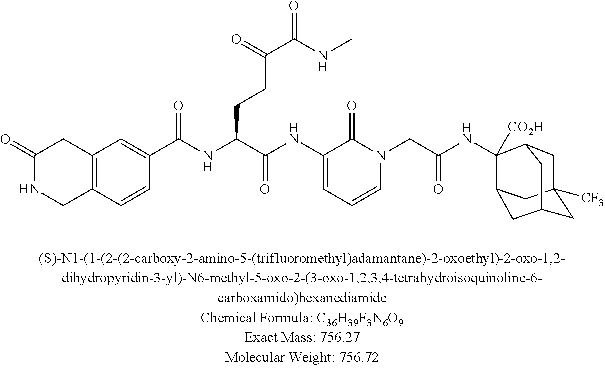 [SMILES: CNC(=O)C(=O)CC[C@H](NC(=O)c1ccc2c(c1)CC(=O)NC2)C(=O)Nc1cccn(CC(=O)NC2(C(=O)O)C3CC4CC2CC(C(F)(F)F)(C4)C3)c1=O]

The synthesis of compound II-186 was performed according to compound II-3, using 2-amino-5-(trifluoromethyl) adamantane-2-carboxylic acid instead of 2-adamantanamine in step 2 (according to ZED3905) and 3-oxo-1,2,3,4-tetrahydroisoquinoline-6-carboxylic acid instead of 3-methylbenzo[b]furan-2-carboxylic acid in step 6 (according to ZED3264).
Yield: 12 mg, 36% (last step)
ESI-MS: 757.4 [M+H]⁺

### 177 Preparation of Compound II-187

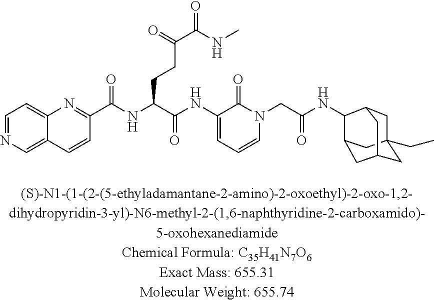 [SMILES: CCC12CC3CC(C1)C(NC(=O)Cn1cccc(NC(=O)[C@H](CCC(=O)C(=O)NC)NC(=O)c4ccc5cnccc5n4)c1=O)C(C3)C2]

The synthesis of compound II-187 was performed according to compound II-3, using 5-ethyladamantane-2-amine instead of 2-adamantanamine in step 2 (according to ZED3905) and 1,6-naphthyridine-2-carboxylic acid instead of 3-methylbenzo[b]furan-2-carboxylic acid in step 6 (according to ZED3264).
Yield: 26 mg, 51% (last step)
ESI-MS: 656.5 [M+H]⁺

178 Preparation of Compound II-188

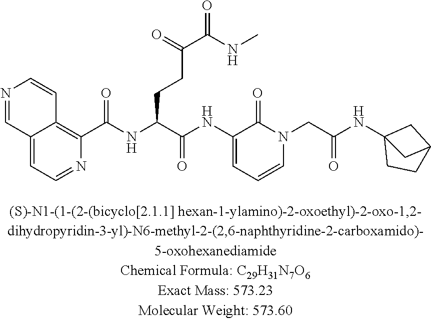 [SMILES: CNC(=O)C(=O)CC[C@H](NC(=O)c1nccc2cnccc12)C(=O)Nc1cccn(CC(=O)NC23CCC(C2)C3)c1=O]

The synthesis of compound II-188 was performed according to compound II-169, using 2,6-naphthyridine-1-carboxylic acid instead of 3-methylbenzo[b]furan-2-carboxylic acid in step 6 (according to ZED3264).
Yield: 33 mg, 56% (last step)
ESI-MS: 574.4 [M+H]⁺

### 179 Preparation of Compound II-189

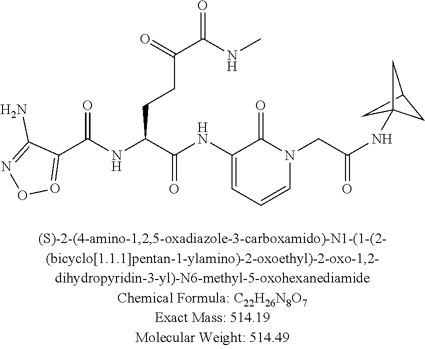

The synthesis of compound II-189 was performed according to compound II-167, using 4-Boc-amino-1,2,5-oxadiazole-3-carboxylic acid instead of 3-methylbenzo[b]furan-2-carboxylic acid in step 6 (according to ZED3264). The amine was deprotected by TFA in the final step.
Yield: 16 mg, 69% (last step)
ESI-MS: 515.3 [M+H]⁺

### 180 Preparation of Compound II-190

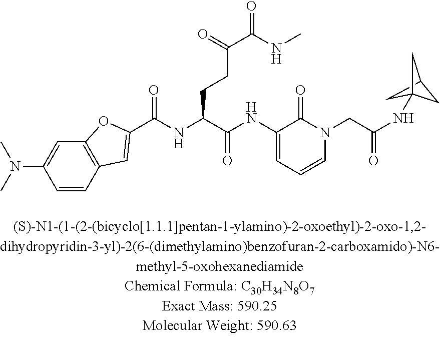 [SMILES: CNC(=O)C(=O)CC[C@H](NC(=O)c1cc2ccc(N(C)C)cc2o1)C(=O)Nc1cccn(CC(=O)NC23CC(C2)C3)c1=O]

The synthesis of compound II-190 was performed according to compound II-167, using 6-(dimethylamino) benzofuran-2-carboxylic acid instead of 3-methylbenzo[b]furan-2-carboxylic acid in step 6 (according to ZED3264).
Yield: 38 mg, 55% (last step)
ESI-MS: 591.4 [M+H]⁺

### 181 Preparation of Compound II-191

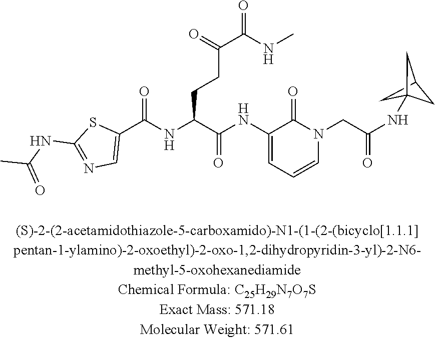 [SMILES: CNC(=O)C(=O)CC[C@H](NC(=O)c1cnc(NC(C)=O)s1)C(=O)Nc1cccn(CC(=O)NC23CC(C2)C3)c1=O]

The synthesis of compound II-191 was performed according to compound II-167, using 2-acetylamino-5-thiazolecarboxylic acid instead of 3-methylbenzo[b]furan-2-carboxylic acid in step 6 (according to ZED3264).
Yield: 21 mg, 46% (last step)
ESI-MS: 572.3 [M+H]⁺

### 182 Preparation of Compound II-192

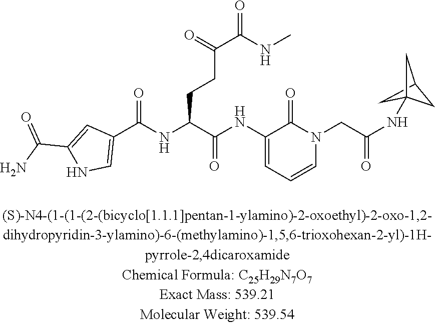 [SMILES: CNC(=O)C(=O)CC[C@H](NC(=O)c1c[nH]c(C(N)=O)c1)C(=O)Nc1cccn(CC(=O)NC23CC(C2)C3)c1=O]

The synthesis of compound II-192 was performed according to compound II-167, using 5-carbamoyl-1H-pyrrole-3-carboxylic acid instead of 3-methylbenzo[b]furan-2-carboxylic acid in step 6 (according to ZED3264).
Yield: 13 mg, 38% (last step)
ESI-MS: 540.4 [M+H]⁺

### 183 Preparation of Compound II-193

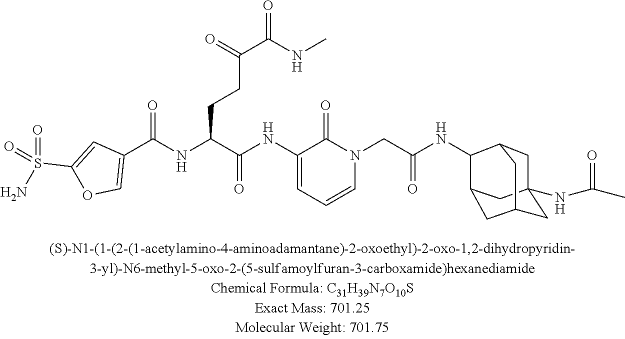 [SMILES: CNC(=O)C(=O)CC[C@H](NC(=O)c1coc(S(N)(=O)=O)c1)C(=O)Nc1cccn(CC(=O)NC2C3CC4CC2CC(NC(C)=O)(C4)C3)c1=O]

The synthesis of compound II-193 was performed according to compound II-3, using 1-acetylamino-4-aminoadamantane instead of 2-adamantanamine in step 2 (according to ZED3905) and 5-sulfamoylfuran-3-carboxylic acid instead of 3-methylbenzo[b]furan-2-carboxylic acid in step 6 (according to ZED3264).
Yield: 10 mg, 27% (last step)
ESI-MS: 702.4 [M+H]⁺

### 184 Preparation of Compound II-194

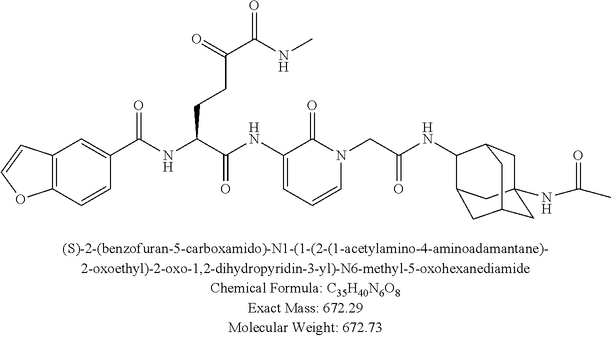 [SMILES: CNC(=O)C(=O)CC[C@H](NC(=O)c1ccc2occc2c1)C(=O)Nc1cccn(CC(=O)NC2C3CC4CC2CC(NC(C)=O)(C4)C3)c1=O]

The synthesis of compound II-194 was performed according to compound II-3, using 1-acetylamino-4-aminoadamantane instead of 2-adamantanamine in step 2 (according to ZED3905) and benzofuran-5-carboxylic acid instead of 3-methylbenzo[b]furan-2-carboxylic acid in step 6 (according to ZED3264).
Yield: 23 mg, 52% (last step)
ESI-MS: 673.5 [M+H]⁺

### 185 Preparation of Compound II-195

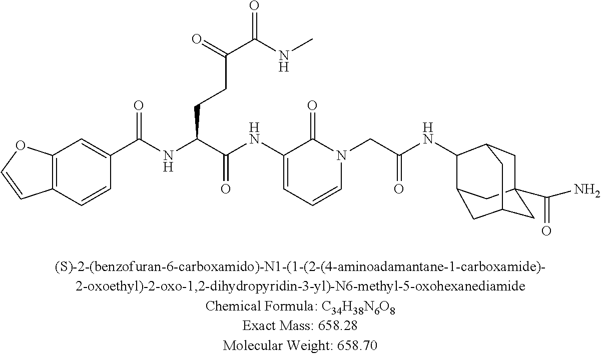 [SMILES: CNC(=O)C(=O)CC[C@H](NC(=O)c1ccc2ccoc2c1)C(=O)Nc1cccn(CC(=O)NC2C3CC4CC2CC(C(N)=O)(C4)C3)c1=O]

The synthesis of compound II-195 was performed according to compound II-3, using 4-aminoadamantane-1-carboxamide instead of 2-adamantanamine in step 2 (according to ZED3905) and benzofuran-6-carboxylic acid instead of 3-methylbenzo[b]furan-2-carboxylic acid in step 6 (according to ZED3264).
Yield: 26 mg, 57% (last step)
ESI-MS: 659.4 [M+H]⁺

### 186 Preparation of Compound II-196

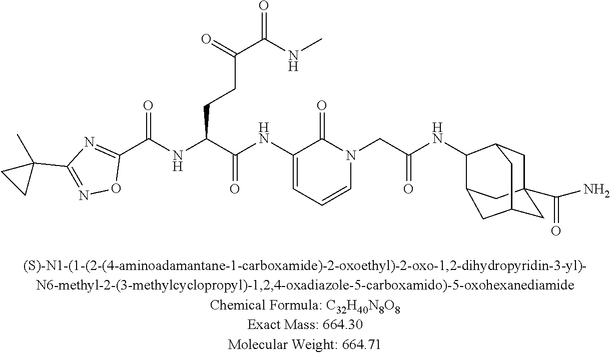 [SMILES: CNC(=O)C(=O)CC[C@H](NC(=O)c1nc(C2(C)CC2)no1)C(=O)Nc1cccn(CC(=O)NC2C3CC4CC2CC(C(N)=O)(C4)C3)c1=O]

The synthesis of compound II-196 was performed according to compound II-3, using 4-aminoadamantane-1-carboxamide instead of 2-adamantanamine in step 2 (according to ZED3905) and 3-(1-methylcyclopropyl)-1,2,4-oxadiazole-5-carboxylic acid instead of 3-methylbenzo[b]furan-2-carboxylic acid in step 6 (according to ZED3264).
Yield: 15 mg, 38% (last step)
ESI-MS: 665.5 [M+H]⁺

### 187 Preparation of Compound II-197

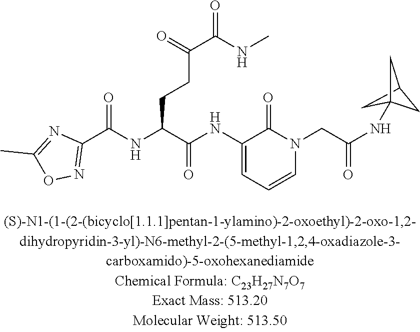 [SMILES: CNC(=O)C(=O)CC[C@H](NC(=O)c1noc(C)n1)C(=O)Nc1cccn(CC(=O)NC23CC(C2)C3)c1=O]

The synthesis of compound II-197 was performed according to compound II-167, using 5-methyl-1,2,4-oxadiazole-3-carboxylic acid instead of 3-methylbenzo[b]furan-2-carboxylic acid in step 6 (according to ZED3264).
Yield: 36 mg, 58% (last step)
ESI-MS: 514.4 [M+H]⁺

### 188 Preparation of Compound II-198

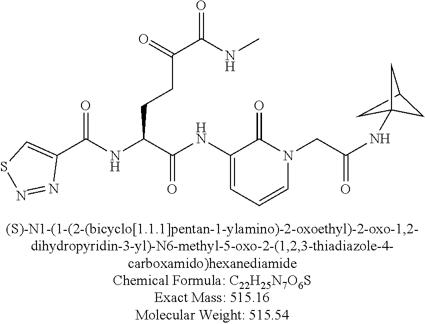 [SMILES: CNC(=O)C(=O)CC[C@H](NC(=O)c1csnn1)C(=O)Nc1cccn(CC(=O)NC23CC(C2)C3)c1=O]

The synthesis of compound II-198 was performed according to compound II-167, using 1,2,3-thiadiazole-4-carboxylic acid instead of 3-methylbenzo[b]furan-2-carboxylic acid in step 6 (according to ZED3264).
Yield: 29 mg, 48% (last step)
ESI-MS: 516.3 [M+H]⁺

### 189 Preparation of Compound II-199

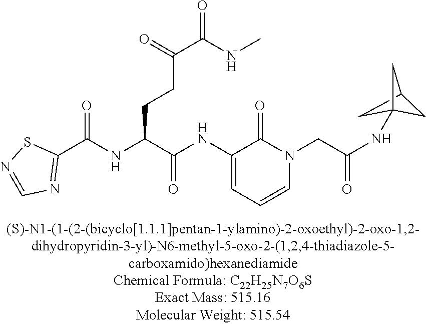 [SMILES: CNC(=O)C(=O)CC[C@H](NC(=O)c1ncns1)C(=O)Nc1cccn(CC(=O)NC23CC(C2)C3)c1=O]

The synthesis of compound II-199 was performed according to compound II-167, using 1,2,4-thiadiazole-5-carboxylic acid instead of 3-methylbenzo[b]furan-2-carboxylic acid in step 6 (according to ZED3264).
Yield: 35 mg, 56% (last step)
ESI-MS: 516.3 [M+H]⁺

### 190 Preparation of Compound II-200

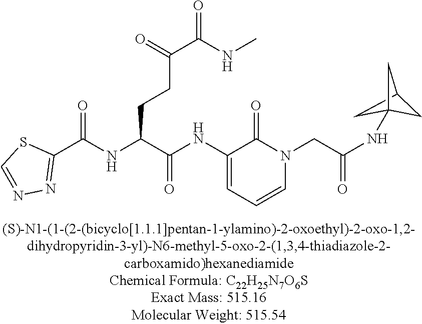 [SMILES: CNC(=O)C(=O)CC[C@H](NC(=O)c1nncs1)C(=O)Nc1cccn(CC(=O)NC23CC(C2)C3)c1=O]

The synthesis of compound II-200 was performed according to compound II-167, using 1,3,4-thiadiazole-2-carboxylic acid instead of 3-methylbenzo[b]furan-2-carboxylic acid in step 6 (according to ZED3264).
Yield: 24 mg, 41% (last step)
ESI-MS: 516.3 [M+H]⁺

### 191 Preparation of Compound II-201

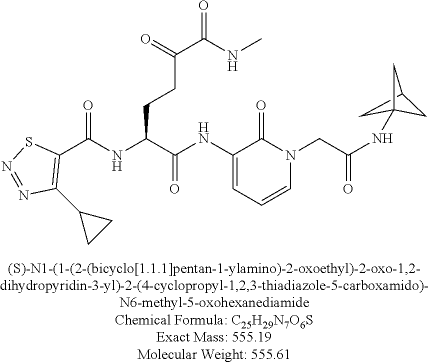 [SMILES: CNC(=O)C(=O)CC[C@H](NC(=O)c1snnc1C1CC1)C(=O)Nc1cccn(CC(=O)NC23CC(C2)C3)c1=O]

The synthesis of compound II-201 was performed according to compound II-167, using 4-cyclopropyl-[1,2,3]thiadiazole-5-carboxylic acid instead of 3-methylbenzo[b]furan-2-carboxylic acid in step 6 (according to ZED3264).
Yield: 43 mg, 67% (last step)
ESI-MS: 556.4 [M+H]⁺
   192 Preparation of compound II-202

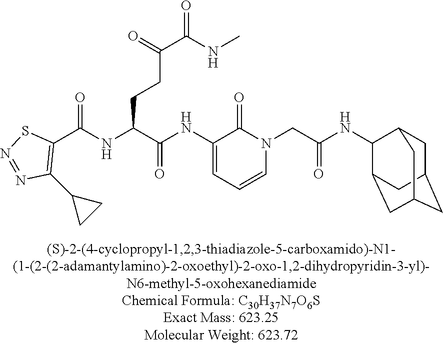 [SMILES: CNC(=O)C(=O)CC[C@H](NC(=O)c1snnc1C1CC1)C(=O)Nc1cccn(CC(=O)NC2C3CC4CC(C3)CC2C4)c1=O]

The synthesis of compound II-202 was performed according to compound II-3, using 4-cyclopropyl-[1,2,3]thiadiazole-5-carboxylic acid instead of 3-methylbenzo[b]furan-2-carboxylic acid in step 6 (according to ZED3264).
Yield: 42 mg, 56% (last step)
ESI-MS: 624.4 [M+H]⁺

### 193 Preparation of Compound II-203

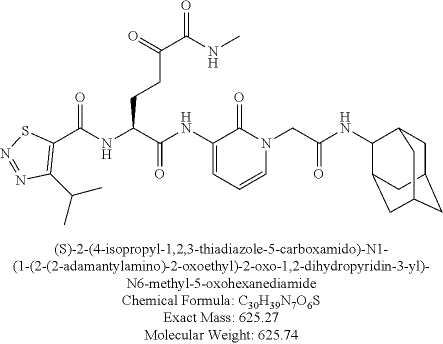 [SMILES: CNC(=O)C(=O)CC[C@H](NC(=O)c1snnc1C(C)C)C(=O)Nc1cccn(CC(=O)NC2C3CC4CC(C3)CC2C4)c1=O]

The synthesis of compound II-203 was performed according to compound II-3, using 4-(propan-2-yl)-1,2,3-thiadiazole-5-carboxylic acid instead of 3-methylbenzo[b]furan-2-carboxylic acid in step 6 (according to ZED3264).
Yield: 36 mg, 52% (last step)
ESI-MS: 626.4 [M+H]⁺

### 194 Preparation of Compound II-204

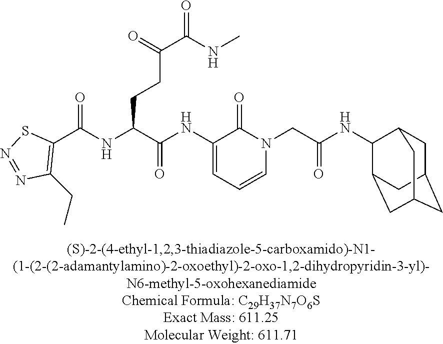 [SMILES: CCc1nnsc1C(=O)N[C@@H](CCC(=O)C(=O)NC)C(=O)Nc1cccn(CC(=O)NC2C3CC4CC(C3)CC2C4)c1=O]

The synthesis of compound II-204 was performed according to compound II-3, using 4-ethyl-1,2,3-thiadiazole-5-carboxylic acid instead of 3-methylbenzo[b]furan-2-carboxylic acid in step 6 (according to ZED3264).
Yield: 33 mg, 47% (last step)
ESI-MS: 612.4 [M+H]⁺

### 195 Preparation of Compound II-205

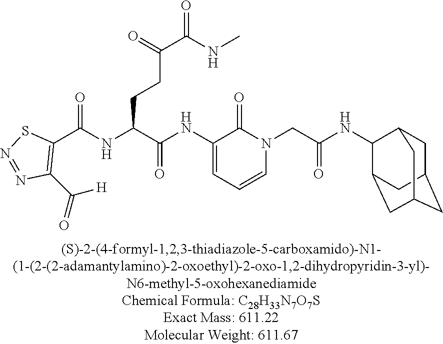 [SMILES: CNC(=O)C(=O)CC[C@H](NC(=O)c1snnc1C=O)C(=O)Nc1cccn(CC(=O)NC2C3CC4CC(C3)CC2C4)c1=O]

The synthesis of compound II-205 was performed according to compound II-3, using 4-(hydroxymethyl)-1,2,3-thiadiazole-5-carboxylic acid instead of 3-methylbenzo[b]furan-2-carboxylic acid in step 6 (according to ZED3264).
Yield: 13 mg, 32% (last step)
ESI-MS: 612.4 [M+H]⁺
   196 Preparation of compound II-206

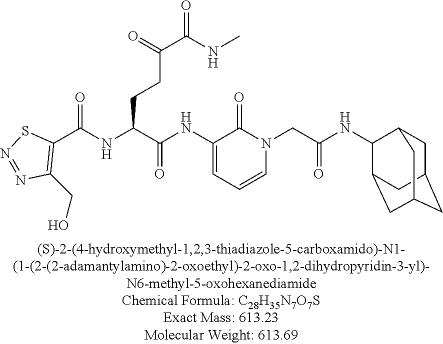 [SMILES: CNC(=O)C(=O)CC[C@H](NC(=O)c1snnc1CO)C(=O)Nc1cccn(CC(=O)NC2C3CC4CC(C3)CC2C4)c1=O]

The synthesis of compound II-206 was performed according to compound II-3, using 4-((tetrahydro-2H-pyran-2-yloxy) methyl)-1,2,3-thiadiazole-5-carboxylic acid instead of 3-methylbenzo[b]furan-2-carboxylic acid in step 6 (according to ZED3264). The tetrahydropyranyl (Thp) protecting group was cleaved by TFA in the final step.
Yield: 9 mg, 46% (last step)
ESI-MS: 614.4 [M+H]⁺

### 197 Preparation of Compound II-207

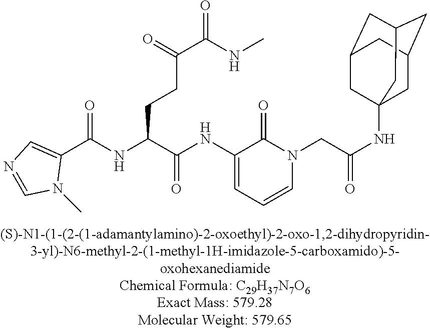 [SMILES: CNC(=O)C(=O)CC[C@H](NC(=O)c1cncn1C)C(=O)Nc1cccn(CC(=O)NC23CC4CC(CC(C4)C2)C3)c1=O]

The synthesis of compound II-207 was performed according to compound II-3, using 1-adamantanamine instead of 2-adamantanamine in step 2 (according to ZED3905) and 1-methyl-1H-imidazole-5-carboxylic acid instead of 3-methylbenzo[b]furan-2-carboxylic acid in step 6 (according to ZED3264).
Yield: 64 mg, 71% (last step)
ESI-MS: 580.4 [M+H]⁺

### 198 Preparation of Compound II-208

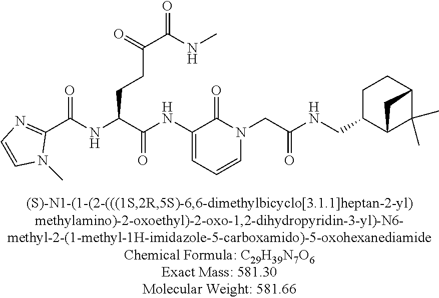 [SMILES: CNC(=O)C(=O)CC[C@H](NC(=O)c1nccn1C)C(=O)Nc1cccn(CC(=O)NC[C@@H]2CC[C@H]3C[C@@H]2C3(C)C)c1=O]

The synthesis of compound II-208 was performed according to compound II-3, using (−)-cis-myrtanylamine instead of 2-adamantanamine in step 2 (according to ZED3905) and 1-methyl-1H-imidazole-2-carboxylic acid instead of 3-methylbenzo[b]furan-2-carboxylic acid in step 6 (according to ZED3264).
Yield: 43 mg, 59% (last step)
ESI-MS: 582.5 [M+H]⁺

### 199 Preparation of Compound II-209

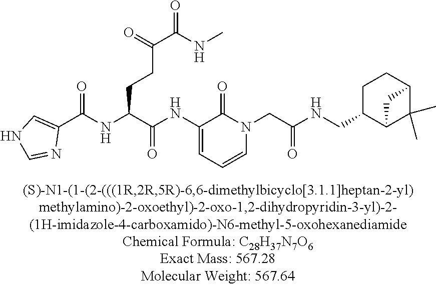 [SMILES: CNC(=O)C(=O)CC[C@H](NC(=O)c1c[nH]cn1)C(=O)Nc1cccn(CC(=O)NC[C@@H]2CC[C@@H]3C[C@H]2C3(C)C)c1=O]

The synthesis of compound II-209 was performed according to compound II-3, using (−)-cis-myrtanylamine instead of 2-adamantanamine in step 2 (according to ZED3905) and 1-methyl-1H-imidazole-2-carboxylic acid instead of 3-methylbenzo[b]furan-2-carboxylic acid in step 6 (according to ZED3264).
Yield: 31 mg, 55% (last step)
ESI-MS: 568.4 [M+H]⁺

### 200 Preparation of Compound II-210

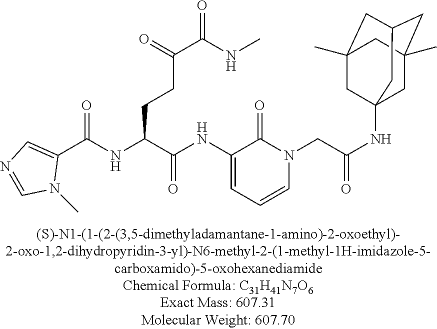 [SMILES: CNC(=O)C(=O)CC[C@H](NC(=O)c1cncn1C)C(=O)Nc1cccn(CC(=O)NC23CC4CC(C)(CC(C)(C4)C2)C3)c1=O]

The synthesis of compound II-210 was performed according to compound II-3, using 3,5-dimethyl-1-adamantanamine instead of 2-adamantanamine in step 2 (according to ZED3905) and 1-methyl-1H-imidazole-5-carboxylic acid instead of 3-methyl-benzo[b]furan-2-carboxylic acid in step 6 (according to ZED3264).
Yield: 46 mg, 66% (last step)
ESI-MS: 608.5 [M+H]⁺

### 201 Preparation of Compound II-211

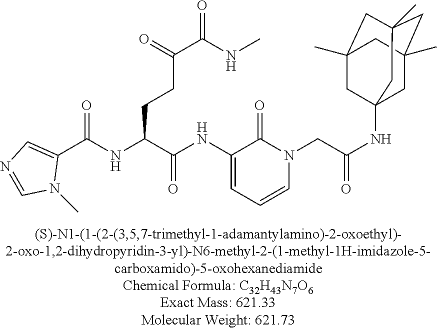 [SMILES: CNC(=O)C(=O)CC[C@H](NC(=O)c1cncn1C)C(=O)Nc1cccn(CC(=O)NC23CC4(C)CC(C)(CC(C)(C4)C2)C3)c1=O]

The synthesis of compound II-211 was performed according to compound II-3, using 3,5,7-trimethyl-1-adamantanamine instead of 2-adamantanamine in step 2 (according to ZED3905) and 1-methyl-1H-imidazole-5-carboxylic acid instead of 3-methyl-benzo[b]furan-2-carboxylic acid in step 6 (according to ZED3264).
Yield: 53 mg, 70% (last step)
ESI-MS: 622.5 [M+H]⁺

### 202 Preparation of Reference Compound 6

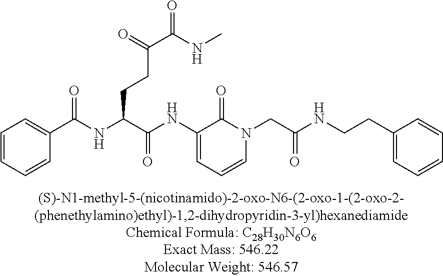 [SMILES: CNC(=O)C(=O)CC[C@H](NC(=O)c1ccccc1)C(=O)Nc1cccn(CC(=O)NCCc2ccccc2)c1=O]

The synthesis of reference compound 6 was performed according to compound II-3, using 2-phenylethylamine instead of 2-adamantanamine in step 2 (according to ZED3905) and nicotinic acid instead of 3-methylbenzo[b]furan-2-carboxylic acid in step 6 (according to ZED3264).
Yield: 89 mg, 79% (last step)
ESI-MS: 547.4 [M+H]⁺

### Biological Examples

### Example B-1. Inhibitory Effect of the Compounds According to the Invention

### Transglutaminase Assay

For the determination of potency of inhibitors against tissue transglutaminase, the incorporation of dansylcadaverine into dimethylcasein (Zedira product T036, Lorand et al., Anal Biochem, 1971, 44:221-31) was measured using recombinant human transglutaminase 2 (Zedira Product T022).

The tissue transglutaminase is diluted in buffer (50 mM Tris-HCl, 7.5 mM CaCl₂), 150 mM NaCl, pH=7.4). The final concentration of TG2 in the assay is 10 nM.

A 10 mM inhibitor stock solution is prepared in DMSO, and from this a serial 1:2-fold dilution series is prepared also in DMSO. Each of the initial dilutions is subsequently diluted 1:50-fold with buffer (50 mM Tris-HCl, 7.5 mM CaCl₂), 150 mM NaCl, pH=7.4) to yield the final working dilutions containing 2% (v/v) DMSO.

15 μl of inhibitor working dilution are added per well of a 96 well microtiter plate. As control, 15 μl of a 2% (v/v) DMSO solution prepared using the buffer mentioned above are added per well.

Immediately before starting the assay, 600 μl transglutaminase working solution are added to 11.4 ml assay buffer (50 mM Tris-HCl, 10 mM CaCl₂), 10 mM glutathione, 2.5% glycerol, 16.7 UM dansylcadaverine, 4 uM N,N-dimethylcasein, 200 mM NaCl, pH=8.0). 285 μl of this reaction mix are added per well containing the inhibitor.

Increase in fluorescence is measured using λₑₓ=330 nm and λₑₘ=500 nm at 37° C. for 30 min. A slope of the increase in fluorescence between 20 and 30 min is calculated for determination of the IC₅₀ value (inhibitor concentration at which 50% of the initial activity is blocked).

Analysis of enzymatic activity is performed by calculation of the slope of an increase in fluorescence intensity. IC₅₀ values are calculated by plotting the enzymatic activity (as percentage from control containing 2% DMSO instead of inhibitor) against the inhibitor concentration. IC₅₀ is defined as the inhibitor concentration blocking 50% of initial enzyme activity.

The inhibitory activity of the inventive compounds in regard to tissue transglutaminase (TG2) is shown in the following table 1 using IC₅₀-values.

TABLE 1
efficacy of reversible TG2 inhibitors
A: IC₅₀ < 40 nM,
B: 40 nM ≤ IC₅₀ < 400 nM,
C: 400 nM ≤ IC₅₀ < 2,500 nM,
D: 2,500 nM ≤ IC₅₀ < 10,000 nM

| | IC₅₀ |
|---|---|
| Compound | TG2 [nM] |
| II-2 | A |
| II-3 | A |
| II-4 | A |
| II-5 | A |
| II-6 | A |
| II-7 | A |
| II-8 | A |
| II-9 | A |
| II-10 | A |
| II-11 | A |
| II-12 | B |
| II-13 | A |
| II-14 | A |
| II-15 | A |
| II-16 | A |
| II-17 | B |
| II-18 | B |
| II-19 | B |
| II-20 | C |
| II-21 | A |
| II-22 | A |
| II-23 | B |
| II-24 | B |
| II-25 | B |
| II-26 | B |
| II-27 | B |
| II-28 | A |
| II-29 | A |
| II-30 | A |
| II-31 | B |
| II-32 | B |
| II-33 | B |
| II-34 | B |
| II-35 | B |
| II-36 | B |
| II-37 | B |
| II-38 | C |
| II-39 | B |
| II-40 | A |
| II-41 | B |
| II-42 | B |
| II-43 | C |
| II-44 | A |
| II-45 | A |
| II-46 | B |
| II-47 | A |
| II-48 | A |
| II-49 | A |
| II-50 | A |
| II-51 | B |
| II-52 | C |
| II-53 | B |
| II-54 | B |
| II-55 | B |
| II-56 | B |
| II-58 | B |
| II-59 | B |
| II-60 | B |
| II-61 | B |
| II-62 | A |
| II-63 | A |
| II-64 | B |
| II-65 | B |
| II-66 | C |
| II-67 | C |
| II-68 | B |
| II-69 | A |
| II-70 | A |
| II-71 | A |
| II-72 | A |
| II-73 | B |
| II-74 | C |
| II-87 | B |
| II-88 | B |
| II-90 | B |
| II-92 | B |
| II-94 | A |
| II-95 | B |
| II-96 | B |
| II-97 | B |
| II-98 | B |
| II-99 | B |
| II-100 | B |
| II-101 | B |
| II-103 | B |
| II-104 | B |
| II-105 | B |
| II-107 | B |
| II-108 | B |
| II-109 | B |
| II-110 | B |
| II-111 | A |
| II-112 | A |
| II-113 | B |
| II-114 | B |
| II-115 | B |
| II-116 | B |
| Ref. 6 | D |
| II-117 | B |
| II-118 | A |
| II-119 | B |
| II-120 | B |
| II-121 | B |
| II-122 | C |
| II-123 | C |
| II-124 | B |
| II-125 | B |
| II-126 | B |
| II-127 | B |
| II-128 | B |
| II-129 | B |
| II-130 | B |
| II-131 | B |
| II-132 | C |
| II-133 | C |
| II-134 | C |
| II-135 | B |
| II-136 | B |
| II-137 | B |
| II-138 | C |
| II-139 | C |
| II-140 | B |
| II-141 | B |
| II-142 | B |
| II-143 | C |
| II-144 | C |
| II-145 | B |
| II-146 | A |
| II-147 | A |
| II-148 | A |
| II-149 | A |
| II-150 | B |
| II-151 | B |
| II-152 | B |
| II-153 | C |
| II-154 | B |
| II-155 | A |
| II-156 | B |
| II-157 | B |
| II-158 | B |
| II-159 | B |
| II-160 | B |
| II-161 | B |
| II-162 | B |
| II-163 | B |
| II-164 | C |
| II-165 | C |
| II-166 | C |
| II-167 | A |
| II-168 | B |
| II-169 | A |
| II-170 | B |
| II-171 | A |
| II-172 | B |
| II-173 | B |
| II-174 | B |
| II-175 | C |
| II-176 | C |
| II-177 | C |
| II-178 | B |
| II-179 | C |
| II-180 | C |
| II-181 | C |
| II-182 | B |
| II-183 | B |
| II-184 | C |
| II-185 | C |
| II-186 | C |
| II-187 | C |
| II-188 | C |
| II-189 | C |
| II-190 | C |
| II-191 | C |
| II-192 | C |
| II-193 | C |
| II-194 | B |
| II-195 | B |
| II-196 | C |
| II-197 | C |
| II-198 | B |
| II-199 | B |
| II-200 | B |
| II-201 | B |
| II-202 | B |
| II-203 | B |
| II-204 | B |
| II-205 | B |
| II-206 | B |
| II-207 | B |
| II-208 | C |
| II-209 | C |
| II-210 | B |
| II-211 | B |
| Ref. 1 | C |
| Ref. 2 | B |
| Ref. 3 | D |

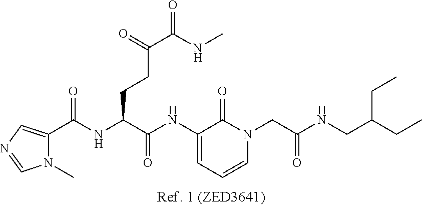 [SMILES: CCC(CC)CNC(=O)Cn1cccc(NC(=O)[C@H](CCC(=O)C(=O)NC)NC(=O)c2cncn2C)c1=O]

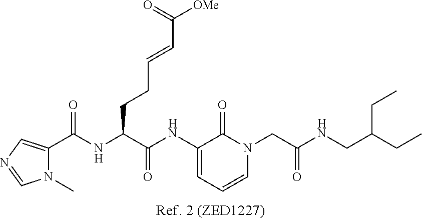 [SMILES: CCC(CC)CNC(=O)Cn1cccc(NC(=O)[C@H](CC/C=C/C(=O)OC)NC(=O)c2cncn2C)c1=O]

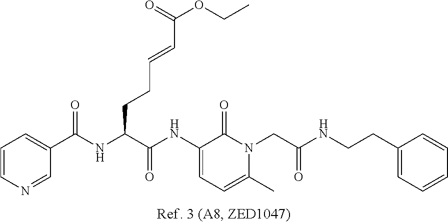 [SMILES: CCOC(=O)/C=C/CC[C@H](NC(=O)c1cccnc1)C(=O)Nc1ccc(C)n(CC(=O)NCCc2ccccc2)c1=O]

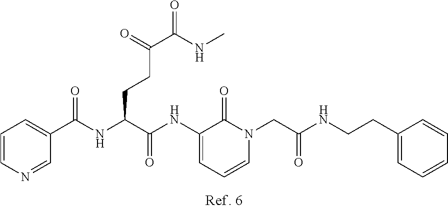 [SMILES: CNC(=O)C(=O)CC[C@H](NC(=O)c1cccnc1)C(=O)Nc1cccn(CC(=O)NCCc2ccccc2)c1=O]

### Example B-2. logD Values of the Inventive Compounds

In order to classify the inventive compounds according to their lipophilicity, LogD values (distribution coefficient) were determined by means of the well-established shake flask method, measuring the partition of a compound between an octanol and phosphate-buffered saline (PBS, pH 7.4) by HPLC.

The LogD is pH dependent and is a “predictor” for in-vivo properties. LogD combines lipophilicity (intrinsic structural property of the molecule, logP) and ionizability (pKa). Compounds with a moderate lipophilicity (LogD values from 0 to 3) are usually advantaged for oral absorption, being in balance between solubility and permeability. However, sophisticated formulation of a compound might improve oral bioavailability for highly lipophilic compounds.

TABLE 2
logD values of
reversible TG2 inhibitors

| Compound | logD |
|---|---|
| II-2 | B |
| II-3 | B |
| II-4 | B |
| II-5 | C |
| II-6 | C |
| II-7 | C |
| II-8 | C |
| II-9 | B |
| II-10 | C |
| II-11 | B |
| II-12 | C |
| II-13 | B |
| II-14 | B |
| II-15 | B |
| II-16 | B |
| II-17 | B |
| II-18 | B |
| II-19 | C |
| II-20 | B |
| II-21 | C |
| II-22 | C |
| II-23 | C |
| II-24 | B |
| II-25 | B |
| II-26 | B |
| II-27 | B |
| II-28 | B |
| II-29 | C |
| II-30 | C |
| II-31 | B |
| II-32 | B |
| II-33 | B |
| II-34 | B |
| II-35 | B |
| II-36 | B |
| II-37 | B |
| II-38 | C |
| II-39 | B |
| II-40 | C |
| II-41 | C |
| II-42 | B |
| II-43 | B |
| II-44 | C |
| II-45 | C |
| II-46 | C |
| II-47 | B |
| II-48 | C |
| II-49 | C |
| II-50 | A |
| II-51 | A |
| II-52 | A |
| II-53 | B |
| II-54 | B |
| II-55 | A |
| II-56 | B |
| II-58 | B |
| II-59 | B |
| II-60 | A |
| II-61 | B |
| II-62 | B |
| II-63 | B |
| II-64 | B |
| II-65 | B |
| II-66 | B |
| II-67 | C |
| II-68 | A |
| II-69 | B |
| II-70 | B |
| II-71 | B |
| II-72 | C |
| II-73 | B |
| II-74 | B |
| II-87 | C |
| II-88 | C |
| II-90 | B |
| II-92 | C |
| II-94 | B |
| II-95 | B |
| II-96 | B |
| II-97 | B |
| II-98 | B |
| II-99 | C |
| II-100 | C |
| II-101 | C |
| II-103 | B |
| II-104 | B |
| II-105 | C |
| II-107 | C |
| II-108 | B |
| II-109 | C |
| II-110 | C |
| II-111 | B |
| II-112 | B |
| II-113 | A |
| II-114 | A |
| II-115 | C |
| II-116 | C |
| II-117 | C |
| II-118 | B |
| II-119 | C |
| II-120 | C |
| II-121 | C |
| II-122 | C |
| II-123 | C |
| II-124 | B |
| II-125 | B |
| II-126 | A |
| II-127 | A |
| II-128 | B |
| II-129 | B |
| II-130 | A |
| II-131 | A |
| II-132 | A |
| II-133 | A |
| II-134 | A |
| II-135 | A |
| II-136 | B |
| II-137 | C |
| II-138 | A |
| II-139 | A |
| II-140 | A |
| II-141 | A |
| II-142 | B |
| II-143 | A |
| II-144 | A |
| II-145 | A |
| II-146 | A |
| II-147 | A |
| II-148 | A |
| II-149 | A |
| II-150 | B |
| II-151 | B |
| II-152 | A |
| II-153 | A |
| II-154 | B |
| II-155 | B |
| II-156 | A |
| II-157 | A |
| II-158 | A |
| II-159 | B |
| II-160 | B |
| II-161 | A |
| II-162 | A |
| II-163 | A |
| II-164 | C |
| II-165 | C |
| II-166 | B |
| II-167 | A |
| II-168 | A |
| II-169 | A |
| II-170 | A |
| II-171 | A |
| II-172 | A |
| II-173 | A |
| II-174 | A |
| II-175 | B |
| II-176 | C |
| II-177 | C |
| II-178 | C |
| II-179 | B |
| II-180 | C |
| II-181 | B |
| II-182 | B |
| II-183 | B |
| II-184 | B |
| II-185 | A |
| II-186 | A |
| II-187 | C |
| II-188 | A |
| II-189 | A |
| II-190 | A |
| II-191 | A |
| II-192 | A |
| II-193 | A |
| II-194 | A |
| II-195 | A |
| II-196 | A |
| II-197 | A |
| II-198 | A |
| II-199 | A |
| II-200 | A |
| II-201 | A |
| II-202 | A |
| II-203 | B |
| II-204 | B |
| II-205 | A |
| II-206 | A |
| II-207 | A |
| II-208 | B |
| II-209 | B |
| II-210 | B |
| II-211 | B |
| Ref. 1 | A |
| Ref. 2 | B |
| Ref. 3 | B |
| Ref. 6 | A |

A: logD < 1,
B: 1 ≤ logD < 3,
C: 3 ≤ logD < 5

### Example B-3. Caco-2 Permeability Assay of the Inventive Compounds

Permeability coefficients (Pₐₚₚ values) were obtained from Caco-2 barrier studies predicting oral/intestinal bioavailability of the tested compounds. The assays were performed by using CacoReady™ ready-to-use kits from ReadyCell according to the manufacturers protocol.

It is considered that compounds bearing Pₐₚₚ values above 1×10⁻⁶ cm/s are classified as permeable whereas compounds bearing Pₐₚₚ values below 1×10⁻⁶ cm/s are classified as not permeable.

TABLE 3
Caco2 permeability assay of
reversible TG2 inhibitors

| | Pₐₚₚ [cm/s] |
|---|---|
| Compound | ×10⁻⁶ |
| II-2 | B |
| II-3 | B |
| II-4 | B |
| II-5 | B |
| II-6 | B |
| II-7 | B |
| II-8 | B |
| II-9 | B |
| II-10 | A |
| II-11 | B |
| II-12 | A |
| II-13 | B |
| II-14 | B |
| II-15 | B |
| II-16 | B |
| II-17 | B |
| II-18 | A |
| II-19 | A |
| II-20 | B |
| II-21 | B |
| II-22 | B |
| II-23 | B |
| II-24 | B |
| II-25 | B |
| II-26 | B |
| II-27 | B |
| II-28 | B |
| II-29 | B |
| II-30 | B |
| II-31 | B |
| II-32 | B |
| II-33 | C |
| II-34 | C |
| II-35 | B |
| II-36 | B |
| II-37 | B |
| II-38 | B |
| II-39 | B |
| II-40 | A |
| II-41 | B |
| II-42 | B |
| II-43 | B |
| II-44 | B |
| II-45 | B |
| II-46 | A |
| II-47 | B |
| II-48 | B |
| II-49 | B |
| II-50 | A |
| II-51 | B |
| II-52 | A |
| II-53 | A |
| II-54 | A |
| II-55 | A |
| II-56 | A |
| II-58 | A |
| II-59 | A |
| II-60 | A |
| II-61 | A |
| II-62 | B |
| II-63 | A |
| II-64 | A |
| II-65 | A |
| II-66 | A |
| II-67 | B |
| II-68 | A |
| II-69 | B |
| II-70 | B |
| II-71 | B |
| II-72 | A |
| II-73 | A |
| II-74 | A |
| II-87 | B |
| II-88 | A |
| II-90 | A |
| II-92 | B |
| II-94 | B |
| II-95 | B |
| II-96 | B |
| II-97 | B |
| II-98 | B |
| II-99 | B |
| II-100 | B |
| II-101 | B |
| II-103 | B |
| II-104 | B |
| II-105 | B |
| II-107 | B |
| II-108 | A |
| II-109 | B |
| II-110 | B |
| II-111 | A |
| II-112 | A |
| II-113 | A |
| II-114 | A |
| II-115 | B |
| II-116 | B |
| II-117 | B |
| II-118 | B |
| II-119 | B |
| II-120 | B |
| II-121 | B |
| II-122 | B |
| II-123 | B |
| II-124 | B |
| II-125 | B |
| II-126 | B |
| II-127 | A |
| II-128 | B |
| II-129 | A |
| II-130 | A |
| II-131 | A |
| II-132 | A |
| II-133 | B |
| II-134 | A |
| II-135 | B |
| II-136 | B |
| II-137 | B |
| II-138 | A |
| II-139 | A |
| II-140 | A |
| II-141 | A |
| II-142 | A |
| II-143 | A |
| II-144 | A |
| II-145 | A |
| II-146 | B |
| II-147 | B |
| II-148 | B |
| II-149 | B |
| II-150 | B |
| II-151 | B |
| II-152 | A |
| II-153 | A |
| II-154 | B |
| II-155 | B |
| II-156 | A |
| II-157 | A |
| II-158 | A |
| II-159 | B |
| II-160 | B |
| II-161 | A |
| II-162 | A |
| II-163 | A |
| II-164 | B |
| II-165 | B |
| II-166 | B |
| II-167 | B |
| II-168 | A |
| II-169 | B |
| II-170 | B |
| II-171 | B |
| II-172 | B |
| II-173 | A |
| II-174 | A |
| II-175 | A |
| II-176 | B |
| II-177 | B |
| II-178 | B |
| II-179 | B |
| II-180 | A |
| II-181 | A |
| II-182 | B |
| II-183 | B |
| II-184 | A |
| II-185 | A |
| II-186 | A |
| II-187 | B |
| II-188 | A |
| II-189 | A |
| II-190 | A |
| II-191 | A |
| II-192 | A |
| II-193 | A |
| II-194 | A |
| II-195 | A |
| II-196 | A |
| II-197 | A |
| II-198 | A |
| II-199 | A |
| II-200 | A |
| II-201 | A |
| II-202 | A |
| II-203 | A |
| II-204 | A |
| II-205 | A |
| II-206 | A |
| II-207 | A |
| II-208 | A |
| II-209 | A |
| II-210 | A |
| II-211 | A |
| Ref. 1 | A |
| Ref. 2 | A |
| Ref. 3 | A |
| Ref. 6 | A |

A: Pₐₚₚ < 1 × 10⁻⁶ cm/s,
B: Pₐₚₚ ≥ 1 × 10⁻⁶ cm/s ≤ Pₐₚₚ < 10 × 10⁻⁶ cm/s
C: Pₐₚₚ ≥ 10 × 10⁻⁶ cm/s

### Bioavailability Studies

Presumed from promising Pₐₚₚ values (permeability coefficients, see below), the 5 inventors had proven the oral bioavailability of the inhibitors of the present application by the representative compounds II-3, II-15, and II-28. For this selected set of representative compounds, pharmacokinetic profiles were determined in male C57BL/6 mice (N=3, each). Briefly, the compounds were administered as single-dose soluble, oral formulation [20 mg/ml in PBS/(2—Hydroxypropyl)-β-cyclodextrin formulation] at 200 mg/kg. Plasma samples were taken at (0, 0.25, 0.5, 1, 2, 4, and 6 hours) and analyzed by LC-MS to determine the concentration of the representative compounds.

The calculated pharmacokinetic parameters are summarized in the table.

| | Cₘₐₓ | Cₘₐₓ | AUCt | Kₑₗ | MRT | t_{1/2} | | | |
|---|---|---|---|---|---|---|---|---|---|
| | [ng/ml] | [nM] | [ng/ml*h] | [1/h] | [h] | [h] | CL/F [L/h/kg] | Vd/F [L/h/kg] | R² |
| II-3 | 13,594 | 21,588 | 12,023 | 1.0 | 1.0 | 0.7 | 16.6 | 16.3 | 0.87 |
| II-28 | 15,143 | 23,277 | 14,468 | 0.6 | 1.6 | 1.1 | 14.8 | 23.8 | 0.98 |
| II-15 | 32,322 | 48,626 | 36,878 | 0.9 | 1.1 | 0.9 | 6.1 | 7.5 | 0.93 |

Cₘₐₓ: maximum plasma concentration.
AUCt: area under the plasma concentration time curve from administration to last observed concentration at time t measured by trapezoidal rules.
Kₑₗ: is estimated by the linear regression of the logarithm of the terminal concentration as a function of time. Point used to calculate the Kel are selected the ‘Best Fit’ option of Winnonlin.
MRT: mean residence time.
t_{1/2}: is calculated by application of the equation In2/Kₑₗ.
CL/F: apparent plasma clearance calculated as follow: Dose/AUCinf.
V_{d}/F (L/kg): apparent volume of distribution following administration. The parameter are
calculated as V_{d}/F = (CL/F)/Kₑₗ.
R²: correlation coefficient.

| | | | Pₐₚₚ [cm/s] |
|---|---|---|---|
| | IC₅₀ [nM] | IC₉₀ [nM] | ×10⁻⁶ |
| II-3 | 23 | 180 | 8.4 |
| II-28 | 24 | 240 | 7.4 |
| II-15 | 35 | 375 | 5.9 |

The corresponding PK profiles reveal that the plasma levels for all representative compounds exceed the IC₉₀ for 3.5 hours and the IC₅₀ during the whole study (6 hours). In summary, the high Cₘₐₓ values observed in the PK-studies exceed the IC₉₀ levels >100-times. We therefore expect to occupy all active TG2 accessible.

Additionally, in a multiple-dosing PK study, II-3 was orally administered to 3 mice with 200 mg/kg doses (dose volume 10 mL/kg) twice daily (12 h intervals). After the animals were sacrificed, the liver and lung were removed. The homogenates of the respective tissues were analyzed by LC-MS to determine the concentration of the compound. The tissue concentration in lung and liver after the eighth dose (four days) was 6,800 and 10,400 ng/g, respectively, showing that the compound reaches the tissue at pharmacological active concentration.

1. A compound of the general formula (I): 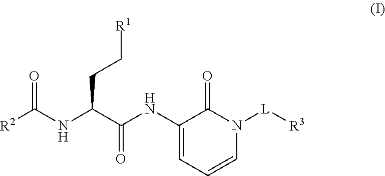
wherein
L represents -L¹- or -L¹-L²-; preferably -L¹-L²-;
L¹ represents —CH₂—, —CH₂CH₂, —CH₂CH₂CH₂, —CH₂CO—, or —CH₂CH₂CO—;
L² represents a bond, —NR^{N1}, —NR^{N1}CH₂—, —NR^{N1}CH₂CH₂—, or —NR^{N1}CH(CH₃)—;
R¹ represents 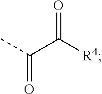
R² represents 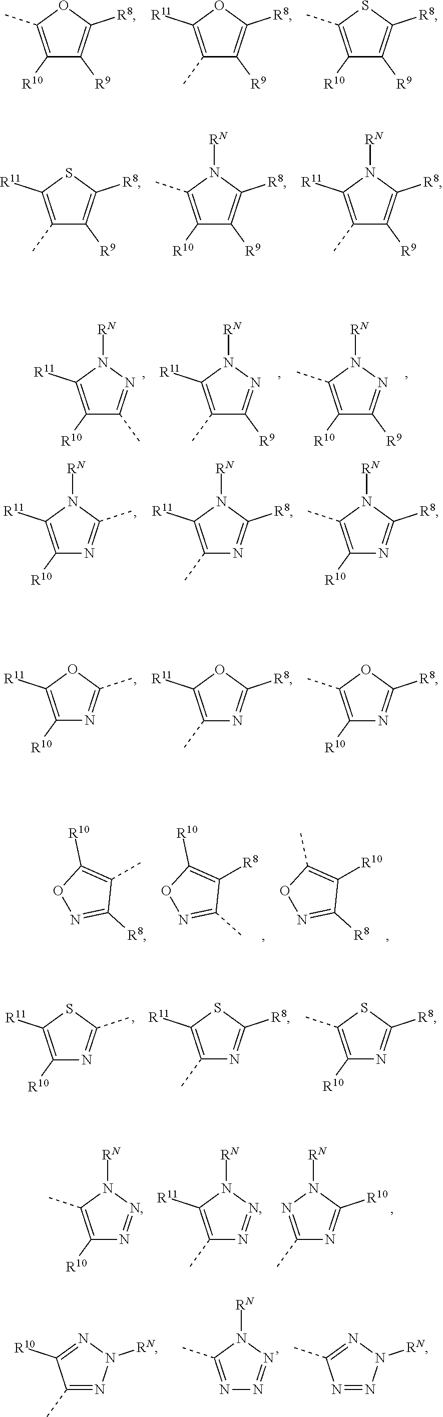 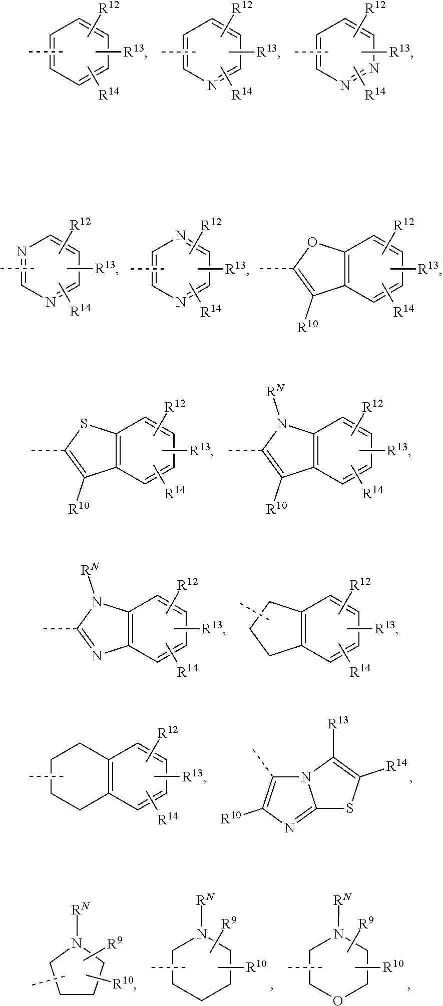 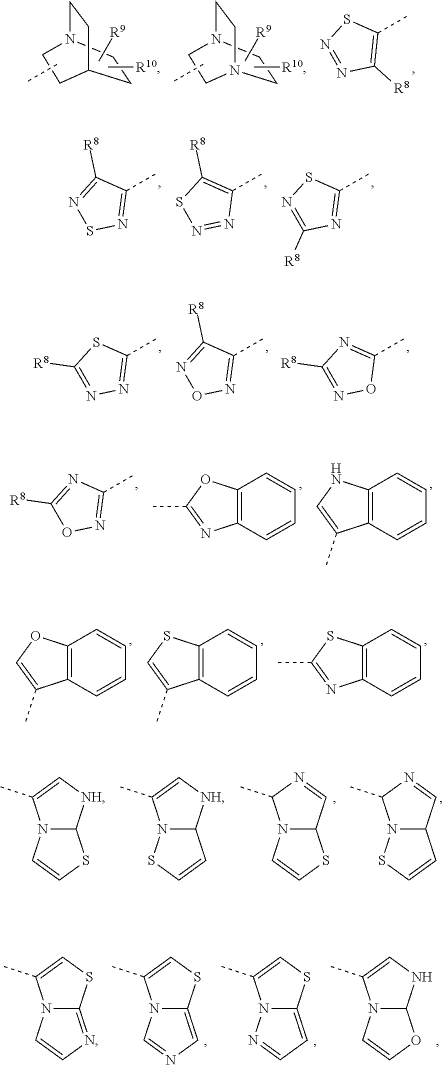 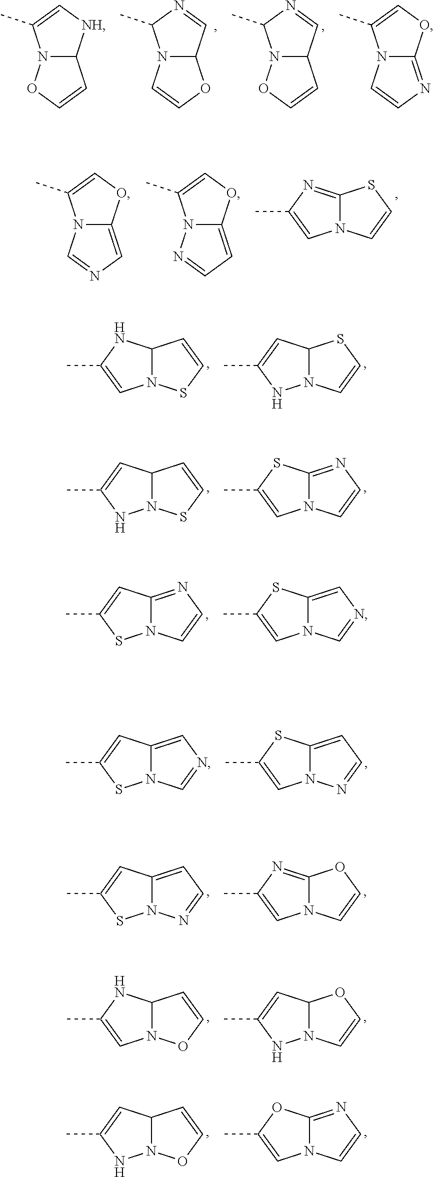 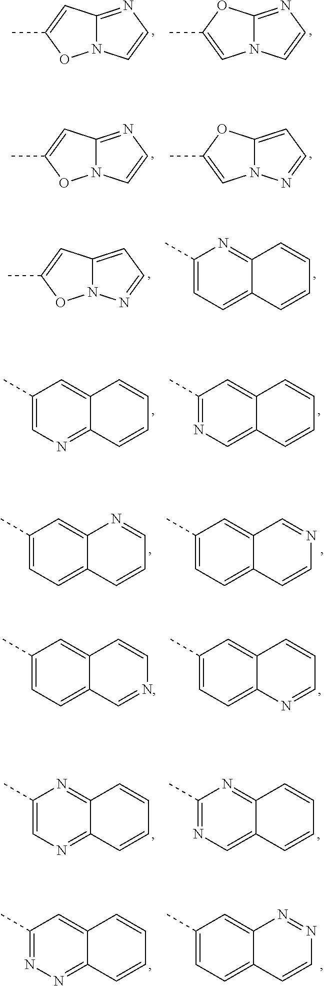 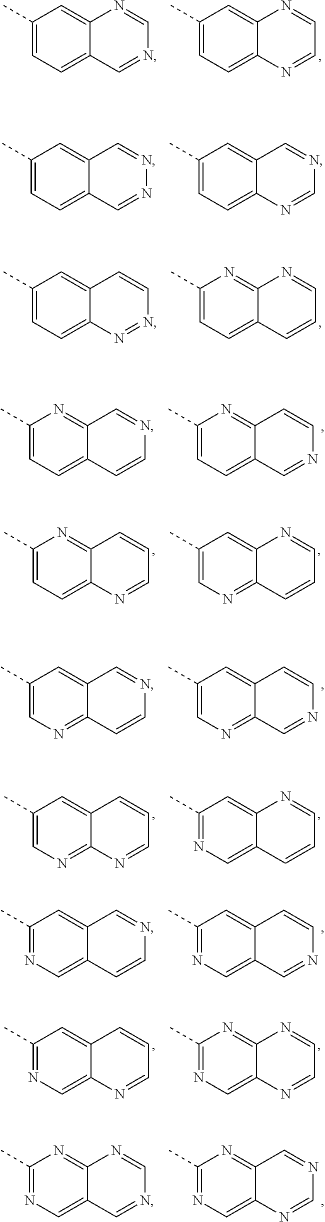 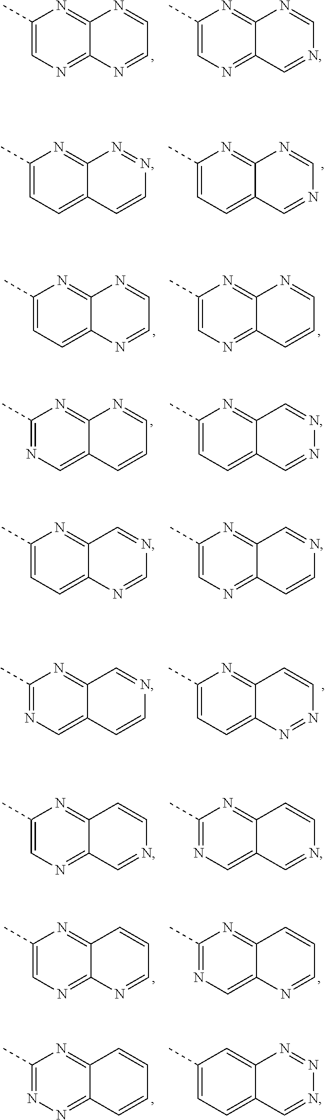 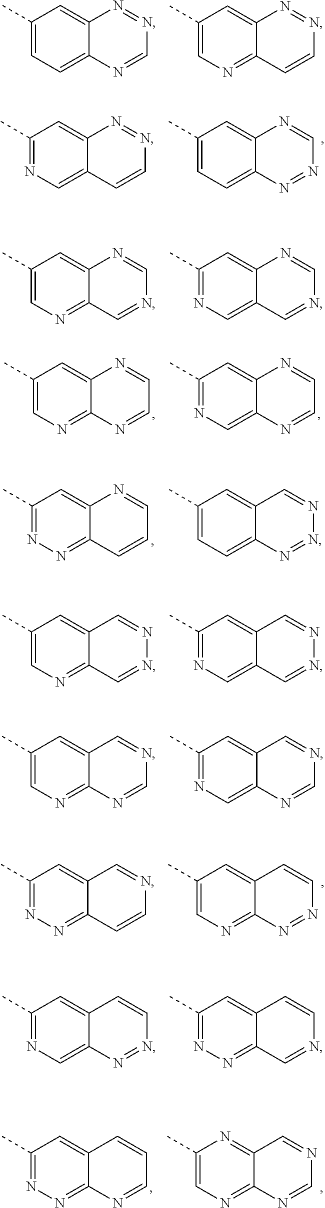 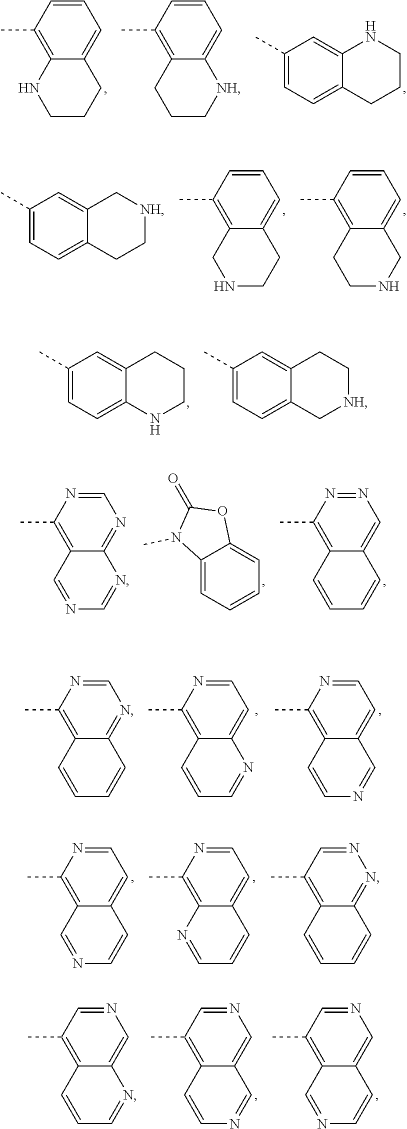 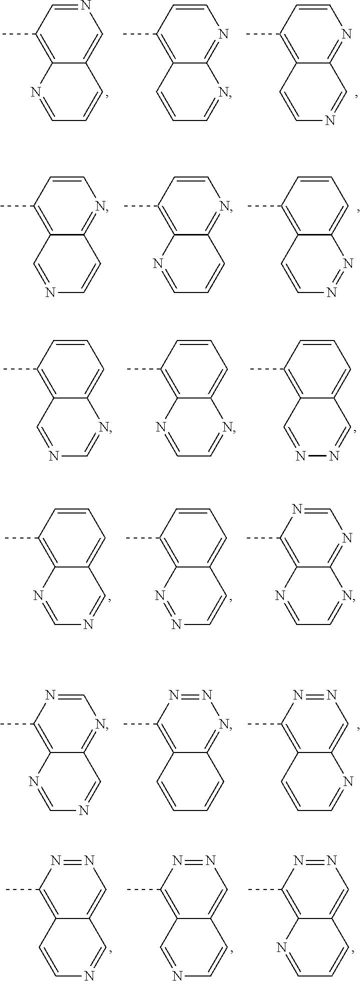 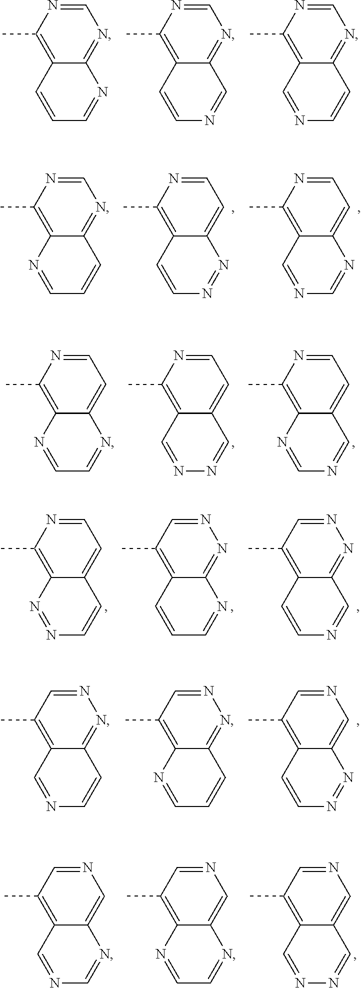 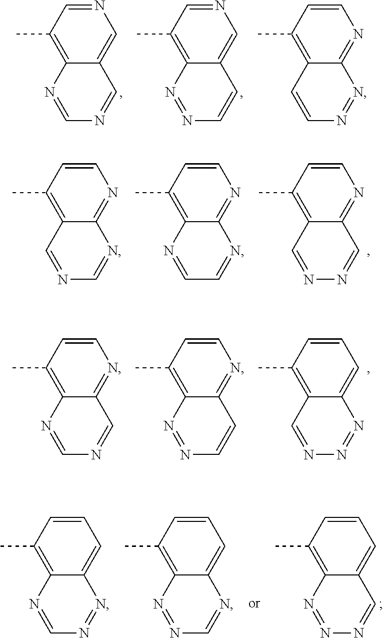
wherein the unsubstituted bicyclic residues can be substituted with 1 to 5 of the substituents R⁹-R¹⁴ and R^{N}; and preferably with 1 to 3 of the substituents R¹¹-R¹³,
R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl and the afore-mentioned residues optionally contain one or more C═C double bond(s) and/or are optionally substituted by one or more of R^{a}, R^{b}, R^{c}, R^{d}, and R^{e};
R^{a}, R^{b}, R^{e}, R^{d}, and R^{e} represent independently of each other —H, —F, —Cl, —Br, CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, —CONHCH₃, —CON(CH₃)₂, —CONHC₂H₅, —CH₂CO₂H, —CH₂CO₂CH₃, —CH₂CO₂C₂H₅, CH₂CONH₂, —CH₂CONHCH₃, —CH₂CON(CH₃)₂, CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, or —NHSO₂CH₂CF₃;
R⁴ represents —NR⁶R⁷;
R⁶ and R⁷ represent independently of each other —H, —CH₃, —CH₂CH₂CH₃, —CH(CH₃)₂, CH₂CH₂CH₂CH₃, —CH₂CH₂CH₂CH₂CH₃, —CH₂CH(CH₃)₂, —C(CH₃)₃, —CH₂CH₂CH₂, —CH₂CH═CH(CH₃), —CH₂CH═C(CH₃)₂, CH₂CH═CHCH₂CH₃, -cyclo-C₃H₅, -cyclo-C₄H₇, -cyclo-CsH₉, -cyclo-C₆H₁₁, CH₂-cyclo-C₃H₅, —CH₂-cyclo-C₄H₇, CH₂-cyclo-C₅H₉, —CH₂-cyclo-C₆H₁₁, -Ph, —CH₂-Ph, CH₂OCH₃, —CH₂OCH₂CH₃, —CH₂CH₂OCH₃, —CH₂CH₂OCH₂CH₃, —CH₂CH₂NHCH₃, or —CH₂CH₂N(CH₃)₂, or —NR⁶R⁷ is —N(C₂H₅)₂, 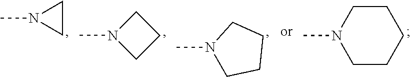
R⁸, R⁹, R¹⁰, R¹¹, R¹², R¹³, and R¹⁴ represent independently of each other —H, —F, —Cl, —Br, -I, —OH, —CN, —NO₂, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, CH₂-cyclo-C₃H₅, —CH₂OH, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —OCH₃, —OC₂H₅, —OC₃H₇, —OCH(CH₃)₂, —OC(CH₃)₃, —OC₄H₉, —OCHF₂, —OCF₃, —OCH₂CF₃, —OC₂F₅, —OCH₂OCH₃, —O-cyclo-C₃H₅, OCH₂-cyclo-C₃H₅, O—C₂H₄-cyclo-C₃H₅, CHO, —COCH₃, —COCF₃, —COC₂H₅, —COC₃H₇, —COCH(CH₃)₂, —COC(CH₃)₃, —COOH, —COOCH₃, —COOC₂H₅, —COOC₃H₇, —COOCH(CH₃)₂, —COOC(CH₃)₃, —OOC—CH₃, —OOC—CF₃, —OOC—C₂H₅, —OOC—C₃H₇, —OOC—CH(CH₃)₂, —OOC—C(CH₃)₃, —NH₂, —NHCH₃, —NHC₂H₅, —NHC₃H₇, —NHCH(CH₃)₂, —NHC(CH₃)₃, —N(CH₃)₂, —N(C₂H₅)₂, —N(C₃H₇)₂, —N[CH(CH₃)₂]₂, —N[C(CH₃)₃]₂, —NHCOCH₃, —NHCOCF₃, —NHCOC₂H₅, —NHCOC₃H₇, —NHCOCH(CH₃)₂, —NHCOC(CH₃)₃, CONH₂, —CONHCH₃, —CONHC₂H₅, CONHC₃H₇, CONHCH(CH₃)₂, —CONH-cyclo-C₃H₅, —CONHC(CH₃)₃, —CON(CH₃)₂, —CON (C₂H₅)₂, —CON(C₃H₇)₂, —CON[CH(CH₃)₂]₂, —CON[C(CH₃)₃]₂, —SO₂NH₂, —SO₂NHCH₃, —SO₂NHC₂H₅, —SO₂NHC₃H₇, —SO₂NHCH(CH₃)₂, —SO₂NH-cyclo-C₃H₅, —SO₂NHC(CH₃)₃, —SO₂N(CH₃)₂, —SO₂N (C₂H₅)₂, —SO₂N(C₃H₇)₂, —SO₂N[CH(CH₃)₂]₂, —SO₂N[C(CH₃)₃]₂, —NHSO₂CH₃, —NHSO₂CF₃, —NHSO₂C₂H₅, —NHSO₂C₃H₇, —NHSO₂CH(CH₃)₂, —NHSO₂C(CH₃)₃, —CH═CH₂, —CH₂—CH—CH₂, —C(CH₃)—CH₂, —CH═CH—CH₃, —C≡CH, —C≡C—CH₃, —CH₂—C≡CH, -Ph, —O-Ph, —O—CH₂-Ph, 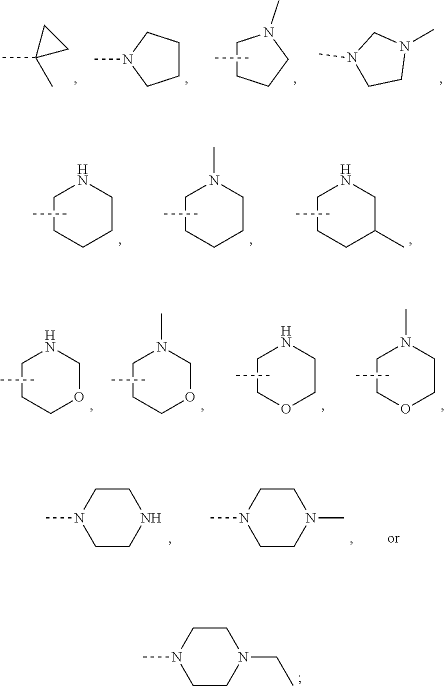
or R⁸ and R⁹ or R⁹ and R¹⁰ can form together one of the following five-membered or six-membered rings: 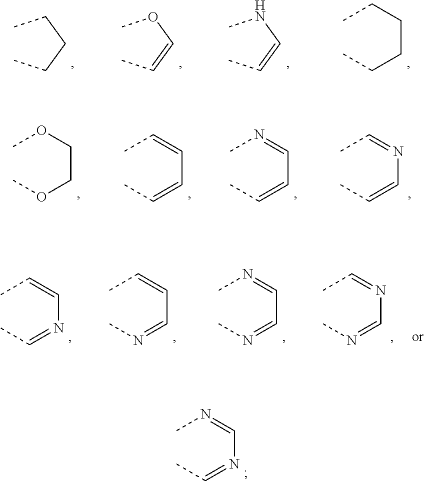
or R¹² and R¹³ or R¹³ and R¹⁴ can form together one of the following five-membered or six-membered rings: 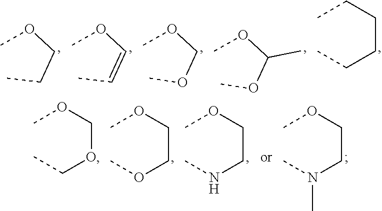
R^{N} represents —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, -cyclo-C₃H₅, -cyclo-C₄H₇, -cyclo-C₅H₉, —CH₂-cyclo-C₃H₅, —CH₂-cyclo-C₄H₇, —CH₂-cyclo-C₅H₉, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₂—CH═CH₂, —CH₂—C≡CH, —CHO, —COCH₃, —COC₂H₅, —COC₃H₇, —COCH(CH₃)₂, —COC(CH₃)₃, —CO-cyclo-C₃H₅, —CO-cyclo-C₄H₇, —CO-cyclo-C₅H₉, —COOCH₃, —COOC₂H₅, —COOC₃H₇, —COOCH(CH₃)₂, —COOC(CH₃)₃, —COOCH₂Ph, —SO₂CH₃, —SO₂CF₃, —SO₂C₂H₅, —SO₂C₃H₇, —SO₂CH(CH₃)₂, —SO₂-cyclo-C₃H₅, or —SO₂C(CH₃)₃;
R^{N1} represents —H, —CH₃, or —CH₂CH₃;
or a diastereomer, an enantiomer, a mixture of diastereomers, a mixture of enantiomers, a racemate, a solvate, a hydrate, or a pharmaceutically acceptable salt thereof.

2. The compound according to claim 1, wherein R² represents 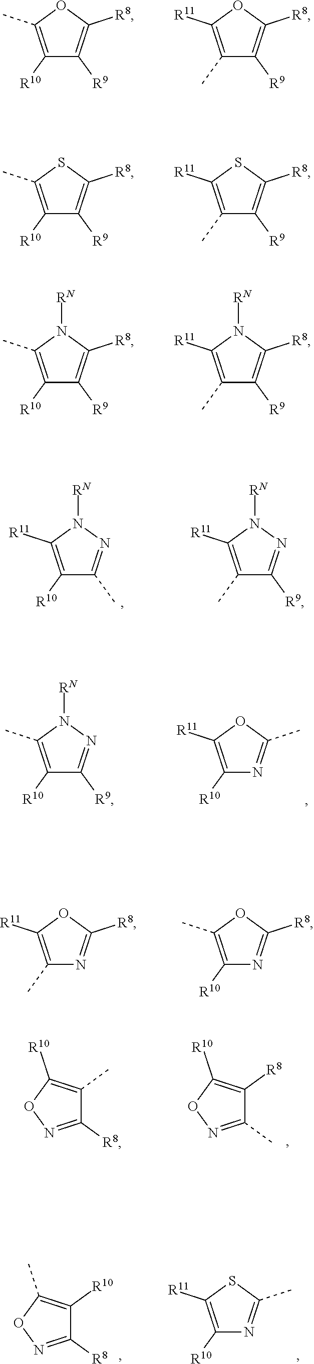 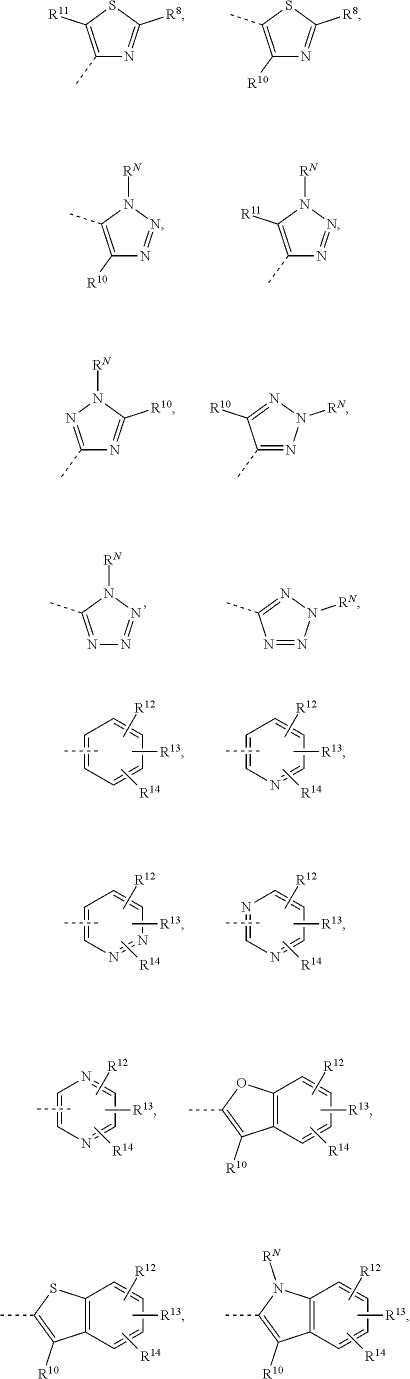 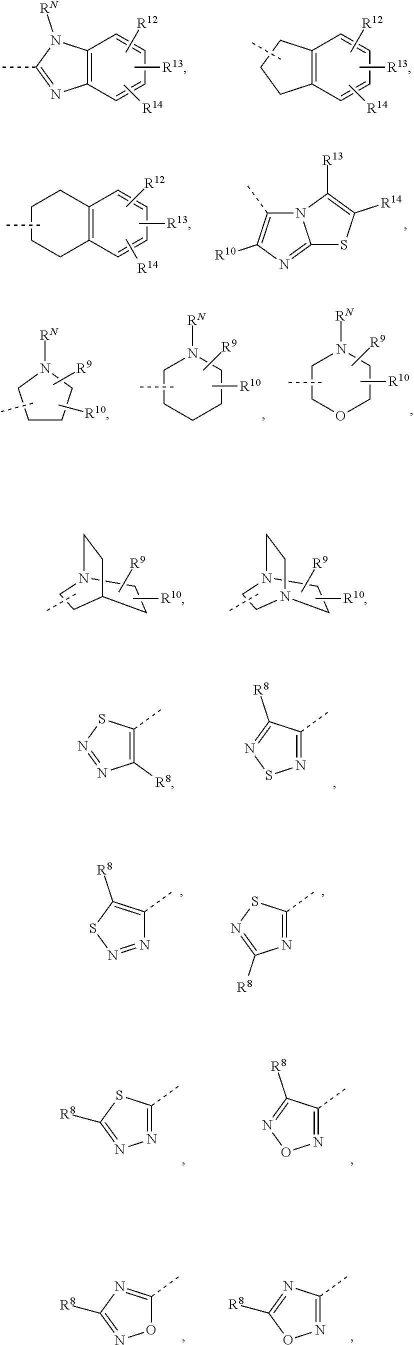 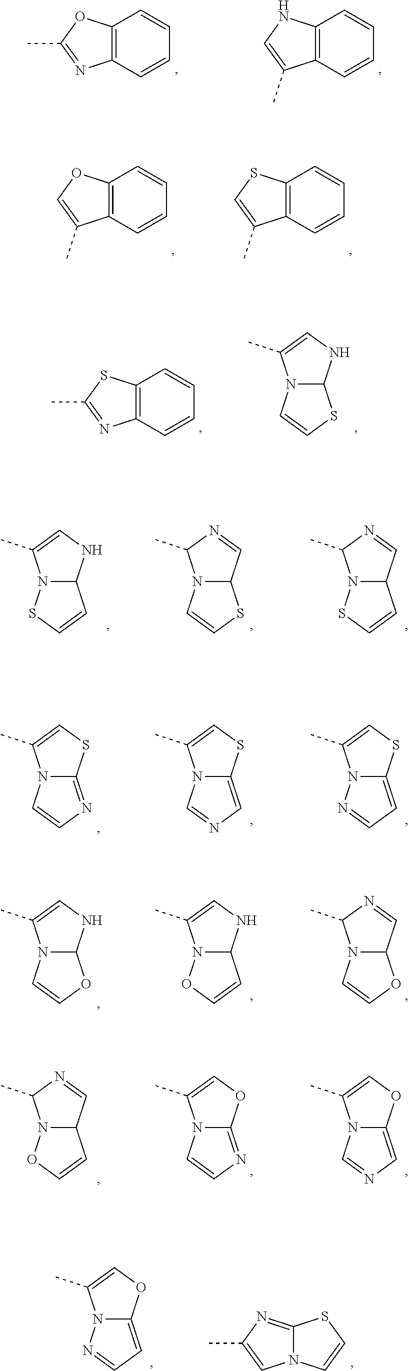 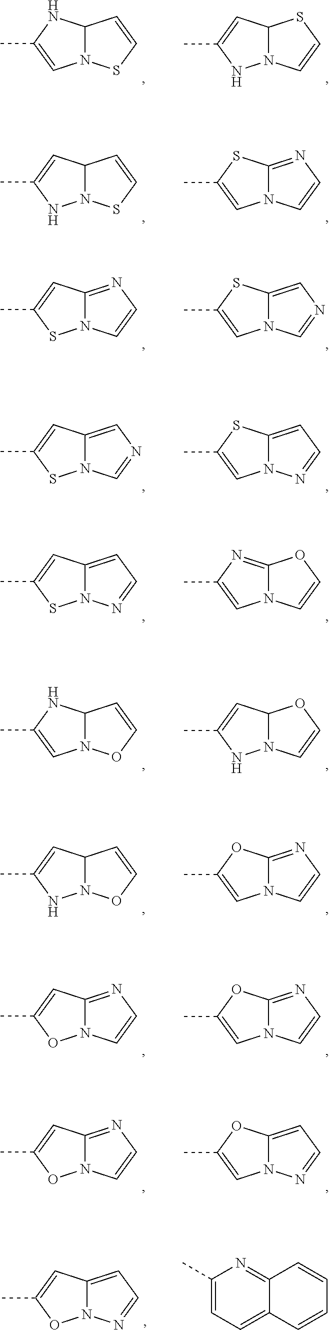 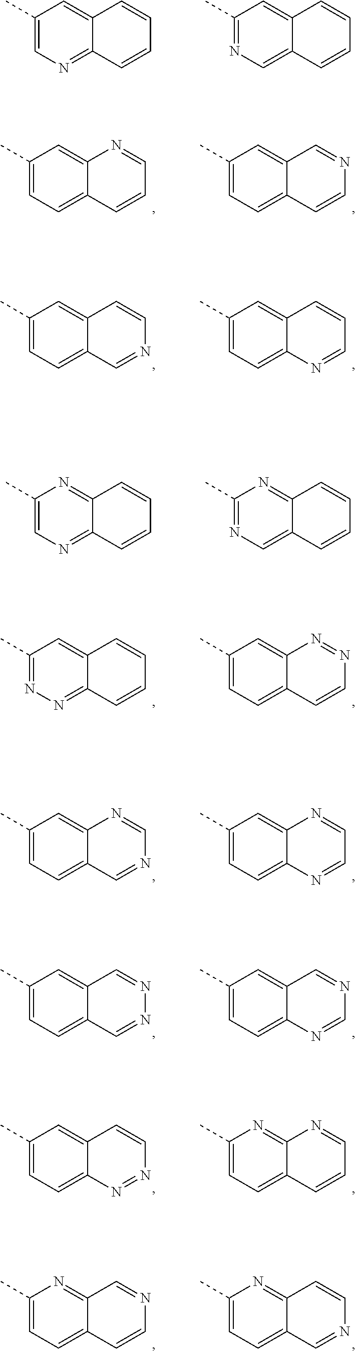 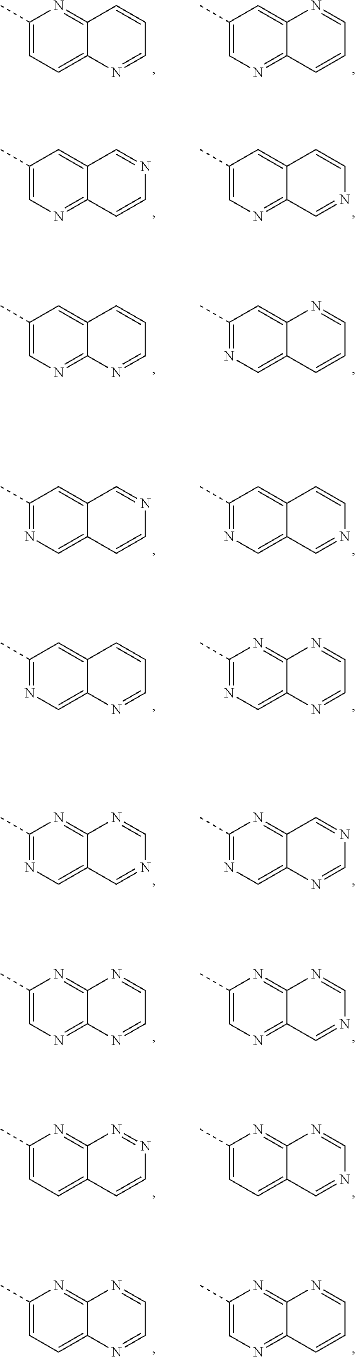 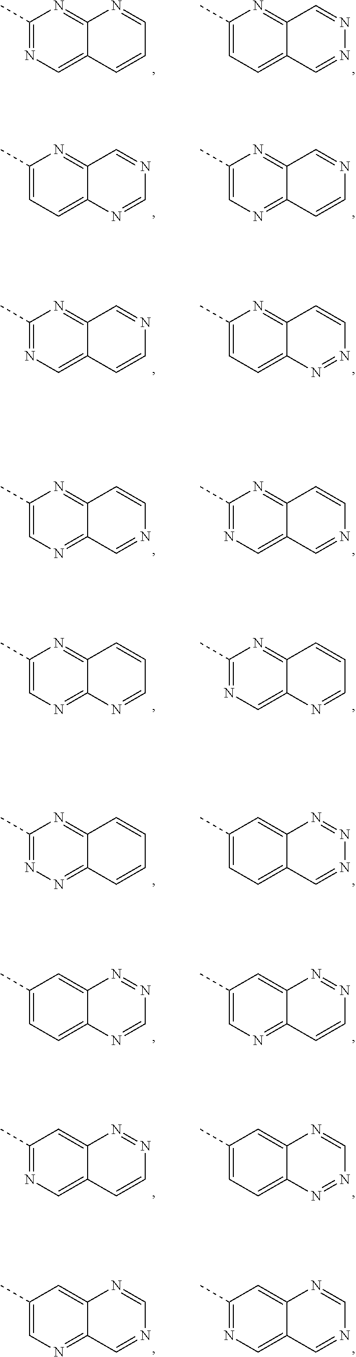 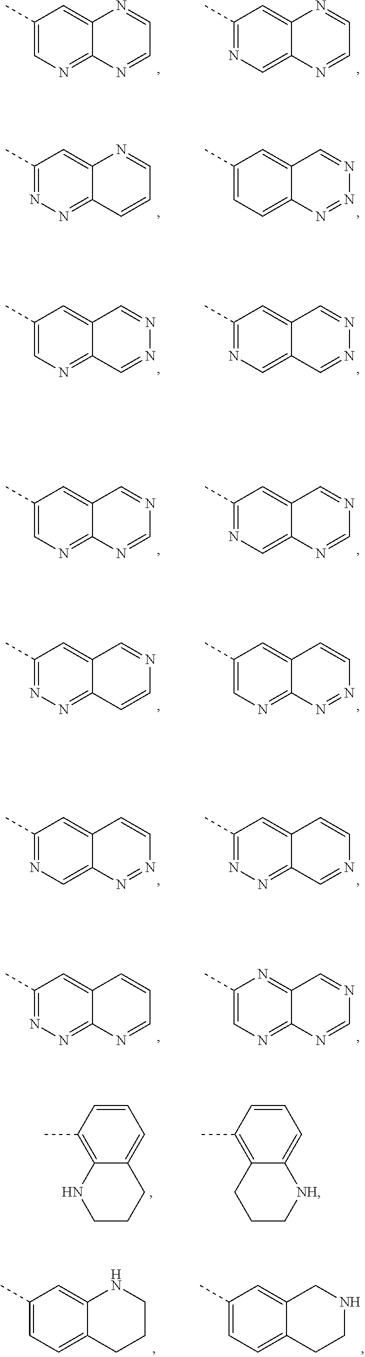 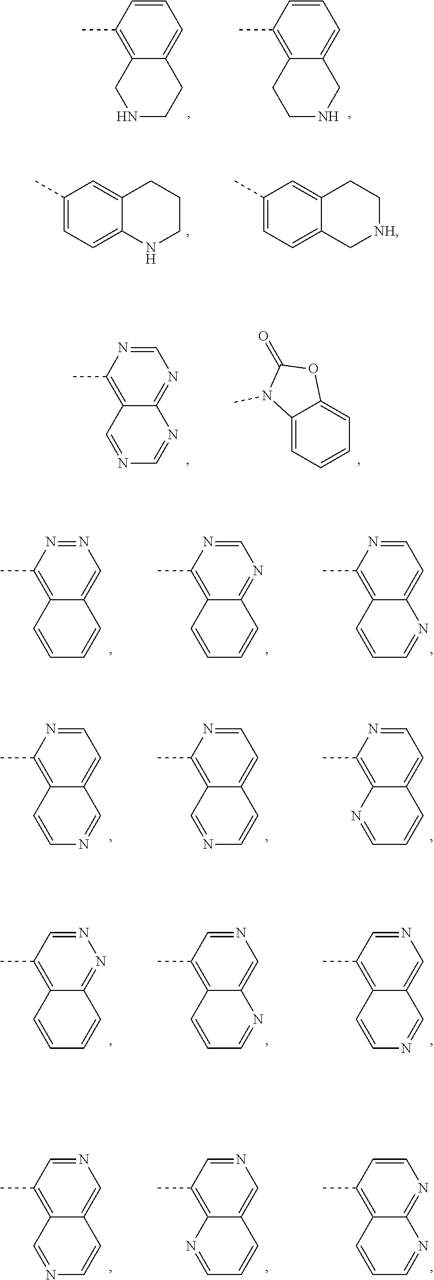 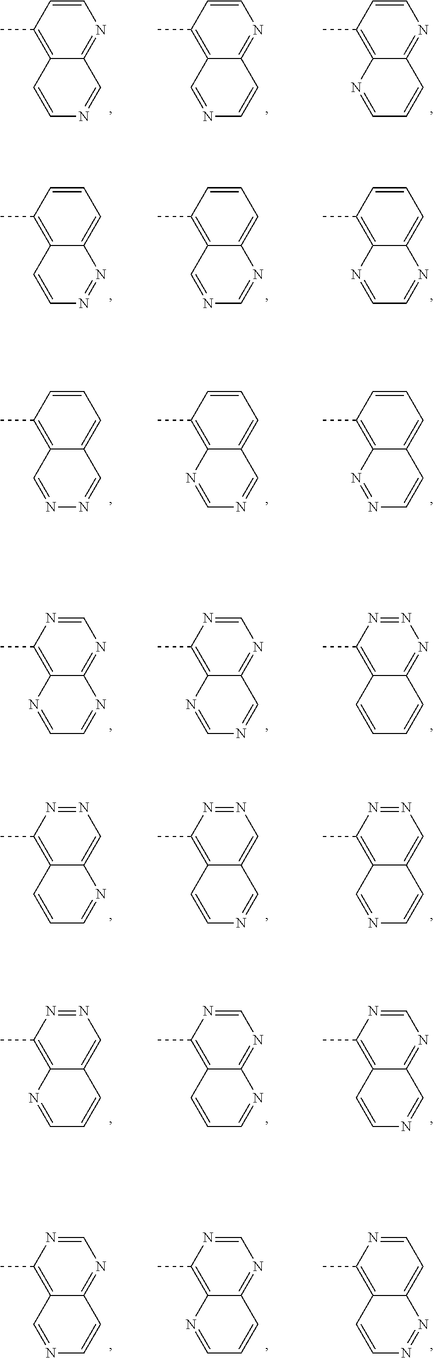 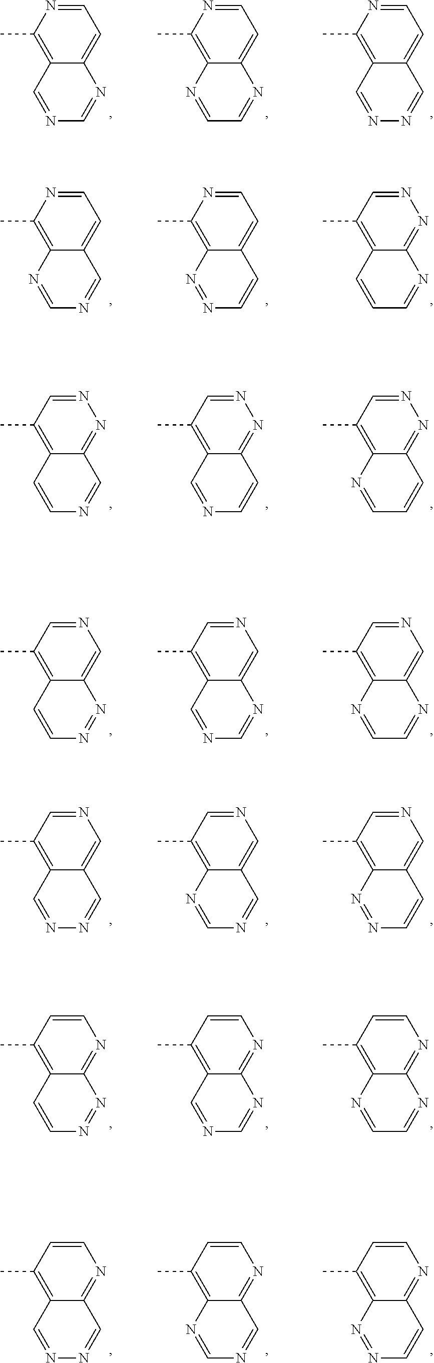 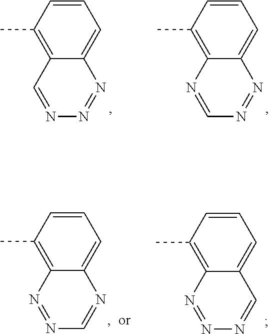
wherein the unsubstituted bicyclic residues can be substituted with 1 to 5 of the substituents R⁹-R¹⁴ and R^{N}; and preferably with 1 to 3 of the substituents R¹¹-R¹³ and the substituents R⁹-R¹⁴ and R^{N} have the meanings as defined in claim 1.

3. The compound according to claim 1, wherein R² represents 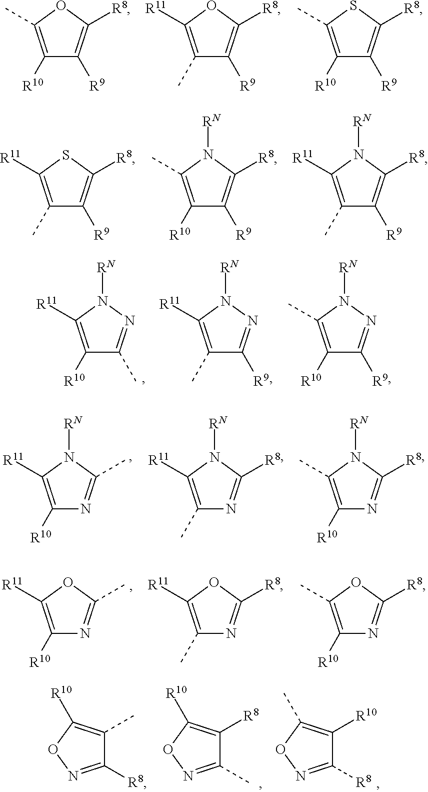 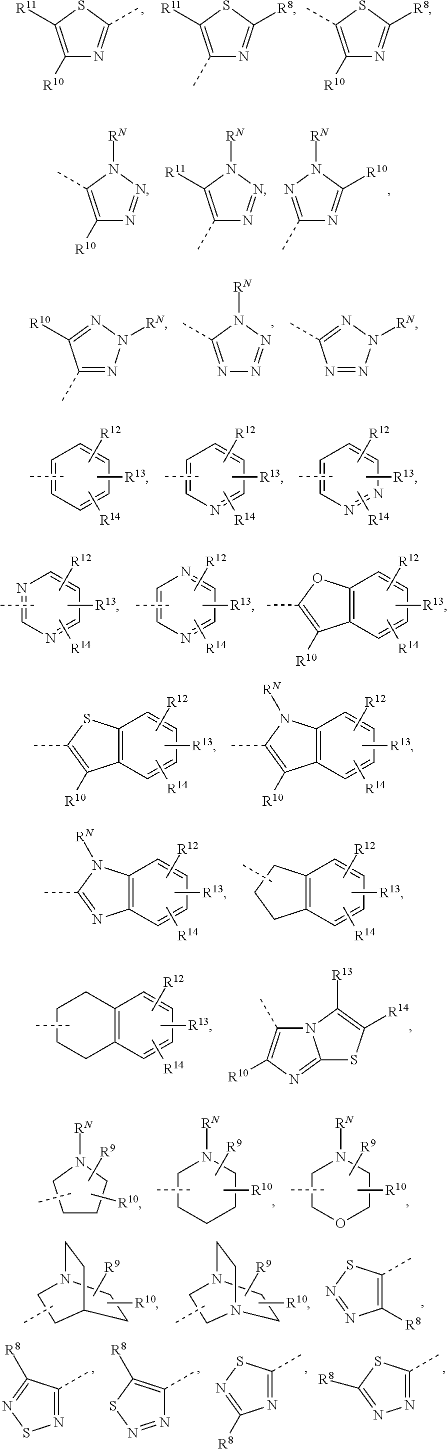 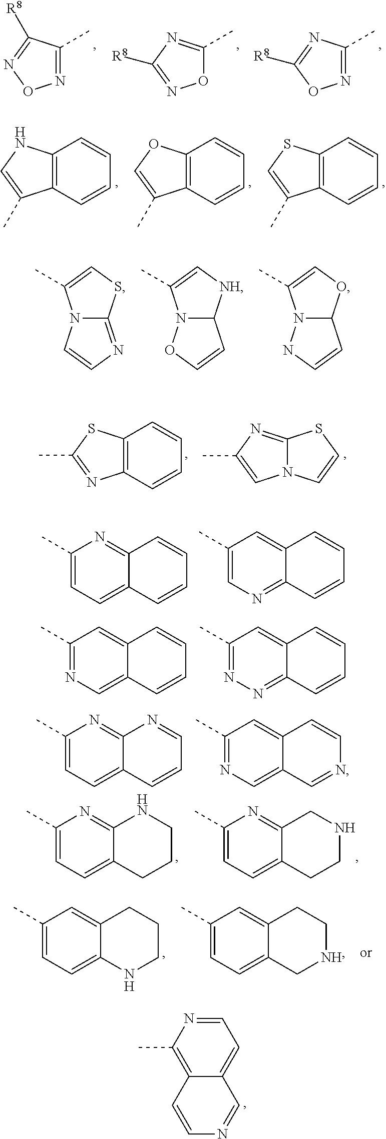
wherein the unsubstituted bicyclic residues can be substituted with 1 to 5 of the substituents R⁹-R¹⁴ and R^{N}; and preferably with 1 to 3 of the substituents R¹¹-R¹³ and the substituents R⁹-R¹⁴ and R^{N} have the meanings as defined in claim 1.

4. The compound according to claim 1, wherein the compound has the formula (Ib) 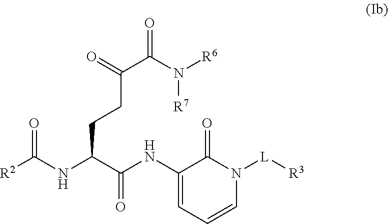
wherein
L represents -L¹- or -L¹-L²-; preferably -L¹-L²-;
L¹ represents —CH₂—, —CH₂CH₂—, —CH₂CH₂CH₂—, —CH₂CO—, or —CH₂CH₂CO—;
L² represents a bond, —NR^{N1}—, —NR^{N1}CH₂—, —NR^{N1}CH₂CH₂—, or —NR^{N1}CH(CH₃)—;
R² represents 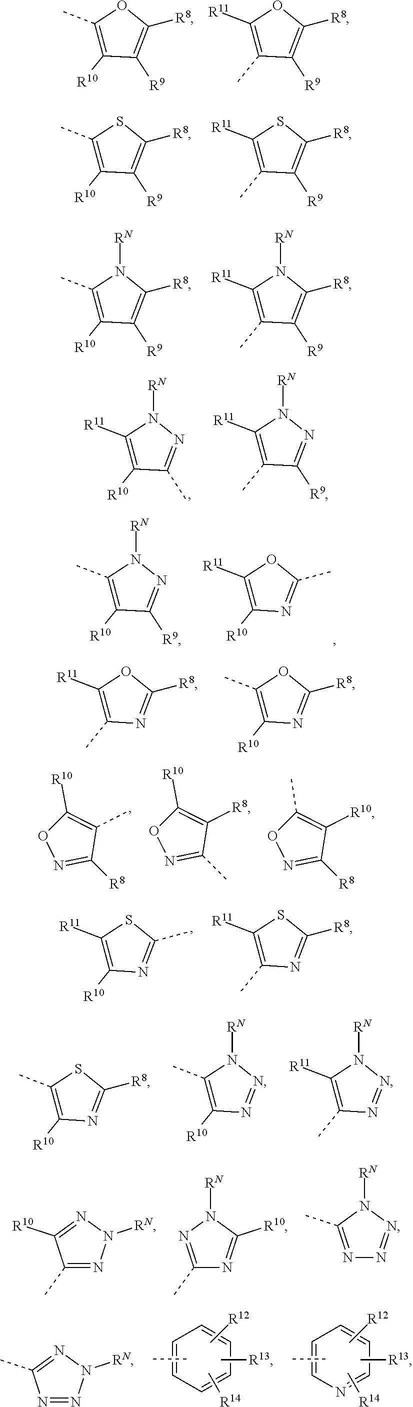 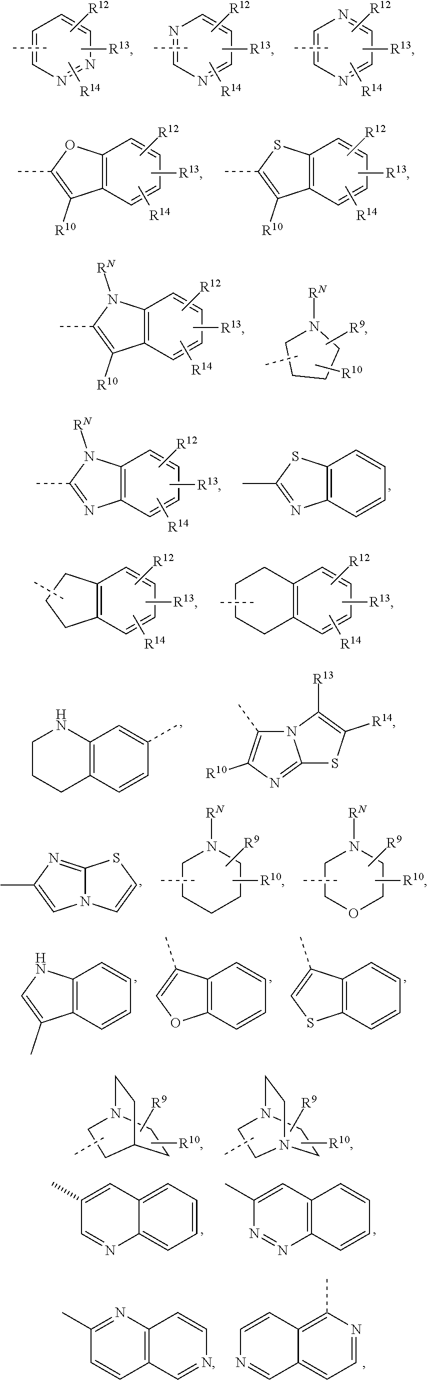 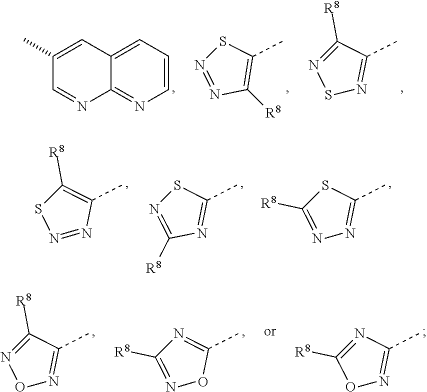
R³ represents bicyclo[1.1.1]pentyl, bicyclo[2.1.1]hexyl, bicyclo[2.2.1]heptyl, bicyclo[3.1.1]heptyl, bicyclo[2.2.2]octyl, bicyclo[3.2.1]octyl, bicyclo[3.2.2]nonyl, bicyclo[3.3.2]decyl, bicyclo[3.3.3]undecyl, 4-homoisotwistyl, adamantyl, diamantyl, hexamethylenetetraminyl, and the afore-mentioned residues optionally contain one or more C═C double bond(s) and/or are substituted by one or more of R^{a}, R^{b}, R^{c}, R^{d}, and R^{e};
R^{a}, R^{b}, R^{c}, R^{d}, and R^{e} represent independently of each other —H, —F, —Cl, —Br, —CN, —OH, —CH₃, —CH₂CH₃, —CH₂CH₂CH₃, —CH(CH₃)₂, —CHF₂, —CF₃, —CH₂CF₃, —COCH₃, —COCH₂CH₃, —CO₂H, —CO₂CH₃, —CO₂C₂H₅, —CONH₂, CONHCH₃, —CON(CH₃)₂, —CONHC₂H₅, CH₂CO₂H, —CH₂CO₂CH₃, CH₂CO₂C₂H₅, CH₂CONH₂, —CH₂CONHCH₃, CH₂CON(CH₃)₂, —CH₂CONHC₂H₅, —NHCOCH₃, —NHCOC₂H₅, —NHCOCF₃, —NHCOCH₂CF₃, —NHSO₂CH₃, —NHSO₂C₂H₅, —NHSO₂CHF₂, —NHSO₂CF₃, or —NHSO₂CH₂CF₃;
R⁴ represents —NR⁶R⁷;
R⁶ and R⁷ represent independently of each other —H, —CH₃, —CH₂CH₂CH₃, —CH(CH₃)₂, CH₂CH₂CH₂CH₃, CH₂CH₂CH₂CH₂CH₃, CH₂CH(CH₃)₂, —C(CH₃)₃, CH₂CH—CH₂, CH₂CH═CH(CH₃), CH₂CH═C(CH₃)₂, CH₂CH═CHCH₂CH₃, -cyclo-C₃H₅, -cyclo-C₄H₇, -cyclo-C₅H₉, -cyclo-C₆H₁₁, —CH₂-cyclo-C₃H₅, —CH₂-cyclo-C₄H₇, —CH₂-cyclo-C₅H₉, —CH₂-cyclo-C₆H₁₁, -Ph, —CH₂-Ph, —CH₂OCH₃, —CH₂OCH₂CH₃, —CH₂CH₂OCH₃, —CH₂CH₂OCH₂CH₃, —CH₂CH₂NHCH₃, —CH₂CH₂N(CH₃)₂,
or —NR⁶R⁷ is —N(C₂H₅)₂, 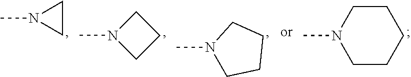
R^{N} represents —H, —CH₃, —C₂H₅, —C₃H₇, —CH(CH₃)₂, —C₄H₉, —CH₂—CH(CH₃)₂, CH(CH₃)—C₂H₅, —C(CH₃)₃, -cyclo-C₃H₅, -cyclo-C₄H₇, -cyclo-C₅H₉, —CH₂-cyclo-C₃H₅, —CH₂F, —CHF₂, —CF₃, —CH₂Cl, —CH₂Br, —CH₂I, —CH₂—CH₂F, —CH₂—CHF₂, —CH₂—CF₃, —CH₂—CH₂Cl, —CH₂—CH₂Br, —CH₂—CH₂I, —CH₂—CH═CH₂, —CH₂—C≡CH, CHO, —COCH₃, —COC₂H₅, —COC₃H₇, —COCH(CH₃)₂, —COC(CH₃)₃, —COOCH₃, —COOC₂H₅, —COOC₃H₇, —COOCH(CH₃)₂, COOC(CH₃)₃, —COOCH₂Ph, —SO₂CH₃, —SO₂CF₃, —SO₂C₂H₅, —SO₂C₃H₇, —SO₂CH(CH₃)₂, or —SO₂C(CH₃)₃;
R^{N1} represent —H, —CH₃, or —CH₂CH₃;
and R⁸-R¹⁴ have the meanings as defined in formula (I);
or a diastereomer, an enantiomer, a mixture of diastereomers, a mixture of enantiomers, a racemate, a solvate, a hydrate, or a pharmaceutically acceptable salt thereof.

5. The compound according to claim 1, wherein
L¹ represents —CH₂—, or —CH₂CO—;
L² represents a bond, —NR^{N1}—, —NR^{N1}CH₂—, or —NR^{N1}CH(CH₃)—;
R³ represents bicyclo[1.1.1]pentyl, bicyclo[2.1.1]hexyl, bicyclo[2.2.1]heptyl, bicyclo[3.1.1]heptyl, bicyclo[2.2.2]octyl, 4-homoisotwistyl, adamantyl, or diamantyl, and the afore-mentioned residues optionally contain one or more C═C double bond(s) and/or are substituted by one or more of R^{a}, R^{b}, R^{e}, R^{d}, and R^{e};
and R^{a}, R^{b}, R^{c}, R^{d}, R^{e} and R^{N1} have the same meanings as defined in claim 1.

6. The compound according to claim 1, wherein R² represents 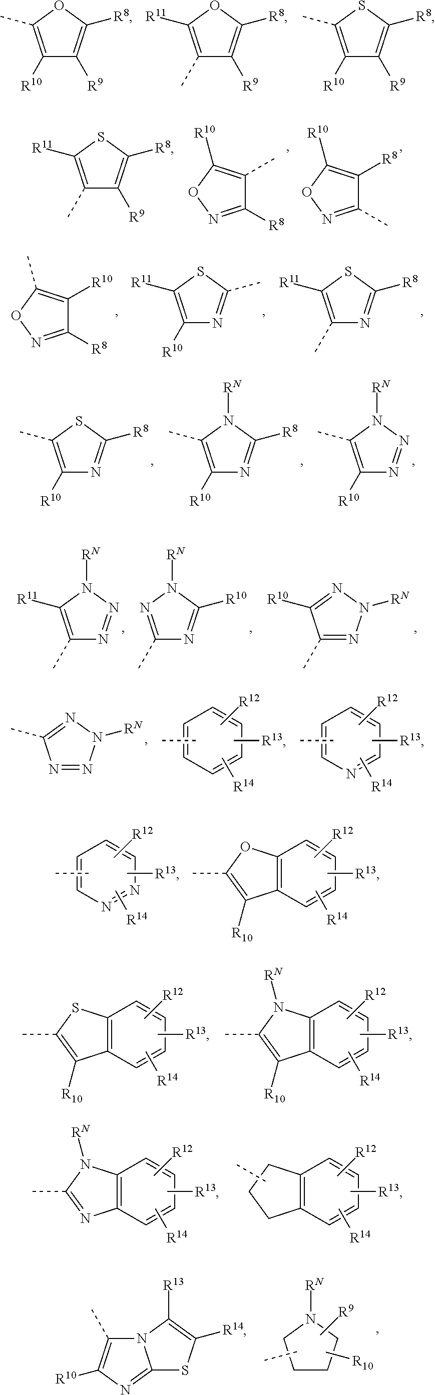 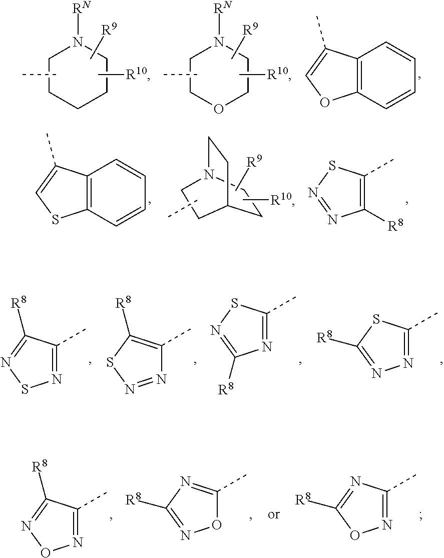
and R⁸-R¹⁴ and RN have the meanings as defined in claim 1 or 2.

7. The compound according to claim 1, wherein the compound has any one of the formulae (IV-a)-(IV-o) and (V-a)-(V-d): 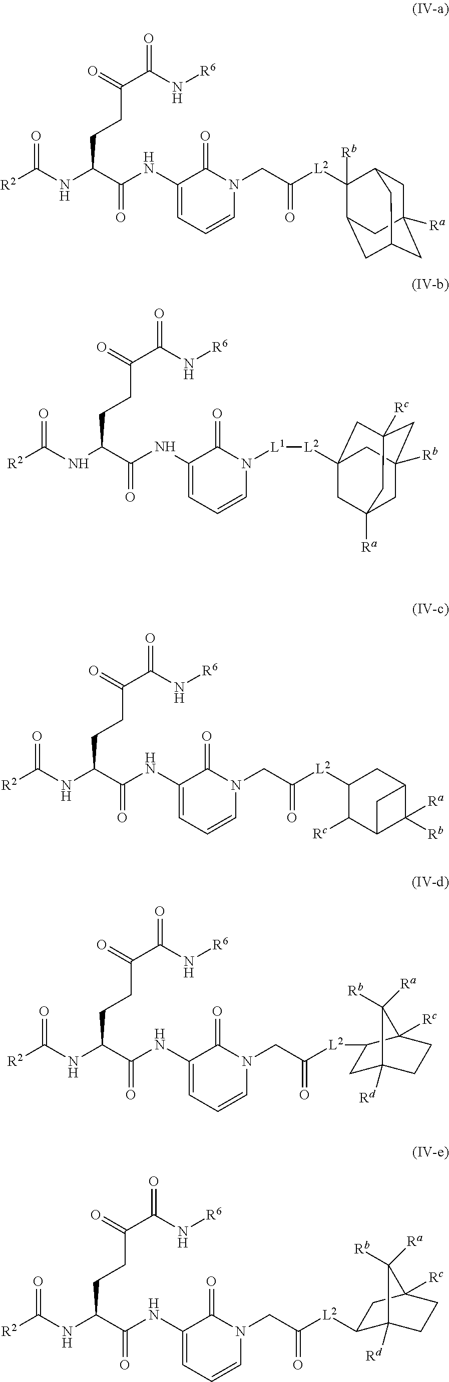 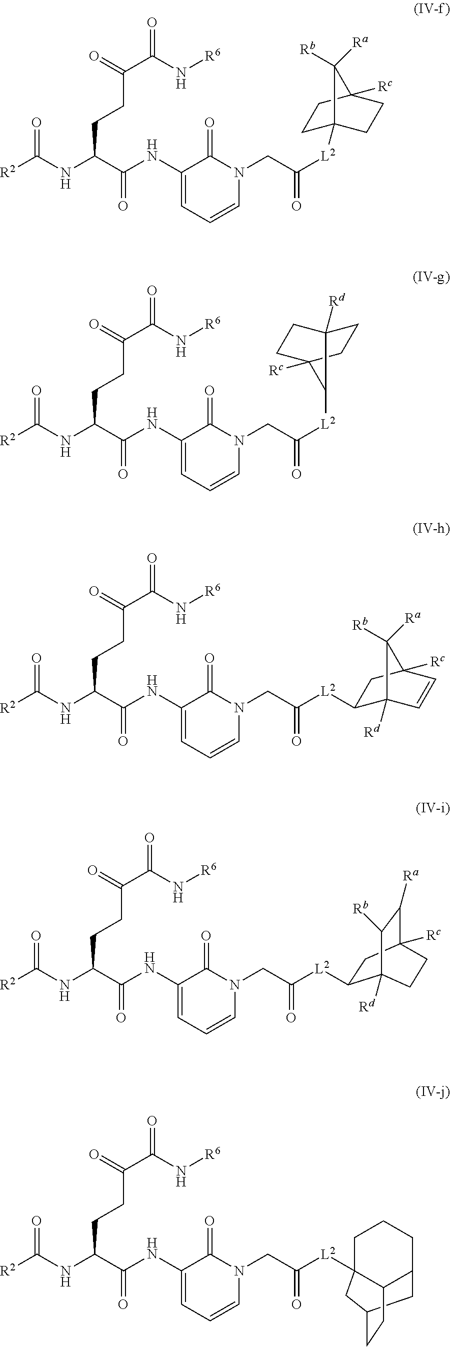 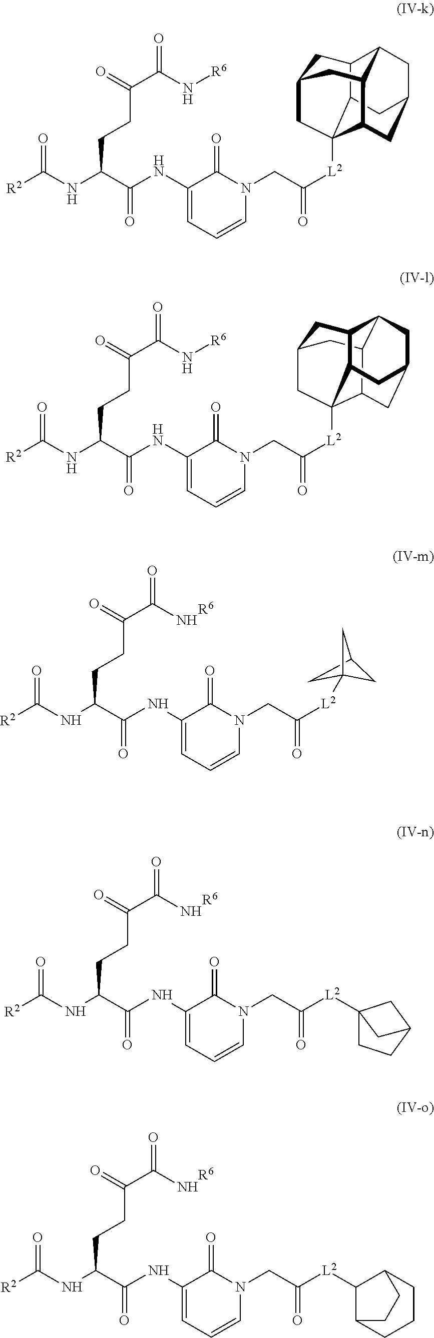 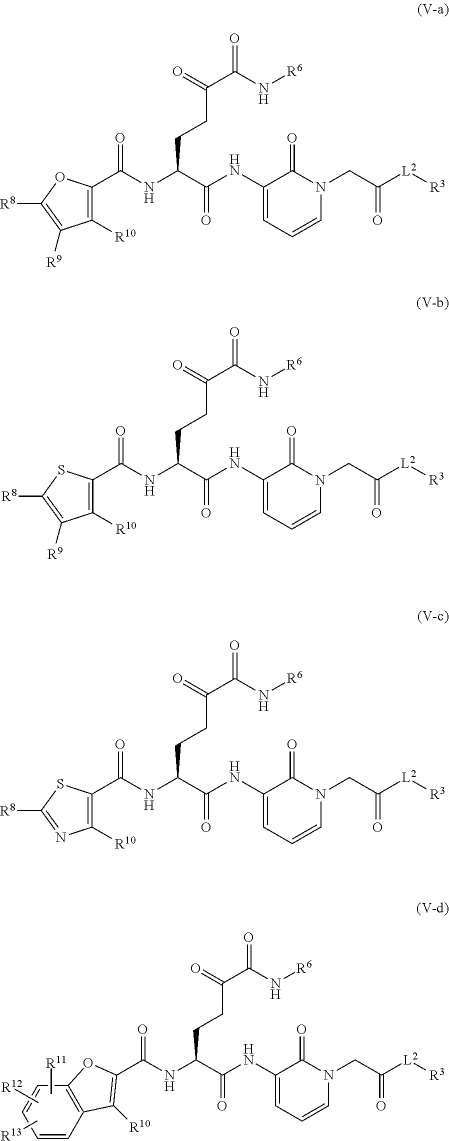
and R², R³, R⁶, R⁸, R⁹, R¹⁰, R¹¹, R¹², R¹³, R^{a}, R^{b}, R^{e}, R^{d} and L² have the same meanings as defined in claim 1.

8. The compound according to claim 1, wherein
R² represents 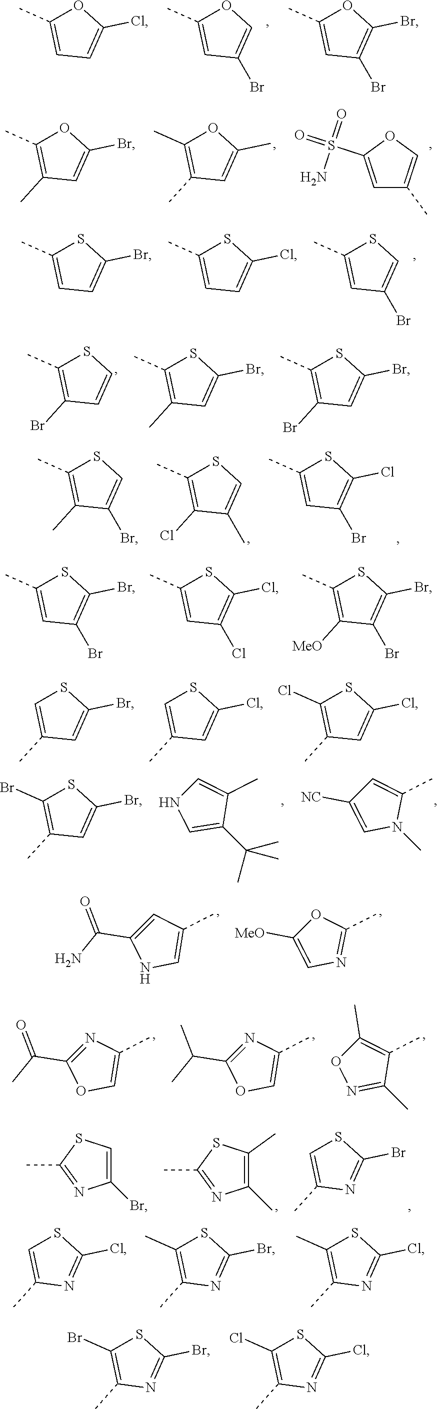 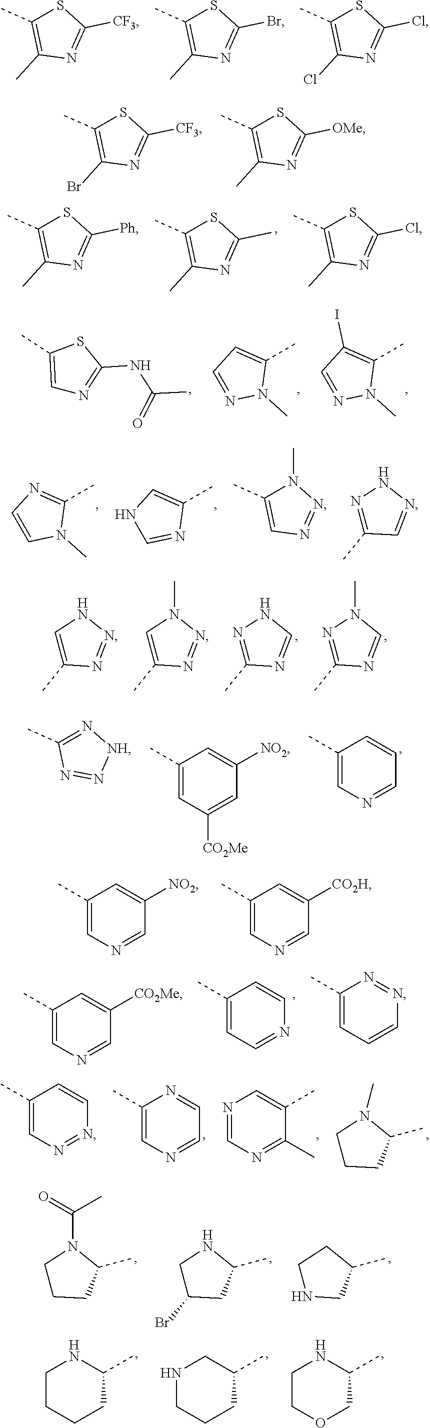 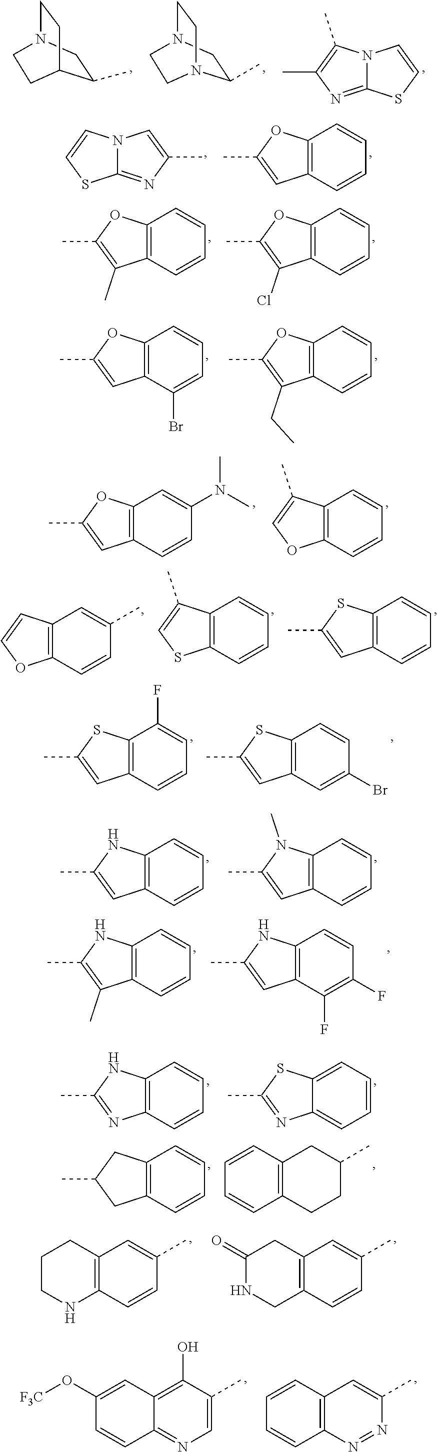 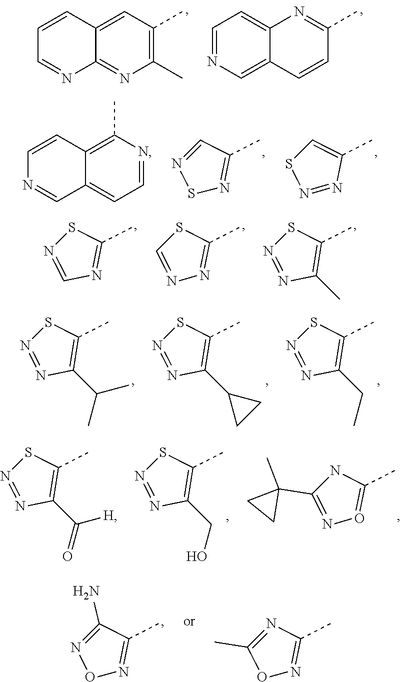
and
R⁶ represents —H, —CH₃, —CH(CH₃)₂, —CH₂CH₂CH₃, —CH—CH═CH₂, —CH₂CH₂CH CH₃, —CH₂CH(CH₃)₂, —C(CH₃)₃, —CH₂CH₂CH₂CH₂CH₃, -cyclo-C₃H₅, -cyclo-C₅H₉, -cyclo-C₆H₁₁ or —CH₂-cyclo-C₃H₅.

9. The compound according to claim 1 selected from the group consisting of:

| Compound | Name |
|---|---|
| II-2: | (S)-2-(benzofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-3: | (S)-N1-(1-(2-(2-adamantylamino)-2- |
| | oxoethyl)-2-oxo-1,2-dihydropyridin-3- |
| | yl)-N6-methyl-2-(3-methylbenzofuran- |
| | 2-carboxamido)-5- |
| | oxohexanediamide |
| II-4: | (S)-2-(3-chlorobenzofuran-2-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-5: | (S)-2-(4-bromobenzofuran-2-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-6: | (S)-2-(4-bromobenzofuran-2-carboxamido)- |
| | N1-(1-(2-(2-adamantylamino)- |
| | 2-oxoethyl)-2-oxo-1,2- |
| | dihydropyridin-3-yl)-N6-methyl-5- |
| | oxohexanediamide |
| II-7: | (S)-2-(benzo[b]thiophene-2-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-8: | (S)-2-(5-bromobenzo[b]thiophene- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-9: | (S)-2-(1H-indole-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-10: | (S)-2-(4,5-difluoro-1H-indole-2- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-11: | (S)-N1-(1-(2-(2-adamantylamino)-2- |
| | oxoethyl)-2-oxo-1,2-dihydropyridin-3- |
| | yl)-N6-methyl-2-(3-methyl-1H- |
| | indole-2-carboxamido)-5- |
| | oxohexanediamide |
| II-12: | (S)-2-(1H-benzo[d]imidazole-2- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-13: | (S)-2-(2,3-dihydro-1H-indene-2- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-14: | (S)--(2-bromo-4-methylthiazole- |
| | 5-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-15: | (S)-N1-(1-(2-(2-adamantylamino)-2- |
| | oxoethyl)-2-oxo-1,2-dihydropyridin-3- |
| | yl)-N6-methyl-2-(4-methyl-2- |
| | (trifluoromethyl)thiazole-5-carboxamido)-5- |
| | oxohexanediamide |
| II-16: | (S)-2-(4-bromo-2-(trifluoromethyl)thiazole- |
| | 5-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-17: | (S)-2-(2,4-dichlorothiazole-5-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-18: | (S)-N1-(1-(2-(2-adamantylamino)-2-oxoethyl)- |
| | 2-oxo-1,2-dihydropyridin-3- |
| | yl)-2-(2-methoxy-4-methylthiazole- |
| | 5-carboxamido)-N6-methyl-5- |
| | oxohexanediamide |
| II-19: | (S)-N1-(1-(2-(2-adamantylamino)-2- |
| | oxoethyl)-2-oxo-1,2-dihydropyridin-3- |
| | yl)-N6-methyl-2-(4-methyl-2- |
| | phenylthiazole-5-carboxamido)-5- |
| | oxohexanediamide |
| II-20: | (S)-2-(2,4-dimethylthiazole-5- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-21: | (S)-2-(5-bromo-3-methylthiophene- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-22: | (S)-2-(3,5-dibromothiophene-2- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-23: | (S)-2-(5-bromothiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-24: | (S)-2-(5-chlorothiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-25: | (S)-2-(5-bromo-3-methylfuran- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-26: | (S)-2-(5-chlorofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-27: | (S)-2-(5-chlorothiophene-3-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-28: | (S)-2-(2,5-dichlorothiophene-3- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-29: | (S)-2-(2,5-dibromothiophene-3- |
| | carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-30: | (S)-2-(5-bromothiophene-3-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-31: | (S)-2-(2-chloro-5-methylthiazole- |
| | 4-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-32: | (S)-2-(2,5-dichlorothiazole-4-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-33: | (S)-2-(2,5-dibromothiazole-4-carboxamido)- |
| | N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-34: | (S)-2-(2-bromo-5-methylthiazole- |
| | 4-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-35: | (S)-2-(2-bromothiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-36: | (S)-2-(2-chlorothiazole-4-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-37: | (S)-2-(2,5-dimethylfuran-3-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-38: | (S)-2-(4,5-dimethylthiazole- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-39: | (S)-2-(4-bromothiazole-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-40: | (S)-2-(4-bromothiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-41: | (S)-2-(4-bromo-3-methylthiophene- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-42: | (S)-2-(3-bromothiophene-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-43: | (S)-2-(3-chloro-4-methylthiophene- |
| | 2-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-44: | (S)-2-(4-bromo-5-chlorothiophene- |
| | 2-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-45: | (S)-2-(4,5-dibromothiophene-2- |
| | carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-46: | (S)-2-(4,5-dibromo-3-methoxythiophene- |
| | 2-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-47: | (S)-2-(4-bromofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-48: | (S)-2-(4,5-dibromofuran-2-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-49: | (S)-2-(4,5-dichlorothiophene- |
| | 2-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-50: | (S)-2-((S)-1-acetylpyrrolidine- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-51: | (S)-N1-(1-(2-(2-adamantylamino)- |
| | 2-oxoethyl)-2-oxo-1,2-dihydropyridin-3- |
| | yl)-N6-methyl-2-(1-methyl-1H-1,2,3- |
| | triazole-5-carboxamido)-5- |
| | oxohexanediamide |
| II-52: | (S)-N1-(1-(2-(2-adamantylamino)-2- |
| | oxoethyl)-2-oxo-1,2-dihydropyridin-3- |
| | yl)-N6-methyl-5-oxo-2-(2H-tetrazole-5- |
| | carboxamido)hexanediamide |
| II-53: | (S)-N1-(1-(2-(2-adamantylamino)- |
| | 2-oxoethyl)-2-oxo-1,2-dihydropyridin-3- |
| | yl)-N6-methyl-5-oxo-2-(pyrazine- |
| | 2-carboxamido)hexanediamide |
| II-54: | (S)-N1-(1-(2-(2-adamantylamino)-2- |
| | oxoethyl)-2-oxo-1,2-dihydropyridin-3- |
| | yl)-N6-methyl-2-((S)-1-methylpyrrolidine- |
| | 2-carboxamido)-5- |
| | oxohexanediamide |
| II-55: | (S)-N1-(1-(2-(2-adamantylamino)-2- |
| | oxoethyl)-2-oxo-1,2-dihydropyridin-3- |
| | yl)-N6-methyl-5-oxo-2-((S)-pyrrolidine- |
| | 3-carboxamido)hexanediamide |
| II-56: | (S)-2-((2S,4S)-4-bromopyrrolidine- |
| | 2-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-58: | (S)-N1-(1-(2-(2-adamantylamino)-2- |
| | oxoethyl)-2-oxo-1,2-dihydropyridin-3- |
| | yl)-N6-methyl-5-oxo-2-((S)-piperidine-2- |
| | carboxamido)hexanediamide |
| II-59: | (S)-N1-(1-(2-(2-adamantylamino)-2- |
| | oxoethyl)-2-oxo-1,2-dihydropyridin-3- |
| | yl)-N6-methyl-5-oxo-2-((R)-piperidine-3- |
| | carboxamido)hexanediamide |
| II-60: | (S)-N1-(1-(2-(2-adamantylamino)-2- |
| | oxoethyl)-2-oxo-1,2-dihydropyridin-3- |
| | yl)-N6-methyl-2-((R)-morpholine-3- |
| | carboxamido)-5-oxohexanediamide |
| II-61: | (S)-N1-(1-(2-(2-adamantylamino)-2- |
| | oxoethyl)-2-oxo-1,2-dihydropyridin-3- |
| | yl)-N6-methyl-5-oxo-2-(quinuclidine- |
| | 3-carboxamido)hexanediamide |
| II-62: | (S)-methyl 3-(1-(1-(2-(2-adamantylamino)- |
| | 2-oxoethyl)-2-oxo-1,2- |
| | dihydropyridin-3-ylamino)-6- |
| | (methylamino)-1,5,6-trioxohexan-2- |
| | ylcarbamoyl)-5-nitrobenzoate |
| II-63: | (S)-N1-(1-(2-(2-adamantylamino)- |
| | 2-oxoethyl)-2-oxo-1,2-dihydropyridin-3- |
| | yl)-N6-methyl-2-(5-nitronicotinamido)- |
| | 5-oxohexanediamide |
| II-64: | (S)-5-(1-(1-(2-(2-adamantylamino)- |
| | 2-oxoethyl)-2-oxo-1,2-dihydropyridin- |
| | 3-ylamino)-6-(methylamino)-1,5,6- |
| | trioxohexan-2-ylcarbamoyl)nicotinic |
| | acid |
| II-65: | (S)-methyl 5-(1-(1-(2-(2-adamantylamino)- |
| | 2-oxoethyl)-2-oxo-1,2- |
| | dihydropyridin-3-ylamino)-6- |
| | (methylamino)-1,5,6-trioxohexan-2- |
| | ylcarbamoyl)nicotinate |
| II-66: | (S)-N1-(1-(2-(2-adamantylamino)-2- |
| | oxoethyl)-2-oxo-1,2-dihydropyridin-3- |
| | yl)-N6-methyl-2-(6-methylimidazo[2,1- |
| | b]thiazole-5-carboxamido)-5- |
| | oxohexanediamide |
| II-67: | (S)-N1-(1-(2-(2-adamantyl(methyl)amino)- |
| | 2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-68: | (S)-N1-(1-(2-(5-hydroxyadamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-69: | (S)-N1-(1-(2-(5-fluoroadamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-70: | (S)-N1-(1-(2-(5-chloroadamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-71: | (S)-N1-(1-(2-(5-bromoadamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-72: | (S)-N1-(1-(2-(5-methyladamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-73: | (S)-N1-(1-(2-(2-carbonitrileadamantane- |
| | 2-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-74: | (S)-N1-(1-(2-(2-methyl adamantane- |
| | 2-carboxylate-2-amino)-2-oxoethyl)-2- |
| | ox0-1,2-dihydropyridin-3-yl)-N6- |
| | methyl-2-(3-methylbenzofuran-2- |
| | carboxamido)-5-oxohexanediamide |
| II-87: | (S)-N1-(1-(2-(1-adamantylmethylamino)- |
| | 2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-88: | (S)-N1-(1-(2-(1-(1-adamantyl)ethanamino)- |
| | 2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-90: | (S)-N1-(1-(2-((1R,2S,4S)-bicyclo[2.2.1]heptan- |
| | 2-ylamino)-2-oxoethyl)-2- |
| | oxo-1,2-dihydropyridin-3-yl)-N6-methyl- |
| | 2-(3-methylbenzofuran-2- |
| | carboxamido)-5-oxohexanediamide |
| II-92: | (S)-N1-methyl-5-(3-methylbenzofuran- |
| | 2-carboxamido)-2-oxo-N6-(2-oxo- |
| | 1-(2-oxo-2-((1S,2S,4R)-1,7,7- |
| | trimethylbicyclo[2.2.1 ]heptan-2- |
| | ylamino)ethyl)-1,2-dihydropyridin- |
| | 3-yl)hexanediamide |
| II-94: | (S)-N1-(1-(2-((1R,2R,4S)-bicyclo[2.2.1]heptan- |
| | 2-ylamino)-2-oxoethyl)-2- |
| | ox0-1,2-dihydropyridin-3-yl)-N6-methyl- |
| | 2-(3-methylbenzofuran-2- |
| | carboxamido)-5-oxohexanediamide |
| II-95: | (S)-N1-(1-(2-(bicyclo[2.2.1]heptan- |
| | 1-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-96: | (S)-N1-(1-(2-(bicyclo[2.2.1]heptan- |
| | 7-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-97: | (S)-N1-(1-(2-(bicyclo[2.2.1]hept- |
| | 5-en-2-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-98: | (2S)-N1-(1-(2-(bicyclo[2.2.2]octan- |
| | 2-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-99: | (S)-N1-methyl-5-(3-methylbenzofuran- |
| | 2-carboxamido)-2-oxo-N6-(2-oxo- |
| | 1-(2-oxo-2-((1R,2R,4R)-1,7,7- |
| | trimethylbicyclo[2.2.1 ]heptan-2- |
| | ylamino)ethyl)-1,2-dihydropyridin- |
| | 3-yl)hexanediamide |
| II-100: | (S)-N1-methyl-5-(3-methylbenzofuran- |
| | 2-carboxamido)-2-oxo-N6-(2-oxo- |
| | 1-(2-oxo-2-((1R,2R,3R,5S)-2,6,6- |
| | trimethylbicyclo[3.1.1]heptan-3- |
| | ylamino)ethyl)-1,2-dihydropyridin- |
| | 3-yl)hexanediamide |
| II-101: | (S)-N1-methyl-5-(3-methylbenzofuran- |
| | 2-carboxamido)-2-oxo-N6-(2-oxo- |
| | 1-(2-oxo-2-((1S,2S,3S,5R)-2,6,6- |
| | trimethylbicyclo[3.1.1]heptan-3- |
| | ylamino)ethyl)-1,2-dihydropyridin- |
| | 3-yl)hexanediamide |
| II-103: | (S)-N1-(1-(2-(4-homoisotwistane- |
| | 3-amino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-104: | (S)-N1-(1-(2-(diamantane-1-amino)- |
| | 2-oxoethyl)-2-oxo-1,2-dihydropyridin- |
| | 3-yl)-N6-methyl-2-(3-methylbenzofuran- |
| | 2-carboxamido)-5- |
| | oxohexanediamide |
| II-105: | (S)-N1-(1-(2-(diamantane-4-amino)- |
| | 2-oxoethyl)-2-oxo-1,2-dihydropyridin- |
| | 3-yl)-N6-methyl-2-(3-methylbenzofuran- |
| | 2-carboxamido)-5- |
| | oxohexanediamide |
| II-107: | (S)-N1-(1-(1-adamantylmethyl)- |
| | 2-oxo-1,2-dihydropyridin-3-yl)-N6- |
| | methyl-2-(3-methylbenzofuran-2- |
| | carboxamido)-5-oxohexanediamide |
| II-108: | (2S)-N1-(1-((3-hydroxy-1-adamantyl)methyl)- |
| | 2-oxo-1,2-dihydropyridin-3- |
| | yl)-N6-methyl-2-(3-methylbenzofuran- |
| | 2-carboxamido)-5- |
| | oxohexanediamide |
| II-109: | (2S)-N1-(1-((3-bromo-1-adamantyl)methyl)- |
| | 2-oxo-1,2-dihydropyridin-3- |
| | yl)-N6-methyl-2-(3-methylbenzofuran- |
| | 2-carboxamido)-5- |
| | oxohexanediamide |
| II-110: | (S)-N1-(1-(2-adamantylmethyl)-2- |
| | oxo-1,2-dihydropyridin-3-yl)-N6- |
| | methyl-2-(3-methylbenzofuran-2- |
| | carboxamido)-5-oxohexanediamide |
| II-111: | (S)-N1-(1-(2-(2-adamantylamino)- |
| | 2-oxoethyl)-2-oxo-1,2-dihydropyridin-3- |
| | yl)-N6-methyl-2-(nicotinamido)- |
| | 5-oxohexanediamide |
| II-112: | (S)-2-(isonicotinamido)-N1-(1-(2- |
| | (2-adamantylamino)-2-oxoethyl)-2-oxo- |
| | 1,2-dihydropyridin-3-yl)-N6-methyl- |
| | 5-oxohexanediamide |
| II-113: | (S)-N1-(1-(2-(2-adamantylamino)- |
| | 2-oxoethyl)-2-oxo-1,2-dihydropyridin-3- |
| | yl)-N6-methyl-5-oxo-2-(pyridazine-4- |
| | carboxamido)hexanediamide |
| II-114: | (S)-N1-(1-(2-(2-adamantylamino)- |
| | 2-oxoethyl)-2-oxo-1,2-dihydropyridin-3- |
| | yl)-N6-methyl-5-oxo-2-(pyridazine-3- |
| | carboxamido)hexanediamide |
| II-115: | (S)-N1-cyclopropyl-N6-(1-(2-(2- |
| | adamantylamino)-2-oxoethyl)-2-oxo-1,2- |
| | dihydropyridin-3-yl)-5-(3- |
| | methylbenzofuran-2-carboxamido)-2- |
| | oxohexanediamide |
| II-116: | (S)-N1-(1-(2-(2-adamantylamino)-2- |
| | oxoethyl)-2-oxo-1,2-dihydropyridin-3- |
| | yl)-2-(3-methylbenzofuran-2- |
| | carboxamido)-5-oxo-N6- |
| | pentylhexanediamide |
| II-117: | (S)-N1-allyl-N6-(1-(2-(2-adamantylamino)- |
| | 2-oxoethyl)-2-oxo-1,2- |
| | dihydropyridin-3-yl)-5-(3- |
| | methylbenzofuran-2-carboxamido)-2- |
| | oxohexanediamide |
| II-118: | (S)-N1-(1-(2-(2-adamantylamino)-2- |
| | oxoethyl)-2-oxo-1,2-dihydropyridin-3- |
| | yl)-2-(3-methylbenzofuran-2- |
| | carboxamido)-5-oxohexanediamide |
| II-119: | (S)-N1-allyl-5-(benzofuran-2-carboxamido)- |
| | N6-(1-(2-(2-adamantylamino)- |
| | 2-oxoethyl)-2-oxo-1,2-dihydropyridin- |
| | 3-yl)-2-oxohexanediamide |
| II-120: | (S)-2-(benzofuran-2-carboxamido)- |
| | N6-isopropyl-N1-(1-(2-(2- |
| | adamantylamino)-2-oxoethyl)-2-oxo- |
| | 1,2-dihydropyridin-3-yl)-5- |
| | oxohexanediamide |
| II-121: | (S)-2-(benzofuran-2-carboxamido)- |
| | N6-cyclopropyl-N1-(1-(2-(2- |
| | adamantylamino)-2-oxoethyl)-2- |
| | oxo-1,2-dihydropyridin-3-yl)-5- |
| | oxohexanediamide |
| II-122: | (S)-2-(benzofuran-2-carboxamido)- |
| | N1-(1-(2-(2-adamantylamino)-2- |
| | oxoethyl)-2-oxo-1,2-dihydropyridin-3- |
| | yl)-5-oxo-N6-phenylhexanediamide |
| II-123: | (S)-2-(benzofuran-2-carboxamido)- |
| | N6-benzyl-N1-(1-(2-(2- |
| | adamantylamino)-2-oxoethyl)-2- |
| | oxo-1,2-dihydropyridin-3-yl)-5- |
| | oxohexanediamide |
| II-124: | (S)-2-(benzofuran-2-carboxamido)- |
| | N1-(1-(2-(2-adamantylamino)-2- |
| | oxoethyl)-2-oxo-1,2-dihydropyridin- |
| | 3-yl)-5-oxohexanediamide |
| II-125: | (S)-2-(2,5-dichlorothiophene- |
| | 3-carboxamido)-N1-(1-(2-(2- |
| | adamantylamino)-2-oxoethyl)-2-oxo- |
| | 1,2-dihydropyridin-3-yl)-5- |
| | oxohexanediamide |
| II-126: | (S)-N1-(1-(2-(2-adamantylamino)-2- |
| | oxoethyl)-2-oxo-1,2-dihydropyridin-3- |
| | yl)-2-(4-methyl-2-(trifluoromethyl)thiazole- |
| | 5-carboxamido)-5- |
| | oxohexanediamide |
| II-127: | (S)-N1-(1-(2-(2-adamantylamino)-2- |
| | oxoethyl)-2-oxo-1,2-dihydropyridin-3- |
| | yl)-2-(1-methyl-1H-1,2,3-triazole-5- |
| | carboxamido)-5-oxohexanediamide |
| II-128: | (2S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-2-(2,5- |
| | dichlorothiophene-3-carboxamido)- |
| | N6-methyl-5-oxohexanediamide |
| II-129: | (2S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-N6- |
| | methyl-2-(4-methyl-2- |
| | (trifluoromethyl)thiazole-5- |
| | carboxamido)-5-oxohexanediamide |
| II-130: | (2S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-N6-methyl- |
| | 2-(1-methyl-1H-1,2,3-triazole-5- |
| | carboxamido)-5-oxohexanediamide |
| II-131: | (S)-N1-(1-(2-(2-adamantylamino)-2- |
| | oxoethyl)-2-oxo-1,2-dihydropyridin-3- |
| | yl)-N6-methyl-5-oxo-2-(2H-1,2,3-triazole- |
| | 4-carboxamido)hexanediamide |
| II-132: | (S)-N1-(1-(2-(2-adamantylamino)-2- |
| | oxoethyl)-2-oxo-1,2-dihydropyridin-3- |
| | yl)-N6-methyl-5-oxo-2-(1H-1,2,3-triazole- |
| | 4-carboxamido)hexanediamide |
| II-133: | (S)-N1-(1-(2-(2-adamantylamino)-2- |
| | oxoethyl)-2-oxo-1,2-dihydropyridin-3- |
| | yl)-N6-methyl-2-(1-methyl-1H-1,2,3- |
| | triazole-4-carboxamido)-5- |
| | oxohexanediamide |
| II-134: | (S)-N1-(1-(2-(2-adamantylamino)-2- |
| | oxoethyl)-2-oxo-1,2-dihydropyridin-3- |
| | yl)-N6-methyl-5-oxo-2-(1H-1,2,4-triazole- |
| | 3-carboxamido)hexanediamide |
| II-135: | (S)-N1-(1-(2-(2-adamantylamino)-2- |
| | oxoethyl)-2-oxo-1,2-dihydropyridin-3- |
| | yl)-N6-methyl-2-(1-methyl-1H-1,2,4- |
| | triazole-3-carboxamido)-5- |
| | oxohexanediamide |
| II-136: | (S)-2-(benzofuran-3-carboxamido)- |
| | N1-(1-(2-(2-adamantylamino)-2- |
| | oxoethyl)-2-oxo-1,2-dihydropyridin-3-yl)- |
| | N6-methyl-5-oxohexanediamide |
| II-137: | (S)-2-(benzo[b]thiophene-3- |
| | carboxamido)-N1-(1-(2-(2-adamantylamino)- |
| | 2-oxoethyl)-2-oxo-1,2-dihydropyridin- |
| | 3-yl)-N6-methyl-5- |
| | oxohexanediamide |
| II-138 | (S)-N1-(1-(2-(2-adamantylamino)- |
| | 2-oxoethyl)-2-oxo-1,2-dihydropyridin-3- |
| | yl)-N6-methyl-2-(1-methyl-1H- |
| | pyrazole-3-carboxamido)-5- |
| | oxohexanediamide |
| II-139 | (S)-N1-(1-(2-(2-adamantylamino)-2- |
| | oxoethyl)-2-oxo-1,2-dihydropyridin-3- |
| | yl)-N6-methyl-2-(1-methyl-1H- |
| | pyrazole-4-carboxamido)-5- |
| | oxohexanediamide |
| II-140 | (S)-N1-(1-(2-(2-adamantylamino)-2- |
| | oxoethyl)-2-oxo-1,2-dihydropyridin-3- |
| | yl)-N6-methyl-2-(1-methyl-1H- |
| | pyrazole-5-carboxamido)-5- |
| | oxohexanediamide |
| II-141 | (S)-N1-(1-(2-(2-adamantylamino)-2- |
| | oxoethyl)-2-oxo-1,2-dihydropyridin-3- |
| | yl)-N6-methyl-2-(4-methyl-1,2,3- |
| | thiadiazole-5-carboxamido)-5- |
| | oxohexanediamide |
| II-142 | (S)-N1-(1-(2-(2-adamantylamino)-2- |
| | oxoethyl)-2-oxo-1,2-dihydropyridin-3- |
| | yl)-N6-methyl-5-oxo-2-(1,2,5-thiadiazole- |
| | 3-carboxamido)hexanediamide |
| II-143 | (S)-2-(4-iodo-1-methyl-1H-pyrazole- |
| | 5-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-144 | (S)-N1-(1-(2-(2-adamantylamino)-2- |
| | oxoethyl)-2-oxo-1,2-dihydropyridin-3- |
| | yl)-2-(1-methyl-1H-pyrazole-5- |
| | carboxamido)-5-oxohexanediamide |
| II-145 | (S)-N1-(1-(2-(2-adamantylamino)-2- |
| | oxoethyl)-2-oxo-1,2-dihydropyridin-3- |
| | yl)-2-(4-methyl-1,2,3-thiadiazole-5- |
| | carboxamido)-5-oxohexanediamide |
| II-146 | (S)-2-(benzofuran-2-carboxamido)- |
| | N1-(1-(2-((1R,2R,4S)- |
| | bicyclo[2.2.1]heptan-2-ylamino)-2- |
| | oxoethyl)-2-oxo-1,2-dihydropyridin-3- |
| | yl)-5-oxohexanediamide |
| II-147 | (S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-2-(3- |
| | methylbenzofuran-2-carboxamido)-5- |
| | oxohexanediamide |
| II-148 | (S)-2-(benzofuran-2-carboxamido)- |
| | N1-(1-(2-((1S,2R,4R)- |
| | bicyclo[2.2.1 ]heptan-2-ylamino)-2- |
| | oxoethyl)-2-oxo-1,2-dihydropyridin-3- |
| | yl)-5-oxohexanediamide |
| II-149 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-2-(3- |
| | methylbenzofuran-2-carboxamido)-5- |
| | oxohexanediamide |
| II-150 | (S)-2-(benzofuran-2-carboxamido)- |
| | 5-oxo-N1-(2-oxo-1-(2-oxo-2- |
| | ((1R,2S,4R)-1,7,7-trimethylbicyclo[2.2.1]heptan- |
| | 2-ylamino)ethyl)-1,2- |
| | dihydropyridin-3-yl)hexanediamide |
| II-151 | (S)-2-(3-methylbenzofuran-2-carboxamido)- |
| | 5-oxo-N1-(2-oxo-1-(2-oxo-2- |
| | ((1R,2S,4R)-1,7,7-trimethylbicyclo[2.2.1]heptan- |
| | 2-ylamino)ethyl)-1,2- |
| | dihydropyridin-3-yl)hexanediamide |
| II-152 | (S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-N6-methyl- |
| | 2-(4-methyl-1,2,3-thiadiazole-5- |
| | carboxamido)-5-oxohexanediamide |
| II-153 | (S)-N1-(1-(2-((1R,2R,4S)-bicyclo[2.2.1]heptan- |
| | 2-ylamino)-2-oxoethyl)-2- |
| | oxo-1,2-dihydropyridin-3-yl)-N6-methyl- |
| | 2-(1-methyl-1H-pyrazole-5- |
| | carboxamido)-5-oxohexanediamide |
| II-154 | (S)-N1-(1-(2-((1S,2R,4R)-bicyclo[2.2.1]heptan- |
| | 2-ylamino)-2-oxoethyl)-2- |
| | ox0-1,2-dihydropyridin-3-yl)- |
| | N6-methyl-2-(4-methyl-2- |
| | (trifluoromethyl)thiazole-5-carboxamido)- |
| | 5-oxohexanediamide |
| II-155 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-2-(2,5- |
| | dichlorothiophene-3-carboxamido)- |
| | N6-methyl-5-oxohexanediamide |
| II-156 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-N6-methyl- |
| | 2-(4-methyl-1,2,3-thiadiazole-5- |
| | carboxamido)-5-oxohexanediamide |
| II-157 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-N6-methyl- |
| | 2-(1-methyl-1H-1,2,3-triazole-5- |
| | carboxamido)-5-oxohexanediamide |
| II-158 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-N6-methyl- |
| | 2-(1-methyl-1H-pyrazole-5- |
| | carboxamido)-5-oxohexanediamide |
| II-159 | (S)-N1-methyl-5-(4-methyl-2- |
| | (trifluoromethyl)thiazole-5-carboxamido)-2- |
| | oxo-N6-(2-oxo-1-(2-oxo-2-((1R,2S,4R)-1,7,7- |
| | trimethylbicyclo[2.2.1]heptan-2- |
| | ylamino)ethyl)-1,2-dihydropyridin-3- |
| | yl)hexanediamide |
| II-160 | (S)-2-(2,5-dichlorothiophene-3- |
| | carboxamido)-N6-methyl-5-oxo-N1-(2- |
| | 0x0-1-(2-oxo-2-((1R,2S,4R)-1,7,7- |
| | trimethylbicyclo[2.2.1]heptan-2- |
| | ylamino)ethyl)-1,2-dihydropyridin- |
| | 3-yl)hexanediamide |
| II-161 | (S)-N1-methyl-5-(4-methyl-1,2,3- |
| | thiadiazole-5-carboxamido)-2-oxo-N6- |
| | (2-oxo-1-(2-oxo-2-((1R,2S,4R)-1,7,7- |
| | trimethylbicyclo[2.2.1]heptan-2- |
| | ylamino)ethyl)-1,2-dihydropyridin- |
| | 3-yl)hexanediamide |
| II-162 | (S)-N1-methyl-5-(1-methyl-1H-1,2,3- |
| | triazole-5-carboxamido)-2-oxo-N6- |
| | (2-oxo-1-(2-oxo-2-((1R,2S,4R)-1,7,7- |
| | trimethylbicyclo[2.2.1]heptan-2- |
| | ylamino)ethyl)-1,2-dihydropyridin- |
| | 3-yl)hexanediamide |
| II-163 | (S)-N1-methyl-5-(1-methyl-1H-pyrazole- |
| | 5-carboxamido)-2-oxo-N6-(2- |
| | 0x0-1-(2-oxo-2-((1R,2S,4R)-1,7,7- |
| | trimethylbicyclo[2.2.1]heptan-2- |
| | ylamino)ethyl)-1,2-dihydropyridin- |
| | 3-yl)hexanediamide |
| II-164 | (2S)-2-(4-tert-butyl-1H-pyrrole-3- |
| | carboxamido)-N6-methyl-N1-(1-(2-(1- |
| | adamantylamino)ethyl)-2-oxo- |
| | 1,2-dihydropyridin-3-yl)-5- |
| | oxohexanediamide |
| II-165 | (2S)-2-(4-cyano-1-methyl-1H-pyrrole- |
| | 2-carboxamido)-N6-methyl-N1-(1- |
| | (3-(1-adamantylamino)propyl)-2-oxo- |
| | 1,2-dihydropyridin-3-yl)-5- |
| | oxohexanediamide |
| II-166 | (S)-N1-(1-(3-(2-adamantylamino)- |
| | 3-oxopropyl)-2-oxo-1,2-dihydropyridin- |
| | 3-yl)-2-(5-methoxyoxazole-2- |
| | carboxamido)-N6-methyl-5- |
| | oxohexanediamide |
| II-167 | (S)-N1-(1-(2-(bicyclo[1.1.1]pentan- |
| | 1-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-168 | (S)-2-(2-acetyloxazole-4-carboxamido)-N1- |
| | (1-(2-(bicyclo[1.1.1]pentan-1- |
| | ylamino)-2-oxoethyl)-2-oxo-1,2- |
| | dihydropyridin-3-yl)-N6-methyl-5- |
| | oxohexanediamide |
| II-169 | (S)-N1-(1-(2-(bicyclo[2.1.1]hexan- |
| | 1-ylamino)-2-oxoethyl)-2-oxo-1,2- |
| | dihydropyridin-3-yl)-N6-methyl-2-(3- |
| | methylbenzofuran-2-carboxamido)- |
| | 5-oxohexanediamide |
| II-170 | (S)-N1-(1-(2-(bicyclo[2.1.1]hexan- |
| | 1-ylamino)-2-oxoethyl)-2-oxo-1,2- |
| | dihydropyridin-3-yl)-2-(2-isopropyloxazole- |
| | 5-carboxamido)-N6-methyl-5- |
| | oxohexanediamide |
| II-171 | (2S)-2-(benzofuran-2-carboxamido)-N1- |
| | (1-(2-(bicyclo[3.2.1]octan-8- |
| | ylamino)-2-oxoethyl)-2-oxo-1,2- |
| | dihydropyridin-3-yl)-N6-methyl-5- |
| | oxohexanediamide |
| II-172 | (2S)-N1-(1-(2-(bicyclo[3.2.1]octan- |
| | 8-ylamino)-2-oxoethyl)-2-oxo-1,2- |
| | dihydropyridin-3-yl)-2-(3,5-dimethylisoxazole- |
| | 4-carboxamido)-N6- |
| | methyl-5-oxohexanediamide |
| II-173 | (S)-N1-(1-(2-(5-carboxy-2-aminoadamantane)- |
| | 2-oxoethyl)-2-oxo-1,2- |
| | dihydropyridin-3-yl)-N6-methyl-2-(4- |
| | methylpyrimidine-5-carboxamido)-5- |
| | oxohexanediamide |
| II-174 | (2S)-N1-(1-(2-(4-aminoadamantane- |
| | N,N-dimethyl-1-carboxamide)-2- |
| | oxoethyl)-2-oxo-1,2-dihydropyridin-3- |
| | yl)-N6-methyl-5-oxo-2-(1,2,3,4- |
| | tetrahydronaphthalene-2- |
| | carboxamido)hexanediamide |
| II-175 | (S)-2-((S)-1,4-diazabicyclo[2.2.2]octane- |
| | 2-carboxamido)-N6-tert-butyl- |
| | N1-(1-(2-(2-adamantylamino)-2-oxoethyl)- |
| | 2-oxo-1,2-dihydropyridin-3-yl)- |
| | 5-oxohexanediamide |
| II-176 | (S)-N1-tert-butyl-5-(1H-indole- |
| | 3-carboxamido)-N6-(1-(2-(2- |
| | adamantylamino)-2-oxoethyl)-2-oxo- |
| | 1,2-dihydropyridin-3-yl)-2- |
| | oxohexanediamide |
| II-177 | (S)-N1-tert-butyl-N6-(1-(2-(2- |
| | adamantylamino)-2-oxoethyl)-2-oxo-1,2- |
| | dihydropyridin-3-yl)-5-(6-methylimidazo[2,1- |
| | b]thiazole-3-carboxamido)- |
| | 2-oxohexanediamide |
| II-178 | (S)-2-(benzo[d]thiazole-2- |
| | carboxamido)-N1-(1-(2-((1S,2R,4R)- |
| | bicyclo[2.2.1 ]heptan-2-ylamino)-2- |
| | oxoethyl)-2-oxo-1,2-dihydropyridin-3- |
| | yl)-N6-cyclopentyl-5-oxohexanediamide |
| II-179 | (S)-N1-(1-(2-((1S,2R,4R)-bicyclo[2.2.1]heptan- |
| | 2-ylamino)-2-oxoethyl)-2- |
| | oxo-1,2-dihydropyridin-3-yl)-N6-cyclopentyl- |
| | 2-(imidazo[2,1-b]thiazole-6- |
| | carboxamido)-5-oxohexanediamide |
| II-180 | (S)-N1-(1-(2-((1S,2R,4R)-bicyclo[2.2.1]heptan- |
| | 2-ylamino)-2-oxoethyl)-2- |
| | ox0-1,2-dihydropyridin-3-yl)-N6- |
| | cyclopentyl-2-(4-hydroxy-6- |
| | (trifluoromethoxy)quinoline-3- |
| | carboxamido)-5-oxohexanediamide |
| II-181 | (S)-N1-(1-(2-(bicyclo[1.1.1]pentan- |
| | 1-ylamino)-2-oxoethyl)-2-oxo-1,2- |
| | dihydropyridin-3-yl)-2-(cinnoline-3- |
| | carboxamido)-N6-cyclohexyl-5- |
| | oxohexanediamide |
| II-182 | (S)-N1-(1-(2-(bicyclo[1.1.1]pentan- |
| | 1-ylamino)-2-oxoethyl)-2-oxo-1,2- |
| | dihydropyridin-3-yl)-N6- |
| | cyclohexyl-2-(3-ethylbenzofuran-2- |
| | carboxamido)-5-oxohexanediamide |
| II-183 | (S)-N1-(1-(2-(bicyclo[1.1.1]pentan- |
| | 1-ylamino)-2-oxoethyl)-2-oxo-1,2- |
| | dihydropyridin-3-yl)-N6-cyclohexyl-2- |
| | (1-ethyl-1H-indole-2-carboxamido)- |
| | 5-oxohexanediamide |
| II-184 | (S)-N1-(1-(2-(bicyclo[1.1.1]pentan- |
| | 1-ylamino)-2-oxoethyl)-2-oxo-1,2- |
| | dihydropyridin-3-yl)-N6-cyclohexyl- |
| | 2-(2-methyl-1,8-naphthyridine-3- |
| | carboxamido)-5-oxohexanediamide |
| II-185 | (S)-N1-(1-(2-(bicyclo[2.1.1]hexan- |
| | 1-ylamino)-2-oxoethyl)-2-oxo-1,2- |
| | dihydropyridin-3-yl)-N6-methyl-5-oxo- |
| | 2-(1,2,3,4-tetrahydroquinoline-6- |
| | carboxamido)hexanediamide |
| II-186 | (S)-N1-(1-(2-(2-carboxy-2-amino-5- |
| | (trifluoromethyl)adamantane)-2- |
| | oxoethyl)-2-oxo-1,2-dihydropyridin- |
| | 3-yl)-N6-methyl-5-oxo-2-(3-oxo- |
| | 1,2,3,4-tetrahydroisoquinoline-6- |
| | carboxamido)hexanediamide |
| II-187 | (S)-N1-(1-(2-(5-ethyladamantane- |
| | 2-amino)-2-oxoethyl)-2-oxo-1,2- |
| | dihydropyridin-3-yl)-N6-methyl-2-(1,6- |
| | naphthyridine-2-carboxamido)-5- |
| | oxohexanediamide |
| II-188 | (S)-N1-(1-(2-(bicyclo[2.1.1]hexan-1- |
| | ylamino)-2-oxoethyl)-2-oxo-1,2- |
| | dihydropyridin-3-yl)-N6-methyl-2-(2,6- |
| | naphthyridine-1-carboxamido)-5- |
| | oxohexanediamide |
| II-189 | (S)-2-(4-amino-1,2,5-oxadiazole- |
| | 3-carboxamido)-N1-(1-(2- |
| | (bicyclo[1.1.1]pentan-1-ylamino)-2- |
| | oxoethyl)-2-oxo-1,2-dihydropyridin-3- |
| | yl)-N6-methyl-5-oxohexanediamide |
| II-190 | (S)-N1-(1-(2-(bicyclo[1.1.1]pentan- |
| | 1-ylamino)-2-oxoethyl)-2-oxo-1,2- |
| | dihydropyridin-3-yl)-2-(6- |
| | (dimethylamino)benzofuran-2-carboxamido)- |
| | N6-methyl-5-oxohexanediamide |
| II-191 | (S)-2-(2-acetamidothiazole- |
| | 5-carboxamido)-N1-(1-(2- |
| | (bicyclo[1.1.1]pentan-1-ylamino)-2- |
| | oxoethyl)-2-oxo-1,2-dihydropyridin-3- |
| | yl)-N6-methyl-5-oxohexanediamide |
| II-192 | (S)-N4-(1-(1-(2-(bicyclo[1.1.1]pentan- |
| | 1-ylamino)-2-oxoethyl)-2-oxo-1,2- |
| | dihydropyridin-3-ylamino)-6-(methylamino)- |
| | 1,5,6-trioxohexan-2-yl)-1H- |
| | pyrrole-2,4-dicarboxamide |
| II-193 | (S)-N1-(1-(2-(1-acetylamino-4-aminoadamantane)- |
| | 2-oxoethyl)-2-oxo-1,2- |
| | dihydropyridin-3-yl)-N6-methyl- |
| | 5-oxo-2-(5-sulfamoylfuran-3- |
| | carboxamido)hexanediamide |
| II-194 | (S)-2-(benzofuran-5-carboxamido)- |
| | N1-(1-(2-(1-acetylamino-4- |
| | aminoadamantane)-2-oxoethyl)-2-oxo- |
| | 1,2-dihydropyridin-3-yl)-N6- |
| | methyl-5-oxohexanediamide |
| II-195 | (S)-2-(benzofuran-6-carboxamido)- |
| | N1-(1-(2-(4-aminoadamantane-1- |
| | carboxamide)-2-oxoethyl)-2-oxo-1,2- |
| | dihydropyridin-3-yl)-N6-methyl-5- |
| | oxohexanediamide |
| II-196 | (S)-N1-(1-(2-(4-aminoadamantane- |
| | 1-carboxamide)-2-oxoethyl)-2-oxo-1,2- |
| | dihydropyridin-3-yl)-N6-methyl-2- |
| | (3-(1-methylcyclopropyl)-1,2,4- |
| | oxadiazole-5-carboxamido)-5- |
| | oxohexanediamide |
| II-197 | (S)-N1-(1-(2-(bicyclo[1.1.1]pentan- |
| | 1-ylamino)-2-oxoethyl)-2-oxo-1,2- |
| | dihydropyridin-3-yl)-N6-methyl-2- |
| | (5-methyl-1,2,4-oxadiazole-3- |
| | carboxamido)-5-oxohexanediamide |
| II-198 | (S)-N1-(1-(2-(bicyclo[1.1.1]pentan- |
| | 1-ylamino)-2-oxoethyl)-2-oxo-1,2- |
| | dihydropyridin-3-yl)-N6-methyl-5- |
| | oxo-2-(1,2,3-thiadiazole-4- |
| | carboxamido)hexanediamide |
| II-199 | (S)-N1-(1-(2-(bicyclo[1.1.1]pentan- |
| | 1-ylamino)-2-oxoethyl)-2-oxo-1,2- |
| | dihydropyridin-3-yl)-N6-methyl- |
| | 5-oxo-2-(1,2,4-thiadiazole-5- |
| | carboxamido)hexanediamide |
| II-200 | (S)-N1-(1-(2-(bicyclo[1.1.1]pentan- |
| | 1-ylamino)-2-oxoethyl)-2-oxo-1,2- |
| | dihydropyridin-3-yl)-N6-methyl- |
| | 5-oxo-2-(1,3,4-thiadiazole-2- |
| | carboxamido)hexanediamide |
| II-201 | (S)-N1-(1-(2-(bicyclo[1.1.1]pentan- |
| | 1-ylamino)-2-oxoethyl)-2-oxo-1,2- |
| | dihydropyridin-3-yl)-2-(4-cyclopropyl- |
| | 1,2,3-thiadiazole-5-carboxamido)- |
| | N6-methyl-5-oxohexanediamide |
| II-202 | (S)-2-(4-cyclopropyl-1,2,3-thiadiazole- |
| | 5-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-203 | (S)-2-(4-isopropyl-1,2,3-thiadiazole- |
| | 5-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-204 | (S)-2-(4-ethyl-1,2,3-thiadiazole- |
| | 5-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-205 | (S)-2-(4-formyl-1,2,3-thiadiazole- |
| | 5-carboxamido)-N1-(1-(2-(2- |
| | adamantylamino)-2-oxoethyl)-2-oxo-1,2- |
| | dihydropyridin-3-yl)-N6-methyl- |
| | 5-oxohexanediamide |
| II-206 | (S)-2-(4-(hydroxymethyl)-1,2,3- |
| | thiadiazole-5-carboxamido)-N1-(1-(2-(2- |
| | adamantylamino)-2-oxoethyl)-2-oxo- |
| | 1,2-dihydropyridin-3-yl)-N6-methyl- |
| | 5-oxohexanediamide |
| II-207 | (S)-N1-(1-(2-(1-adamantylamino)- |
| | 2-oxoethyl)-2-oxo-1,2-dihydropyridin-3- |
| | yl)-N6-methyl-2-(1-methyl-1H- |
| | imidazole-5-carboxamido)-5- |
| | oxohexanediamide |
| II-208 | (S)-N1-(1-(2-(((1S,2R,5S)-6,6- |
| | dimethylbicyclo[3.1.1]heptan-2- |
| | yl)methylamino)-2-oxoethyl)-2-oxo- |
| | 1,2-dihydropyridin-3-yl)-N6-methyl- |
| | 2-(1-methyl-1H-imidazole-2-carboxamido)- |
| | 5-oxohexanediamide |
| II-209 | (S)-N1-(1-(2-(((1R,2R,5R)-6,6- |
| | dimethylbicyclo[3.1.1]heptan-2- |
| | yl)methylamino)-2-oxoethyl)-2-oxo- |
| | 1,2-dihydropyridin-3-yl)-2-(1H- |
| | imidazole-4-carboxamido)-N6-methyl- |
| | 5-oxohexanediamide |
| II-210 | (S)-N1-(1-(2-(3,5-dimethyladamantane- |
| | 1-amino)-2-oxoethyl)-2-oxo-1,2- |
| | dihydropyridin-3-yl)-N6-methyl- |
| | 2-(1-methyl-1H-imidazole-5- |
| | carboxamido)-5-oxohexanediamide |
| II-211 | (S)-N1-(1-(2-(3,5,7-trimethyl-1- |
| | adamantylamino)-2-oxoethyl)-2-oxo-1,2- |
| | dihydropyridin-3-yl)-N6-methyl-2- |
| | (1-methyl-1H-imidazole-5- |
| | carboxamido)-5-oxohexanediamide |

or a pharmaceutically acceptable salt thereof.

10. A pharmaceutical composition comprising a compound of claim 1, or a diastereomer, an enantiomer, a mixture of diastereomers, a mixture of enantiomers, a racemate, a solvate, a hydrate, or a pharmaceutically acceptable salt thereof, as an active ingredient, together with at least one pharmaceutically acceptable carrier, excipient and/or diluent.

11. (canceled)

12. A method for treating a disease comprising administering to a subject, a pharmaceutically effective amount of at least one compound of claim 1, or a pharmaceutically acceptable salt thereof, wherein the disease is selected from the group consisting of an autoimmune disease, an inflammatory disease, a vascular disease, a fibrotic disease, a liver disease, a cholestatic liver disease, a cancer, a neurodegenerative disease, an ocular disease and a skin disorder.

13. The method according to claim 12, wherein
the autoimmune disease and the inflammatory disease comprise multiple sclerosis, celiac disease, Duhring-Brocq-disease (dermatitis herpetiformis), gluten ataxia, gluten neuropathy, diabetes, rheumatoid arthritis, Graves' disease, inflammatory bowel disease, systemic lupus erythematosus psoriasis or gingivitis;
the vascular disease comprises atherosclerosis, thrombosis or vascular stiffness;
the fibrotic disease affects an organ selected from the group consisting of a lung, a kidney, a liver, a skin and a gut;
the liver disease comprises alcoholic hepatitis, alcoholic steatohepatitis, nonalcoholic steatohepatitis, non-alcoholic fatty liver disease, liver cirrhosis, autoimmune hepatitis or liver inflammation;
the cholestatic liver disease comprises primary biliary cholangitis or primary sclerosing cholangitis;
the cancer is selected from the group consisting of glioblastoma, melanoma, pancreatic cancer, renal cell carcinoma, meningioma and breast cancer,
the neurodegenerative disease comprises Parkinson's disease, Huntington's disease or Alzheimer's disease,
the ocular disease comprises glaucoma, cataracts, macular degeneration or uveitis;
and the skin disorder comprises acne, psoriasis, scarring or skin aging.

14. The method according to claim 12 for the treatment of celiac disease.

15. A method for producing the compound of formula (Ib) according to claim 1 comprising:
providing a compound 4b 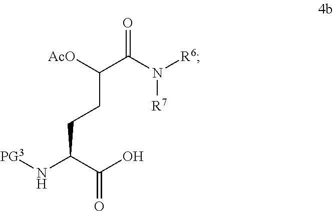
performing coupling reaction of the compound 4b with a compound 5 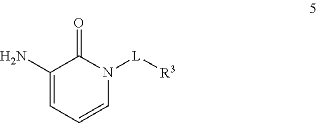
to obtain a compound 6b 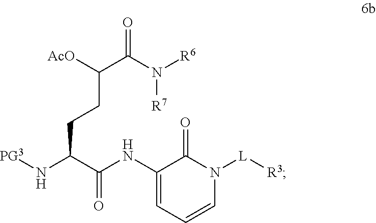
deprotecting an amino protecting group PG³ to obtain a compound 7b 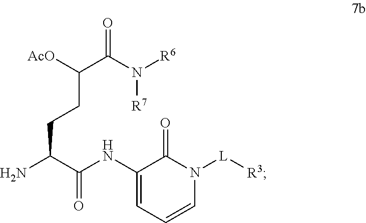
performing coupling reaction of the compound 7b with a carboxylic acid (R²—CO₂H 8) to obtain a compound 9b 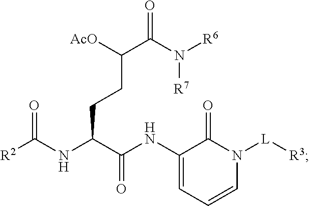
performing oxidation reaction of the compound 9b to produce the compound of the formula (Ib) 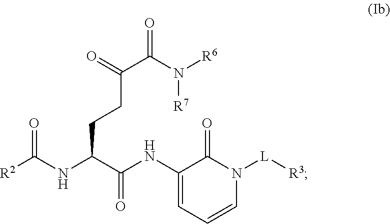
wherein L, R², R³, R⁶ and R⁷ have the same meanings as defined in claim 1, and PG³ is an amino protecting group.

16. The method according to claim 13 for the treatment of celiac disease.

17. The method according to claim 13, wherein the fibrotic disease is selected from the group consisting of cystic fibrosis, kidney fibrosis, diabetic nephropathy, intestinal fibrosis, idiopathic lung fibrosis and liver fibrosis.
